(19) United States
(12) Patent Application Publication
(10) Pub. No.: US 2026/0275385 A1
(43) Pub. Date: Sep. 17, 2026

(54) ENGINEERING CELLS WITH A TRANSGENE IN B2M OR CIITA LOCUS AND ASSOCIATED COMPOSITIONS AND METHODS

(71) Applicant: Sana Biotechnology, Inc., Seattle, WA (US)

(72) Inventors: Terry J. Fry, Denver, CO (US); William Dowdle, South San Francisco, CA (US)

(21) Appl. No.: 18/849,329

(22) PCT Filed: Mar. 21, 2023
(86) PCT No.: PCT/US2023/015788

§ 371 Date: Sep. 20, 2024

Related U.S. Application Data
(60) Provisional application No. 63/269,763, filed on Mar. 22, 2022.
(60) Provisional application No. 63/480,484, filed on Jan. 18, 2023.

(51) Int. Cl. C12N 15/90; A61K 40/11; A61K 40/31; C07K 14/705; C12N 9/22; C12N 15/11

(52) U.S. Cl. CPC C12N 15/907; A61K 40/11; A61K 40/31; C07K 14/70503; C12N 9/226; C12N 15/11; C12N 2310/20

(57) ABSTRACT

Provided herein are methods for generating immune evasive cells by inserting one or more transgenes encoding one or more tolerogenic factors such as CD47 into one or more endogenous gene loci such as B2M locus and CHIA locus. The methods may further include reducing expression of one or more MHC I and/or one or more MHC II molecules. Also disclosed are therapeutic cells and compositions derived from these methods.

### CROSS REFERENCE TO RELATED APPLICATIONS

This application claims priority to and the benefit of U.S. Provisional Application No. 63/269,763, filed Mar. 22, 2022 and U.S. Provisional Application No. 63/480,484, filed Jan. 18, 2023, the contents of which are incorporated herein by reference in their entirety.

### SEQUENCE LISTING

The instant application contains a Sequence Listing (submitted electronically in an XML format and is hereby incorporated by reference in its entirety. Said XML copy, created on Mar. 14, 2023, is named “2017428-0024_SL.xml” and is 21,652,566 bytes in size.

### BACKGROUND

The use of live cells is an important cell therapy approach within the area of adoptive cell transfer (ACT). This approach involves collecting cells from a patient (autologous) or healthy donors (allogeneic), genetically modifying or engineering these live cells to obtain a population of therapeutic cells, and transferring the therapeutic cells into the patient to treat various diseases or conditions. The use of off-the-shelf allogeneic cells has several advantages over the use of autologous cells, as the latter suffers from challenges such as a patient having insufficient healthy cells for harvesting and the patient's experiencing disease progression, co-morbidities, or even death in the time it takes to manufacture the therapeutic cells.

However, in order to make the use of allogeneic cells in ACT feasible, the donor cells (such as primary cells and pluripotent stem cells (PSCs)) must be rendered immune evasive, i.e., not be attacked by the recipient's immune system for being “foreign.” There is substantial evidence in both animal models and human patients that transplantation of immune evasive cells is a scientifically feasible and clinically promising approach to the treatment of numerous disorders, conditions, and diseases. Thus, there is a growing need to efficiently manufacture such immune evasive cells.

### SUMMARY

The present disclosure provides a method for generating an engineered immune evasive cell, such as an engineered immune evasive allogeneic cell, by inserting one or more transgenes encoding one or more tolerogenic factors, and optionally, one or more safety switches, into an endogenous B2 microglobulin (B2M) and/or a class II transactivator (CIITA) gene locus of a cell. In some embodiments, the method further comprises modifying the cell to have reduced or eliminated expression of one or more major histocompatibility complex (MHC) class I and/or class II molecules compared to a wildtype cell, unmodified cell, or control cell. In some embodiments, the expression of one or more MHC I molecules is reduced or eliminated by knocking out B2M and/or the transporter associated with antigen presentation-1 (TAP1). In some embodiments, the expression of one or more MHC II molecules is reduced or eliminated by knocking out CIITA and/or CD74. In some embodiments, the method further comprises selecting for the engineered immune evasive cell by positive selection for the one or more tolerogenic factors. In some of these embodiments, the positive selection utilizes affinity binding, flow cytometry, and/or immunomagnetic selection using antibodies and/or proteins that bind the one or more tolerogenic factors. In some embodiments, the cell being engineered is a donor cell. In some of these embodiments, the donor cell is a primary cell. In some embodiments, the donor cell is a pluripotent stem cell (PSC) such as an embryonic stem cell (ESC) or an induced pluripotent stem cell (iPSC). In some embodiments wherein the donor cell is a PSC, the method further comprises differentiating the engineered immune evasive PSC into a desired type of cell. In some embodiments, the one or more tolerogenic factors include but are not limited to A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9. Compositions comprising engineered immune evasive cells derived from these methods, as well as methods of using these cells and compositions are also provided.

In some aspects, provided is a method of generating a population of therapeutic cells comprising engineered immune evasive cells or cells differentiated from engineered immune evasive cells for cell therapy by inserting one or more transgenes encoding one or more tolerogenic factors, and optionally, one or more safety switches, into an endogenous B2M and/or CIITA gene locus of one or more cells. In some embodiments, the method further comprises modifying one or more immune evasive cells to have reduced or eliminated expression of one or more MHC class I and class II molecules compared to a wildtype cell, unmodified cell, or control cell. In some embodiments, the expression of one or more MHC I molecules is reduced or eliminated by knocking out B2M and/or TAP1. In some embodiments, the expression of one or more MHC II molecules is reduced or eliminated by knocking out CIITA and/or CD74. In some embodiments, the method further comprises selecting for engineered immune evasive cells by positive selection for the one or more tolerogenic factors. In some of these embodiments, the positive selection utilizes affinity binding, flow cytometry, and/or immunomagnetic selection using antibodies and/or proteins that bind the one or more tolerogenic factors. In some embodiments, the cell being engineered is a donor cell. In some embodiments, the donor cell is a primary cell. In some embodiments, the donor cell is a pluripotent stem cell (PSC) such as an embryonic stem cell (ESC) or an induced pluripotent stem cell (iPSC). In some embodiments, cells from two or more different donors are mixed and engineered to generate a population of therapeutic cells. In some embodiments wherein the donor cell is a PSC, the method further comprises differentiating the engineered immune evasive PSC into a desired type of cell. In some embodiments, the one or more tolerogenic factors include but are not limited to A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9. Compositions comprising therapeutic cells derived from these methods, as well as methods of using these cells and compositions are also provided.

In some embodiments, two or more transgenes encoding two or more tolerogenic factors are inserted into the same gene locus. In some embodiments, two or more transgenes encoding two or more tolerogenic factors are inserted into different gene loci. In some embodiments, the transgene encoding the same tolerogenic factor is inserted into two or more different gene loci.

In some embodiments, one or more transgenes encoding one or more tolerogenic factors are inserted into a specific locus of one allele. In some embodiments, one or more transgenes encoding one or more tolerogenic factors are inserted into a specific locus of both alleles.

In some embodiments, the tolerogenic factor is selected from the group consisting of A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9. In some embodiments, the tolerogenic factor is CD47, for example, human CD47.

In some embodiments, the endogenous locus for inserting the one or more transgenes is selected from the group consisting of a B2M locus, a CIITA locus, and a safe harbor locus. In some embodiments, the insertion into the B2M gene locus is in exon 2 or another CDS of the B2M gene. In some embodiments, the insertion into the CIITA gene locus is in exon 3 or another CDS of the CIITA gene.

In some embodiments, the expression of B2M, TAP-1, and/or CIITA is reduced in the engineered immune evasive cell compared to a wildtype cell, unmodified cell, or control cell. In some embodiments, the engineered immune evasive cell does not express B2M. In some embodiments, the engineered immune evasive cell does not express TAP1. In some embodiments, the engineered immune evasive cell does not express CIITA. In some embodiments, the engineered immune evasive cell does not express CD74. In some embodiments, the engineered immune evasive cell expresses neither B2M nor CIITA. In some embodiments, the engineered immune evasive cell expresses neither TAP1 nor CIITA. In some embodiments, the engineered immune evasive cell expresses neither B2M nor CD74. In some embodiments, the engineered immune evasive cell expresses neither TAP1 nor CD74. In some embodiments, the engineered immune evasive cell does not express any of B2M, TAP1, CD74, and CIITA. In some embodiments, the expression of one or more MHC class I molecules is reduced or eliminated by reducing or eliminating the expression of B2M, TAP1, or both. In some embodiments, the expression of one or more MHC class II molecules is reduced or eliminated by reducing or eliminating the expression of CIITA and/or CD74, or both. In some embodiments, the expression of one or more MHC class I molecules, or the expression of one or more MHC class II molecules is reduced or eliminated in the engineered immune evasive cell compared to a wildtype cell, unmodified cell, or control cell.

In some embodiments, at least 50%, at least 55%, at least 60%, at least 65%, at least 70%, at least 75%, at least 80%, at least 85%, at least 90%, at least 95%, or 100% of the cells in the population of therapeutic cells are immune evasive cells. In some embodiments, at least 50%, at least 55%, at least 60%, at least 65%, at least 70%, at least 75%, at least 80%, at least 85%, at least 90%, at least 95%, or 100% of the cells in the population of therapeutic cells have one or more transgenes encoding one or more immune tolerogenic factors inserted into the endogenous B2M locus and/or CIITA locus. In some embodiments, at least 50%, at least 55%, at least 60%, at least 65%, at least 70%, at least 75%, at least 80%, at least 85%, at least 90%, at least 95%, or 100% of the cells in the population of therapeutic cells have reduced or lack of expression of one or more MHC I and/or one or more MHC II molecules compared to a wildtype cell, unmodified cell, or control cell.

In some embodiments, transgene insertion is carried out by homology-directed repair (HDR)-mediated insertion using a site-directed nuclease, for example, one selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some embodiments, the one or more transgenes encoding one or more tolerogenic factors are introduced into a cell by calcium phosphate or lipid-mediated transfection, electroporation, fusogens, or viral transduction. In some embodiments, the virus is a retrovirus such as Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), lentivirus, a Gammretrovirus, an Epsilonretrovirus, an Alpharetrovirus, a Betaretrovirus, a Deltaretrovirus, or a Spumaretrovirus. In some embodiments, the virus is an adeno-associated viral (AAV) vector such as an AAV6 vector or an AAV9 vector.

In some embodiments wherein the tolerogenic factor is CD47, the CD47 is human CD47 comprising an amino acid sequence at least 80% identical to the amino acid sequence set forth in SEQ ID NO:1 or SEQ ID NO:2. In some embodiments, the human CD47 further comprises a leader peptide. In some embodiments, a transgene encoding CD47 comprises a nucleotide sequence that is at least 80%, at least 85%, at least 90%, at least 95%, or 100% identical to the nucleotide sequence set forth in SEQ ID NO:3 or SEQ ID NO:4. In some embodiments, the nucleotide sequence further comprises a sequence encoding a leader peptide. In some embodiments, the nucleotide sequence is codon-optimized. In some embodiments, the nucleotide sequence is at least 80%, at least 85%, at least 90%, at least 95%, or 100% identical to the nucleotide sequence set forth in SEQ ID NO:5.

In some embodiments, a transgene encoding a tolerogenic factor comprises a promoter, an insulator, an enhancer, a polyadenylation (poly(A)) tail, and/or a ubiquitous chromatin opening element. In some embodiments, the promoter is a constitutive promoter, for example, an EF1α, a short EF1α, CMV, SV40, PGK, UBC, CAG, MND, SSFV, or ICOS promoter. In some embodiments, the transgene further comprises the nucleotide sequence encoding a safety switch.

In some embodiments, a construct or vector comprises a transgene encoding one or more tolerogenic factors, and optionally one or more nucleotide sequences encoding one or more safety switches. In some embodiments, a construct or vector comprises a transgene encoding two or more tolerogenic factors. In certain of these embodiments, the transgene and optionally the nucleotide sequence encoding the safety switch are in the form of a polycistronic construct connected by one or more cleavage sites. In some embodiments, one or more tolerogenic factors are co-expressed in the same expression cassette of the construct. In some embodiments, two or more tolerogenic factors are co-expressed in different expression cassettes of the same construct, wherein the expression cassettes are separated by one or more cleavage sites. In the 5′ to 3′ order, the coding sequence for the safety switch can precede the coding sequence for the tolerogenic factor or vice versa. In some embodiments, the one or more cleavage sites comprise a self-cleaving site, for example, a 2A site. In some embodiments, the 2A site comprises a T2A, P2A, E2A, or F2A site. In some embodiments, the one or more cleavage sites further comprise a protease site, for example, a furin site. In some embodiments, the furin site comprises an FC1, FC2, or FC3 site. In some embodiments, the protease site precedes the 2A site in the 5′ to 3′ order.

In some embodiments, the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase (rapaCasp) such as rapaCasp 9, CCR4, CD16, CD19, CD20, CD30, EGFR, GD2, HER1, HER2, MUC1, PSMA, and RQR8. In some embodiments, the nucleotide sequence encoding the safety switch is in the same expression cassette comprising the transgene encoding one or more tolerogenic factors. In some embodiments, the nucleotide sequence encoding the safety switch is in a different expression cassette from the expression cassette comprising the transgene encoding one or more tolerogenic factors. In some embodiments wherein the tolerogenic factor is CD47, any of the agents that can inhibit or block the interaction of CD47 and SIRPa can be used in any combination to serve as safety switches for any of the engineered immune evasive cells disclosed herein.

In some aspects, provided is a population of the therapeutic cells generated by methods according to various embodiments disclosed herein.

In some aspects, provided is a population of therapeutic cells wherein at least 50%, at least 55%, at least 60%, at least 65%, at least 70%, at least 75%, at least 80%, at least 85%, at least 90%, at least 95%, or 100% of the cells in the population have (a) increased expression of one or more tolerogenic factors encoded by one or more transgenes, and/or (b) reduced expression of one or more MHC I and/or one or more MHC II molecules. In some embodiments, the surface expression of one or more tolerogenic factors is increased. In some embodiments, the surface expression or trafficking of one or more MHC I and/or one or more MHC II molecule is reduced. In some aspects, provided is a population of therapeutic cells wherein at least 50%, at least 55%, at least 60%, at least 65%, at least 70%, at least 75%, at least 80%, at least 85%, at least 90%, at least 95%, or 100% of the cells in the population have (a) increased expression of CD47 encoded by a transgene, and/or (b) reduced expression of one or more MHC I and/or one or more MHC II molecules. In some aspects, provided is a population of therapeutic cells wherein at least 50%, at least 55%, at least 60%, at least 65%, at least 70%, at least 75%, at least 80%, at least 85%, at least 90%, at least 95%, or 100% of the cells in the population have (a) increased expression of CD47 encoded by a transgene, and/or (b) reduced expression of B2M, TAP1, CD74, and/or CIITA. In some aspects, provided is a population of therapeutic cells wherein at least 50%, at least 55%, at least 60%, at least 65%, at least 70%, at least 75%, at least 80%, at least 85%, at least 90%, at least 95%, or 100% of the cells have (a) increased expression of CD47 encoded by a transgene, (b) reduced expression of B2M and/or TAP1 and one or more MHC I molecules, and/or (c) reduced expression of CIITA and/or CD74 and one or more MHC II molecules.

In some embodiments, the engineered immune evasive cell is an allogeneic cell. In some embodiments, the engineered immune evasive cell is a primary cell. In some embodiments, the engineered immune evasive cell is a pluripotent stem cell (PSC) such as an induced pluripotent stem cell (iPSC), or an embryonic stem cell (ESC). Various cell types can be differentiated from an engineered immune evasive PSC (e.g., an engineered immune evasive ESC or an engineered immune evasive iPSC). These cell types include but are not limited to pancreatic islet cells including pancreatic beta islet cells, retinal pigment epithelial cells, T cells, B cells, NK cells, thyroid cells, cells producing factors, skin cells, blood cells, plasma cells, platelets, renal cells, hepatocytes, neural cells, neuronal cells, glial progenitor cells, epithelial cells, endothelial cells, cardiac cells, cardiac progenitor cells, and cardiomyocytes. In some embodiments, the engineered immune evasive cells retain pluripotency and/or retain differentiation potential.

In some embodiments, the engineered immune evasive cell is a B2M_{indel/indel}, TAP1^{indel/indel}, CD74^{indel/indel}, and/or CIITA^{indel/indel} cell. In some embodiments, the engineered immune evasive cell is a B2M^{-/-}, TAP1^{-/-}, CD74^{-/-}, and/or CIITA^{-/-} cell. In some embodiments, the engineered immune evasive cell is a B2M^{indel/indel}, TAP1^{indel/indel}, CD74^{indel/indel}, and/or CIITA ^{indel/indel} cell overexpressing one or more tolerogenic factors encoded by one or more transgenes. In some embodiments, the engineered immune evasive cell is a B2M^{-/-}, TAP1^{-/-}, CD74^{-/-}, and/or CIITA^{-/-} cell overexpressing one or more tolerogenic factors encoded by one or more transgenes. In some embodiments, the tolerogenic factor is CD47 such as human CD47.

In some aspects, provided is a pharmaceutical composition comprising a population of the therapeutic cells according to various embodiments disclosed herein. In some embodiments, the pharmaceutical composition comprises one or more types or subtypes of the immune evasive cells disclosed herein. In some embodiments, the pharmaceutical composition comprises immune evasive cardiac progenitor cells (CPCs) and immune evasive epicardial cells. In some embodiments, the pharmaceutical composition comprises two or more immune evasive T cell subtypes. In some embodiments, the pharmaceutical composition comprises immune evasive cells derived from two or more donors. In some embodiments, the pharmaceutical composition comprises immune evasive cells derived from one or more donors and from the recipient who is to be administered with the pharmaceutical composition.

In some aspects, provided are methods of treating a disease or a condition in a subject in need thereof, comprising administering to the subject a population of the therapeutic cells or a pharmaceutical composition according to various embodiments disclosed herein.

In some embodiments, the disease or condition includes cancer, an autoimmune disease, a neurodegenerative disease, a cardiovascular condition or disease, a vascular condition or disease, a corneal condition or disease, a liver condition or disease, a thyroid condition or disease, and/or a kidney condition or disease.

In some embodiments, the disease is cancer such as a hematologic malignancy. In some embodiments, the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

### BRIEF DESCRIPTION OF THE DRAWINGS

FIG. 1 is a flow chart showing a method for generating immune evasive cells according to certain embodiments disclosed herein.

FIG. 2 shows an illustration of the interaction of a MAD7 nuclease with genomic DNA and crRNA and an exemplary crRNA structure and sequence.

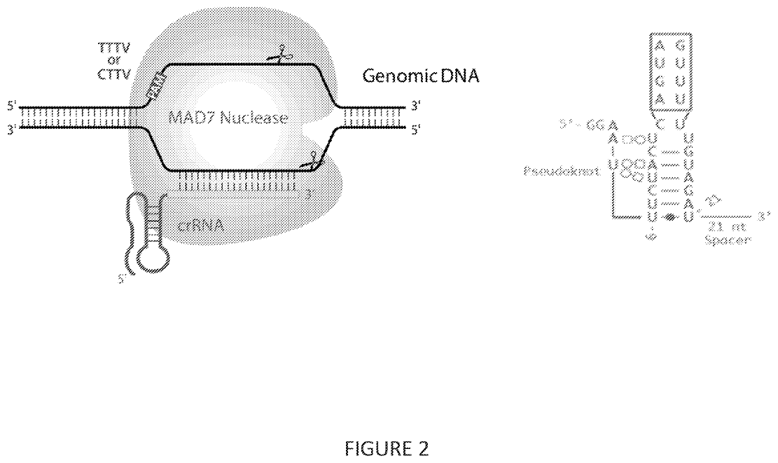

FIG. 3 shows a flow chart illustrating an exemplary MAD7 sgRNA library screening.

FIG. 4 shows a flow chart illustrating an exemplary gRNA library screening workflow.

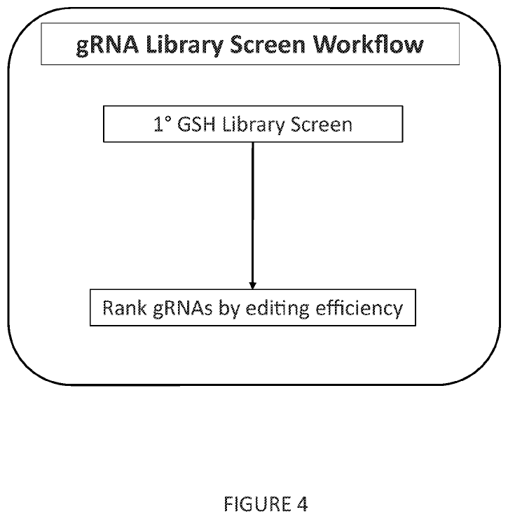

FIG. 5 shows exemplary steps of a T7 Endonuclease-I (T7E1) assay.

FIG. 6 shows an illustration of components for an exemplary on target amplicon (OTA) next generation sequencing (NGS) (OTA-NGS) assay.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents disclosed herein are merely exemplary and that equivalents of such are known in the art.

To the extent any materials incorporated by reference herein conflict with the present disclosure, the present disclosure controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

The term “antibody” is used to denote, in addition to natural antibodies, genetically engineered or otherwise modified forms of immunoglobulins or antigen binding portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)₂ fragment, or diabody. An scFv antibody is derived from an antibody by linking the variable regions of the heavy (V_{H}) and light (V_{L}) chains of the immunoglobulin with a short linker peptide. Similarly, a disulfide linked Fv antibody can be generated by linking the V_{H} and V_{L} using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the V_{H} and V_{L} regions noncovalent connected by a small peptide linker or covalently linked to each other. The antibodies disclosed herein, including those that comprise an immunogenically active portion of an immunoglobulin molecule, retain the ability to bind a specific antigen.

The term “antigen” refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′) 2 fragments, diabodies, or other synthetic polypeptides selected for their specific ability to bind to a biological molecule, a molecular complex, or other target of interest.

As used herein, “clinically effective amount” refers to an amount sufficient to provide a clinical benefit in the treatment and/or management of a disease, disorder, or condition. In some embodiments, a clinically effective amount is an amount that has been shown to produce at least one improved clinical endpoint to the standard of care for the disease, disorder, or condition. In some embodiments, a clinically effective amount is an amount that has been demonstrated, for example in a clinical trial, to be sufficient to provide statistically significant and meaningful effectiveness for treating the disease, disorder, or condition. In some embodiments, the clinically effective amount is also a therapeutically effective amount. In other embodiments, the clinically effective amount is not a therapeutically effective amount.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods is known in the art, and include, for example, methods disclosed in at least U.S. Pat. Nos. 5,786,464 and 6,114,148.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of introducing a specific nucleic acid sequence into a cell or into another nucleic acid sequence, or as a means of transporting another nucleic acid molecule. Vectors may be, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors). The construct optionally comprises one or more safety switches. In some embodiments, the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8. In some embodiments wherein the construct expresses CD47, any of the agents that can inhibit or block the interaction of CD47 and SIRPα can be used in any combination to serve as safety switches for any of the engineered immune evasive cells disclosed herein.

The terms “decreased,” “reduced,” “reduction,” and “decrease” are all used herein generally to mean a decrease by a statistically significant amount. However, for avoidance of doubt, “decreased,” “reduced,” “reduction,” or “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. In some embodiments, the cells are engineered to have reduced expression of one or more genes relative to an unaltered or unmodified wild-type cell. In some embodiments, the cells are engineered to have reduced expression of one or more genes relative to a control cell.

The term “donor” or “donor subject” refers to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor” or “donor subject” also encompasses any vertebrate including but not limited to mammals, reptiles, amphibians and fish. However, advantageously, the donor is a mammal such as a human, or other mammals such as a domesticated mammal, e.g., dog, cat, horse, and the like, or production mammal, e.g., cow, sheep, pig, and the like. A “donor” or “donor subject” can also refer to more than one donor, for example one or more humans or non-human animals or non-human mammals.

The term “endogenous” refers to a referenced molecule or polypeptide that is naturally present in the cell. Similarly, the term when used in reference to expression of an encoding nucleic acid refers to expression of an encoding nucleic acid naturally contained within the cell and not exogenously introduced. Similarly, the term when used in reference to a promoter sequence refers to a promoter sequence naturally contained within the cell and not exogenously introduced.

The term “engineered immune evasive cell,” “engineered cell,” or “immune evasive cell,” disclosed herein may be used interchangeably and refer to a primary cell or a PSC which is modified to have one or more transgenes encoding one or more exogenous tolerogenic factors inserted at a B2M and/or CIITA locus, to reduce or eliminate the expression of B2M and/or CIITA, and/or to reduce or eliminate the expression of one or more MHC I molecules and/or one or more MHC II molecules. As used herein, the term “engineered immune evasive cell,” “engineered cell,” or “immune evasive cell,” also encompasses a cell derived from a PSC (ESC or iPSC) or a progeny thereof, which is modified to have one or more transgenes encoding one or more exogenous tolerogenic factors inserted at a B2M and/or CIITA locus, to reduce or eliminate the expression of B2M and/or CIITA, and/or to reduce or eliminate the expression of one or more MHC I molecules and/or one or more MHC II molecules. As used herein, the term “derived from a PSC or a progeny thereof” encompasses the initial PSC that is generated and any subsequent progeny thereof. As used herein, the term “progeny” encompasses, e.g., a first-generation progeny, i.e., the progeny is directly derived from, obtained from, obtainable from or derivable from the initial PSC by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial PSC or a progeny thereof.

The term “engineered cell,” “modified cell” or “genetically modified cell” as used herein refers to a cell that has been altered in at least some way by human intervention, including, for example, by genetic alterations or modifications, such that the engineered cell differs from a wild-type cell or an unmodified cell.

As used herein, the term “exogenous” in the context of a polynucleotide or polypeptide being expressed is intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences. Genetic modifications include, for example, both transient knock-in and mechanisms that result in permanent knock-in of nucleic acids sequences. Genetic modifications also include, for example, reduced or increased transcription, reduced or increased mRNA stability, reduced or increased translation, and reduced or increased protein stability.

The term “host cell” as used herein refers to a cell or microorganism targeted for genetic modification by introduction of a construct or vector carrying a nucleotide sequence for expression of a protein or polypeptide of interest.

The term “immune evasive” is used to describe a cell being less prone to immune rejection by a subject into which such cell is transplanted. For example, relative to an unaltered or unmodified wild-type cell, such an immune evasive cell may be about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples disclosed herein, genome editing technologies are used to modulate the expression of one or more MHC I and/or one or more MHC II genes, and thus, to generate an immune evasive cell. In other examples disclosed herein, a tolerogenic factor is introduced into a cell and when expressed can modulate or affect the ability of the cell to be recognized by recipient immune system and thus confer immune evasiveness. The degree of immune evasiveness of a cell can be determined by evaluating the cell's ability to elicit adaptive and innate immune responses. Such immune response can be measured using assays recognized by those skilled in the art, for example, by measuring the effect of an immune evasive cell on cell proliferation, cell activation, or other cell activities. Immune evasive cells may undergo decreased killing by T cells and/or NK cells upon administration to a subject or show decreased macrophage engulfment compared to an unmodified or wildtype cell. In some cases, an immune evasive cell elicits a reduced or diminished immune response in a recipient subject compared to a corresponding unmodified wild-type cell. In some cases, an immune evasive cell is nonimmunogenic or fails to elicit an immune response in a recipient subject.

The terms “increased,” “increase,” “enhance” or “activate” are all used herein to generally mean an increase by a statically significant amount; for the avoidance of any doubt, the terms “increased,” “increase,” “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level, also referred to as the basal level, is 0.

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A gene editing (e.g., CRISPR/Cas) system of the present disclosure can be used to induce an indel of any length or a point mutation in a target polynucleotide sequence.

As used herein, “knock down” refers to a reduction in expression of the target mRNA or the corresponding target protein. Knock down is commonly reported relative to levels present following administration or expression of a noncontrol molecule that does not mediate reduction in expression levels of RNA (e.g., a non-targeting control shRNA, siRNA, or miRNA). In some embodiments, knock down of a target gene is achieved by way of conditional or inducible shRNAs, conditional or inducible siRNAs, conditional or inducible miRNAs, or conditional or inducible CRISPR interference (CRISPRi). In some embodiments, knock down of a target gene is achieved by way of a protein-based method, such as a conditional or inducible degron method. In some embodiments, knock down of a target gene is achieved by genetic modification, including shRNAs, siRNAs, miRNAs, or use of gene editing systems (e.g., CRISPR/Cas).

Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use the gene editing systems (e.g., CRISPR/Cas) of the present disclosure to knock out a target polynucleotide sequence or a portion thereof based upon the details disclosed herein.

By “knock in” or “knock-in” herein means a genetic modification resulting from the insertion of a DNA sequence into a chromosomal locus in a host cell. This causes initiation of or increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This may be accomplished by modifying a promoter, adding a different promoter, adding an enhancer, adding other regulatory elements, or modifying other gene expression sequences.

As used herein, “knock out” or “knock-out” includes deleting all or a portion of a target polynucleotide sequence in a way that interferes with the translation or function of the target polynucleotide sequence. For example, a knock-out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g., CRISPR/Cas) of the present disclosure to knock out a target polynucleotide sequence or a portion thereof based upon the details disclosed herein.

In some embodiments, a genetic modification or alteration results in a knock out or knock down of the target polynucleotide sequence or a portion thereof. Knocking out a target polynucleotide sequence or a portion thereof using a gene editing system (e.g., CRISPR/Cas) of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell.

The term “native cell” as used herein refers to a cell that is not otherwise modified (e.g., engineered). In some embodiments, a native cell is a naturally occurring wild-type cell or a control cell. By “wild-type” or “wt” or “control” in the context of a cell means any cell found in nature. Examples of wild type or control cells include primary cells and T cells found in nature. However, by way of example, in the context of an engineered cell, as used herein, “wild-type” or “control” can also mean an engineered cell that may contain nucleic acid changes resulting in reduced expression of one or more MHC I and/or one or more MHC II molecules and/or B2M, TAP1, CD74, and/or CIITA, but did not undergo the gene editing procedures to result in overexpression of CD47 proteins. For example, as used herein, “wild-type” or “control” means an engineered cell that comprises reduced or knocked out expression of B2M, TAP1, CD74, and/or CIITA. As used herein, “wild-type” or “control” also means an engineered cell that may contain nucleic acid changes resulting in overexpression of CD47 proteins, but did not undergo the gene editing procedures to result in reduced expression of one or more MHC I molecules and/or one or more MHC II molecules and/or B2M, TAP1, CD74, and/or CIITA. In the context of an iPSC or a progeny thereof, “wild-type” or “control” also means an iPSC or progeny thereof that may contain nucleic acid changes resulting in pluripotency but did not undergo the gene editing procedures of the present disclosure to achieve reduced expression of one or more MHC I molecules and/or one or more MHC II molecules and/or B2M, TAP1, CD74, and/or CIITA, and/or overexpression of CD47 proteins. For example, as used herein, “wild-type” or “control” means an iPSC or progeny thereof that comprises reduced or knocked out expression of B2M, TAP1, CD74, and/or CIITA. In the context of a primary cell or a progeny thereof, “wild-type” or “control” also means a primary cell or progeny thereof that may contain nucleic acid changes resulting in reduced expression of one or more MHC I molecules and/or one or more MHC II molecules and/or B2M, TAP1, CD74, and/or CIITA, but did not undergo the gene editing procedures to result in overexpression of CD47 proteins. For example, as used herein, “wild-type” or “control” means a primary cell or progeny thereof that comprises reduced or knocked out expression of B2M, TAP1, CD74, and/or CIITA. Also in the context of a primary cell or a progeny thereof, “wild-type” or “control” also means a primary cell or progeny thereof that may contain nucleic acid changes resulting in overexpression of CD47 proteins, but did not undergo the gene editing procedures to result in reduced expression of one or more MHC I molecules and/or one or more MHC II molecules and/or B2M, TAP1, CD74, and/or CIITA. In some embodiments, the cells are engineered to have regulatable reduced or increased expression of one or more target genes relative to a cell of the same cell type that does not comprise the modifications. In some embodiments, the wild-type cell or the control cell is a starting material. In some embodiments, the starting material is a primary cell collected from a donor. In some embodiments, the starting material is a primary blood cell collected from a donor, e.g., via a leukopak. For example, unmodified T cells obtained from a donor is a starting material that are considered wild-type or control cells as contemplated herein. In another example, an iPSC cell line starting material is a starting material that is considered a wild-type or control cell as contemplated herein. In some embodiments, the starting material is otherwise modified or engineered to have altered expression of one or more genes to generate the engineered cell.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “operably linked” refers to the association of two or more nucleic acid molecules on a single nucleic acid fragment so that the function of one is affected by the other.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells” as used herein also encompasses “induced pluripotent stem cells,” or “iPSCs,” or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPSC” cells can be created by inducing the expression of certain regulatory genes or by the exogenous application of certain proteins. Methods for the induction of iPSCs are known in the art and are further disclosed below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is provided below. As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

In some embodiments, the engineered immune evasive cells disclosed herein are propagated from a primary cell or a progeny thereof. As used herein, the term “propagated from a primary cell or a progeny thereof” encompasses the initial primary cell that is isolated from the donor subject and any subsequent progeny thereof. As used herein, the term “progeny” encompasses, e.g., a first-generation progeny, i.e., the progeny is directly derived from, obtained from, obtainable from or derivable from the initial primary cell by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial primary cell or a progeny thereof.

The term “recipient,” “recipient patient,” or “recipient subject” refers to an animal, for example, a human to whom treatment, including prophylactic treatment, with the cells as disclosed herein, is provided. For treatment of those infections, conditions or disease states, which are specific for a specific animal such as a human patient, the term patient refers to that specific animal. The term “recipient,” “recipient patient,” or “recipient subject” also encompasses any vertebrate including but not limited to mammals, reptiles, amphibians and fish. However, advantageously, the recipient, recipient patient, or recipient subject is a mammal such as a human, or other mammals such as a domesticated mammal, e.g., dog, cat, horse, and the like, or production mammal, e.g., cow, sheep, pig, and the like.

The term “safe harbor locus” refers to a gene locus that allows safe expression of a transgene or an exogenous gene. Safe harbors or genomic safe harbors are sites in the genome able to accommodate the integration of new genetic material in a manner that permits the newly inserted genetic elements to: (i) function predictably and (ii) do not cause alterations of the host genome posing a risk to the host cell or organism. Exemplary “safe harbor” loci include an AAVS1 locus, a CCR5 locus, a CXCR4 locus, a PPP1R12C (also known as AAVS1) locus, a CLYBL locus, an albumin locus, an SHS231 locus, an F3 locus, an MICA locus, an MICB locus, a LRP1 locus, an HMGB1 locus, an ABO locus, an RHD locus, an FUT1 locus, a KDM5D locus, and a Rosa locus.

In some embodiments, the cells or vectors disclosed herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells disclosed herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells including pancreatic beta islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a safety switch.

In some embodiments, the cells disclosed herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells disclosed herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells including pancreatic beta islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a suicide gene.

The term “subject” refers to a mammalian subject, preferably a human. A “subject in need thereof” may refer to a subject who has been diagnosed with a disease, or is at an elevated risk of developing a disease, or has received or is going to receive a transplant. The phrases “subject,” “individual,” and “patient” are used interchangeably herein.

The term “therapeutic cell” as used herein refers to an engineered immune evasive primary cell or a cell differentiated from an engineered immune evasive stem cell such as an engineered immune evasive PSC. In certain embodiments, the therapeutic cell does not include an engineered immune evasive stem cell such as an engineered immune evasive PSC because the stem cell needs to be differentiated into a desired cell type to be used as a therapeutic cell.

“A population of therapeutic cells” as used herein means that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the cells in this population are engineered immune evasive cells or cells differentiated from engineered immune evasive cells disclosed herein. In some embodiments wherein 50% or less of the cells in the population of therapeutic cells comprise the engineered immune evasive cells or cells differentiated from engineered immune evasive cells disclosed herein, another therapy, e.g., another cell therapy, can be administered to the subject. In some embodiments, a population of therapeutic cells comprises more than one type or more than one subtype of the immune evasive cells disclosed herein. In some embodiments, a population of therapeutic cells comprises a subpopulation of the cells which have one or more tolerogenic factors inserted at a B2M locus, and another subpopulation of the cells which have one or more tolerogenic factors inserted at a CIITA locus. In some embodiments, a population of therapeutic cells refers to a population of primary cells which are modified to express one or more exogenous tolerogenic factors, to reduce or eliminate expression of B2M, TAP1, CD74, and/or CIITA, and/or to reduce or eliminate expression of one or more MHC I molecules and/or one or more MHC II molecules, and which may or may not be sorted by positive selection such that this population of the cells comprises both modified cells and unmodified cells. In some embodiments, a population of therapeutic cells refers to a population of cells differentiated from a population of PSCs which are modified to express one or more exogenous tolerogenic factors, to reduce or eliminate expression of B2M, TAP1, CD74, and/or CIITA, and/or to reduce or eliminate expression of one or more MHC I molecules and/or one or more MHC II molecules, and which PSCs are not sorted by positive selection such that this population of the cells comprises both modified cells and unmodified cells. In some embodiments, a population of therapeutic cells comprises a mixture of immune evasive cells derived from different donors. In some embodiments, a population of therapeutic cells comprises a mixture of immune evasive cells derived from one or more donors and immune evasive cells derived from the recipient who is to be administered with the population of therapeutic cells. In some embodiments, a population of therapeutic cells comprises cells which have either or both of the following characteristics: (i) increased expression of a tolerogenic factor (e.g., CD47) encoded by a transgene and (ii) reduced expression of one or more MHC I molecules and/or one or more MHC II molecules. In some embodiments, reduced expression of one or more MHC class I molecules and/or one or more MHC class II molecules is achieved by reducing or eliminating expression of B2M and/or TAP1, and CIITA and/or CD74, respectively.

A “therapeutically effective amount” as used herein is an amount that produces a desired effect in a subject for treating a disease. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited, to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or the pharmaceutical composition containing the same, and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, NY, 2011, the entire disclosures of which are incorporated by reference herein.

The term “tolerogenic factor” as used herein includes hypoimmunity factors, complement inhibitors, and other factors that modulate or affect (e.g., reduce) the ability of a cell to be recognized by the immune system of a recipient subject upon administration, transplantation, or engraftment. Tolerogenic factors include but are not limited to A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9.

The terms “treat,” “treating,” and “treatment” as used herein with regard to a disease or a condition refers to alleviating the disease or condition partially or entirely; preventing the onset of the disease or condition; decreasing the likelihood of occurrence or recurrence of the disease or condition; slowing the progression or development of the disease or condition; eliminating, reducing, or slowing the development of one or more symptoms associated with the disease or condition; or increasing progression-free or overall survival of the disease or condition. For example, “treating” may refer to preventing or slowing the existing disease or condition from growing larger; preventing or slowing the formation or spreading of the disease or condition; and/or slowing the development of certain symptoms of the disease or condition. In some embodiments, the term “treat,” “treating,” or “treatment” means that the subject has a reduced number or size of diseased cells comparing to a subject without being administered with the treatment. In some embodiments, the term “treat,” “treating,” or “treatment” means that one or more symptoms of the disease or condition are alleviated in a subject receiving the treatment as disclosed herein comparing to a subject who does not receive such treatment. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

For purposes of this technology, beneficial or desired therapeutic or clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “variable region” or “variable domain” refers to a portion of an antibody heavy or light chain that is involved in antigen binding. Variable domains of antibody heavy (V_{H}) and light (V_{L}) chains each generally comprise four generally conserved framework regions (FRs) and three complementarity determining regions (CDRs). Framework regions separate CDRs, such that CDRs are situated between framework regions.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, sequences which control termination of transcription and translation, and optionally one or more safety switch. The vector may be a plasmid, a phage particle, a virus, or simply a potential genomic insert. Once transformed into a suitable host, the vector may replicate and function independently of the host genome, or may, in some instances, integrate into the genome itself.

### I. Methods of Genetic Modification

In some aspects, the present technology provides methods for generating an engineered immune evasive cell or a population of engineered immune evasive cells for cell therapy. In some embodiments, the method comprises inserting one or more transgenes encoding one or more tolerogenic factors, and optionally, one or more safety switches, into an endogenous B2M and/or CIITA gene locus of the cells. In certain embodiments, the method further comprises selecting for engineered cells that have the transgene inserted by positive selection for the tolerogenic factor (e.g., selection for expression of the tolerogenic factor). Inserting one or more tolerogenic factors at the endogenous B2M gene locus may reduce or eliminate B2M expression, reduce or eliminate the expression of one or more MHC I molecules, and increase expression of the tolerogenic factor in the engineered cells in one manufacturing step, so that the resulting engineered cells can be made immune evasive and not subject to immune rejection when transplanted into a recipient, thereby increasing both the efficiency of the manufacturing process and the effectiveness of cell-based therapies. Likewise, inserting one or more tolerogenic factors at the endogenous CIITA gene locus may reduce or eliminate CIITA expression, reduce or eliminate the expression of one or more MHC I/MHC II molecules, and increase expression of the tolerogenic factor in the engineered cells in one manufacturing step, so that the resulting engineered cells can be made immune evasive and not subject to immune rejection when transplanted into a recipient, thereby increasing both the efficiency of the manufacturing process and the effectiveness of cell-based therapies.

In some embodiments, the surface expression of one or more tolerogenic factors such as CD47 is increased. In some embodiments, the surface expression or trafficking of one or more MHC I and/or one or more MHC II molecules is reduced. In some embodiments, a function of one or more MHC I molecules and/or one or more MHC II molecules is reduced. In some embodiments, the function is antigen presentation.

### A. Insertion of One or More Transgenes Encoding One or More Tolerogenic Factors

### 1. Tolerogenic Factor

As disclosed herein, the exemplary tolerogenic factors include but are not limited to A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9, and truncations, modifications, or fusions of any of the above. One or more tolerogenic factors can be inserted into the B2M locus, CIITA locus, or a safe harbor locus disclosed herein. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, DUX4, CD24, CD27, CD35, CD46, CD55, CD59, CD64, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, and Serpinb9.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM 005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM 005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM 002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM 002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM 000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM 002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Useful genomic, polynucleotide and polypeptide information about human CD64, are provided in, for example, the GeneCard Identifier GC01P151397, HGNC No. 3613, NCBI Gene ID 2209, Uniprot No. P12314, and NCBI RefSeq Nos. NG_007578.1, NM_000566.4, NM_001378804.1, NM_001378805.1, NM_001378806.1, NM_001378807.1, NM_001378808.1, NM_001378809.1, NM_001378810.1, NM_001378811.1, and NR_166121.1.

Useful genomic, polynucleotide and polypeptide information about human CD27, are provided in, for example, the GeneCard Identifier GC12P024792, HGNC No. 11922, NCBI Gene ID 939, Uniprot No. P26842, and NCBI RefSeq Nos. NG_031995.1, NM_001242.5, NM_001413263.1, NM_001413264.1, NM_001413265.1, NM_001413266.1, NM_001413267.1, NM_001413268.1, and NR 182125.1

Useful genomic, polynucleotide and polypeptide information about human TNFAIP3 (also known as A20) are provided in, for example, the GeneCard Identifier GC06P137866, HGNC No. 11896, NCBI Gene ID 7128, Uniprot No. Q8NFZ5, and NCBI RefSeq Nos. NG_032761.1, NM_001270507.2, NM_001270508.2, and NM_006290.4.

Useful genomic, polynucleotide and polypeptide information about human CR1 are provided in, for example, the GeneCard Identifier GC01P207496, HGNC No. 2334, NCBI Gene ID 1378, Uniprot No. P17927, and NCBI RefSeq Nos. NG_007481.1, NM_000573.4, NM_000651.6, and NM_001381851.1.

Useful genomic, polynucleotide and polypeptide information about human MANF are provided in, for example, the GeneCard Identifier GC03P051385, HGNC No. 15461, NCBI Gene ID 7873, Uniprot No. P55145, and NCBI RefSeq Nos. NG_012652.3 and NM 006010.6.

In some embodiments, the present disclosure provides an engineered immune evasive cell or population thereof that has been modified to express the exogenous tolerogenic factor (e.g., immunomodulatory polypeptide) CD47. In some embodiments, the present disclosure provides a method for altering a cell genome to express an exogenous CD47. In some instances, the cell comprises an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of the B2M locus, CIITA locus, or a safe harbor locus. In some instances, the cell expresses a nucleotide sequence encoding a human CD47 polypeptide wherein the nucleotide sequence is inserted into at least one allele of the B2M locus. In some instances, the cell expresses a nucleotide sequence encoding a human CD47 polypeptide wherein the nucleotide sequence is inserted into at least one allele of the CIITA locus. In some instances, the cell expresses a nucleotide sequence encoding a human CD47 polypeptide wherein the nucleotide sequence is inserted into at least one allele of a safe harbor locus, such as, but not limited to, an AAVS1 gene locus, a CCR5 gene locus, a CXCR4 gene locus, a PPP1R12C gene locus, an albumin gene locus, an SHS231 gene locus, a CLYBL gene locus, a Rosa gene locus, an F3 (CD142) gene locus, an MICA gene locus, an MICB gene locus, a LRP1 (CD91) gene locus, an HMGB1 gene locus, an ABO gene locus, an RHD gene locus, an FUT1 locus, and a KDM5D gene locus.

CD47 is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients.

In some embodiments, the cell disclosed herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP 942088.1. In some embodiments, the cell disclosed herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM 001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell disclosed herein comprises a CD47 polypeptide having an amino acid sequence as set forth in NCBI Ref. Sequence Nos. NP_001768.1 and NP_942088.1.

In some embodiments, the CD47 is human CD47, and in some of these embodiments, the human CD47 comprises or consists of an amino acid sequence set forth in SEQ ID NO: 1 or SEQ ID NO: 2 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 1 or SEQ ID NO: 2. In some embodiments, the transgene encoding CD47 comprises a nucleotide sequence corresponding to an mRNA sequence of human CD47. In some embodiments, the transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 3 (coding sequence (CDS) of the nucleotide sequence set forth in NCBI Ref. No. NM_001777.4) or SEQ ID NO: 4 (CDS of the nucleotide sequence set forth in NCBI Ref. No. NM_198793.2).

In some embodiments, the transgene encoding CD47 is codon-optimized for expression in a mammalian cell, for example, a human cell. In some embodiments, the codon-optimized transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 5.

TABLE 1
Exemplary sequences of CD47

| SEQ | | |
|---|---|---|
| ID NO: | Sequence | Description |
| 1 | MWPLVAALLLGSACCGSAQLLFNKTKSVEFT | Amino acid sequence |
| | FCNDTVVIPCFVTNMEAQNTTEVYVKWKFK | encoded by CDS of |
| | GRDIYTFDGALNKSTVPTDFSSAKIEVSQLLK | NM_001777.4 |
| | GDASLKMDKSDAVSHTGNYTCEVTELTREGE | |
| | TIIELKYRVVSWFSPNENILIVIFPIFAILLFWGQ | |
| | FGIKTLKYRSGGMDEKTIALLVAGLVITVIVIV | |
| | GAILFVPGEYSLKNATGLGLIVTSTGILILLHY | |
| | YVFSTAIGLTSFVIAILVIQVIAYILAVVGLSLC | |
| | IAACIPMHGPLLISGLSILALAQLLGLVYMKFV | |
| | ASNQKTIQPPRKAVEEPLNAFKESKGMMNDE | |
| 2 | MWPLVAALLLGSACCGSAQLLFNKTKSVEFT | Amino acid sequence |
| | FCNDTVVIPCFVTNMEAQNTTEVYVKWKFK | encoded by CDS of |
| | GRDIYTFDGALNKSTVPTDFSSAKIEVSQLLK | NM_198793.2 |
| | GDASLKMDKSDAVSHTGNYTCEVTELTREGE | |
| | TIIELKYRVVSWFSPNENILIVIFPIFAILLFWGQ | |
| | FGIKTLKYRSGGMDEKTIALLVAGLVITVIVIV | |
| | GAILFVPGEYSLKNATGLGLIVTSTGILILLHY | |
| | YVFSTAIGLTSFVIAILVIQVIAYILAVVGLSLC | |
| | IAACIPMHGPLLISGLSILALAQLLGLVYMKFV | |
| | ASNQKTIQPPRNN | |
| 3 | atgtggcccctggtagcggcgctgttgctgggctcggcgtgctgcgga | Nucleotide sequence of |
| | tcagctcagctactatttaataaaacaaaatctgtagaattcacgttttgta | NM_001777.4 CDS (nts |
| | atgacactgtcgtcattccatgctttgttactaatatggaggcacaaaaca | 124-1095) |
| | ctactgaagtatacgtaaagtggaaatttaaaggaagagatatttacacc | |
| | tttgatggagctctaaacaagtccactgtccccactgactttagtagtgca | |
| | aaaattgaagtctcacaattactaaaaggagatgcctctttgaagatgga | |
| | taagagtgatgctgtctcacacacaggaaactacacttgtgaagtaaca | |
| | gaattaaccagagaaggtgaaacgatcatcgagctaaaatatcgtgttg | |
| | tttcatggttttctccaaatgaaaatattcttattgttattttcccaatttttgcta | |
| | tactcctgttctggggacagtttggtattaaaacacttaaatatagatccg | |
| | gtggtatggatgagaaaacaattgctttacttgttgctggactagtgatca | |
| | ctgtcattgtcattgttggagccattcttttcgtcccaggtgaatattcatta | |
| | aagaatgctactggccttggtttaattgtgacttctacagggatattaatatt | |
| | acttcactactatgtgtttagtacagcgattggattaacctccttcgtcattg | |
| | ccatattggttattcaggtgatagcctatatcctcgctgtggttggactga | |
| | gtctctgtattgcggcgtgtataccaatgcatggccctcttctgatttcagg | |
| | tttgagtatcttagctctagcacaattacttggactagtttatatgaaatttgt | |
| | ggcttccaatcagaagactatacaacctcctaggaaagctgtagagga | |
| | accccttaatgcattcaaagaatcaaaaggaatgatgaatgatgaataa | |
| 4 | atgtggcccctggtagcggcgctgttgctgggctcggcgtgctgcgga | Nucleotide sequence of |
| | tcagctcagctactatttaataaaacaaaatctgtagaattcacgttttgta | NM_198793.2 CDS (nts |
| | atgacactgtcgtcattccatgctttgttactaatatggaggcacaaaaca | 181-1098) |
| | ctactgaagtatacgtaaagtggaaatttaaaggaagagatatttacacc | |
| | tttgatggagctctaaacaagtccactgtccccactgactttagtagtgca | |
| | aaaattgaagtctcacaattactaaaaggagatgcctctttgaagatgga | |
| | taagagtgatgctgtctcacacacaggaaactacacttgtgaagtaaca | |
| | gaattaaccagagaaggtgaaacgatcatcgagctaaaatatcgtgttg | |
| | tttcatggttttctccaaatgaaaatattcttattgttattttcccaatttttgcta | |
| | tactcctgttctggggacagtttggtattaaaacacttaaatatagatccg | |
| | gtggtatggatgagaaaacaattgctttacttgttgctggactagtgatca | |
| | ctgtcattgtcattgttggagccattcttttcgtcccaggtgaatattcatta | |
| | aagaatgctactggccttggtttaattgtgacttctacagggatattaatatt | |
| | acttcactactatgtgtttagtacagcgattggattaacctccttcgtcattg | |
| | ccatattggttattcaggtgatagcctatatcctcgctgtggttggactga | |
| | gtctctgtattgcggcgtgtataccaatgcatggccctcttctgatttcagg | |
| | tttgagtatcttagctctagcacaattacttggactagtttatatgaaatttgt | |
| | ggcttccaatcagaagactatacaacctcctaggaataactga | |
| 5 | atgtggcccctggtcgccgccctgttgctgggctcggcatgctgcggat | Codon-optimized |
| | cagctcagctactgtttaataaaacaaaatctgtagaattcacgttttgtaa | nucleotide sequence |
| | cgacactgtcgtgatcccatgctttgttactaatatggaggcacaaaaca | encoding CD47 |
| | ccactgaagtgtacgtgaagtggaaattcaaaggcagagacatttacac | |
| | ctttgacggcgccctcaacaagtccaccgtgcccactgactttagtagc | |
| | gcaaaaattgaggtcagccaattactaaaaggagatgcctctttgaagat | |
| | ggacaagagcgatgctgtcagccacacagggaactacacttgtgaagt | |
| | aacagagttaacccgcgaaggtgaaacgatcatcgagctgaagtatcg | |
| | agtggtgtcctggttttctccgaacgagaatatccttatcgtaattttccca | |
| | attttcgctatcctcctgttctggggccagtttggtatcaagacactcaaat | |
| | atcggtccggtgggatggatgagaagacaattgccctgcttgttgctgg | |
| | actcgtgatcaccgtcatcgtgattgttggggccatccttttcgtcccagg | |
| | ggagtacagcctgaagaatgctacgggcctgggattaattgtgacctct | |
| | acagggatactcatcctgcttcactactatgtgttcagtaccgcgattgga | |
| | ctgacctccttcgtcattgccatattggtgattcaggtgatagcctacatcc | |
| | tcgccgtggttggcctgagtctctgtatcgcggcgtgcatacccatgcat | |
| | ggccctcttctgatttcagggttgagtatcctcgcactagcacagttgctg | |
| | ggactggtttatatgaaatttgtggcctccaaccagaagactatacagcc | |
| | tcctaggaaggctgtagaggagcccctgaatgcattcaaggaatcaaa | |
| | aggcatgatgaatgatgaa | |

### 2. Regulatory Elements

In some embodiments, expression of the tolerogenic factor may be operably linked to an endogenous promoter at the B2M or CIITA gene locus. In certain of these embodiments, the transgene encoding the tolerogenic factor to be inserted need not include an exogenous promoter however, in some embodiments, the transgene may include an exogenous insulator and/or an exogenous enhancer.

Alternatively, in other embodiments, the transgene encoding the tolerogenic factor may additionally comprise an exogenous promoter to drive expression of the tolerogenic factor in the host cell. This disclosure encompasses various promoters as well as functional derivatives of these promoters. As used herein, a functional derivative of a promoter means a promoter that is larger or smaller than the wildtype promoter but retains the function of the wildtype promoter.

In certain of these embodiments, the exogenous promoter may be one that drives constitutive gene expression in mammalian cells. Those frequently used include, for example, elongation factor 1 alpha (EF1α) promoter, EF1α short promoter, cytomegalovirus (CMV) immediate-early promoter (Greenaway et al., Gene 18:355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60 (1): 65-74 (1987)), human beta actin promoter, polyubiquitin C gene (UBC) promoter, CAG promoter (Nitoshi et al., Gene 108:193-199 (1991)), MND (MPSV LTR, NCR deleted, and d/587 PBS; Challita et al., J. Virol 69 (2): 748-755 (1995)) promoter, SSFV promoter, and ICOS promoter. An example of a promoter that is capable of expressing a transgene in a mammalian cell is the EF1α promoter. The native EF1α promoter drives expression of the alpha subunit of the elongation factor-1 complex, which is responsible for the enzymatic delivery of aminoacyl tRNAs to the ribosome. As another example, an MND promoter is a synthetic promoter that contains the U3 region of a modified gammaretrovirus-derived MoMuLV LTR with myeloproliferative sarcoma virus enhancer, and this promoter has been shown to be highly and constitutively active in the hematopoietic system and to resist transcriptional silencing. See, e.g., Halene et al., Blood 94 (10): 3349-3357 (1999).

In some embodiments, the transgene encoding the tolerogenic factor may comprise additional regulatory elements operatively linked to the tolerogenic factor sequence and/or promoter, including, for example, insulators, enhancers, polyadenylation (poly(A)) tails, and/or ubiquitous chromatin opening elements. As known to a skilled artisan, these regulatory elements may be needed to affect the expression and processing of coding sequences to which they are operatively linked. Regulatory elements used for transgene expression modulation may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion.

In some embodiments, the transgene encoding the tolerogenic factor may additionally comprise an insulator to modulate the expression of the tolerogenic factor in the host cell. Insulators are DNA elements (usually about 50 nucleotides in length) that can shelter genes from inappropriate regulatory interactions. In some embodiments, insulators insulate genes located in one domain from promiscuous regulation by enhancers or silencers in neighboring domains. Insulators that disrupt communication between an enhancer and its promoter when positioned between the two are called enhancer-blockers, and insulators that are located between a silencer and a promoter and protect the promoter from silencing are called barriers. In some embodiments, insulators that are barriers prevent the advance of nearby condensed chromatin and protect gene expression from positive and negative chromatin effects. Thus, in the design of a transgene, insulators are usually placed upstream of the promoter. Non-limiting examples of insulators include 5′HS5, DMD/ICR, BEAD-1, apoB (−57 kb), apoB (+43 kb), DMI site 1, DMI site 2 (from human); BEAD-1, HS2-6, DMR/ICR, SINE (from mouse); SF1, scs/scs′, gypsy, Fab-7, Fab-8, faswab, eve (from fruit fly); HMR tRNAThr, Chal UAS, UASrpg, STAR (from yeast); Lys 5′A, HS4, or 3′HS (from chicken); sns, URI (from sea urchin); and RO (from frog). Other examples of insulators include Mcp, Neighbor of Homie (Nhomie) insulator and Homing insulator at eve (Homie), and Su(Hw)-dependent insulators. In some embodiments, the first transgene encoding a tolerogenic factor may comprise an insulator having a sequence that is at least 80% identical (e.g., at least 80%, at least 85%, at least 90%, at least 95%, at least 96%, at least 97%, at least 98%, at least 99%, or 100% identical) to any of the disclosed insulators.

In some embodiments, the transgene encoding the tolerogenic factor comprises one copy of an insulator. In some embodiments, the transgene comprises a multimerized insulator. In some embodiments, a transgene comprises two copies of an insulator. In some embodiments, a transgene comprises three copies of an insulator. In some embodiments, a transgene comprises four copies of an insulator. In some embodiments, a transgene comprises five or more copies of an insulator. Insulator effectiveness is influenced by its structure and by the nature of the enhancer, promoter, and genomic context. In some embodiments, the transgene encoding the tolerogenic factor may comprise two or more heterologous insulators. In some embodiments, the two or more heterologous insulators interact with each other. In some embodiments, the transgene encoding the tolerogenic factor comprises an insulator and a regulatory protein that binds to the insulator.

In some embodiments, the first transgene encoding a tolerogenic factor may additionally comprise an enhancer to increase expression of the tolerogenic factor in the host cell. Enhancer sequences are regulatory DNA sequences that, when bound by specific proteins called transcription factors, enhance the transcription of an associated gene. Enhancers are regions of DNA, typically 100 to 1000 bp in size, that contain transcription factor-binding sites that stimulate the initiation and elongation of transcription from promoters. In most housekeeping genes, enhancers are located in close proximity to promoters. Some genes feature complex regulatory regions that can consist of dozens of enhancers located at variable distances from the regulated promoter. During transcriptional activation, enhancers are usually located in close proximity to gene promoters. Some promoters disclosed herein already have an enhancer incorporated; for example, the CAG promoter is constructed by combining the CMV early enhancer element, the chicken beta actin gene promoter, and the splice acceptor of the rabbit beta globin gene.

Enhancers may consist of combinations of short, degenerate sites, 6-12 bp in length, that are recognized by DNA-binding transcription factors, which determine enhancer activity. The combination of DNA-binding transcription factors on a given enhancer creates a platform that attracts co-activators and co-repressors that determine the enhancer activity in each specific group of cells. The ability of an enhancer to stimulate transcription depends on the combination of transcription factor sites that positively or negatively affect enhancer activity and the relative concentrations of enhancer-binding transcription factors within the nuclei of a given group of cells. Recently, super-enhancers have been identified, representing a special class of regulatory elements, characterized by large sizes, sometimes reaching tens of thousands of bp, with a high degree of transcription factor and co-activator enrichment. Super-enhancers are often located adjacent to genes known to be critical for cell differentiation. A more detailed study of super-enhancers has shown that they often consist of separate domains that can either function together to enhance the overall activity of each domain or play independent roles during the simultaneous activation of a large number of promoters.

During the activation of transcription, enhancers recruit several key complexes. The p300/CBP and Mll3/Mll4/COMPASS complexes have acetyltransferase and methyltransferase activities, respectively. The proteins Mll3 and Mll4 both contain a C-terminal SET (suppressor of variegation, enhancer of zeste, trithorax) domain, which is responsible for the monomethylation of lysine 4 of histone H3 (H3K4mel). The complexes formed by Mll3 and Mll4 have partially overlapping and insufficiently studied functions in the regulation of enhancer activity. Mll3 and Mll4 are also known to be involved in the recruitment of the p300/CBP co-activator, which is responsible for the acetylation of histone H3 at lysine 27 (H3K27ac). H3K27ac and H3K4mel histone marks are distinctive features of active enhancers and are used to identify enhancers in genomes.

In some embodiments, the transgene encoding the tolerogenic factor may additionally comprise a poly(A) tail. A poly(A) tail is a long chain of adenine nucleotides that is added to an mRNA molecule during RNA processing to increase the stability of the molecule. Immediately after a gene in a eukaryotic cell is transcribed, the new RNA molecule undergoes several modifications known as RNA processing. These modifications alter both ends of the primary RNA transcript to produce a mature mRNA molecule. The processing of the 3′ end adds a poly-A tail to the RNA molecule. First, the 3′ end of the transcript is cleaved to free a 3′ hydroxyl. Then an enzyme called poly-A polymerase adds a chain of adenine nucleotides to the RNA. This process, called polyadenylation, adds a poly-A tail that is between 100 and 250 residues long. The poly-A tail makes the RNA molecule more stable and prevents its degradation. Additionally, the poly-A tail allows the mature messenger RNA molecule to be exported from the nucleus and translated into a protein by ribosomes in the cytoplasm.

In some embodiments, the transgene encoding the tolerogenic factor may additionally comprise a ubiquitous chromatin opening element (UCOE). The integration of a transgene into a heterochromatic chromatin environment and the methylation of promoter DNA are major mechanisms that are antagonistic to gene expression, resulting in a variegated pattern of gene expression or silencing. Because stable and high-level transgene expression are essential for the efficient and rapid production of clonal cell lines in biomanufacturing as well as for the lifelong expression of a transgene at a therapeutic level in gene therapy, genetic regulatory elements that can prevent gene silencing and maintain high levels of expression for long periods of time are crucial.

Genetic regulatory elements that confer a transcriptionally permissive state can be broadly dichotomized into those that actively function through dominant chromatin remodeling mechanisms and those that function as border or boundary elements to restrict the spread of heterochromatin marks into regions of euchromatin. The latter include insulators, scaffold/matrix attachment regions (S/MARs), and stabilizing anti-repressor (STAR) elements, whilst the former comprise locus control regions (LCRs) and UCOEs. LCRs and UCOEs are defined by their ability to consistently confer site of integration-independent stable transgene expression that is proportional to transgene copy number, even when integrated into heterochromatin. LCRs are tissue-specific regulatory elements that consist of multiple subcomponents characterized by DNase I hypersensitivity and a high density of transcription factor binding sites. In contrast, UCOEs function ubiquitously and neither consist of multiple DNase I hypersensitive sites that are characteristic of LCRs, nor are they required to flank a transgene at both 5′ and 3′ ends in order to exert their function as in the case of insulators and S/MARs. Thus, structurally and functionally UCOEs represent a distinct class of genetic regulatory element. UCOEs have found widespread usage in protein therapeutic biomanufacturing applications as a means to manage costs and resources as well as to reliably expedite the generation of highly expressing recombinant cell clones. In some embodiments, UCOEs provide stable ubiquitous or tissue-specific expression in somatic tissues as well as in adult, embryonic, and induced pluripotent stem cells and their differentiated progeny.

### 3. Polycistronic Constructs

In some embodiments, one or more of the transgenes encoding tolerogenic factors may be in the form of polycistronic constructs. Polycistronic constructs have two or more expression cassettes for co-expression of two or more proteins of interest in a host cell. In some embodiments, the polycistronic construct comprises two expression cassettes, i.e., is bicistronic. In some embodiments, the polycistronic construct comprises three expression cassettes, i.e., is tricistronic. In some embodiments, the polycistronic construct comprises four expression cassettes, i.e., is quadcistronic. In some embodiments, the polycistronic construct comprises more than four expression cassettes. In any of these embodiments, each of the expression cassettes comprises a nucleotide sequence encoding a protein of interest (e.g., a tolerogenic factor, or a safety switch). In certain embodiments, the two or more genes being expressed are under the control of a single promoter and are separated from one another by one or more cleavage sites to achieve co-expression of the proteins of interest from one transcript. In other embodiments, the two or more genes may be under the control of separate promoters.

In some embodiments, the two or more expression cassettes of the polycistronic construct expressing one or more tolerogenic factors and/or one or more safety switches may be separated by one or more cleavage sites. As the name suggests, a polycistronic construct allows simultaneous expression of two or more separate proteins from one mRNA transcript in a host cell. Cleavage sites can be used in the design of a polycistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 2. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 2
Sequences of 2A peptides

| SEQ | | 2A |
|---|---|---|
| ID NO: | Amino Acid Sequence | Peptide |
| 6 | (GSG)EGRGSLLTCGDVEENPGP | T2A |
| 7 | (GSG)ATNFSLLKQAGDVEENPGP | P2A |
| 8 | (GSG)QCTNYALLKLAGDVESNPGP | E2A |
| 9 | (GSG)VKQTLNFDLLKLAGDVESNPGP | F2A |

In some embodiments, the one or more cleavage sites additionally comprise one or more protease sites. The one or more protease sites can either precede or follow the self-cleavage sites (e.g., 2A sites) in the 5′ to 3′ order. The protease site may be cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 3. Similar to the 2A sites, one or more optional glycine-serine-glycine (GSG) sequences can be included for cleavage efficiency.

TABLE 3
Sequences of furin sites

| SEQ ID NO: | Amino Acid Sequence | Furin site |
|---|---|---|
| 10 | RRRR(GSG) | FC1 |
| 11 | RKRR(GSG) | FC2 |
| 12 | RKRR(GSG)TPDPW(GSG) | FC3 |

In some embodiments, the one or more cleavage sites comprise one or more self-cleaving sites, one or more protease sites, and/or any combination thereof. For example, the cleavage site can include a 2A site alone. In another example, the cleavage site can include a FC2 or FC3 site, followed by a 2A site. In these embodiments, the one or more self-cleaving sites may be the same or different. Similarly, the one or more protease sites may be the same or different.

In some embodiments, the polycistronic construct may be in the form of a vector. Any type of vector suitable for introduction of nucleotide sequences into a host cell can be used, including, for example, plasmids, adenoviral vectors, adenoviral-associated vectors, retroviral vectors, lentiviral vectors, phages, and homology-directed repair (HDR)-based donor vectors.

### 4. Safety Switch

In some embodiments, a safety switch is included in the vector or inserted in a gene locus and allows for controlled killing of the cells in the event of cytotoxicity or other negative consequences to the recipient, thus increasing the safety of cell-based therapies, including those using tolerogenic factors. Detailed descriptions of exemplary safety switches can be found, for example, in WO2021/146627, PCT Application No. PCT/US21/54326 filed on Oct. 9, 2021, and U.S. Provisional Application Nos. 63/222,954 filed on Jul. 16, 2021, 63/282,961 filed on Nov. 24, 2021; the disclosures such as the sequence listings, specifications, and figures are herein incorporated in their entirety.

In certain embodiments, the vector may comprise one or more expression cassettes each comprising a nucleotide sequence encoding a safety switch. A safety switch can be used in the polycistronic vector of the present technology to induce death or apoptosis of host cells containing the polycistronic vector, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are disclosed in, for example, Düzgüneş, Origins of Suicide Gene Therapy (2019); Düzgüneş (eds), Suicide Gene Therapy. Methods in Molecular Biology, vol. 1895 (Humana Press, New York, NY) (for HSVtk, cytosine deaminase, nitroreductase, purine nucleoside phosphorylase, and horseradish peroxidase); Zhou and Brenner, Exp Hematol 44 (11): 1013-1019 (2016) (for iCaspase9); Wang et al., Blood 18 (5): 1255-1263 (2001) (for huEGFR); U.S. Patent Application Publication No. 20180002397 (for HER1); and Philip et al., Blood124 (8): 1277-1287 (2014) (for RQR8).

In some embodiments, the safety switch can cause cell death in a controlled manner, for example, in the presence of a drug or prodrug or upon activation by a selective exogenous compound. In some embodiments, expression of the safety switch is regulated either by a promoter of the vector, in the case of genomic location-independent transcriptional regulation, or by an endogenous promoter, in the case of site-specific integration of the construct into target gene locus.

In some embodiments, the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting a cell comprising the vector with the drug or prodrug. In some cases, the safety switch is HSVtk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin-inducible caspase 9 variant is called rapaCasp9. See Stavrou et al., Mol. Ther. 26 (5): 1266-1276 (2018). Thus, iCasp9 can be used as a safety switch in the present polycistronic vector to achieve controlled killing of the host cells.

In some embodiments, the safety switch may be a membrane-expressed protein which allows for cell depletion after administration of a specific antibody to that protein. Safety switches of this category may include, for example, CCR4, CD16, CD19, CD20, CD30, EGFR, GD2, HER1, HER2, MUC1, PSMA, or RQR8. These proteins may have surface epitopes that can be targeted by specific antibodies.

In some embodiments, the safety switch comprises CCR4, which can be recognized by an anti-CCR4 antibody. Non-limiting examples of suitable anti-CCR4 antibodies include mogamulizumab and biosimilars thereof.

In some embodiments, the safety switch comprises CD16 or CD30, which can be recognized by an anti-CD16 or anti-CD30 antibody. Non-limiting examples of such anti-CD16 or anti-CD30 antibody include AFM13 and biosimilars thereof.

In some embodiments, the safety switch comprises CD19, which can be recognized by an anti-CD19 antibody. Non-limiting examples of such anti-CD19 antibody include MOR208 and biosimilars thereof.

In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-RLIb, and biosimilars thereof. Cells that express the safety switch are thus CD20-positive and can be targeted for killing through administration of an anti-CD20 antibody as described.

In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof.

In some embodiments, the safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-RLIc, and biosimilars thereof.

In some embodiments, the safety switch comprises HER1, which can be recognized by an anti-HER1 antibody. Non-limiting examples of such anti-HER1 antibody include cetuximab and biosimilars thereof.

In some embodiments, the safety switch comprises HER2, which can be recognized by an anti-HER2 antibody. Non-limiting examples of such anti-HER2 antibody include margetuximab, trastuzumab, TrasGEX, and biosimilars thereof.

In some embodiments, the safety switch comprises MUC1, which can be recognized by an anti-MUC1 antibody. Non-limiting examples of such anti-MUC1 antibody include gatipotuzumab and biosimilars thereof.

In some embodiments, the safety switch comprises PSMA, which can be recognized by an anti-PSMA antibody. Non-limiting examples of such anti-PSMA antibody include KM2812 and biosimilars thereof.

In some embodiments, the safety switch comprises RQR8, which can be recognized by an anti-RQR8 antibody. Non-limiting examples of such anti-RQR8 antibody include rituximab and biosimilars thereof.

In some embodiments, the safety switch comprises HSVtk and a membrane-expressed protein, for example, CCR4, CD16, CD19, CD20, CD30, EGFR, GD2, HER1, HER2, MUC1, PSMA, and RQR8.

In some embodiments, wherein the modified immune evasive cell is inserted with a transgene encoding CD47 or wherein the vector comprises a CD47 coding sequence, a CD47-SIRPα blockade agent can be used as a safety switch.

Without wishing to be bound by theory, it is believed that the modifications of the engineered cells “cloak” them from the recipient immune system's effector cells that are responsible for the clearance of infected, malignant or non-self cells. “Cloaking” of a cell from the immune system allows for existence and persistence of specific cells, e.g., allogeneic cells within the body. In some instances, engineered cells described herein may no longer be therapeutically effective or may induce undesired adverse effects in the recipient. Non-limiting examples of an adverse event include hyperproliferation, transformation, tumor formation, cytokine release syndrome, GVHD, immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD), and the like. Controlled removal of the engineered cells from the body is crucial for patient safety and can be achieved by uncloaking the cells from the immune system. Uncloaking serves as a safety switch and can be achieved through the downregulation of the immunosuppressive molecules or the upregulation of immune signaling molecules. The level of expression of any of the immunosuppressive molecules described can be controlled on the protein level, mRNA level, or DNA level in the cells. Similarly, the level of expression of any of the immune signaling molecules described can be controlled on the protein level, mRNA level, or DNA level in the cells. In an example of uncloaking Hypo-Immune cells Through Genetic, Post-Transcriptional, and Post-Translational Regulation, hypoimmunity is achieved through the overexpression of hypoimmune molecules such as CD47, complement inhibitors accompanied with the repression or genetic disruption of the HLA-I and HLA-II loci. These modifications cloak the cell from the immune system's effector cells that are responsible for the clearance of infected, malignant or non-self cells, such as T-cells, B-cells, NK cells and macrophages. Cloaking of a cell from the immune system allows for existence and persistence of allogeneic cells within the body. Removal of the engineered cells from the body is crucial for patient safety and can be achieved by uncloaking the cells from the immune system. Uncloaking serves as a safety switch and can be achieved through the downregulation of the hypoimmune molecules (for example CD47, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, and Serpinb9) or the upregulation of immune signaling molecules (for example B2M, MIC-A/B, HLA-A, HLA-B, HLA-C, HLA-D, HLA-E, RFXANK, CTLA-4, PD-1, CIITA, HLA-DP, HLA-DM, HLA-DOA, HLA-DOB, HLA-DQ, HLA-DR, and ligands of NKG2D (e.g., MICA, MICB, RAET1E/ULBP4, RAET1G/ULBP5, RAET1H/ULBP2, RAET1/ULBP1, RAETIL/ULBP6, or RAETIN/ULBP3). Either of these activities, or a combination of the both, will avail the cell to native effector cells, resulting in clearance of the allogeneic cell.

In some embodiments, upon contacting the cells with a CD47-SIRPα blockade agent, the cells are recognized by the recipient's immune system. In some embodiments, the engineered cells express the immunosuppressive factor CD47 such that the cells are immune evasive or have reduced immunogenicity until one or more CD47-SIRPα blockade agents are administered to the recipient. In the presence of a CD47-SIRPα blockade agent, the cells are uncloaked and are recognized by immune cells to be targeted by cell death or clearance.

In some embodiments, administration of a CD47-SIRPα blockade agent to the recipient facilitates phagocytosis, cell clearance and/or cell death of these cells and derivatives thereof (e.g., progeny cells). In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. Such CD47-SIRPα blockade agents are useful as safety switches to modulate the activity of administered or engrafted cells, thereby improving the safety of these cell-based therapies.

### CD47-SIRPα Blockade Agents

In some embodiments, a recipient subject is treated with a therapeutic agent that inhibits or blocks the interaction of CD47 and SIRPα. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some aspects, the CD47-SIRPα blockade agent reduces in the recipient patient the number of cells exogenously expressing CD47 polypeptides, including, but not limited to, cells that also exogenously express one or more chimeric antigen receptors. In some embodiments, the CD47-SIRPα blockade agent decreases the number of CD47-expressing immune evasive cells in the patient, independent of the level of CAR expression by such cells. In some instances, the level of CAR expression by the cells is less (e.g., 1%, 5%, 10%, 15%, 20%, 25%, 30%, 40%, 50%, 60%, 70%, 80%, 90%, 95%, or 99% less) than the level by a control CAR-T cell, such as, but not limited to, a tisagenlecleucel biosimilar, tisagenlecleucel surrogate and the like. In certain instances, the level of CAR expression by the cells is more (e.g., 1%, 5%, 10%, 15%, 20%, 25%, 30%, 40%, 50%, 60%, 70%, 80%, 90%, 95%, 100%, 150%, 200%, 300%, or a higher percentage more) than the level by a control CAR-T cell, such as, but not limited to, a tisagenlecleucel biosimilar, tisagenlecleucel surrogate and the like.

### (i) CD47-Binding Blockade Agents

In some embodiments, the CD47-SIRPα blockade agent is an agent that binds CD47. The agent can be a CD47 blocking, neutralizing, antagonizing or interfering agent. In some embodiments, the CD47-SIRPα blockade agent is selected from a group that includes an antibody or fragment thereof that binds CD47, a bispecific antibody that binds CD47, and an immunocytokine fusion protein that binds CD47.

Useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Useful bispecific antibodies that bind CD47 comprise a first antigen binding domain that binds CD47 and a second antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, c-Met, PTHR2, HAVCR2 (TIM3), and an antigen expressed on a cancer cell.

In some embodiments, a CD47-SIRPα blockade agent is an immunocytokine fusion protein comprising a cytokine and either an antigen binding domain, antibody, or fragment thereof that binds CD47.

Detailed descriptions of exemplary CD47 binding molecules (e.g., antigen binding domains, antibodies, nanobodies, diabodies, antibody mimetic proteins (e.g., DARPins), and fragments thereof that recognize or bind CD47) including sequences of the heavy chain, light chain, VH region, VL region, CDRs, and framework regions can be found, for example, in WO2009091601; WO2011143624; WO2013119714; WO201414947; WO2014149477; WO2015138600; WO2016033201; WO2017049251; Pietsch et al., Blood Cancer J, 2017, 7 (2), e536; van Brommel et al., 2018, 7 (2), e1386361; Yu et al., Biochimie, 2018, 151, 54-66; and Andrechak et al., Phil Trans R Soc, 2019, 374, 20180217; the disclosures such as the sequence listings, specifications, and figures are herein incorporated in their entirety.

### (ii) SIRPα-Binding Blockade Agents

In some embodiments, the CD47-SIRPα blockade agent administered to the recipient subject is an agent that binds SIRPα. The agent can be an SIRPα blocking, neutralizing, antagonizing or inactivating agent. In some embodiments, the CD47-SIRPα blockade agent is selected from a group that includes, but is not limited to, an antibody or fragment thereof that binds SIRPα, a bispecific antibody that binds SIRPα, and an immunocytokine fusion protein that bind SIRPα.

Useful antibodies or fragments thereof that bind SIRPα can be selected from a group that includes, but is not limited to, ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University). In some embodiments, the antibody or fragment thereof does not compete for SIRPα binding with an antibody selected from a group that includes ADU-1805, CC-95251, OSE-172 (BI 765063), KWAR23, and P362. In some embodiments, the antibody or fragment thereof competes for SIRPα binding with an antibody selected from a group that includes ADU-1805, CC-95251, OSE-172 (BI 765063), KWAR23, and P362.

In some embodiments, the antibody or fragment thereof that binds SIRPα is selected from a group that includes a single-chain Fv fragment (scFv) against SIRPα, a Fab against SIRPα, a VHH nanobody against SIRPα, a DARPin against SIRPα, and variants thereof. In some embodiments, the scFv against SIRPα, a Fab against SIRPα, and variants thereof are based on the antigen binding domains of any of the antibodies selected from a group that includes ADU-1805, CC-95251, OSE-172 (BI 765063), KWAR23, and P362.

In some embodiments, the bispecific antibody that binds SIRPα and an antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, C-Met, PTHR2, HAVCR2 (TIM3), and an antigen expressed on a cancer cell. In some instances, the bispecific antibody binds SIRPα and a tumor associated antigen. In some instances, the bispecific antibody binds SIRPα and an antigen expressed on the surface of an immune cell.

In some embodiments, a CD47-SIRPα blockade agent is an immunocytokine fusion protein comprises a cytokine and either an antigen binding domain, antibody, or fragment thereof that binds SIRPα.

Detailed descriptions of exemplary SIRPα binding molecules (e.g., antigen binding domains, antibodies, nanobodies, diabodies, antibody mimetic proteins (e.g., DARPins), and fragments thereof that recognize or bind SIRPα) including sequences of the heavy chain, light chain, VH region, VL region, CDRs, and framework regions can be found, for example, in WO2019226973; WO2018190719; WO2018057669; WO2017178653; WO2016205042; WO2016033201; WO2016022971; WO2015138600; and WO2013109752; the disclosures including the sequence listings, specifications, and figures are herein incorporated in their entirety.

(iii) CD47- and/or SIRP-Containing Fusion Proteins

As disclosed herein, a CD47-SIRPα blockade agent can comprise a CD47-containing fusion protein that binds SIRPα. In some embodiments, such CD47-containing fusion protein that binds SIRPα is an agent administered to a recipient subject. In some embodiments, the CD47-containing fusion protein comprises a CD47 extracellular domain or variants thereof that bind SIRPα. In some embodiments, the fusion protein comprises an Fc region. Detailed descriptions of exemplary CD47 fusion proteins including sequences can be found, for example, in US20100239579, the disclosure is herein incorporated in its entirety including the sequence listing, specification, and figure.

In some embodiments, a CD47-SIRPα blockade agent can comprise an SIRPα-containing fusion protein that binds CD47. The sequence of SIRPα is set forth in SEQ ID NO:13 (UniProt P78324). Generally, SIRPα-containing fusion proteins comprise a domain of SIRPα including any one of (a) the immunoglobulin-like domain of human SIRPα (e.g., the membrane distal (D1) loop containing an IgV domain of SIRP, (b) the first membrane proximal loop containing an IgC domain, and (c) the second membrane proximal loop containing an IgC domain). In some instances, the SIRPα domain binds CD47. In some embodiments, the SIRPα-containing fusion protein comprises an SIRPα extracellular domain or variants thereof that bind CD47. In some embodiments, the fusion protein comprises an Fc region, including but not limited to a human IgG1 Fc region (e.g., UniProtKB/Swiss-Prot P01857, SEQ ID NO: 14) or IgG4 Fc region (e.g., UniProt P01861, SEQ ID NO: 15; GenBank CAC20457.1, SEQ ID NO:16). Optionally, the Fc region may comprise one or more substitutions. In some embodiments, the SIRPα-containing fusion proteins are selected from a group that includes TTI-621 (Trillium Therapeutics), TTI-622 (Trillium Therapeutics), and ALX148 (ALX Oncology). TTI-621 (SEQ ID NO:17) is a fusion protein made up of the N-terminal V domain of human SIRPα fused to a human IgG1 Fc region (Petrova et al. Clin Cancer Res 23 (4): 1068-1079 (2017)), while TTI-622 (SEQ ID NO:18) is a fusion protein made up of the N-terminal V domain of human SIRPα fused to a human IgG4 Fc region with a single substitution.

TABLE 4
Exemplary sequences of SIRPα, IgG1/IgG4, and CD47 fusion proteins

| SEQ ID NO: | Sequence | Description |
|---|---|---|
| 13 | MEPAGPAPGRLGPLLCLLLAASCAWSGVAGE | SIRPα (UniProt P78324) |
| | EELQVIQPDKSVSVAAGESAILHCTVTSLIPVG | |
| | PIQWFRGAGPARELIYNQKEGHFPRVTTVSES | |
| | TKRENMDFSISISNITPADAGTYYCVKFRKGS | |
| | PDTEFKSGAGTELSVRAKPSAPVVSGPAARAT | |
| | PQHTVSFTCESHGFSPRDITLKWFKNGNELSD | |
| | FQTNVDPVGESVSYSIHSTAKVVLTREDVHSQ | |
| | VICEVAHVTLQGDPLRGTANLSETIRVPPTLE | |
| | VTQQPVRAENQVNVTCQVRKFYPQRLQLTW | |
| | LENGNVSRTETASTVTENKDGTYNWMSWLL | |
| | VNVSAHRDDVKLTCQVEHDGQPAVSKSHDL | |
| | KVSAHPKEQGSNTAAENTGSNERNIYIVVGV | |
| | VCTLLVALLMAALYLVRIRQKKAQGSTSSTR | |
| | LHEPEKNAREITQVQSLDTNDITYADLNLPKG | |
| | KKPAPQAAEPNNHTEYASIQTSPQPASEDTLT | |
| | YADLDMVHLNRTPKQPAPKPEPSFSEYASVQ | |
| | VPRK | |
| 14 | ASTKGPSVFPLAPSSKSTSGGTAALGCLVKDY | Human IgG1 |
| | FPEPVTVSWNSGALTSGVHTFPAVLQSSGLYS | (UniProtKB/Swiss-Prot |
| | LSSVVTVPSSSLGTQTYICNVNHKPSNTKVDK | P01857) |
| | KVEPKSCDKTHTCPPCPAPELLGGPSVFLFPP | |
| | KPKDTLMISRTPEVTCVVVDVSHEDPEVKEN | |
| | WYVDGVEVHNAKTKPREEQYNSTYRVVSVL | |
| | TVLHQDWLNGKEYKCKVSNKALPAPIEKTIS | |
| | KAKGQPREPQVYTLPPSRDELTKNQVSLTCL | |
| | VKGFYPSDIAVEWESNGQPENNYKTTPPVLD | |
| | SDGSFFLYSKLTVDKSRWQQGNVFSCSVMHE | |
| | ALHNHYTQKSLSLSPGK | |
| 15 | ASTKGPSVFPLAPCSRSTSESTAALGCLVKDY | Human IgG4 (UniProt |
| | FPEPVTVSWNSGALTSGVHTFPAVLQSSGLYS | P01861) |
| | LSSVVTVPSSSLGTKTYTCNVDHKPSNTKVD | |
| | KRVESKYGPPCPSCPAPEFLGGPSVFLFPPKPK | |
| | DTLMISRTPEVTCVVVDVSQEDPEVQFNWYV | |
| | DGVEVHNAKTKPREEQFNSTYRVVSVLTVLH | |
| | QDWLNGKEYKCKVSNKGLPSSIEKTISKAKG | |
| | QPREPQVYTLPPSQEEMTKNQVSLTCLVKGF | |
| | YPSDIAVEWESNGQPENNYKTTPPVLDSDGSF | |
| | FLYSRLTVDKSRWQEGNVFSCSVMHEALHN | |
| | HYTQKSLSLSLGK | |
| 16 | ASFKGPSVFPLVPCSRSTSESTAALGCLVKDY | Human IgG4 (GenBank |
| | FPEPVTVSWNSCALTSGVHTFPAVLQSSGLYS | CAC20457.1) |
| | LSSVVTVPSSSLGTKTYTCNVDHKPSNTKVD | |
| | KRVESKYGPPCPSCPAPEFLGGPSVFLFPPKPK | |
| | DTLMISRTPEVTCVVVDVSQEDPEVQFNWYV | |
| | DGVEVHNAKTKPREEQFNSTYRVVRVLTVLH | |
| | QDWLNGKEYKCKVSNKGLPSSIEKTISKAKG | |
| | QPREPQVYTLPPSQEEMTKNQVSLTCLVKGF | |
| | YPSDIAVEWESNGQPEDNYKTTPPVLDSDGSF | |
| | FLYSRLTVDKSRWQEGNVFSCSVMHEALHN | |
| | HYTQKSLSLSPGK | |
| 17 | EEELQVIQPDKSVSVAAGESAILHCTVTSLIPV | TTI-621 |
| | GPIQWFRGAGPARELIYNQKEGHFPRVTTVSE | |
| | STKRENMDFSISISNITPADAGTYYCVKFRKG | |
| | SPDTEFKSGAGTELSVRAKPSDKTHTCPPCPA | |
| | PELLGGPSVFLFPPKPKDTLMISRTPEVTCVVV | |
| | DVSHEDPEVKFNWYVDGVEVHNAKTKPREE | |
| | QYNSTYRVVSVLTVLHQDWLNGKEYKCKVS | |
| | NKALPAPIEKTISKAKGQPREPQVYTLPPSRDE | |
| | LTKNQVSLTCLVKGFYPSDIAVEWESNGQPE | |
| | NNYKTTPPVLDSDGSFFLYSKLTVDKSRWQQ | |
| | GNVFSCSVMHEALHNHYTQKSLSLSPGK | |
| 18 | EEELQVIQPDKSVSVAAGESAILHCTVTSLIPV | TTI-622 |
| | GPIQWFRGAGPARELIYNQKEGHFPRVTTVSE | |
| | STKRENMDFSISISNITPADAGTYYCVKFRKG | |
| | SPDTEFKSGAGTELSVRAKPSESKYGPPCPPCP | |
| | APEFLGGPSVFLFPPKPKDTLMISRTPEVTCVV | |
| | VDVSQEDPEVQFNWYVDGVEVHNAKTKPRE | |
| | EQFNSTYRVVSVLTVLHQDWLNGKEYKCKV | |
| | SNKGLPSSIEKTISKAKGQPREPQVYTLPPSQE | |
| | EMTKNQVSLTCLVKGFYPSDIAVEWESNGQP | |
| | ENNYKTTPPVLDSDGSFFLYSRLTVDKSRWQ | |
| | EGNVFSCSVMHEALHNHYTQKSLSLSLGK | |

TTI-621, TTI-622, and other related fusion proteins are disclosed in PCT Publ. No. WO14/94122, the contents of which are hereby incorporated by reference herein with regard to said proteins. AL148 is a fusion protein made up of the N-terminal D1 domain of SIRPα fused to a modified human IgG1 Fc domain (Kauder et al. PLOS One (13 (8): e0201832 (2018)). Detailed descriptions of exemplary SIRPα fusion proteins including sequences can be found, for example, in PCT Publ. Nos. WO14/94122; WO16/23040; WO17/27422; WO17/177333; and WO18/176132, the disclosures of which are hereby incorporated herein in their entirety, including the sequence listings, specifications, and figures.

SIRPα-containing fusion proteins, including TTI-621, are being developed for the treatment of cancer, such as hematologic malignancies, alone or in combination with other cancer therapy drugs. A phase 1 trial evaluating dosage and safety (NCT02663518) of intravenous TTI-621 administration in patients with relapsed/refractory hematologic malignancies and selected solid tumors found that TTI-621 was well tolerated and demonstrated activity both as a monotherapy and in combination with other cancer treatment agents (Ansell et al. Clin Cancer Res 27 (8): 2190-2199 (2021)). In the initial escalation phase, subjects received TTI-621 at dosages of 0.05, 0.1, 0.3, 1, 3, and 10 mg/kg to evaluate safety and maximum tolerated dose (MTD). In the expansion phase, subjects received the MTD of 0.2 mg/kg as a monotherapy or 0.1 mg/kg in combination with rituximab or nivolumab.

### 5. Site-Directed Genomic Insertion

In some embodiments, the one or more transgenes encoding one or more tolerogenic factors and/or regulatory elements may be delivered into a host cell for targeted genomic insertion in the form of a vector, e.g., by viral transduction. The delivery vector can be any type of vector suitable for introduction of nucleotide sequences into a cell, including, for example, plasmids, adenoviral vectors, adeno-associated viral (AAV) vectors such as an AAV6 vector and an AAV9 vector, retroviral vectors, lentiviral vectors (e.g., pseudotyped, self-inactivating lentiviral vectors), phages, and HDR-based donor vectors. Additional AAV vectors for gene delivery are disclosed in, for example, Wang et al., “Adeno-associated virus vector as a platform for gene therapy deliver,” Nature Reviews Drug Discovery 18:358-378 (2019), the disclosure is incorporated herein by reference in its entirety.

The different components may be introduced into a cell together or separately, and may be delivered in a single vector or multiple vectors. The vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods. Insertion of the one or more transgenes encoding one or more tolerogenic factors and/or regulatory elements into an endogenous B2M and/or CIITA gene locus may be carried out using any of the site-directed insertion methods and/or systems disclosed herein, including, for example, zinc finger nucleases (ZFNs), transcription activator-like effector nucleases (TALENs), meganucleases, transposases, and clustered regularly interspaced short palindromic repeat (CRISPR)/Cas systems. In cases where a homology directed repair (HDR)-based approach as disclosed is used, the transgene is usually flanked by homology arms (i.e., left homology arm (LHA) and right homology arm (RHA)) that are specific to the target site of insertion. The homology arms are specifically designed for the target genomic locus for the fragment to serve as a template for HDR. The length of each homology arm is generally dependent on the size of the insert being introduced, with larger insertions requiring longer homology arms.

### B. Reducing or Eliminating the Expression of MHC I/MHC II Molecules

In some embodiments, the methods disclosed herein for generating an engineered immune evasive cell or a population thereof comprise reducing or eliminating the expression of one or more MHC class I (MHC I) and/or one or more MHC class II (MHC II) molecules to reduce the immunogenicity of these cells, in order to reduce potential graft-versus-host risks after infusion into the recipient or risks of being eliminated by the recipient's innate immune system.

MHC I and/or MHC II genes encode cell surface molecules specialized to present antigenic peptides to immune cells. Reduced expression of one or more MHC I and/or one or more MHC II molecules in allogeneic cells may prevent recognition of these cells by the immune cells of the recipient and thus rejection of the graft. The MHC in humans is called human leukocyte antigen (HLA). Class I HLA (corresponding to MHC class I) include the HLA-A, HLA-B, and HLA-C genes, and Class II HLA (corresponding to MHC class II) include the HLA-DR, HLA-DQ, HLA-DP, HLA-DM, and HLA-DO genes.

In some embodiments, reduced or eliminated expression of one or more MHC I molecules is caused by reducing or eliminating expression of B2M, TAP1, or both. In some embodiments, reduced or eliminated expression of one or more MHC II molecules is caused by reducing or eliminating expression of CIITA, CD74, or both.

Thus, in some embodiments, expression can be reduced via a gene and/or function thereof, RNA expression and/or function thereof, protein expression and/or function thereof, reduction of surface expression, reduction of trafficking, or a combination thereof.

In some embodiments, reduced expression of a target is such that expression in an engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a source cell (i.e. a cell of the same cell type) prior to being engineered to reduce expression of the target. In some embodiments, reduced expression of a target is such that expression in an engineered cell is reduced to a level that is about 60% or less (such as any of about 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a reference cell or a reference cell population (such as a cell or population of the same cell type). In some embodiments, reduced expression of a target is such that expression in an engineered cell is reduced to a level that is at or less than a measured level of expression. In some embodiments, the level of a target is assessed in an engineered cell, a reference cell, or reference cell population in a stimulated or non-stimulated state. In some embodiments, the level of a target is assessed in an engineered cell, a reference cell, or reference cell population in a stimulated state such that the target is expressed (or will be if it is a capability of the cell in response to the stimulus). In some embodiments, the stimulus represents an in vivo stimulus.

In some embodiments, a genetic editing system is used to modify one or more target polynucleotide sequences that regulate the expression of either MHC class I molecules, MHC class II molecules, or both MHC class I molecule and MHC class II molecules. In certain embodiments, the genome of the cell has been altered to reduce or delete components require or involved in facilitating HLA expression, such as expression of one or more MHC class I molecules and/or one or more MHC class II molecules on the surface of the cell. For instance, in some embodiments, B2M, a component of MHC class I molecules, is reduced or eliminated in the cell, thereby reducing or eliminating the cell surface expression of one or more MHC class I molecules by the engineered cell. In some embodiments, TAP1 is reduced or eliminated in the cell, thereby reducing or eliminating the expression of one or more MHC class I molecules by the engineered cell. In some embodiments, TAP1 is reduced or eliminated in the cell, thereby reducing or eliminating the cell surface expression of one or more MHC class I molecules by the engineered cell.

In some embodiments, reduction of the expression of one or more MHC class I molecules and/or one or more MHC class II molecules can be accomplished, for example, by one or more of the following: (1) removal of B2M, which will reduce surface trafficking of all MHC class I molecules; (2) removal of TAP1, which will disrupt the expression of HLA-A, -B, and -C genes; and/or (3) deletion of one or more components of the MHC enhanceosomes, such as CD74, and CIITA that are critical for MHC class II expression.

In certain embodiments, HLA expression is interfered with. In some embodiments, HLA expression is interfered with by targeting transcriptional regulators of HLA expression (e.g., knocking out expression of TAP1, CIITA, and/or CD74), and/or blocking surface trafficking of MHC class I molecules (e.g., knocking out expression of B2M). In some embodiments, expression of HLA class I molecules is interfered with by reducing or knocking out expression of TAP1 and/or B2M. In some embodiments, expression of HLA class II molecules is interfered with by reducing or knocking out expression of CIITA and/or CD74.

### 1. B2M

In some embodiments, the technologies disclosed herein modulate (e.g., reduce or eliminate) the expression of one or more MHC-I genes by targeting and modulating (e.g., reducing or eliminating) expression of the accessory chain B2M. The B2M gene locus is located on chromosome 15 at position 44,711,358-44,718,851 (GRCG38: CM000677.2). In some embodiments, the modulation occurs using a CRISPR/Cas system. In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of B2M expression includes, but is not limited to, reduced transcription, decreased mRNA stability (such as by way of RNAi mechanisms), and reduced protein levels.

By modulating (e.g., reducing or deleting) expression of B2M, surface trafficking of one or more MHC-I molecules is blocked and the cell rendered immune evasive. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells disclosed herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is disclosed in NCBI Gene ID No. 567. In certain cases, the amino acid sequence of B2M is depicted as NCBI GenBank No. BAA35182.1. Additional disclosure of the B2M protein and gene locus can be found in Uniprot No. P61769, HGNC Ref. No. 914, and OMIM Ref. No. 109700.

In some embodiments, the engineered immune evasive cells disclosed herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS: 81240-85644 of Table 15, Appendix 8 of WO2016183041, which is herein incorporated by reference. In some embodiments, an exogenous nucleic acid encoding a polypeptide as disclosed herein (e.g., CD47, or another tolerogenic factor disclosed herein) is inserted at the B2M gene locus, such as exon 2 or another CDS of the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using a lentivirus based viral vector.

Assays to test whether the B2M gene has been inactivated are known and disclosed herein. In some embodiments, the resulting genetic modification of the B2M gene by PCR and the reduction of HLA-I expression can be assayed by FACS analysis. In another embodiment, B2M protein expression is detected using a Western blot of cells lysates probed with antibodies to the B2M protein. In another embodiment, RT-PCR is used to confirm the presence of the inactivating genetic modification.

### 2. CIITA

In some embodiments, the technologies disclosed herein modulate (e.g., reduces or eliminates) the expression of one or more MHC II genes by targeting and modulating (e.g., reducing or eliminating) Class II transactivator (CIITA) expression. The CIITA gene locus is located on chromosome 16 at position 10,866,222-10,943,021 (GRCH38: CM000678.2). In some embodiments, the modulation occurs using a CRISPR/Cas system. In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of CIITA expression includes, but is not limited to, reduced transcription, decreased mRNA stability (such as by way of RNAi mechanisms), and reduced protein levels.

CIITA is a member of the LR or nucleotide binding domain (NBD) leucine-rich repeat (LRR) family of proteins and regulates the transcription of one or more MHC I/MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following: HLA-DP, HLA-DM, HLA-DOA, HLA-DOB, HLA-DQ, and HLA-DR.

In some embodiments, the cells disclosed herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is disclosed in NCBI Gene ID No. 4261. In certain embodiments, the amino acid sequence of CIITA is depicted as NCBI GenBank No. AAA88861.1. Additional disclosure of the CIITA protein and gene locus can be found in Uniprot No. P33076, HGNC Ref. No. 7067, and OMIM Ref. No. 600005.

In some embodiments, the engineered cells disclosed herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Table 12, Appendix 5 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., CD47, or another tolerogenic factor disclosed herein) is inserted at the CIITA gene locus such as exon 3 or another CDS of the CIITA gene.

Assays to test whether the CIITA gene has been inactivated are known and disclosed herein. In some embodiments, the resulting genetic modification of the CIITA gene by PCR and the reduction of HLA-II expression can be assayed by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### 3. TAP1

In some embodiments, the technologies disclosed herein modulate (e.g., reduce or eliminate) the expression of one or more MHC I genes by targeting and modulating (e.g., reducing or eliminating) expression of TAP1, an ER-resident peptide transporter. In some embodiments, the modulation occurs using a CRISPR/Cas system. In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of TAP1 expression includes, but is not limited to, reduced transcription, decreased mRNA stability (such as by way of RNAi mechanisms), and reduced protein levels.

TAP1 is a transcriptional regulator. When TAP1 is disrupted, expression of MHC class I genes (HLA-A, HLA-B, and HLA-C) is similarly disrupted. Thus, in some embodiments, decreasing expression of, or knocking out, TAP1 interferes with expression of MHC class I genes, such that expression of one or more MHC class I molecules (HLA-A, HLA-B and HLA-C) is reduced or decreased by virtue of reduced expression of the genes encoding the same. Thus, in some embodiments, reducing expression of one or more MHC class I molecules is achieved by reducing expression of MHC class I encoding genes, such as by reducing expression of, or knocking out, TAP1. In some embodiments, the engineered cell has a reduced ability to induce an innate and/or an adaptive immune response in a recipient subject.

In some embodiments, the target polynucleotide sequence of the present disclosure is a variant of TAP1. In some embodiments, the target polynucleotide sequence is a homolog of TAP1. In some embodiments, the target polynucleotide sequence is an ortholog of TAP1.

In some embodiments, decreased or eliminated expression of TAP1 reduces or eliminates expression of one or more of the following MHC I molecules: HLA-A, HLA-B, and HLA-C.

In some embodiments, the engineered immune evasive cells disclosed herein comprise a genetic modification targeting the TAP1 gene. In some embodiments, the genetic modification targeting the TAP1 gene by the rare-cutting endonuclease comprises a Cas protein or a polynucleotide encoding a Cas protein, and at least one guide ribonucleic acid sequence for specifically targeting the TAP1 gene. In some embodiments, the at least one guide ribonucleic acid sequence for specifically targeting the TAP1 gene is selected from the group consisting of SEQ ID NOS: 182814-188371 of Table 24, Appendix 17 of WO2016183041, which is herein incorporated by reference.

Assays to test whether the TAP1 gene has been inactivated are known and disclosed herein. In some embodiments, the resulting genetic modification of the TAP1 gene by PCR and the reduction of HLA-I expression can be assayed by FACS analysis. In another embodiment, TAP1 protein expression is detected using a Western blot of cells lysates probed with antibodies to the TAP1 protein. In another embodiment, RT-PCR is used to confirm the presence of the inactivating genetic modification.

In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, one or more tolerogenic factors are inserted into the B2M locus. In some embodiments, inserting the exogenous nucleic acid encoding the tolerogenic factor at the B2M locus disrupts the expression of the B2M gene. In some embodiments, the engineered cells having reduced expression of one or more MHC I genes have a reduced ability to induce an immune response in a recipient subject. In some embodiments, reduced expression of B2M reduces or eliminates expression of one or more of the HLA-A, HLA-B, and HLA-C genes.

In some embodiments, one or more tolerogenic factors are inserted into the CIITA locus. In some embodiments, inserting the exogenous nucleic acid encoding the tolerogenic factor at the CIITA locus disrupts the expression of the CIITA gene. In some embodiments, the engineered cells having reduced expression of one or more MHC II genes have a reduced ability to induce an immune response in a recipient subject. In some embodiments, reduced expression of CIITA reduces or eliminates expression of one or more of the HLA-DR, HLA-DQ, HLA-DP, HLA-DM, and HLA-DO genes.

In some embodiments, one or more transgenes encoding one or more tolerogenic factors are inserted in B2M locus, and one or more transgenes encoding one or more tolerogenic factors are inserted in CIITA locus such that the expression of both B2M and CIITA genes is disrupted thereby to reduce or eliminate the expression of one or more MHC I and one or more MHC II molecules. In some embodiments, different transgenes encoding different tolerogenic factors are inserted in B2M locus and CIITA locus. In some embodiments, the same transgene encoding the same tolerogenic factor is inserted in B2M locus and CIITA locus. For example, the transgene encoding CD47 is inserted in both B2M locus and CIITA locus.

In some embodiments, one or more transgenes encoding one or more tolerogenic factors are inserted in the B2M locus only to disrupt B2M expression, and an additional genetic modification targeting the CIITA locus occurs through insertion-deletion (indel) modifications of the CIITA locus, for example, by using the CRISPR/Cas system as disclosed herein. In some embodiments, one or more transgenes encoding one or more tolerogenic factors are inserted in the B2M locus only to disrupt B2M expression, and CIITA and/or CD74 is knocked out. In some embodiments, one or more transgenes encoding one or more tolerogenic factors are inserted in the CIITA locus only to disrupt CIITA expression, and an additional genetic modification targeting the B2M locus occurs through insertion-deletion (indel) modifications of the B2M locus, for example, by using the CRISPR/Cas system as disclosed herein. In some embodiments, one or more transgenes encoding one or more tolerogenic factors are inserted in the CIITA locus only to disrupt CIITA expression, and B2M and/or TAP1 is knocked out. The B2M, TAP1, CD74, and/or CIITA knockout can occur at one allele, or both alleles, of the respective gene locus. In these embodiments, the engineered immune evasive cells have reduced expression of one or more MHC I and/or one or more MHC II genes (HLA I and/or HLA II in humans) as a result of B2M, TAP1, CD74, and/or CIITA disruption, deletion or knockout. In some embodiments wherein one or more transgenes encoding one or more tolerogenic factors are inserted in one or more safe harbor loci, any or all of B2M, TAP1, CD74, and CIITA are knocked out or knocked down to reduce or eliminate the expression of one or more MHC I and/or one or more MHC II molecules.

In some embodiments, RNA interference is employed to reduce or inhibit the expression of B2M, TAP1, CD74, and/or CIITA. For example, RNA silencing or RNA interference (RNAi) can be used to knock down (e.g., decrease, eliminate, or inhibit) the expression of B2M, TAP1, CD74, or CIITA. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knock down methods recognized by those skilled in the art. Reagents for RNAi including sequence specific shRNAs, siRNA, miRNAs and the like are commercially available. For instance, B2M, TAP1, CD74, or CIITA can be knocked down in a pluripotent stem cell by introducing a B2M, TAP1, CD74, or CIITA siRNA or transducing a B2M, TAP1, CD74, or CIITA shRNA-expressing virus into the cell.

In some embodiments, at least 50%, at least 55%, at least 60%, at least 65%, at least 70%, at least 75%, at least 80%, at least 85%, at least 90%, at least 95%, or 100% of the engineered cells generated by methods according to various embodiments of the present technology have reduced expression of one or more MHC I molecules and/or one or more MHC II molecules. In some embodiments, at least 50%, at least 55%, at least 60%, at least 65%, at least 70%, at least 75%, at least 80%, at least 85%, at least 90%, at least 95%, or 100% of the engineered cells generated by methods according to various embodiments of the present technology have reduced expression of B2M, TAP1, CD74, and/or CIITA. In some embodiments, at least 50%, at least 55%, at least 60%, at least 65%, at least 70%, at least 75%, at least 80%, at least 85%, at least 90%, at least 95%, or 100% of the engineered cells generated by methods according to various embodiments of the present technology have B2M, TAP1, CD74, and/or CIITA knockout.

In some embodiments, at least 50%, at least 55%, at least 60%, at least 65%, at least 70%, at least 75%, at least 80%, at least 85%, at least 90%, at least 95%, or 100% of the cells in a population of therapeutic cells have one or more of: (i) increased expression of a tolerogenic factor (e.g., CD47) encoded by a transgene; and (ii) reduced expression of one or more MHC I molecules and/or one or more MHC II molecules. In some embodiments, reduced expression of one or more MHC I and/or MHC II molecules is caused by reducing expression of B2M, TAP1, CD74, and/or CIITA; and/or knocking out B2M, TAP1, CD74, and/or CIITA. In any of these embodiments, the remainder cells in the population (e.g., cells that do not possess all of the disclosed characteristic(s)) may be a heterogeneous population, and each of the remainder cells may possess none, one, or more (but not all) of the characteristics.

### C. Positive Selection for the Tolerogenic Factor

In some embodiments, the methods disclosed herein for generating an engineered immune evasive cell or a population of therapeutic cells comprise selecting for cells containing one or more transgenes encoding one or more tolerogenic factors integrated into an endogenous B2M gene locus, an endogenous CIITA gene locus, or both, wherein integration of the one or more transgenes into the B2M or CIITA gene locus reduces or eliminates the expression of B2M or CIITA, which in turn reduces or eliminates the expression of one or more MHC I molecules and/or one or more MHC II molecules, respectively. In some embodiments, the selecting step comprises positive selection for the tolerogenic factor (e.g., selection for expression of the tolerogenic factor). For example, the expression of one or more tolerogenic factor is detected using a Western blot of cell lysates probed with antibodies against the tolerogenic protein. In another example, RT-PCR is used to confirm the presence of the exogenous mRNA of the tolerogenic factor. In some embodiments, positive selection for the tolerogenic factor (e.g., CD47) comprises selecting for the cells that express the tolerogenic factor on the cell surface, for example, at a higher level than endogenous expression levels of the tolerogenic factor. In some embodiments, positive selection for the tolerogenic factor comprises selecting for the cells that express the tolerogenic factor on the cell surface, for example, at a higher level than endogenous expression levels of the tolerogenic factor if the cell expresses any endogenous tolerogenic factor. In these embodiments, antibodies and/or proteins that bind the tolerogenic factor are selected based on a desired affinity and/or avidity for the tolerogenic factor. For example, antibodies and/or proteins having higher affinities and/or avidities for the tolerogenic factor may be selected over lower affinities and/or avidities for use with cells which express endogenous levels of the tolerogenic factor. In some embodiments, the cells expressing the tolerogenic factor on the cell surface bind to antibodies and/or proteins that bind to the tolerogenic factor. In some embodiments, the cells expressing the tolerogenic factor on the cell surface bind to a column and/or a sorting surface with attached antibodies and/or other proteins binding the tolerogenic factor. In some embodiments, the positive selection for the tolerogenic factor comprises selecting for the cells that express the tolerogenic factor on the cell surface by affinity binding, flow cytometry, and/or immunomagnetic selection using antibodies and/or other proteins that bind the tolerogenic factor. In some embodiments, the tolerogenic factor is CD47.

Several methods of sorting living cells based on whether and/or how much they express or do not express a specific protein on their cell surface are known to those of skill in the art. For example, fluorescence activated cell sorting (FACS) of live cells separates a population of cells into sub-populations based on fluorescent labeling using a flow cytometer. Cells stained using fluorophore-conjugated antibodies to an antigen or marker of interest, such as CD47, can be separated from one another depending on which fluorophore they have been stained with. For example, a cell expressing one cell marker may be detected using an FITC-conjugated antibody that recognizes the marker, and another cell type expressing a different marker could be detected using a PE-conjugated antibody specific for that marker.

Another example of a cell sorting method is magnetic-activated cell sorting (MACS). MACS is a method for separation of various cell populations depending on their surface antigens, such as CD47. The method uses superparamagnetic nanoparticles and columns. The superparamagnetic nanoparticles are of the order of 100 nm. They are used to tag the targeted cells in order to capture them inside the column. The column is placed between permanent magnets so that when the magnetic particle-cell complex passes through it, the tagged cells can be captured. The column consists of steel wool which increases the magnetic field gradient to maximize separation efficiency when the column is placed between the permanent magnets. The MACS method allows cells to be separated by using magnetic nanoparticles coated with antibodies against a particular surface antigen, such as CD47. This causes the cells expressing this antigen to attach to the magnetic nanoparticles. After incubating the beads and cells, the solution is transferred to a column in a strong magnetic field. In this step, the cells attached to the nanoparticles (expressing the antigen) stay on the column, while other cells (not expressing the antigen) flow through. With this method, the cells can be separated positively or negatively with respect to the particular antigen(s). With positive selection, the cells expressing the antigen(s) of interest, which are attached to the magnetic column, are washed out to a separate vessel, after removing the column from the magnetic field. In some embodiments, positive selection methods can be used to distinguish cells expressing endogenous tolerogenic factors from cells expressing tolerogenic factors encoded by transgenes. For example, endogenous expression levels of tolerogenic factors are generally lower than expression levels of tolerogenic factors encoded by transgenes. In these instances, a positive selection method could include contacting the cells with beads conjugated to a first antibody against the tolerogenic factor having a first avidity and/or a first affinity which may bind preferentially to cells expressing both exogenous transgene encoded tolerogenic factors as well as endogenous tolerogenic factor molecules. Any cells expressing mostly the endogenous tolerogenic factor would flow through the column. With negative selection, the antibody used is against surface antigen(s) which are known to be present on cells that are not of interest. After administration of the cells/magnetic nanoparticles solution onto the column the cells expressing these antigens bind to the column and the fraction that goes through is collected, as it contains almost no cells with these undesired antigens.

A cell sorting method that can be used when the engineered immune evasive cells are T cells is the Streptamer technology, which allows reversible isolation and staining of antigen-specific T cells. In principle, the T cells are separated by establishing a specific interaction between the T cell of interest and a molecule that is conjugated to a marker, which enables the isolation. The reversibility of this interaction and the fact that it is performed at low temperatures is the reason for the successful isolation and characterization of functional T cells. Because T cells remain phenotypically and functionally indistinguishable from untreated cells, this method offers new strategies in clinical and basic T cell research. The Streptamer staining principle combines the classic method of T cell isolation by MHC-multimers with the Strep-tag/Strep-Tactin technology. The Strep-tag is a short peptide sequence that displays moderate binding affinity for the biotin-binding site of a mutated streptavidin molecule, called Strep-Tactin. For the Streptamer technology, the Strep-Tactin molecules are multimerized, thus creating a platform for binding to strep-tagged proteins. Further, the Strep-Tactin backbone has a fluorescent label to allow flow cytometry analysis. Incubation of MHC-Strep-tag fusion proteins with the Strep-Tactin backbone results in the formation of an MHC-multimer, which is capable for antigen-specific staining of T cells.

Other examples of cell separation using methodological standards that ensure high purity are rapid and label-free separation procedures based on surface marker density. Exemplary procedures involve the use of an anti-surface marker antibody-immobilized cell-rolling column, that can separate cells depending on the surface marker density of the cell surfaces. Various conditions for the cell-rolling column can be optimized including adjustment of the column tilt angle and medium flow rate.

In some embodiments, at least 50%, at least 55%, at least 60%, at least 65%, at least 70%, at least 75%, at least 80%, at least 85%, at least 90%, at least 95%, or 100% of the cells in a population of therapeutic cells generated by methods according to various embodiments of the present technology have one or more transgenes encoding one or more tolerogenic factors (e.g., CD47) inserted into an endogenous B2M gene locus, an endogenous CIITA locus, or both. In some embodiments, at least 50%, at least 55%, at least 60%, at least 65%, at least 70%, at least 75%, at least 80%, at least 85%, at least 90%, at least 95%, or 100% of the cells in the population have increased expression of the one or more tolerogenic factors (e.g., CD47) encoded by one or more transgenes, and/or reduced expression of one or more MHC I molecules and/or one or more MHC II molecules. In any of these embodiments, the remainder cells in the population do not possess all characteristic(s) including (i) increased expression of one or more exogenous tolerogenic factors (e.g., CD47), (ii) reduced expression of one or more MHC I molecules, and (iii) reduced expression of one or more MHC II molecules.

### II. Gene Editing Systems

In some aspects, the one or more transgenes encoding one or more tolerogenic factors can be integrated into the genome of a host cell (e.g., an allogeneic donor cell) using certain methods and compositions disclosed herein.

### A. Vectors

In some embodiments, a vector herein is a nucleic acid molecule capable of transferring or transporting another nucleic acid molecule, including into the cell or into the genome of a cell. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. Non-viral vectors may require a delivery vehicle to facilitate entry of the nucleic acid molecule into a cell.

A viral vector can comprise a nucleic acid molecule that includes virus-derived nucleic acid elements that typically facilitate transfer of the nucleic acid molecule or integration into the genome of a cell or to a viral particle that mediates nucleic acid transfer. Viral particles typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus.

In some vectors disclosed herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

In some embodiments, the retroviral nucleic acid comprises one or more of or all of: a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively. Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Spumaretrovirus. In some embodiments the retrovirus is an endogenous retrovirus. In some embodiments the retrovirus is a lentivirus.

In some embodiments, a retroviral or lentivirus vector further comprises one or more insulator elements, e.g., an insulator element disclosed herein. In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE. In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Illustrative lentiviruses include but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used. A lentivirus vector can comprise a viral vector or plasmid containing structural and functional genetic elements, or portions thereof, including LTRs that are primarily derived from a lentivirus.

In some embodiments, a lentivirus vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, a lentivirus vector is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In some embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as disclosed in PCT patent application WO 99/15683, which is herein incorporated by reference in its entirety.

In some embodiments, the lentivirus vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as disclosed in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included.

### B. Recombinant Expression

For all of these technologies, well-known recombinant techniques are used, to generate recombinant nucleic acids as disclosed herein. In certain embodiments, the recombinant nucleic acids encoding one or more tolerogenic factors may be operably linked to one or more regulatory nucleotide sequences in an expression construct. Regulatory nucleotide sequences are generally appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. In some embodiments, an expression vector comprises a nucleotide sequence encoding a variant polypeptide operably linked to at least one regulatory sequence. Regulatory sequence for use herein include promoters, enhancers, and other expression control elements. In some embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, CAG promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273:113-120 (1978)). The immediate early promoter of the human cytomegalovirus is conveniently obtained as a HindIII restriction enzyme fragment (Greenaway et al., Gene 18:355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides disclosed herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., AAV transduction, lentiviral transduction) or otherwise delivered on a viral vector (e.g., fusogen-mediated delivery). In some of these embodiments, the AAV vector is an AAV6 vector or an AAV9 vector. Additional AAV vectors for gene delivery are disclosed in, for example, Wang et al., “Adeno-associated virus vector as a platform for gene therapy deliver,” Nature Reviews Drug Discovery 18:358-378 (2019), the disclosure is incorporated herein by reference in its entirety. In some embodiments, the polynucleotides are introduced into a cell via a fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the cells provided herein are genetically modified to include one or more exogenous polynucleotides inserted into one or more genomic loci of the cell. In some embodiments, the exogenous polynucleotide encodes a protein of interest, e.g., a tolerogenic factor. Any suitable method can be used to insert the exogenous polynucleotide into the genomic locus of the cell including the gene editing methods disclosed herein (e.g., a CRISPR/Cas system). In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### C. Site-Directed Insertion (Knock-In)

In some embodiments, the one or more transgenes encoding one or more tolerogenic factors can be inserted into a specific genomic locus of a host cell (e.g., an allogeneic donor cell). A number of gene editing methods can be used to insert a transgene into a specific genomic locus of choice. Gene editing is a type of genetic engineering in which a nucleotide sequence may be inserted, deleted, modified, or replaced in the genome of a living organism.

In some embodiments, a rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as disclosed herein. In some embodiments, the nucleic acid comprises an mRNA. In some embodiments, the nucleic acid comprises a modified mRNA, as disclosed herein (e.g., a synthetic, modified mRNA).

The present disclosure contemplates altering target polynucleotide sequences in any manner which is available to the skilled artisan utilizing a gene editing system (e.g., CRISPR/Cas) of the present disclosure. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems of the present disclosure can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present disclosure can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present disclosure includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein, isoform thereof, or any Cas-like protein with similar function or activity of any Cas protein or isoform thereof. In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises type V Cas protein. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019). Examples of Cas proteins include, but are not limited to: Cas3, Cas8a, Cas5, Cas8b, Cas8c, Cas10d, Cse1, Cse2, Csy1, Csy2, Csy3, and/or GSU0054. In some embodiments, a Cas protein comprises Cas3, Cas8a, Cas5, Cas8b, Cas8c, Cas10d, Cse1, Cse2, Csy1, Csy2, Csy3, and/or GSU0054. Examples of Cas proteins include, but are not limited to: Cas9, Csn2, and/or Cas4. In some embodiments, a Cas protein comprises Cas9, Csn2, and/or Cas4. In some embodiments, examples of Cas proteins include, but are not limited to: Cas10, Csm2, Cmr5, Cas10, Csx11, and/or Csx10. In some embodiments, a Cas protein comprises a Cas10, Csm2, Cmr5, Cas10, Csx11, and/or Csx10. In some embodiments, examples of Cas proteins include, but are not limited to: Csf1. In some embodiments, a Cas protein comprises Csf1. In some embodiments, examples of Cas proteins include, but are not limited to: Cas12a, Cas12b, Cas12c, C2c4, C2c8, C2c5, C2c10, and C2c9; as well as CasX (Cas12e) and CasY (Cas12d). Also see, e.g., Koonin et al., Curr Opin Microbiol. 2017; 37:67-78: “Diversity, classification and evolution of CRISPR-Cas systems.” In some embodiments, a Cas protein comprises Cas12a, Cas12b, Cas12c, Cas12d, Cas12e, Cas12d, and/or Cas12e. In some embodiments, a Cas protein comprises Cas13, Cas13a, C2c2, Cas13b, Cas13c, and/or Cas13d. In some embodiments, the CRISPR/Cas system comprises a Cas effector protein selected from the group consisting of: a) Cas3, Cas8a, Cas5, Cas8b, Cas8c, Cas10d, Cse1, Cse2, Csy1, Csy2, Csy3, and GSU0054; b) Cas9, Csn2, and Cas4; c) Cas10, Csm2, Cmr5, Cas10, Csx11, and Csx10; d) Csf1; e) Cas12a, Cas12b, Cas12c, C2c4, C2c8, C2c5, C2c10, C2c9, CasX (Cas12e), and CasY (Cas12d); and f) Cas13, Cas13a, C2c2, Cas13b, Cas13c, and Cas13d.

In some embodiments, a Cas protein comprises any one of the Cas proteins disclosed herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In many embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5 (8): 747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas 12a polypeptide fused to an oligoarginine domain. In some embodiments, the Cas12a protein comprises a Cas 12a polypeptide fused to a penetratin domain. In some embodiments, the Cas12a protein comprises a Cas12a polypeptide fused to a superpositively charged GFP

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as disclosed herein. In some embodiments, the nucleic acid comprises mRNA. In some embodiments, the nucleic acid comprises a modified mRNA, as disclosed herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as disclosed herein (e.g., a synthetic, modified mRNA).

The methods of the present disclosure contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present disclosure can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as disclosed herein (e.g., a synthetic, modified mRNA).

Exemplary gRNA sequences useful for CRISPR/Cas-based targeting of genes disclosed herein are provided in Table 8. The sequences can be found in WO2016183041 filed May 9, 2016, the disclosure including the Tables, Appendices, and Sequence Listing is incorporated herein by reference in its entirety.

Other exemplary gRNA sequences useful for CRISPR/Cas-based targeting of genes disclosed herein are provided in U.S. Provisional Patent Application No. 63/190,685, filed May 19, 2021, and in U.S. Provisional Patent Application No. 63/221,887, filed Jul. 14, 2021, the disclosures of which, including the Tables, Appendices, and Sequence Listings, are incorporated herein by reference in their entireties.

In some embodiments, the cells of the technology are made using Transcription Activator-Like Effector Nucleases (TALEN) methodologies. TALEN is a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In numerous embodiments, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI disclosed in WO2012138927. TALEs are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells of the present disclosure are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the technology may for example correspond to a LAGLIDADG endonuclease, to a HNH endonuclease, or to a GIY-YIG endonuclease. Preferred homing endonuclease according to the present disclosure can be an I-CreI variant.

In some embodiments, the cells of the technology are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

Current gene editing techniques generally utilize the innate mechanism for cells to repair double-strand breaks (DSBs) in DNA. Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### 1. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### 2. TALENS

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### 3. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### 4. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### 5. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and MAD7. See, e.g., Jinek et al., Science (2012) 337 (6096): 816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is disclosed herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those disclosed to this point. For instance, Cpf1 (CRISPR from Prevotella and franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA: tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 5 below.

TABLE 5
Exemplary Cas nuclease variants and their PAM sequences

| CRISPR | | PAM Sequence |
|---|---|---|
| Nuclease | Source Organism | (5′→3′) |
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas12a (Cpf1) | Acidaminococcus sp. | tttv |
| AacCas12b | Alicyclobacillus acidiphilus | ttn |
| BhCas12b v4 | Bacillus hisashii | attn, tttn, or gttn |
| ErCas12a (MAD7) | Eubacterium rectale | yttn |

r = a or g;
y = c or t;
w = a or t;
v = a or c or g;
n = any base

MAD7 recognizes a PAM 5′ to 21 nucleotide spacer sequence. MAD7 associates with a single, small crRNA of 56 nucleotides in total (35 nucleotide scaffold sequence and 21 nucleotide space sequence). Cleavage of DNA by MAD7 results in a staggered cut 19 base pairs and 23 base pairs distal to the PAM. In some embodiments, a MAD7 crRNA comprises one or more chemical modifications known in the art and/or as described herein.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In one exemplary embodiment, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TCAC. In some embodiments, a TAM sequence comprises TCAG. In some embodiments, a TAM sequence comprises TCAT. In some embodiments, a TAM sequence comprises TCAA. In some embodiments, a TAM sequence comprises TTCAN. In some embodiments, a TAM sequence comprises TTCAA. In some embodiments, a TAM sequence comprises TTCAG. In some embodiments, a TAM sequence comprises TTGAT.

In certain embodiments, the transgene may function as a DNA repair template to be integrated into the target site through HDR in associated with a gene editing system (e.g., the CRISPR/Cas system) as disclosed herein. Generally, the transgene to be inserted would comprise at least the expression cassette encoding the protein of interest (e.g., the tolerogenic factor) and would optionally also include one or more regulatory elements (e.g., promoters, insulators, enhancers). In certain of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, prime editing may be used to engineer exogenous genes, such as exogenous transgenes encoding a tolerogenic factor (e.g., CD47) into specific loci. Prime editing uses an enzyme and a guide RNA. The enzyme is a catalytically impaired Cas9 endonuclease fused to an engineered reverse transcriptase. The guide RNA is a prime editing guide RNA (pegRNA) that includes RNA specified for the target site and encoding the edit, such as insertion of the transgene. See Anzelone et al., Nature (2019) 576:149-157.

In some embodiments, the base editing technology may be used to introduce single-nucleotide variants (SNVs) into DNA or RNA in living cells. Base editing is a CRISPR-Cas9-based genome editing technology that allows the introduction of point mutations in RNAs or DNAs without generating DSBs. Two major classes of base editors have been developed: cytidine base editors (CBEs) allowing C:G to T:A conversions and adenine base editors (ABEs) allowing A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction.

### 6. Nickases

Nuclease domains of the Cas, in particular the Cas9, nuclease can be mutated independently to generate enzymes referred to as DNA “nickases.” Nickases are capable of introducing a single-strand cut with the same specificity as a regular CRISPR/Cas nuclease system, including for example CRISPR/Cas9. Nickases can be employed to generate double-strand breaks which can find use in gene editing systems (Mali et al., Nat Biotech, 31 (9): 833-838 (2013); Mali et al. Nature Methods, 10:957-963 (2013); Mali et al., Science, 339 (6121): 823-826 (2013)). In some instances, when two Cas nickases are used, long overhangs are produced on each of the cleaved ends instead of blunt ends which allows for additional control over precise gene integration and insertion (Mali et al., Nat Biotech, 31 (9): 833-838 (2013); Mali et al. Nature Methods, 10:957-963 (2013); Mali et al., Science, 339 (6121): 823-826 (2013)). As both nicking Cas enzymes must effectively nick their target DNA, paired nickases can have lower off-target effects compared to the double-strand-cleaving Cas-based systems (Ran et al., Cell, 155 (2): 479-480 (2013); Mali et al., Nat Biotech, 31 (9): 833-838 (2013); Mali et al. Nature Methods, 10:957-963 (2013); Mali et al., Science, 339 (6121): 823-826 (2013)).

### D. Genomic Loci for Insertion of the Transgene

In some embodiments, the genomic locus for site-directed insertion of one or more transgenes encoding one or more tolerogenic factors is an endogenous B2M gene locus. In some embodiments, the genomic locus for site-directed insertion of one or more transgenes encoding one or more tolerogenic factors is an endogenous CIITA gene locus. In some embodiments, the one or more transgenes encoding one or more tolerogenic factors are inserted into both B2M and CIITA loci. The specific site for insertion within a gene locus may be located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the endogenous gene in that locus. In some embodiments, two or more transgenes are inserted in the same locus such that the two or more transgenes are carried by a polycistronic vector. Exemplary genomic loci for insertion of a transgene are depicted in Tables 6 and 7.

TABLE 6
Exemplary genomic loci for insertion of exogenous polynucleotides

| | | | Target region |
|---|---|---|---|
| Species | Gene Locus | Ensembl ID | for cleavage |
| human | B2M | ENSG00000166710 | CDS |
| human | CIITA | ENSG00000179583 | CDS |

TABLE 7
Non-limiting examples of Cas9 guide RNAs

| | SEQ | | | | |
|---|---|---|---|---|---|
| | ID | | | Target | gRNA cut |
| Gene | NO: | guide sequence | PAM | site | location |
| B2M | 19 | CGUGAGUAAACCUGAAUCUU | TGG | Exon 2 | chr15: 44,715,434 |
| CIITA | 20 | GAUAUUGGCAUAAGCCUCCC | TGG | Exon 3 | chr16: 10,895,747 |

E. Guide RNAs (gRNAs) for Site-Directed Insertion

In some embodiments, provided are gRNAs for use in site-directed insertion of a transgene in a B2M and/or CIITA locus according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 8. See, e.g., Jinek et al., Science (2012) 337 (6096): 816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 21-24. In some embodiments, the gRNA comprises all or a portion of the nucleotide sequences set forth in SEQ ID NOs: 25-28. In some embodiments, the gRNA comprises all or a portion of the nucleotide sequences set forth in SEQ ID NOs: 29-32. In some embodiments, the gRNA comprises all or a portion of the nucleotide sequences set forth in SEQ ID NOs: 33-36.

In some embodiments, the gRNA comprises a crRNA repeat region comprising, consisting of, or consisting essentially of the nucleotide sequence set forth in SEQ ID NO:22, SEQ ID NO: 26, SEQ ID NO:30, or SEQ ID NO:35. In some embodiments, the gRNA comprises a tetraloop comprising, consisting of, or consisting essentially of the nucleotide sequence set forth in SEQ ID NO:23 or SEQ ID NO:34. In some embodiments, the gRNA comprises a tracrRNA comprising, consisting of, or consisting essentially of the nucleotide sequence set forth in SEQ ID NO: 24, SEQ ID NO:28, SEQ ID NO:32, or SEQ ID NO:33.

TABLE 8
Exemplary gRNA structure and sequence for CRISPR/Cas

| SEQ ID NO: | Sequence (5′→3′) | Description |
|---|---|---|
| 21 | nnnnnnnnnnnnnnnnnnnn | Exemplary spCas9 1 |
| | | Complementary |
| | | region (spacer) |
| 22 | guuuuagagcua | Exemplary spCas9 1 |
| | | crRNA repeat region |
| 23 | gaaa | Exemplary spCas9 1 |
| | | tetraloop |
| 24 | uagcaaguuaaaauaaggcuaguccguuaucaacuugaaaaagu | Exemplary spCas9 1 |
| | ggca | tracrRNA |
| 25 | nnnnnnnnnnnnnnnnnnnn | Exemplary spCas9 2 |
| | | Complementary |
| | | region (spacer) |
| 26 | guuusagagcuaugcug | Exemplary spCas9 2 |
| | | crRNA repeat region |
| 27 | gaaa | Exemplary spCas9 2 |
| | | tetraloop |
| 28 | cagcauagcaaguusaaauaaggcuaguccguuaucaacuugaa | Exemplary spCas9 2 |
| | aaag | tracrRNA |
| 29 | nnnnnnnnnnnnnnnnnnnn | Exemplary saCas9 |
| | | Complementary |
| | | region (spacer) |
| 30 | guuuuaguacucug | Exemplary saCas9 |
| | | crRNA repeat region |
| 31 | gaaa | Exemplary saCas9 |
| | | tetraloop |
| 32 | cagaaucuacuaaaacaaggcaaaaugccguguuuaucucguca | Exemplary saCas9 |
| | acuuguuggcgagauuuuuu | tracrRNA |
| 33 | gucgucuauaggacggcgaggacaacgggaagugccaaugugcu | Exemplary AkCas12b |
| | cuuuccaagagcaaacaccccguuggcuucaagaugaccgcucg | tracrRNA |
| 34 | aaaa | Exemplary AkCas12b |
| | | tetraloop |
| 35 | cgagcggucugagaaguggcacu | Exemplary AkCas12b |
| | | crRNA repeat region |
| 36 | nnnnnnnnnnnnnnnnnnnn | Exemplary AkCas12b |
| | | Complementary |
| | | region (spacer) |

s = c or g; n = any base

In some embodiments, the gRNA comprises a complementary region specific to a target gene locus of interest, for example, the B2M locus (e.g., exon 2 of B2M), or the CIITA locus (e.g., exon 3 of CIITA). The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as disclosed herein. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as disclosed herein. For example, for CRISPR/Cas systems, when an existing gRNA for a particular locus (e.g., within an endogenous B2M or CIITA gene locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is disclosed as illustrative, any gene editing approaches as disclosed can be used in this method of identifying new loci, including those using ZFNs, TALENS, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs disclosed herein can contain one or more modified nucleosides or nucleotides which introduce stability toward nucleases. While not being bound by a particular theory, it is believed that certain modified gRNAs disclosed herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly(A) tract comprising one or more (and typically 5-200) adenine (A) residues. The poly(A) tract can be contained in the nucleic acid sequence encoding the gRNA or can be added to the gRNA during chemical synthesis, or following in vitro transcription using a polyadenosine polymerase (e.g., E. coli poly(A) polymerase). In vivo, poly(A) tracts can be added to sequences transcribed from DNA vectors through the use of polyadenylation signals. Examples of such signals are provided in Tian et al., “Signals for pre-mRNA cleavage and polyadenylation,” Wiley Interdiscip Rev RNA 3 (3): 385-396 (2012). Other suitable gRNA modifications include, without limitations, those disclosed in U.S. Patent Application No. US 2017/0073674 A1 and International Publication No. WO 2017/165862 A1, the entire contents of each of which are incorporated by reference herein.

F. Delivery of Gene Editing Systems into a Host Cell

In some embodiments, provided are compositions comprising one or more components of a gene editing system disclosed herein, including one or more gRNAs, a site-directed nuclease (e.g., a Cas nuclease) or a nucleotide sequence encoding a site-directed nuclease protein, and a transgene for targeted insertion. In some embodiments, these compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., a transgene encoding a tolerogenic factor) for targeted insertion, may be delivered into a cell in the form of a delivery vector. The delivery vector can be any type of vector suitable for introduction of nucleotide sequences into a cell, including, for example, plasmids, adenoviral vectors, adeno-associated viral (AAV) vectors such as an AAV6 vector and an AAV9 vector, retroviral vectors, lentiviral vectors, phages, and HDR-based donor vectors. Additional AAV vectors for gene delivery are disclosed in, for example, Wang et al., “Adeno-associated virus vector as a platform for gene therapy deliver,” Nature Reviews Drug Discovery 18:358-378 (2019), the disclosure is incorporated herein by reference in its entirety. The different components may be introduced into a cell together or separately, and may be delivered in a single vector or multiple vectors.

In some embodiments, the delivery vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system disclosed herein, including one or more gRNAs, a site-directed nuclease (e.g., a Cas nuclease) or a nucleotide sequence encoding a site-directed nuclease protein, and a transgene for targeted insertion.

### III. Methods of Cell Maintenance, Differentiation and Manufacture

In those embodiments of the methods provided herein where the cells being engineered are PSCs, the engineered cells can be maintained in an undifferentiated state using methods known in the art. For example, the cells can be cultured on Matrigel using culture media that prevents differentiation and maintains pluripotency. In addition, they can be maintained in culture medium under conditions to maintain pluripotency.

In other embodiments, engineered PSCs may be further differentiated to provide immune evasive cells suitable for use in adoptive cell therapy. Accordingly, in some aspects the present technology provides immune evasive cells that are differentiated from engineered PSCs according to various embodiments disclosed herein. In some embodiments, the differentiated cells are suitable for use in adoptive cell therapy, as they have been made to be immune evasive (e.g., by inserting one or more tolerogenic factors into an endogenous B2M gene locus, an endogenous CIITA locus, or both in the PSCs).

### A. Generation of Induced Pluripotent Stem Cells

The disclosed technology provides methods of producing immune evasive pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques disclosed therein. Since then, a number of methods have been developed; see Seki et al, World J. Stem Cells 7 (1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors in the host cell, usually introduced using episomal vectors. Under these conditions, small amounts of the cells are induced to become iPSCs (in general, the efficiency of this step is low, as no selection markers are used). Once the cells are “reprogrammed,” and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency,” e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used, which reprogramming factors are selected from SOKMNLT; SOX2, OCT4 (POU5F1), KLF4, MYC, NANOG, LIN28, and SV40L T antigen. In general, these reprogramming factor genes are provided on episomal vectors which are known in the art and commercially available.

In general, as is known in the art, iPSCs are made from non-pluripotent cells such as, but not limited to, blood cells, fibroblasts, etc., by transiently expressing the reprogramming factors as disclosed herein.

### B. Assays for Immune Evasive Phenotypes and Retention of Pluripotency

Once the engineered cells have been generated, they may be assayed for their immune evasiveness and/or retention of pluripotency as is disclosed in WO2016183041 and WO2018132783.

In some embodiments, immune evasiveness is assayed using a number of techniques as exemplified in FIG. 13 and FIG. 15 of WO2018132783. These techniques include transplantation into allogeneic recipients and monitoring for immune evasive pluripotent cell growth (e.g., teratomas) that escape the recipient immune system. In some instances, immune evasive pluripotent cell derivatives are transduced to express luciferase and can then followed using bioluminescence imaging. Similarly, the T cell and/or B cell response of the recipient to such cells are tested to confirm that the cells do not cause an immune reaction in the recipient. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells disclosed herein and determining the expression levels of T cell activation markers in the T cells.

In vivo assays can be performed to assess the immunogenicity of the cells disclosed herein. In some embodiments, the survival and immunogenicity of immune evasive cells are determined using an allogenic humanized immunodeficient mouse model. In some instances, the immune evasive pluripotent stem cells are transplanted into an allogenic humanized NSG-SGM3 mouse and assayed for cell rejection, cell survival, and teratoma formation. In some instances, grafted immune evasive pluripotent stem cells or differentiated cells thereof display long-term survival in the mouse model.

Additional techniques for determining immunogenicity including immune evasiveness of the cells are disclosed in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally disclosed herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as disclosed herein; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of MHC I and II (or HLA I and II), the engineered cells of the technology have a reduced susceptibility to macrophage phagocytosis and NK cell killing. The resulting immune evasive cells “escape” the immune macrophage and innate pathways due to reduction or lack of B2M and/or CIITA and the expression of one or more transgenes such as CD47.

### C. Method of Manufacture

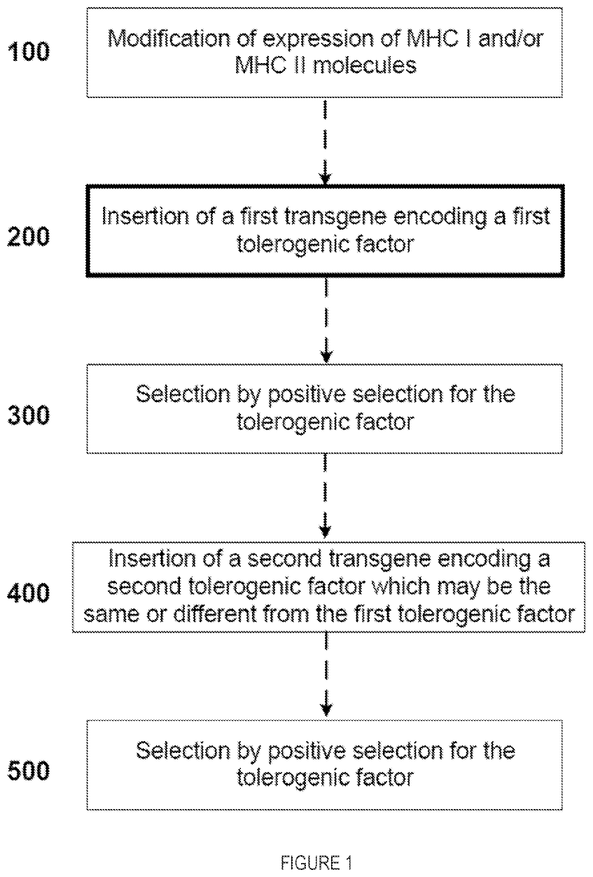

In some aspects, the present technology provides methods for generating a population of immune evasive cells for cell therapy (FIG. 1). A flow chart of certain embodiments of the methods is shown in FIG. 1. In some embodiments, the method comprises inserting a transgene encoding one or more tolerogenic factors into an endogenous B2M gene locus and/or CIITA gene locus of the cells (FIG. 1, step 200). Optionally, the method comprises selecting for cells that have the transgene inserted by positive selection for the tolerogenic factor (e.g., selection for expression of the tolerogenic factor) (FIG. 1, step 300). Inserting one or more tolerogenic factors at the endogenous B2M or CIITA gene locus may achieve the dual purposes of increasing expression of the one or more tolerogenic factors and reducing or eliminating B2M or CIITA expression in the cells in one manufacturing step, so that the resulting cells can be made immune evasive and not subject to immune rejection when transplanted into a recipient, thereby increasing both the efficiency of the manufacturing process and the effectiveness of cell-based therapies. Reducing or eliminating B2M or TAP1 expression results in reducing or eliminating one or more MHC I molecules, and reducing CD74 or CIITA expression or increasing CD47 expression results in reducing or eliminating one or more MHC II molecules. Accordingly, in some embodiments, the methods further comprise modifying the expression of one or more MHC class I and/or one or more MHC class II molecules in the cells, for example, by knocking out or knocking down B2M, TAP1, CD74, and/or CIITA which does not have the insertion of the transgene encoding the tolerogenic factor (FIG. 1, step 100). In some embodiments, methods further comprise inserting one or more additional tolerogenic factors into CIITA locus, B2M locus, or a safe harbor locus which is not used in step 200 (FIG. 1, step 400). Optionally, the method further comprises selecting for cells that have the transgene inserted by positive selection for the tolerogenic factor (e.g., selection for expression of the tolerogenic factor) (FIG. 1, step 500). In some embodiments, step 100 can be carried out before step 200. In some embodiments, step 100 can be carried out after step 200, after step 300, after step 400, or after step 500.

### IV. Cells and Compositions Thereof

In some aspects, the present technology provides engineered immune evasive cells, such as immune evasive allogeneic cells, that are derived from or generated by methods according to various embodiments disclosed herein. In some embodiments, the generated cells are suitable for use in adoptive cell therapy, as they have been made to be immune evasive (e.g., by inserting one or more tolerogenic factors into an endogenous B2M gene locus, an endogenous CIITA locus, or both).

In some embodiments, the cells generated by the methods disclosed herein or used in the methods disclosed herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a recipient's immune system. In other words, the cells administered to a recipient in accordance with the methods disclosed herein are not detectable by immune cells of the recipient's immune system. In some embodiments, the cells are cloaked and therefore avoid immune rejection.

Methods of determining whether a cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

In some embodiments, the engineered cell is an autologous cell, i.e., obtained from the subject who will receive the engineered cell after modification. In some embodiments, the engineered cell is an allogeneic cell, i.e., obtained from someone other than the subject who will receive the engineered cell after modification. In either of these embodiments, the cells can be primary cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the primary cells are pluripotent. In some embodiments, the primary cells comprise pluripotent stem cells. In some embodiments, the primary cells are human primary cells. In some embodiments, the human primary cells are human pluripotent stem cells (hPSCs). In other embodiments, especially in the case of allogeneic cells, the cells can be derived or differentiated from embryonic stem cells (ESCs) or induced pluripotent cells (iPSCs).

In some embodiments, the modified pluripotent stem cells (e.g., modified iPSCs) include one or more genomic modifications that reduce expression of MHC class I molecules and a modification that increases expression of CD47. In other words, the modified pluripotent stem cells comprise exogenous CD47 proteins and exhibit reduced or silenced surface expression of one or more MHC class I molecules. In some embodiments, the cells include one or more genomic modifications that reduce expression of MHC class II molecules and a modification that increases expression of CD47. In some instances, the modified cells comprise exogenous CD47 nucleic acids and proteins and exhibit reduced or silenced surface expression of one or more MHC class I molecules. In some embodiments, the cells include one or more genomic modifications that reduce or eliminate expression of MHC class II molecules, one or more genomic modifications that reduce or eliminate expression of MHC class II molecules, and a modification that increases expression of CD47. In some embodiments, the modified pluripotent stem cells comprise exogenous CD47 proteins, exhibit reduced or silenced surface expression of one or more MHC class I molecules and exhibit reduced or lack surface expression of one or more MHC class II molecules. In many embodiments, the cells are B2M^{indel/indel}, CIITA^{indel/indel}, or CD47^{tg} cells.

In some embodiments, the primary cell or the differentiated cell disclosed herein is a cell type selected from a group that includes a cardiac cell, a cardiac progenitor cell, a cardiomyocyte, a neural cell, an endothelial cell, a T cell (including subtypes of T cells), a B cell, a NK cell, a pancreatic islet cell including pancreatic beta islet cells, a retinal pigmented epithelium cell, a hepatocyte, a thyroid cell, a skin cell, a blood cell, a plasma cell, a platelet, a renal cell, a glial progenitor cell, an endothelial cell, and an epithelial cell. In some embodiments, the engineered cells or the progeny thereof are cells of any organ or tissue of the body including, but not limited to, the heart, brain, skin, eye, pancreas, bladder, spleen, liver, lung, kidney, thyroid, cardiovascular system, respiratory system, nervous system, and immune system. In some embodiments, the pluripotent stem cells are differentiated into cells of any organ or tissue of the body using a specific differentiation condition.

In some embodiments, the population of therapeutic cells disclosed herein comprises: (a) cells selected from the group consisting of glial progenitor cells, oligodendrocytes, astrocytes, and dopaminergic neurons, optionally wherein the dopaminergic neurons are selected from the group consisting of neural stem cells, neural progenitor cells, immature dopaminergic neurons, and mature dopaminergic neurons; (b) hepatocytes or hepatic progenitor cells; (c) corneal endothelial progenitor cells or corneal endothelial cells; (d) cardiomyocytes or cardiac progenitor cells; (e) pancreatic islet cells, including pancreatic beta islet cells, optionally wherein the pancreatic islet cells are selected from the group consisting of a pancreatic islet progenitor cell, an immature pancreatic islet cell, and a mature pancreatic islet cell; (f) endothelial cells; (g) thyroid progenitor cells; and (h) renal precursor cells or renal cells.

In some embodiments, one or more populations of the engineered immune evasive cells or one or more types or subtypes of the engineered immune evasive cells disclosed herein are formulated into pharmaceutical compositions for treating various conditions or diseases.

A. Therapeutic Cells from Primary Cells

Provided herein are immune evasive cells including, but not limited to, primary cells that evade immune recognition. In some embodiments, the engineered cells are produced (e.g., generated, cultured, or derived) from cells such as primary cells. In some instances, the primary cells are obtained (e.g., harvested, extracted, removed, or taken) from a subject or an individual. In some embodiments, the primary cells are produced from a pool of cells such that the cells are from one or more subjects (e.g., one or more human including one or more healthy humans). In some embodiments, the pool of primary cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is a live donor. In some embodiments, the donor subject is a cadaveric donor. In some embodiments, the donor subject is different from the patient (e.g., the recipient that is administered the therapeutic cells). In some embodiments, the pool of cells does not include cells from the patient. In some embodiments, one or more of the donor subjects from which the pool of cells is obtained are different from the patient. In some embodiments, the pool of cells comprises cells from the patient and cells from one or more donors different from the patient (e.g., the recipient that is administered the therapeutic cells).

In some embodiments, the types of primary cells include but are not limited to pancreatic islet cells including pancreatic beta islet cells, retinal pigment epithelial cells, T cells, B cells, NK cells, thyroid cells, cells producing factors, skin cells, blood cells, plasma cells, platelets, renal cells, hepatocytes, neural cells, neuronal cells, glial progenitor cells, epithelial cells, endothelial cells, cardiac cells, cardiac progenitor cells, and cardiomyocytes.

In some embodiments, the engineered immune evasive cells do not activate an innate and/or an adaptive immune response in the patient (e.g., recipient) upon administration. Provided are methods of treating a disorder by administering a population of therapeutic cells comprising the engineered immune evasive cells to a subject or patient in need thereof (e.g., recipient). In some embodiments, the engineered immune evasive cells disclosed herein comprise cells engineered or modified to express one or more tolerogenic factors disclosed herein.

In some embodiments, the present disclosure is directed to engineered immune evasive primary cells that overexpress one or more tolerogenic factors such as CD47, have reduced expression or lack of expression of one or more MHC class I and/or one or more MHC class II molecules and/or have reduced expression or lack of expression of B2M, TAP1, CD74, and/or CIITA. In some embodiments, the primary cells display reduced levels or activity of one or more MHC class I antigens, one or more MHC class II antigens, or both. In certain embodiments, the primary cells overexpress one or more exogenous tolerogenic factors such as CD47 and harbor a genomic modification in the B2M gene that reduces or eliminates the expression of B2M. In some embodiments, the primary cells overexpress one or more exogenous tolerogenic factors such as CD47 and harbor a genomic modification in the CIITA gene that reduces or eliminates the expression of CIITA.

### 1. Primary T Cells

In some embodiments, primary T cells from one or more subjects are pooled. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the pool of T cells does not include cells from the patient. In some embodiments, one or more of the donor subjects from which the pool of T cells is obtained are different from the patient.

In some embodiments, the immune evasive T cells do not activate an immune response in the patient (e.g., recipient upon administration). Provided are methods of treating a disorder by administering a population of immune evasive T cells to a subject (e.g., recipient) or patient in need thereof. In some embodiments, the immune evasive cells disclosed herein comprise primary T cells engineered (e.g., are modified) to express a CAR including but not limited to a CAR disclosed herein. In some instances, the T cells are populations or subpopulations of primary T cells from one or more individuals. In some embodiments, the T cells disclosed herein such as the engineered or modified CAR-T cells comprise reduced expression of an endogenous T cell receptor.

Immune evasive T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

B. Therapeutic Cells Differentiated from Immune Evasive PSCs

Provided herein are immune evasive cells including cells derived from stem cells that evade immune recognition. In some embodiments, the stem cells are mesenchymal stem cells. In some embodiments, the stem cells are embryonic stem cells. In some embodiments, the stem cells are pluripotent stem cells, optionally the pluripotent stem cells are induced pluripotent stem cells. In some embodiments, the cells do not activate an innate and/or an adaptive immune response in the patient or subject (e.g., recipient) upon administration. Provided are methods of treating a disorder comprising a single dosing or repeat dosing of a population of immune evasive cells to a recipient subject in need thereof.

In some embodiments, the pluripotent stem cell and any cell differentiated from such a pluripotent stem cell is modified to exhibit increased expression of one or more tolerogenic factors such as CD47. In some instances, the cell overexpresses the one or more tolerogenic factors by harboring one or more transgenes encoding one or more tolerogenic factors in an endogenous B2M locus, an endogenous CIITA locus, or both.

In some embodiments, the pluripotent stem cell and any cell differentiated from such a pluripotent stem cell is modified to exhibit reduced expression of B2M, TAP1, CD74, and/or CIITA. In some embodiments, the pluripotent stem cell and any cell differentiated from such a pluripotent stem cell is modified to exhibit reduced expression of one or more MHC class I human leukocyte antigens. In other embodiments, the pluripotent stem cell and any cell differentiated from such a pluripotent stem cell is modified to exhibit reduced expression of one or more MHC class II human leukocyte antigens. In some embodiments, the pluripotent stem cell and any cell differentiated from such a pluripotent stem cell is modified to exhibit reduced expression of one or more MHC class I and one or more MHC class II human leukocyte antigens. In some embodiments, the pluripotent stem cell and any cell differentiated from such a pluripotent stem cell is modified to exhibit reduced expression of one or more MHC class I and one or more MHC class II human leukocyte antigens and B2M and CIITA.

Such pluripotent stem cells are immune evasive stem cells. Such differentiated cells are immune evasive cells as well. The immune evasive stem cells can differentiate into various cell types, including but not limited to pancreatic islet cells including pancreatic beta islet cells, retinal pigment epithelial cells, T cells, B cells, NK cells, thyroid cells, cells producing factors, skin cells, blood cells, plasma cells, platelets, renal cells, hepatocytes, neural cells, neuronal cells, glial progenitor cells, epithelial cells, endothelial cells, cardiac cells, cardiac progenitor cells, and cardiomyocytes.

Any of the pluripotent stem cells disclosed herein can be differentiated into any cells of an organism and tissue. In some embodiments, the differentiated cells exhibit increased expression of one or more tolerogenic factors such as CD47. In some instances, expression of one or more tolerogenic factors is increased in the differentiated cells encompassed by the present disclosure as compared to unmodified cells, wild-type cells, or control cells of the same cell type. In some embodiments, the differentiated cells exhibit reduced expression of B2M, TAP1, CD74, and/or CIITA and reduced expression of one or more MHC class I and/or one or more MHC class II human leukocyte antigens. In some instances, expression of B2M, TAP1, CD74, and/or CIITA is reduced compared to unmodified cell, wild-type cell, or control cell of the same cell type. In some instances, expression of one or more MHC class I and/or one or more MHC class II human leukocyte antigens is reduced compared to unmodified cell, wild-type cell, or control cell of the same cell type.

1. Cardiac Cells Differentiated from Immune Evasive PSCs

Provided herein are cardiac cell types differentiated from engineered immune evasive PSCs for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiomyocyte progenitor cell, cardiac stem cell, cardiac muscle cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, cardiac cells disclosed herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods disclosed herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the cardiomyocyte precursor includes a cell that is capable of giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 2, 3, 4 or 5 markers) from the following list: cardiac troponin I (cTnl), cardiac troponin T (cTnT), sarcomeric myosin heavy chain (MHC), GATA-4, Nkx2.5, N-cadherin, B2-adrenoceptor, ANF, the MEF-2 family of transcription factors, creatine kinase MB (CK-MB), myoglobin, and atrial natriuretic factor (ANF). In some embodiments, the cardiac cells demonstrate spontaneous periodic contractile activity. In some embodiments, when cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are immune evasive cardiac cells.

In some embodiments, the method of producing a population of immune evasive cardiac cells from a population of engineered immune evasive PSCs by in vitro differentiation comprises: (a) culturing a population of engineered immune evasive PSCs in a culture medium comprising a GSK inhibitor; (b) culturing the population of engineered immune evasive PSCs in a culture medium comprising a WNT antagonist to produce a population of pre-cardiac cells; and (c) culturing the population of pre-cardiac cells in a culture medium comprising insulin to produce a population of immune evasive cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some embodiments, the WNT antagonist is at a concentration ranging from about 2 mM to about 10 mM.

In some embodiments, the population of immune evasive cardiac cells is isolated from non-cardiac cells. In some embodiments, the isolated population of immune evasive cardiac cells are expanded prior to administration. In certain embodiments, the isolated population of immune evasive cardiac cells are expanded and cryopreserved prior to administration.

In some embodiments, the engineered immune evasive PSCs are differentiated into cardiomyocytes to address cardiovascular diseases. Differentiation can be assayed as is known in the art, generally by evaluating the presence of cardiomyocyte associated or specific markers or by measuring functionally; see, for example Loh et al., Cell, 2016, 166, 451-467, hereby incorporated by reference in its entirety and specifically for the methods of differentiating stem cells including cardiomyocytes.

Other useful methods for differentiating induced pluripotent stem cells or pluripotent stem cells into cardiac cells are disclosed, for example, in US2017/0152485; US2017/0058263; US2017/0002325; US2016/0362661; US2016/0068814; U.S. Pat. Nos. 9,062,289; 7,897,389; and 7,452,718. Additional methods for producing cardiac cells from induced pluripotent stem cells or pluripotent stem cells are disclosed in, for example, Xu et al., Stem Cells and Development, 2006, 15 (5): 631-9, Burridge et al., Cell Stem Cell, 2012, 10:16-28, and Chen et al., Stem Cell Res, 2015, 15 (2): 365-375.

In various embodiments, immune evasive cardiac cells can be cultured in culture medium comprising a BMP pathway inhibitor, a WNT signaling activator, a WNT signaling inhibitor, a WNT agonist, a WNT antagonist, a Src inhibitor, an EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, SO3031 (KY01-I), SO2031 (KY02-I), and SO3042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells provided herein can be cultured on a surface, such as a synthetic surface to support and/or promote differentiation of immune evasive PSCs into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}]decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, Pyrex glass, Vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as disclosed herein can be assessed in animal models for cardiac cryoinjury, which causes 55% of the left ventricular wall tissue to become sCAR-Tissue without treatment (Li et al., Ann. Thorac. Surg. 62:654, 1996; Sakai et al., Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

2. Neural Cells Differentiated from Immune Evasive PSCs

Provided herein are different neural cell types differentiated from engineered immune evasive PSCs that are useful for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells disclosed herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are disclosed in PCT Application No. WO2010144696 and U.S. Pat. Nos. 9,057,053; 9,376,664; and 10,233,422. Additional disclosure of methods for differentiating immune evasive pluripotent cells can be found, for example, in Deuse et al., Nature Biotechnology, 2019, 37, 252-258 and Han et al., Proc Natl Acad Sci USA, 2019, 116 (21), 10441-10446. Methods for determining the effect of neural cell transplantation in an animal model of a neurological disorder or condition are disclosed in the following references: for spinal cord injury-Curtis et al., Cell Stem Cell, 2018, 22, 941-950; for Parkinson's disease—Kikuchi et al., Nature, 2017, 548:592-596; for ALS—Izrael et al., Stem Cell Research, 2018, 9 (1): 152 and Izrael et al., IntechOpen, DOI: 10.5772/intechopen. 72862; for epilepsy-Upadhya et al., PNAS, 2019, 116 (1): 287-296.

a. Cerebral Endothelial Cells

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells disclosed herein are administered to a subject to treat or ameliorate stroke. In some embodiments, the neurons and glial cells are administered to a subject with amyotrophic lateral sclerosis (ALS). In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

In some embodiments, cerebral endothelial cells (ECs), precursors, and progenitors thereof are differentiated from immune evasive PSCs (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF (bFGF), and Y-27632. In some embodiments, the medium includes a supplement designed to promote survival and functionality for neural cells.

In some embodiments, cerebral endothelial cells (ECs), precursors, and progenitors thereof are differentiated from immune evasive PSCs on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation end product-specific receptor AGER, receptor for retinol uptake STRA6, large neutral amino acids transporter small subunit 1 SLC7A5, excitatory amino acid transporter 3 SLCIA1, sodium-coupled neutral amino acid transporter 5 SLC38A5, solute carrier family 16 member 1 SLC16A1, ATP-dependent translocase ABCB1, ATP-ABCC2-binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

b. Dopaminergic Neurons

In some embodiments, the engineered immune evasive PSCs disclosed herein are differentiated into dopaminergic neurons include neuronal stem cells, neuronal progenitor cells, immature dopaminergic neurons, and mature dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor). In certain cases, the term “neural stem cells” includes a population of pluripotent cells that have partially differentiated along a neural cell pathway and express one or more neural markers including, for example, nestin. Neural stem cells may differentiate into neurons or glial cells (e.g., astrocytes and oligodendrocytes). The term “neural progenitor cells” includes cultured cells which express FOXA2 and low levels of b-tubulin, but not tyrosine hydroxylase. Such neural progenitor cells have the capacity to differentiate into a variety of neuronal subtypes; particularly a variety of dopaminergic neuronal subtypes, upon culturing the appropriate factors, such as those disclosed herein.

In some embodiments, the DA neurons derived from immune evasive PSCs are administered to a patient, e.g., human patient to treat a neurodegenerative disease or condition. In some embodiments, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments, DA neurons, precursors, and progenitors thereof are differentiated from engineered immune evasive PSCs by culturing the stem cells in medium comprising one or more factors or additives. Useful factors and additives that promote differentiation, growth, expansion, maintenance, and/or maturation of DA neurons include, but are not limited to, Wnt1, FGF2, FGF8, FGF8a, sonic hedgehog (SHH), brain derived neurotrophic factor (BDNF), transforming growth factor a (TGF-a), TGF-b, interleukin 1 beta, glial cell line-derived neurotrophic factor (GDNF), a GSK-3 inhibitor (e.g., CHIR-99021), a TGF-β inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, and the like. Differentiation protocols and detailed descriptions thereof are provided in, e.g., U.S. Pat. Nos. 9,968,637, 7,674,620, Kim et al., Nature, 2002, 418, 50-56; Bjorklund et al., PNAS, 2002, 99 (4), 2344-2349; Grow et al., Stem Cells Transl Med. 2016, 5 (9): 1133-44, and Cho et al., PNAS, 2008, 105:3392-3397, the disclosures in their entirety including the detailed description of the examples, methods, figures, and results are herein incorporated by reference.

In some embodiments, the population of immune evasive dopaminergic neurons is isolated from non-neuronal cells. In some embodiments, the isolated population of immune evasive dopaminergic neurons are expanded prior to administration. In certain embodiments, the isolated population of immune evasive dopaminergic neurons are expanded and cryopreserved prior to administration.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, and LIM homeobox transcription factor 1-beta (LMX1B), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from immune evasive PSCs are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11 (1): 171-182; Lorenz Studer, “Chapter 8-Strategies for Bringing Stem Cell-Derived Dopamine Neurons to the clinic—The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication application No. 20160115448, and U.S. Pat. Nos. 8,252,586; 8,273,570; 9,487,752 and 10,093,897, the contents are incorporated herein by reference in their entirety.

In addition to DA neurons, other neuronal cells, precursors, and progenitors thereof can be differentiated from the immune evasive PSCs disclosed herein by culturing the cells in medium comprising one or more factors or additive. Non-limiting examples of factors and additives include GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-11014, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, DKK-4, SFRP-1, SFRP-2, SFRP-3, SFRP-4, SFRP-5, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg—Ag and/or derivatives thereof.

In some embodiments, the neurons express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein 01 FOXO1, Forkhead box protein A2 FOXA2, Forkhead box protein 04 FOX04, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and/or any combination thereof.

c. Glial Cells

In some embodiments, the neural cells disclosed herein including glial cells such as, but not limited to, microglia, astrocytes, oligodendrocytes, ependymal cells and Schwann cells, glial precursors, and glial progenitors thereof are produced by differentiating engineered immune evasive PSCs into therapeutically effective glial cells and the like. Differentiation of engineered immune evasive PSCs produces immune evasive neural cells, such as immune evasive glial cells.

In some embodiments, glial cells, precursors, and progenitors thereof generated by culturing engineered immune evasive PSCs in medium comprising one or more agents selected from the group consisting of retinoic acid, IL-34, M-CSF, FLT3 ligand, GM-CSF, CCL2, a TGF-β inhibitor, a BMP signaling inhibitor, a SHH signaling activator, FGF, platelet derived growth factor PDGF, PDGFR-α, HGF, IGF1, noggin, SHH, dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in U.S. Pat. Nos. 7,579,188; 7,595,194; 8,263,402; 8,206,699; 8,252,586; 9,193,951; 9,862,925; 8,227,247; 9,709,553; US2018/0187148; US2017/0198255; US2017/0183627; US2017/0182097; US2017/253856; US2018/0236004; WO2017/172976; and WO2018/093681. Methods for differentiating pluripotent stem cells are disclosed in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

The efficacy of neural cell transplants for spinal cord injury can be assessed in, for example, a rat model for acutely injured spinal cord, as disclosed by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells disclosed herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intra-arterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure is herein incorporated by reference in its entirety.

3. Endothelial Cells Differentiated from Immune Evasive PSCs

Provided herein are engineered immune evasive PSCs that are differentiated into various endothelial cell types for subsequent transplantation or engraftment into subjects (e.g., recipients). As will be appreciated by those in the art, the methods for differentiation depend on the desired cell type using known techniques.

In some embodiments, the endothelial cells differentiated from the engineered immune evasive PSCs are administered to a patient, e.g., a human patient in need thereof. The endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In certain embodiments, the patient has suffered from or is suffering from a transient ischemic attack or stroke, which in some cases, may be due to cerebrovascular disease. In some embodiments, the immune evasive endothelial cells are administered to treat tissue ischemia e.g., as occurs in atherosclerosis, myocardial infarction, and limb ischemia and to repair of injured blood vessels. In some instances, the immune evasive cells are used in bioengineering of grafts.

For instance, the immune evasive endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the immune evasive endothelial cells can be further modified to deliver agents to target and treat tumors.

In some embodiments, provided herein is a method of repair or replacement for tissue in need of vascular cells or vascularization. The method involves administering to a human patient in need of such treatment, a composition containing the isolated immune evasive endothelial cells to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In some embodiments, vascular diseases, which may be associated with cardiac diseases or disorders can be treated by administering endothelial cells, such as but not limited to, definitive vascular endothelial cells and endocardial endothelial cells derived as disclosed herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels.

In certain embodiments, the immune evasive endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the immune evasive endothelial cells are used to cover the surface of prosthetic heart valves to decrease the risk of the formation of emboli by making the valve surface less thrombogenic.

The immune evasive endothelial cells disclosed herein can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the endothelial cells includes, but is not limited to, subcutaneous or parenteral including intravenous, intraarterial (e.g., intracoronary), intramuscular, intraperitoneal, intramyocardial, trans-endocardial, trans-epicardial, intranasal administration as well as intrathecal, and infusion techniques.

As will be appreciated by those in the art, the immune evasive PSC derivatives are transplanted using techniques known in the art that depend on both the cell type and the ultimate use of these cells. In some embodiments, the cells are transplanted either intravenously or by injection at particular locations in the patient. When transplanted at particular locations, the cells may be suspended in a gel matrix to prevent dispersion while they take hold.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The immune evasive endothelial cells disclosed herein can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLl-1, Flk-1 (KDR, VEGFR-2), FLT-1 (VEGFR-1), GATA2, GBP-1 (guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), thrombomodulin (TM, CD141), VCAM-1 (vascular cell adhesion molecule-1) (CD106), VEGF, vWF (von Willebrand factor), ZO-1, endothelial cell-selective adhesion molecule (ESAM), CD102, CD93, CD184, CD304, and DLL4.

In some embodiments, the immune evasive endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are disclosed, e.g., in U.S. Pat. No. 5,674,722.

Such immune evasive endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the immune evasive endothelial cells can be modified to secrete insulin, a blood clotting factor (e.g., Factor VIII or von Willebrand Factor), alpha-l antitrypsin, adenosine deaminase, tissue plasminogen activator, interleukins (e.g., IL-1, IL-2, IL-3), and the like.

In some embodiments, the immune evasive endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the immune evasive endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the immune evasive endothelial cells are genetically modified to contain a gene that disrupts or inhibits angiogenesis when expressed by endothelial cells in a vascularizing tumor. In some cases, the immune evasive endothelial cells can also be genetically modified to express any one of the selectable suicide genes disclosed herein which allows for negative selection of grafted endothelial cells upon completion of tumor treatment.

In some embodiments, the immune evasive endothelial cells disclosed herein are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, and/or other vascular condition or disease.

In some embodiments, the engineered immune evasive PSCs are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of immune evasive endothelial cells from a population of engineered immune evasive PSCs by in vitro differentiation comprises: (a) culturing a population of engineered immune evasive PSCs in a first culture medium comprising a GSK inhibitor; (b) culturing the population of the engineered immune evasive PSCs in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of immune evasive endothelial cells.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 μM SB-431542. In certain embodiments, the third culture medium further comprises VEGF and bFGF. In some instances, the first culture medium and/or the second medium is absent of insulin.

The cells provided herein can be cultured on a surface, such as a synthetic surface to support and/or promote differentiation of immune evasive pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}]decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the immune evasive endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some embodiments, a glass includes soda-lime glass, Pyrex glass, Vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

In some embodiments, the population of immune evasive endothelial cells is isolated from non-endothelial cells. In some embodiments, the isolated population of immune evasive endothelial cells is expanded prior to administration. In certain embodiments, the isolated population of immune evasive endothelial cells is expanded and cryopreserved prior to administration.

Additional descriptions of endothelial cells for use in the methods provided herein are found in WO2020/018615, the disclosure of which is hereby incorporated by reference in its entirety.

4. Thyroid Cells Differentiated from Immune Evasive PSCs

In some embodiments, the engineered immune evasive PSCs are differentiated into thyroid progenitor cells and thyroid follicular organoids that can secrete thyroid hormones to address autoimmune thyroiditis. Techniques to differentiate thyroid cells are known the art. See, e.g., Kurmann et al., Cell Stem Cell, 2015 Nov. 5; 17 (5): 527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

5. Hepatocytes Differentiated from Immune Evasive PSCs

In some embodiments, the engineered immune evasive PSCs are differentiated into hepatocytes to address loss of the hepatocyte functioning or cirrhosis of the liver. There are a number of techniques that can be used to differentiate PSCs into hepatocytes; see for example, Pettinato et al, doi: 10.1038/spre32888, Snykers et al., Methods Mol Biol, 2011 698:305-314, Si-Tayeb et al., Hepatology, 2010, 51:297-305 and Asgari et al., Stem Cell Rev, 2013, 9 (4): 493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

6. Pancreatic Islet Cells Differentiated from Immune Evasive PSCs

In some embodiments, pancreatic islet cells (also referred to as pancreatic beta cells) are derived from the engineered immune evasive PSCs disclosed herein. In some instances, the engineered immune evasive PSCs are differentiated into various pancreatic islet cell types and transplanted or engrafted into subjects (e.g., recipients). As will be appreciated by those in the art, the methods for differentiation depend on the desired cell type using known techniques. Exemplary pancreatic islet cell types include, but are not limited to, pancreatic islet progenitor cell, immature pancreatic islet cell, mature pancreatic islet cell, and the like. In some embodiments, pancreatic cells disclosed herein are administered to a subject to treat diabetes.

In some embodiments, pancreatic islet cells are derived from the engineered immune evasive PSCs disclosed herein. Useful method for differentiating pluripotent stem cells into pancreatic islet cells are disclosed, for example, in U.S. Pat. Nos. 9,683,215; 9,157,062; and 8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Sox17, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 μm to about 25 μm.

In some embodiments, the engineered immune evasive PSCs are differentiated into beta-like cells or islet organoids for transplantation to address type I diabetes mellitus (T1DM). Cell systems are a promising way to address TIDM, see, e.g., Ellis et al., Nat Rev Gastroenterol Hepatol. 2017 October; 14(10):612-628, incorporated herein by reference. Additionally, Pagliuca et al. (Cell, 2014, 159(2): 428-39) reports on the successful differentiation of B-cells from hiPSCs, the contents incorporated herein by reference in its entirety and in particular for the methods and reagents disclosed there for the large-scale production of functional human β cells from human pluripotent stem cells). Furthermore, Vegas et al. shows the production of human β cells from human pluripotent stem cells followed by encapsulation to avoid immune rejection by the recipient; Vegas et al., Nat Med, 2016, 22(3):306-11, incorporated herein by reference in its entirety and in particular for the methods and reagents disclosed there for the large-scale production of functional human β cells from human pluripotent stem cells.

In some embodiments, the method of producing a population of immune evasive pancreatic islet cells from a population of engineered immune evasive pluripotent cells by in vitro differentiation comprises: (a) culturing a population of engineered immune evasive PSCs in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of immune evasive pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the population of immune evasive pancreatic islet cells is isolated from non-pancreatic islet cells. In some embodiments, the isolated population of immune evasive pancreatic islet cells is expanded prior to administration. In certain embodiments, the isolated population of immune evasive pancreatic islet cells is expanded and cryopreserved prior to administration.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26; 3(4): 385-394.e3, hereby incorporated by reference in its entirety, and specifically for the biomarkers disclosed there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional disclosure of pancreatic islet cells including pancreatic beta islet cells for use in the present technology are found in WO2020/018615, the disclosure is herein incorporated by reference in its entirety.

7. Retinal Pigmented Epithelium (RPE) Cells Differentiated from Immune Evasive PSCs

Provided herein are retinal pigmented epithelium (RPE) cells derived from the engineered immune evasive PSCs disclosed herein. For instance, human RPE cells can be produced by differentiating immune evasive human PSCs. In some embodiments, engineered immune evasive PSCs are differentiated into various RPE cell types and transplanted or engrafted into subjects (e.g., recipients). As will be appreciated by those in the art, the methods for differentiation depend on the desired cell type using known techniques.

The term “RPE” cells refers to pigmented retinal epithelial cells having a genetic expression profile similar or substantially similar to that of native RPE cells. Such RPE cells derived from pluripotent stem cells may possess the polygonal, planar sheet morphology of native RPE cells when grown to confluence on a planar substrate.

The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-real ted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

Exemplary RPE cell types include, but are not limited to, retinal pigmented epithelium (RPE) cell, RPE progenitor cell, immature RPE cell, mature RPE cell, functional RPE cell, and the like.

Useful methods for differentiating pluripotent stem cells into RPE cells are disclosed in, for example, U.S. Pat. Nos. 9,458,428 and 9,850,463, the disclosures are herein incorporated by reference in their entirety, including the specifications. Additional methods for producing RPE cells from human induced pluripotent stem cells can be found in, for example, Lamba et al., PNAS, 2006, 103 (34): 12769-12774; Mellough et al., Stem Cells, 2012, 30 (4): 673-686; Idelson et al., Cell Stem Cell, 2009, 5 (4): 396-408; Rowland et al., Journal of Cellular Physiology, 2012, 227 (2): 457-466, Buchholz et al., Stem Cells Trans Med, 2013, 2 (5): 384-393, and da Cruz et al., Nat Biotech, 2018, 36:328-337.

Human pluripotent stem cells have been differentiated into RPE cells using the techniques disclosed in Kamao et al, Stem Cell Reports 2014:2:205-18, hereby incorporated by reference in its entirety and in particular for the methods and reagents disclosed there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014, 2 (2): 205-18, the contents incorporated herein by reference in its entirety and specifically for the markers disclosed in the first paragraph of the results section.

In some embodiments, the method of producing a population of immune evasive retinal pigmented epithelium (RPE) cells from a population of engineered immune evasive PSCs by in vitro differentiation comprises: (a) culturing a population of engineered immune evasive PSCs in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of immune evasive RPE cells. In some embodiments, the ALK inhibitor is SB-431542, a derivative thereof, or a variant thereof. In some instances, the ALK inhibitor is at a concentration ranging from about 2 mM to about 10 μM. In some embodiments, the ROCK inhibitor is Y-27632, a derivative thereof, or a variant thereof. In some instances, the ROCK inhibitor is at a concentration ranging from about 1 μM to about 10 μM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2 (2): 205-18, the contents are herein incorporated by reference in its entirety and specifically for the results section.

Additional descriptions of RPE cells for use in the present technology are found in WO2020/018615, the disclosure is herein incorporated by reference in its entirety.

For therapeutic applications, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

8. T Lymphocyte Derived from Immune Evasive PSCs

Provided herein are therapeutic T lymphocytes (T cells, such as chimeric antigen receptor (CAR) T cells) produced (e.g., generated, cultured, or derived) from the engineered immune evasive PSCs disclosed herein (e.g., immune evasive ESCs, immune evasive iPSCs). Methods for generating T cells, including CAR-T-cells, from pluripotent stem cells (e.g., iPSC) are disclosed, for example, in Iriguchi et al., Nature Communications 12, 430 (2021); Themeli et al. 16 (4): 357-366 (2015); Themeli et al., Nature Biotechnology 31:928-933 (2013).

In some embodiments, the immune evasive PSC-derived T cells do not activate an immune response in the patient (e.g., recipient upon administration). Provided are methods of treating a disorder by administering a population of immune evasive PSC-derived T cells to a subject (e.g., recipient) or patient in need thereof. In some embodiments, the immune evasive cells disclosed herein comprise PSC-derived T cells engineered (e.g., are modified) to express a CAR including but not limited to a CAR disclosed herein. In some instances, the PSC-derived T cells are populations or subpopulations of T cells. In some embodiments, the PSC-derived T cells disclosed herein such as the engineered or modified CAR-T cells comprise reduced expression of an endogenous T cell receptor.

Immune evasive PSC-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

9 NK Cells Derived from Immune Evasive PSCs

Provided herein are natural killer (NK) cells derived from the engineered immune evasive PSCs disclosed herein (e.g., immune evasive iPSCs).

NK cells (also defined as “large granular lymphocytes”) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that can be used to generate NK cells from pluripotent stem cells (e.g., iPSC); see, for example, Zhu et al., Methods Mol Biol. 2019; 2048:107-119; Knorr et al., Stem Cells Transl Med. 2013 2 (4): 274-83. doi: 10.5966/sctm.2012-0084; Zeng et al., Stem Cell Reports. 2017 Dec. 12; 9 (6): 1796-1812; Ni et al., Methods Mol Biol. 2013; 1029:33-41; Bernareggi et al., Exp Hematol. 2019 71:13-23; Shankar et al., Stem Cell Res Ther. 2020; 11 (1): 234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the NK cells do not activate an immune response in the patient (e.g., recipient upon administration). Provided are methods of treating a disorder by administering a population of NK cells to a subject (e.g., recipient) or patient in need thereof.

### C. Compositions Comprising Therapeutic Cells

In some aspects, the present technology provides pharmaceutical compositions comprising one or more populations of the engineered cells and/or cells differentiated from the engineered cells according to various embodiments disclosed herein. For example, immune evasive cells derived from different donors can be mixed and formulated into a composition. For example, immune evasive cells from recipient patient and immune evasive cells from one or more donors who are not the recipient patient can be mixed and formulated into a composition. For example, different types or different subtypes of immune evasive cells can be mixed and formulated into a composition.

In some embodiments, the pharmaceutical compositions can have various formulations, for example, injectable formulations, lyophilized formulations, liquid formulations, oral formulations, etc., depending on the suitable routes of administration.

In some embodiments, the pharmaceutical compositions can be co-formulated in the same dosage unit or can be individually formulated in separate dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e., 1 to 10, 1 to 8, 1 to 6, 1 to 4, or 1 to 2) of times per day, or as many times as needed to elicit a therapeutic response.

In some embodiments, a single dosage unit includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells.

### 1. Pharmaceutically Acceptable Carriers

In some embodiments, the pharmaceutical composition provided herein further includes a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline).

In some embodiments, the pharmaceutical composition includes one or more electrolyte base solutions selected from the group consisting of lactated CryoStor®, Ringer's solution, PlasmaLyte-ATM, Iscove's Modified Dulbecco's Medium, Normosol-R™, Veen-D™, Polysal® and Hank's Balanced Salt Solution (containing no phenol red). These base solutions closely approximate the composition of extracellular mammalian physiological fluids.

In some embodiments, the pharmaceutical composition includes one or more cryoprotective agents selected from the group consisting of arabinogalactan, glycerol, polyvinylpyrrolidone (PVP), dextrose, dextran, trehalose, sucrose, raffinose, hydroxyethyl starch (HES), propylene glycol, human serum albumin (HSA), and dimethylsulfoxide (DMSO). In some embodiments, the pharmaceutically acceptable buffer is neutral buffer saline or phosphate buffered saline. In some embodiments, pharmaceutical compositions provided herein include one or more of CryoStor® CSB, Plasma-Lyte-A™, HSA, DMSO, and trehalose.

CryoStor® is an intracellular-like optimized solution containing osmotic/oncotic agents, free radical scavengers, and energy sources to minimize apoptosis, minimize ischemia/reperfusion injury and maximize the post-thaw recovery of the greatest numbers of viable, functional cells. CryoStor® is serum- and protein-free, and non-immunogenic. CryoStor® is cGMP-manufactured from raw materials of USPgrade or higher. CryoStor® is a family of solutions pre-formulated with 0%, 2%, 5% or 10% DMSO. CryoStor® CSB is a DMSO-free version of CryoStor®. In some embodiments, the pharmaceutical composition includes a base solution of CryoStor® CSB at a concentration of about 0-100%, 5-95%, 10-90%, 15-85%, 20-80%, 30-80%, 40-80%, 50-80%, 60-80%, 70-80%, 25-75%, 30-70%, 35-65%, 40-60%, or 45-55% w/w. In some embodiments, the pharmaceutical composition includes a base solution of CryoStor® CSB at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

PlasmaLyte-A™ is a non-polymeric plasma expander and contains essential salts and nutrients similar to those found in culture medium but does not contain additional constituents found in tissue culture medium which are not approved for human infusion, e.g., phenol red, or are unavailable in U.S.P. grade. PlasmaLyte-A™ contains about 140 mEq/liter of sodium (Na), about 5 mEq/liter of potassium (K), about 3 mEq/liter of magnesium (Mg), about 98 mEq/liter of chloride (C1), about 27 mEq/liter of acetate, and about 23 mEq/liter of gluconate. PlasmaLyte-A™ is commercially available from Baxter, Hyland Division, Glendale Calif., product No. 2B2543. In some embodiments, the pharmaceutical composition includes a base solution of PlasmaLyte-A™ at a concentration of about 0-100%, 5-95%, 10-90%, 15-85%, 15-80%, 15-75%, 15-70%, 15-65%, 15-60%, 15-55%, 15-50%, 15-45%, 15-40%, 15-35%, 15-30%, 15-25%, 20-80%, 20-75%, 20-70%, 20-65%, 20-60%, 20-55%, 20-50%, 20-45%, 20-40%, 20-35%, 20-30%, 25-75%, 30-70%, 35-65%, 40-60%, or 45-55% w/w. In some embodiments, the pharmaceutical composition includes a base solution of PlasmaLyte-A™ at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

In some embodiments, the pharmaceutical composition includes human serum albumin (HSA) at a concentration of about 0-10%, 0.3-9.3%, 0.3-8.3%, 0.3-7.3%, 0.3-6.3%, 0.3-5.3%, 0.3-4.3%, 0.3-3.3%, 0.3-2.3%, 0.3-1.3%, 0.6-8.3%, 0.9-7.3%, 1.2-6.3%, 1.5-5.3%, 1.8-4.3%, or 2.1-3.3% w/v. In some embodiments, the pharmaceutical composition includes HSA at a concentration of about 0%, 0.3%, 0.6%, 0.9%, 1.2%, 1.5%, 1.8%, 2.1%, 2.4%, 2.7%, 3.0%, 3.3%, 3.6%, 3.9%, 4.3%, 4.6%, 4.9%, 5.3%, 5.6%, 5.9%, 6.3%, 6.6%, 6.9%, 7.3%, 7.6%, 7.9%, 8.3%, 8.6%, 8.9%, 9.3%, 9.6%, 9.9%, or 10% w/v.

In some embodiments, the pharmaceutical composition includes DMSO at a concentration of about 0-10%, 0.5-9.5%, 1-9%, 1.5-8.5%, 2-8%, 3-8%, 4-8%, 5-8%, 6-8%, 7-8%, 2.5-7.5%, 3-7%, 3.5-6.5%, 4-6%, or 4.5-5.5% v/v. In some embodiments, the pharmaceutical composition includes HSA at a concentration of about 0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0% v/v.

In some embodiments, the pharmaceutical composition includes trehalose at a concentration of about 0-500 mM, 50-450 mM, 100-400 mM, 150-350 mM, or 200-300 mM. In some embodiments, the pharmaceutical composition includes trehalose at a concentration of about 0 mM, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM.

Exemplary pharmaceutical composition components are shown in Table 9.

TABLE 9
Exemplary pharmaceutical composition components

| For- | | | Additional | |
|---|---|---|---|---|
| mulation | Base Solution | c[DMSO] | c[HSA]* | c[trehalose] |
| A | 75% | 7.5% | 0.3% | |
| B | CroStor ® | 3.75% | 0.3% | |
| C | CSB + 25% | | 5.3% | |
| D | PlasmaLyte | | 0.3% | 250 mM |
| | A ™ + | | | |
| | 1.2% HSA | | | |
| E | 100% | 7.5% | 0.3% | |
| F | PlasmaLyte | 7.5% | 5.3% | |
| G | A ™ + | 7.5% | 5.3% | 250 mM |
| | 1.2% HSA | | | |

* Additional HSA in addition to PlasmaLyte.

In some embodiments, the pharmaceutical composition comprises immune evasive cells disclosed herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

### 2. Formulations and Dosage Regimens

Any therapeutically effective amount of cells disclosed herein can be included in the pharmaceutical composition, depending on the indication being treated. Non-limiting examples of the cells include immune evasive primary cells, and cells differentiated from immune evasive PSCs such as induced pluripotent stem cells disclosed herein. In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, or 1×10¹⁰-5×10¹⁰ cells. In exemplary embodiments, the pharmaceutical composition includes from about 1.0×10⁶ to about 2.5×10⁸ cells. In certain embodiments, the pharmaceutical composition includes from about 2.0×10⁶ to about 5.0×10⁸ cells, such as but not limited to, immune evasive primary cells, cells differentiated from immune evasive induced pluripotent stem cells.

In some embodiments, the pharmaceutical composition has a volume of at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In certain embodiments, the pharmaceutical composition has a volume that ranges from about 10 ml to about 50 ml.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

In some embodiments, a therapeutically effective dose or a clinically effective dose of the pharmaceutical composition includes about 1.0×10⁵ to about 2.5×10⁸ cells at a volume of about 10 ml to 50 ml and the pharmaceutical composition is administered as a single therapeutically effective dose or clinically effective dose. In some cases, the therapeutically effective dose or clinically effective dose includes about 1.0×10⁵ to about 2.5×10⁸ immune evasive primary cells disclosed herein at a volume of about 10 ml to 50 ml. In some cases, the therapeutically effective dose or clinically effective dose includes about 1.0×10⁵ to about 2.5×10⁸ immune evasive primary cells that have been disclosed herein at a volume of about 10 ml to 50 ml. In various cases, the therapeutically effective dose or clinically effective dose includes about 1.0×10⁵ to about 2.5×10⁸ immune evasive cells differentiated from engineered immune evasive PSCs such as induced pluripotent stem cells disclosed herein at a volume of about 10 ml to 50 ml. In some embodiments, the therapeutically effective dose or clinically effective dose is 1.0×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, or 2.5×10⁸ cells differentiated from engineered immune evasive PSCs such as induced pluripotent stem cells disclosed herein at a volume of about 10 ml to 50 ml. In other cases, the therapeutically effective dose or clinically effective dose is at a range that is lower than about 1.0×10⁵ to about 2.5×10⁸ cells, including immune evasive primary cells or cells differentiated from engineered immune evasive PSCs such as induced pluripotent stem cells. In yet other embodiments, the therapeutically effective dose or clinically effective dose is at a range that is about 1.0×10⁵ to about 2.5×10⁸ cells or higher, including immune evasive primary cells and cells differentiated from engineered immune evasive PSCs such as induced pluripotent stem cells.

In some embodiments, the pharmaceutical composition is administered as a single therapeutically effective dose or clinically effective dose of from about 1.0×10⁵ to about 1.0×10⁷ cells (such as immune evasive primary cells and cells differentiated from engineered immune evasive PSCs such as induced pluripotent stem cells) per kg body weight for subjects 50 kg or less. In some embodiments, the pharmaceutical composition is administered as a single therapeutically effective dose or clinically effective dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the therapeutically effective dose or clinically effective dose is 0.5×10⁵, 0.6×10⁵, 0.7×10⁵, 0.8×10⁵, 0.9×10⁵, 1.0×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 2.6×10⁵, 2.7×10⁵, 2.8×10⁵, 2.9×10⁵, 3.0×10⁵, 3.1×10⁵, 3.2×10⁵, 3.3×10⁵, 3.4×10⁵, 3.5×10⁵, 3.6×10⁵, 3.7×10⁵, 3.8×10⁵, 3.9×10⁵, 4.0×10⁵, 4.1×10⁵, 4.2×10⁵, 4.3×10⁵, 4.4×10⁵, 4.5×10⁵, 4.6×10⁵, 4.7×10⁵, 4.8×10⁵, 4.9×10⁵, 5.0×10⁵, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 0.5×10⁷, 0.6×10⁷, 0.7×10⁷, 0.8×10⁷, 0.9×10⁷, or 1.0×10⁷ cells per kg body weight for subjects 50 kg or less. In some embodiments, the therapeutically effective dose or clinically effective dose is from about 0.2×10⁶ to about 5.0×10⁶ cells per kg body weight for subjects 50 kg or less. In certain embodiments, the therapeutically effective dose or clinically effective dose is at a range that is lower than from about 0.2×10⁶ to about 5.0×10⁶ cells per kg body weight for subjects 50 kg or less. In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the cells are administered in a single therapeutically effective dose of from about 1.0×10⁶ to about 5.0×10⁸ cells (such as immune evasive primary cells and cells differentiated from engineered immune evasive PSCs such as induced pluripotent stem cells) for subjects above 50 kg. In some embodiments, the pharmaceutical composition is administered as a single therapeutically effective dose or clinically effective dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×107, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or more. In some embodiments, the therapeutically effective dose or clinically effective dose is 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, or 5.0×10⁸ cells per kg body weight for subjects 50 kg or more. In certain embodiments, the cells are administered in a single therapeutically effective dose or clinically effective dose of about 1.0×10⁷ to about 2.5×10⁸ cells for subjects above 50 kg. In some embodiments, the cells are administered in a single therapeutically effective dose or clinically effective dose of a range that is less than about 1.0×10⁷ to about 2.5×10⁸ cells for subjects above 50 kg. In some embodiments, the cells are administered in a single therapeutically effective dose or clinically effective dose of a range that is higher than about 1.0×10⁷ to about 2.5×10⁸ cells for subjects above 50 kg. In some embodiments, the dose is administered intravenously. In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the therapeutically effective dose or clinically effective dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single therapeutically effective dose or clinically effective dose of the pharmaceutical composition is present in a single infusion bag. In other embodiments, a single therapeutically effective dose or clinically effective dose of the pharmaceutical composition is divided into 2, 3, 4 or 5 separate infusion bags.

In some embodiments, the cells disclosed herein are administered in a plurality of doses such as 2, 3, 4, 5, 6 or more doses, wherein the plurality of doses together constitute a therapeutically effective dose or clinically effective dose regimen. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In certain embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different. The first dosage regimen can be administered to the subject at least 1 month, 2 months, 3 months, 4 months, 5 months, 6 months, 7 months, 8 months, 9 months, 10 months, 11 months, 12 months, 1-3 months, I-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of immune evasive cells disclosed herein. In other embodiments, at least two of the plurality of dosage regimens comprise different types of immune evasive cells disclosed herein.

### V. Methods of Treatment

In some aspects, the present technology provides methods for treating and/or preventing a disease in a subject in need thereof using a population of therapeutic cells derived from or generated by methods according to various embodiments disclosed herein. The method entails administering to the subject a therapeutically effective amount of the immune evasive cells, or a pharmaceutical composition containing the same.

The immune evasive cell can be an autologous cell, i.e., obtained from the subject who will receive the cell after modification. Alternatively, the immune evasive cell can be an allogeneic cell, i.e., obtained from someone other than the subject who will receive the engineered cell after modification. In either of these embodiments, the immune evasive cell can be a primary cell obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, especially in the case of allogeneic cells, the immune evasive cell can be derived from an ESC or an iPSC.

Therapeutic cells disclosed herein are useful to treat a disorder such as, but not limited to, a cancer, a genetic disorder, a chronic infectious disease, an autoimmune disorder, a neurological disorder, a cardiac disease or condition, and the like.

In some embodiments, the therapeutic cells disclosed herein are administered for treatment of a cellular deficiency or as a cellular therapy for the treatment of a condition or disease in a tissue or organ selected from the group consisting of heart, lung, kidney, liver, pancreas, intestine, stomach, cornea, bone marrow, blood vessel, heart valve, brain, spinal cord, and bone.

Candidates for cellular therapy include any patient having a disease or condition that may potentially benefit from the therapeutic effects of the immune evasive therapeutic cells provided herein. In some embodiments, the patient has a cellular deficiency. A candidate who benefits from the therapeutic effects of the immune evasive therapeutic cells provided herein exhibit an elimination, reduction or amelioration of ta disease or condition. As used herein, a “cellular deficiency” refers to any disease or condition that causes a dysfunction or loss of a population of cells in the patient, wherein the patient is unable to naturally replace or regenerate the population of cells. Exemplary cellular deficiencies include, but are not limited to, autoimmune diseases (e.g., multiple sclerosis, myasthenia gravis, rheumatoid arthritis, diabetes, systemic lupus and erythematosus), neurodegenerative diseases (e.g., Huntington's disease and Parkinson's disease), cardiovascular conditions and diseases, vascular conditions and diseases, corneal conditions and diseases, liver conditions and diseases, thyroid conditions and diseases, and/or kidney conditions and diseases.

In some embodiments, the therapeutic cells disclosed herein are administered for treatment of a cellular deficiency or as a cellular therapy, wherein: (a) the cellular deficiency is associated with a neurodegenerative disease or the cellular therapy is for the treatment of a neurodegenerative disease; (b) the cellular deficiency is associated with a liver disease or the cellular therapy is for the treatment of liver disease; (c) the cellular deficiency is associated with a corneal disease or the cellular therapy is for the treatment of corneal disease; (d) the cellular deficiency is associated with a cardiovascular condition or disease or the cellular therapy is for the treatment of a cardiovascular condition or disease; (e) the cellular deficiency is associated with diabetes or the cellular therapy is for the treatment of diabetes; (f) the cellular deficiency is associated with a vascular condition or disease or the cellular therapy is for the treatment of a vascular condition or disease; (g) the cellular deficiency is associated with autoimmune thyroiditis or the cellular therapy is for the treatment of autoimmune thyroiditis; or (h) the cellular deficiency is associated with a kidney disease or the cellular therapy is for the treatment of a kidney disease.

In some embodiments, the therapeutic cells disclosed herein are administered for treatment of: (a) a neurodegenerative disease selected from the group consisting of leukodystrophy, Huntington's disease, Parkinson's disease, multiple sclerosis, transverse myelitis, and Pelizaeus-Merzbacher disease (PMD); (b) a liver disease comprises cirrhosis of the liver; (c) a corneal disease that is Fuchs dystrophy or congenital hereditary endothelial dystrophy; or (d) a cardiovascular disease that is myocardial infarction or congestive heart failure.

In some embodiments, the disease is cancer, such as a hematologic malignancy, liver cancer, pancreatic cancer, breast cancer, ovarian cancer, colorectal cancer, lung cancer, non-small cell lung cancer, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. Non-limiting examples of hematologic malignancies include myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), acute myeloid lymphoid leukemia, chronic myelogenous leukemia (CML), blast crisis chronic myelogenous leukemia (bcCML), B-cell acute lymphoid leukemia (B-ALL), diffuse large B-cell lymphoma, T-cell acute lymphoid leukemia (T-ALL), T-cell lymphoma, and B-cell lymphoma.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

In some embodiments, the patient who is treated by the therapeutic cells disclosed herein is receiving a tissue or organ transplant, optionally wherein the tissue or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and a partial cornea transplant.

In some embodiments, the tissue or organ transplant is an allograft transplant. In some embodiments, the tissue or organ transplant is an autograft transplant.

In some embodiments, the therapeutic cells are administered for the treatment of a cellular deficiency in a tissue or organ and the tissue or organ transplant is for the replacement of the same tissue or organ. In some embodiments, the therapeutic cells are administered for the treatment of a cellular deficiency in a tissue or organ and the tissue or organ transplant is for the replacement of a different tissue or organ. In some embodiments, the organ transplant is a kidney transplant, a pancreas transplant, and/or a liver transplant, and the population of cells is a population of pancreatic islet cells which includes pancreatic beta islet cells or the population of cells is a population of pancreatic beta islet cells. In some embodiments, the patient has diabetes and the population of cells is a population of pancreatic islet cells including pancreatic beta islet cells or the population of cells is a population of pancreatic beta islet cells. In some embodiments, the organ transplant is a heart transplant and the population of cells is a population of pacemaker cells. In some embodiments, the organ transplant is a partial liver transplant and the population of cells is a population of hepatocytes or hepatic progenitor cells.

In some embodiments, the therapeutic cells, or a pharmaceutical composition containing the same, according to the present technology may be administered in a manner appropriate to the disease, condition, or disorder to be treated as determined by persons skilled in the medical art. In any of the above embodiments, the therapeutic cells, or a pharmaceutical composition containing the same, can be administered intravenously, intraperitoneally, intratumorally, into the bone marrow, into a lymph node, or into the cerebrospinal fluid, so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

As will be appreciated by those in the art, the therapeutic cells can be transplanted using techniques known in the art that depends on both the cell type and the ultimate use of these cells. In general, the therapeutic cells disclosed herein can be transplanted either intravenously or by injection at particular locations in the patient. When transplanted at particular locations, the therapeutic cells may be suspended in a gel matrix to prevent dispersion while they take hold.

In some embodiments, the amount of the immune evasive cells in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰ cells, or more.

In some embodiments, the methods comprise administering to the subject the therapeutic cells, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the engineered cells or the pharmaceutical composition containing the same can be administered every day, every other day, every third day, weekly, biweekly (i.e., every other week), every third week, monthly, every other month, or every third month.

In some embodiments, the therapeutic cells, or a pharmaceutical composition containing the same, may be administered over a pre-determined time period. Alternatively, the therapeutic cells, or a pharmaceutical composition containing the same, may be administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the engineered cell, or the pharmaceutical composition containing the same.

In some embodiments, the method further entails administering one or more other cancer therapies such as surgery, immunotherapy, radiotherapy, and/or chemotherapy to the subject, sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent (see, for example, Akkin et al., Molecules 26:3382 (2021), and Esfahani et al., Curr Oncol. 27 (S2): 87-97 (2020), the disclosures of which about immunotherapy agents are incorporated by reference), a chemotherapy agent, and a biologic agent. Examples of chemotherapy agents include but are not limited to alkylating agents, antimetabolites, antimicrotubular agents, antibiotics, and others. In some embodiments, alkylating agents include nitrogen mustard (e.g., bendamustine, cyclophosphamide, and ifosfamide), nitrosoureas (e.g., carmustine, and lomustine), platinum analogs (e.g., carboplatin, cisplatin, and oxaliplatin), triazenes (e.g., dacarbazine, procarbazine, temozolamide), alkyl sulfonate (e.g., busulfan), and ethyleneimine (e.g., thiotepa). In some embodiments, antimetabolites include cytidine analogs (e.g., azacitidine, decitabine, cytarabine, and gemcitabine), folate antagonists (e.g., methotrexate, and pemetrexed), purine analogs (e.g., cladribine, clofarabine, and nelarabine), and pyrimidine analogs (e.g., fluorouracil (5-FU), and capecitabine (prodrug of 5-FU)). In some embodiments, antimicrotubular agents include topoisomerase II inhibitors (e.g., anthracyclines such as doxorubicin, daunorubicin, idarubicin, and mitoxantrone), topoisomerase I inhibitors (e.g., Irinotecan, and Topotecan), taxanes (e.g., paclitaxel, docetaxel, and cabazitaxel), and Vinca alkaloids (e.g., vinblastine, vincristine, and vinorelbine). In some embodiments, antibiotics include actinomycin D, bleomycin, and daunomycin. In some embodiments, other chemotherapy agents include hydroxyurea, tretinoin, arsenic trioxide, and proteasome inhibitors.

In some embodiments, the subject was administered the one or more additional therapeutic agents before administration of the engineered cell, or a pharmaceutical composition containing the same. In some embodiments, the subject is co-administered the one or more additional therapeutic agents and the engineered cell, or a pharmaceutical composition containing the same. In some embodiments, the subject was administered the one or more additional therapeutic agents after administration of the engineered cell, or a pharmaceutical composition containing the same.

As one of ordinary skill in the art would understand, the one or more additional therapeutic agents and the therapeutic cells, or a pharmaceutical composition containing the same, can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

In some embodiments, the method further comprises administering a CD47-SIRPα blockade agent to a patient that has been previously administered therapeutic cells comprising exogenously expressing CD47 proteins. As such, without wishing to be bound by theory, it is believed that the cells can no longer evade immune recognition and thus are recognized by the patient's immune cells and targeted for cell death and/or cell clearance. In some instances, the patient's innate immune cells are activated or mobilized to decrease the number of the previously administered cells and their derivatives (e.g., progeny).

Any of the CD47-SIRPα blockade agents disclosed herein are useful for treating a patient with a condition or disease that is responsive to cell therapy. For instance, such a condition or disease can be characterized by the presence of unhealthy cells or tissue (e.g., diseased cells or tissue) that can be replaced by therapeutic interventions comprising healthy cell, including cells that are not in a diseased state. In some embodiments, the patient having the condition or disease is administered a cell therapy that is expected to ameliorate one or more symptoms of the condition or disease. Any of the CD47-SIRPα blockade agents can be used for the treatment, reduction or amelioration of an adverse effect adverse effect subsequent to administration of a population of cells comprising exogenously expressed CD47 polypeptides. In some embodiments, the agent is used for the control of an effect of a cell therapy in a patient, to modulate an activity of a cell therapy in a patient, or to reduce the number of cells comprising exogenously expressed CD47 polypeptides in the patient.

### VI. Conclusion

The above detailed description of embodiments of the technology are not intended to be exhaustive or to limit the technology to the precise forms disclosed above. Although specific embodiments of, and examples for, the technology are disclosed herein for illustrative purposes, various equivalent modifications are possible within the scope of the technology as those skilled in the relevant art will recognize. For example, although steps are presented in a given order, alternative embodiments may perform steps in a different order. The various embodiments disclosed herein may also be combined to provide further embodiments.

From the foregoing, it will be appreciated that specific embodiments of the technology have been disclosed herein for purposes of illustration, but well-known components and functions have not been shown or disclosed in detail to avoid unnecessarily obscuring the description of the embodiments of the technology. Where the context permits, singular or plural terms may also include the plural or singular term, respectively. Further, while advantages associated with some embodiments of the technology have been disclosed in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or disclosed herein.

### EXAMPLES

### Example 1: MAD7 sgRNA Library Screening

I. sgRNA Library Design

MAD7 sgRNAs targeting regions of interest were designed using the following custom parameters:

| | Guide parameters |
|---|---|
| Design Type | Single Guide |
| Guide Length | 21 |
| Genome | GRCh38.p13 EnsemblRelease_103 (Human (Homo sapiens)) |
| PAM | 5′ YTTN |
| | Mad7 crRNA Scaffold Sequences |
| Mad 7 scaffold 1 | GUCAAAAGACCUUUUUAAUUUCUACUCUUGUAGAU (SEQ ID |
| | No. 24455) |
| Mad 7 scaffold 2 | GUCAAAAGACCUUUGGAAUUUCUACUCUUGUAGAU (SEQ ID |
| | No. 24456) |
| Mad 7 scaffold 3 | GGAAUUUCUACUCUUGUAGAU (SEQ ID No. 24457) |

The MAD7 scaffold 1 sequence was appended to the 5′ end of the single gRNA. Library synthesis was performed and standard 2′-O-methyl analogs and 3′ phosphorothioate internucleotide linkages were made at the first three 5′ and 3′ terminal RNA residues.

II. sgRNA Library Screening

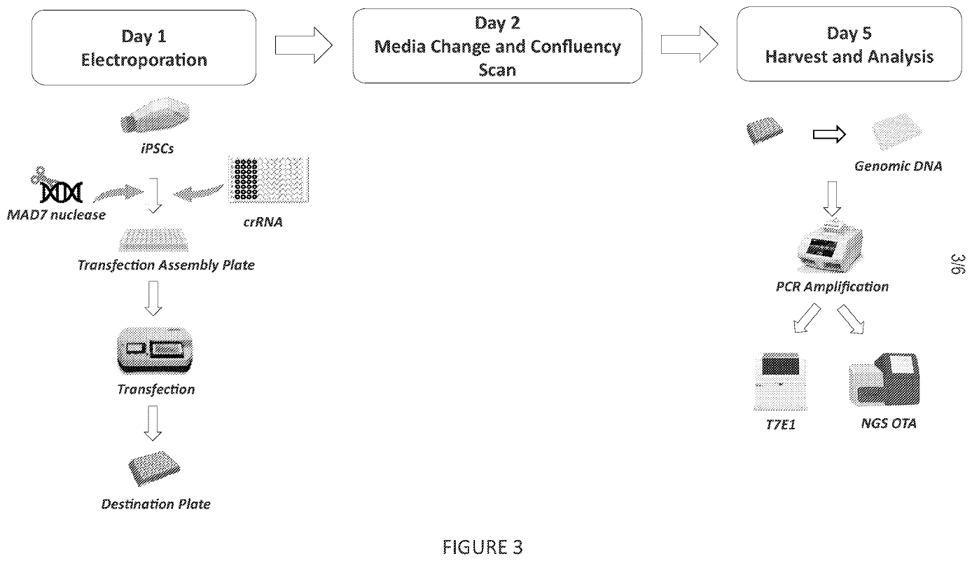

As shown in FIG. 3, gene editing occurred over several days. On Day 1, prior to electroporation, iPSCs were fed with standard iPSC medium plus 5 μM Rock-Inhibitor. Additionally, the components for gene editing transfection reactions were assembled in 96 well plates. The 96 well plates were kept on ice prior to transfection. mRNA encoding MAD7 nuclease and crRNA or gRNA were added to the wells. The iPSCs were then re-suspended in buffer and added to each well. iPSCs were then nucleofected (although it will be appreciated that other methods of introducing nucleic acid into cells can be used). After nucleofection, media was added to each well. Wells were then mixed gently and plates were transferred to an incubator to grow the cells.

On Day 2, each well was imaged to measure cell confluence and the media in each well was replaced with fresh media. On Days 3 and 4, media was exchanged in each well. Finally, on Day 5, cells were harvested from the transfection plates and nucleic acid was harvested.

### T7E1 Assay

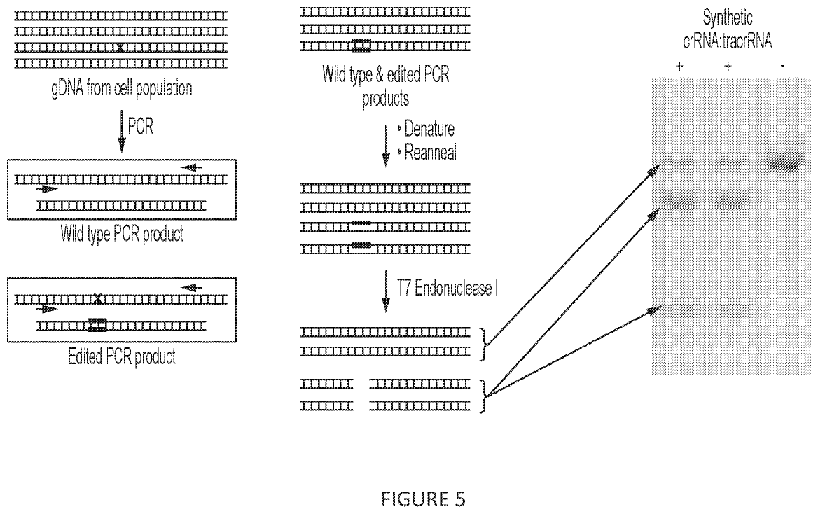

T7 Endonuclease-I (T7E1) recognizes and cleaves structural deformities in DNA heteroduplexes. During a successful gene editing event, non-homologous end joining (NHEJ) repair introduces a mutation around the cut site, which can be detected using the T7E1 assay. Specifically, the T7E1 assay can be used to determine the percentage of gene editing and/or gene modification. In the present example, the T7E1 assay was used to screen MAD7 guide RNA activity. Briefly, the genomic DNA of cells treated with MAD7 nuclease and corresponding guide RNA was amplified by PCR using primers that surround the guide RNA target site. These target amplicons were then denatured and annealed to form heteroduplexes between mutant and wild-type PCR amplicons. Next, the samples were treated with T7 endonuclease I, which recognizes and cleaves DNA mismatches in the heteroduplexes. Finally, the T7-treated DNA was run on the Tapestation to determine which test samples included full length products (uncut bands, which indicate a lack of successful editing/modification) and cleavage products (cut bands, which indicate successful editing/modification). The intensity of the respective bands were then used to calculate gene editing/modification percentages. See FIG. 5 for an exemplary schematic of the T7E1 DNA mismatch detection assay.

### REFERENCES

1. R. D. Mashal, J. Koontz, J. Sklar, Detection of mutations by cleavage of DNA heteroduplexes with bacteriophage resolvases. Nat Genet 9, 177-183 (1995).
2. L. Vouillot, A. Thelie, N. Pollet, Comparison of T7EI and surveyor mismatch cleavage assays to detect mutations triggered by engineered nucleases. G3 (Bethesda) 5, 407-415 (2015).
3. Considerations for T7 Endonuclease I (T7EI) mismatch assays (https://horizondiscovery.com/en/resources/featured-articles/considerations-for-t7-endonuclease-i-t7ei-mismatch-assays)

### OTA-NGS Assay

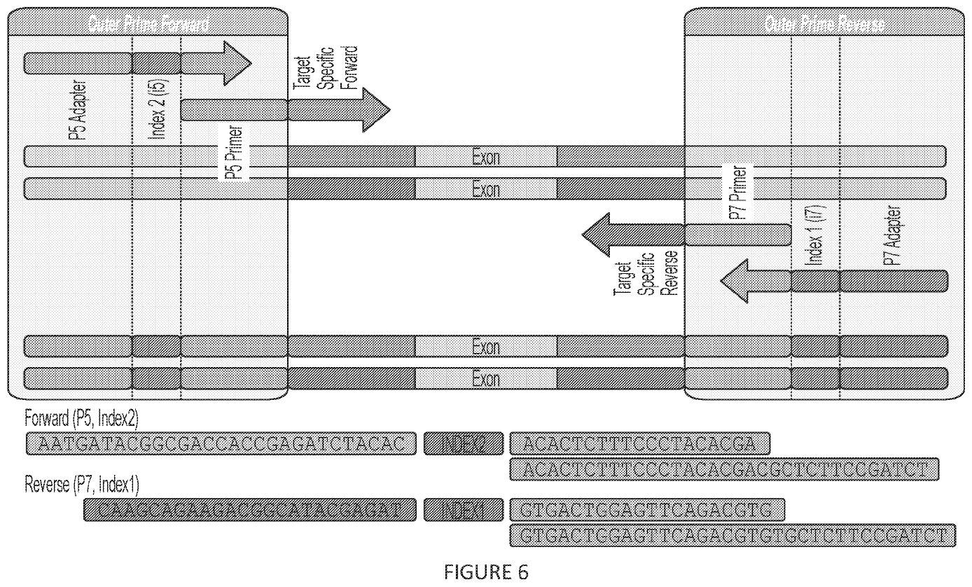

On Target Amplicon (OTA) sequencing uses targeted next generation sequencing (NGS) to analyze the editing efficiency of different guide RNAs. In the present example, OTA-NGS was used to screen MAD7 guide RNA activity. Briefly, the genomic DNA of cells treated with MAD7 nuclease and corresponding guide RNA was amplified by a first round of PCR using primers with NGS sequencing adapters that amplify the target regions. Next, the PCR amplicons were further amplified using index primers that align with the adapter sequences and allow for amplicon barcoding. Then, the amplicons were pooled and purified using a magnetic bead-based purification method, before finally being loaded onto a sequencer for analysis. After sequencing, the target regions were aligned to a reference genome, and editing efficiency was calculated as a sum of all reads divided by the sum of reads with indels. See FIG. 6 for an exemplary schematic of the OTA-NGS assay two step PCR amplification using Illumina adapters.

TABLE 10
Exemplary MAD7 sgRNA sequences targeting B2M gene locus and percent editing
as determined by OTA-NGS assay

| | SEQ ID | | SEQ ID | | % Editing |
|---|---|---|---|---|---|
| Name | NO: | Spacer | NO: | Full Sequence | (NGS-OTA) |
| B2mMad7 | 39 | UGGCCUGGAGG | 78 | GUCAAAAGACCUUUUUAAUUU | 100.0 |
| sgRNA3 | | CUAUCCAGCG | | CUACUCUUGUAGAUUGGCCUG | |
| | | | | GAGGCUAUCCAGCG | |
| B2mMad7 | 41 | CCGAUAUUCCU | 80 | GUCAAAAGACCUUUUUAAUUU | 51.0 |
| sgRNA5 | | CAGGUACUCC | | CUACUCUUGUAGAUCCGAUAU | |
| | | | | UCCUCAGGUACUCC | |
| B2mMad7 | 45 | CUCACGUCAUC | 84 | GUCAAAAGACCUUUUUAAUUU | 51.1 |
| sgRNA9 | | CAGCAGAGAA | | CUACUCUUGUAGAUCUCACGUC | |
| | | | | AUCCAGCAGAGAA | |
| B2mMad7 | 59 | AGUGGGGGUGA | 98 | GUCAAAAGACCUUUUUAAUUU | 99.8 |
| sgRNA23 | | AUUCAGUGUA | | CUACUCUUGUAGAUAGUGGGG | |
| | | | | GUGAAUUCAGUGUA | |
| B2mMad7 | 60 | CAGUGGGGGUG | 99 | GUCAAAAGACCUUUUUAAUUU | 99.8 |
| sgRNA24 | | AAUUCAGUGU | | CUACUCUUGUAGAUCAGUGGG | |
| | | | | GGUGAAUUCAGUGU | |
| B2mMad7 | 61 | UCAGUGGGGGU | 100 | GUCAAAAGACCUUUUUAAUUU | 99.5 |
| sgRNA25 | | GAAUUCAGUG | | CUACUCUUGUAGAUUCAGUGG | |
| | | | | GGGUGAAUUCAGUG | |
| B2mMad7 | 68 | CCCCACUUAAC | 107 | GUCAAAAGACCUUUUUAAUUU | 52.7 |
| sgRNA32 | | UAUCUUGGGC | | CUACUCUUGUAGAUCCCCACUU | |
| | | | | AACUAUCUUGGGC | |
| B2mMad7 | 69 | GUCACAGCCCA | 108 | GUCAAAAGACCUUUUUAAUUU | 68.6 |
| sgRNA33 | | AGAUAGUUAA | | CUACUCUUGUAGAUGUCACAG | |
| | | | | CCCAAGAUAGUUAA | |
| B2mMad7 | 70 | UCACAGCCCAA | 109 | GUCAAAAGACCUUUUUAAUUU | 76.8 |
| sgRNA34 | | GAUAGUUAAG | | CUACUCUUGUAGAUUCACAGCC | |
| | | | | CAAGAUAGUUAAG | |
| B2mMad7 | 75 | AUAGAUCGAGA | 114 | GUCAAAAGACCUUUUUAAUUU | 100.0 |
| sgRNA39 | | CAUGUAAGCA | | CUACUCUUGUAGAUAUAGAUC | |
| | | | | GAGACAUGUAAGCA | |

TABLE 11
Exemplary MAD7 sgRNA sequences targeting CIITA gene locus and percent
editing as determined by OTA-NGS assay

| | SEQ ID | | SEQ ID | | % Editing |
|---|---|---|---|---|---|
| Name | NO: | Spacer | NO: | Full Sequence | (NGS-OTA) |
| CIITAMad | 116 | CCUUGGGGCUC | 159 | GUCAAAAGACCUUUUUAAUUU | 55.40 |
| 7sgRNA2 | | UGACAGGUAG | | CUACUCUUGUAGAUCCUUGGG | |
| | | | | GCUCUGACAGGUAG | |
| CIITAMad | 117 | ACCUUGGGGCU | 160 | GUCAAAAGACCUUUUUAAUUU | 26.27 |
| 7sgRNA3 | | CUGACAGGUA | | CUACUCUUGUAGAUACCUUGG | |
| | | | | GGCUCUGACAGGUA | |
| CIITAMad | 120 | CCGGCCUUUUU | 163 | GUCAAAAGACCUUUUUAAUUU | 100.00 |
| 7sgRNA6 | | ACCUUGGGGC | | CUACUCUUGUAGAUCCGGCCUU | |
| | | | | UUUACCUUGGGGC | |
| CIITAMad | 123 | UAGGGGCCCCA | 166 | GUCAAAAGACCUUUUUAAUUU | 37.90 |
| 7sgRNA9 | | ACUCCAUGGU | | CUACUCUUGUAGAUUAGGGGC | |
| | | | | CCCAACUCCAUGGU | |
| CIITAMad | 127 | UUCUCCAGCCA | 170 | GUCAAAAGACCUUUUUAAUUU | 64.64 |
| 7sgRNA13 | | GGUCCAUCUG | | CUACUCUUGUAGAUUUCUCCA | |
| | | | | GCCAGGUCCAUCUG | |
| CIITAMad | 128 | UAUGACCAGAU | 171 | GUCAAAAGACCUUUUUAAUUU | 99.98 |
| 7sgRNA14 | | GGACCUGGCU | | CUACUCUUGUAGAUUAUGACC | |
| | | | | AGAUGGACCUGGCU | |
| CIITAMad | 131 | CUCCCAGAACC | 174 | GUCAAAAGACCUUUUUAAUUU | 99.97 |
| 7sgRNA17 | | CGACACAGAC | | CUACUCUUGUAGAUCUCCCAGA | |
| | | | | ACCCGACACAGAC | |
| CIITAMad | 137 | CUUGUCUGGGC | 180 | GUCAAAAGACCUUUUUAAUUU | 99.90 |
| 7sgRNA23 | | AGCGGAACUG | | CUACUCUUGUAGAUCUUGUCU | |
| | | | | GGGCAGCGGAACUG | |
| CIITAMad | 140 | CAGGACUCCCA | 183 | GUCAAAAGACCUUUUUAAUUU | 100.00 |
| 7sgRNA26 | | GCUGGAGGGC | | CUACUCUUGUAGAUCAGGACU | |
| | | | | CCCAGCUGGAGGGC | |
| CIITAMad | 144 | UCAAAGUAGAG | 187 | GUCAAAAGACCUUUUUAAUUU | 99.97 |
| 7sgRNA30 | | CACAUAGGAC | | CUACUCUUGUAGAUUCAAAGU | |
| | | | | AGAGCACAUAGGAC | |
| CIITAMad | 145 | UGCUGGCAUCU | 188 | GUCAAAAGACCUUUUUAAUUU | 99.99 |
| 7sgRNA31 | | CCAUACUCUC | | CUACUCUUGUAGAUUGCUGGC | |
| | | | | AUCUCCAUACUCUC | |
| CIITAMad | 146 | UGCCCAACUUC | 189 | GUCAAAAGACCUUUUUAAUUU | 100.00 |
| 7sgRNA32 | | UGCUGGCAUC | | CUACUCUUGUAGAUUGCCCAAC | |
| | | | | UUCUGCUGGCAUC | |
| CIITAMad | 149 | UGACUUUUCUG | 192 | GUCAAAAGACCUUUUUAAUUU | 100.00 |
| 7sgRNA35 | | CCCAACUUCU | | CUACUCUUGUAGAUUGACUUU | |
| | | | | UCUGCCCAACUUCU | |
| CIITAMad | 152 | AGGUCUGCCGG | 195 | GUCAAAAGACCUUUUUAAUUU | 99.84 |
| 7sgRNA38 | | AAGCUCCUCU | | CUACUCUUGUAGAUAGGUCUG | |
| | | | | CCGGAAGCUCCUCU | |
| CIITAMad | 154 | UCUGCAGCCUU | 197 | GUCAAAAGACCUUUUUAAUUU | 54.75 |
| 7sgRNA40 | | CCCAGAGGAG | | CUACUCUUGUAGAUUCUGCAG | |
| | | | | CCUUCCCAGAGGAG | |
| CIITAMad | 155 | CAGUGCUUCAG | 198 | GUCAAAAGACCUUUUUAAUUU | 97.81 |
| 7sgRNA41 | | GUCUGCCGGA | | CUACUCUUGUAGAUCAGUGCU | |
| | | | | UCAGGUCUGCCGGA | |
| CIITAMad | 156 | CCAGAGGAGCU | 199 | GUCAAAAGACCUUUUUAAUUU | 99.87 |
| 7sgRNA42 | | UCCGGCAGAC | | CUACUCUUGUAGAUCCAGAGG | |
| | | | | AGCUUCCGGCAGAC | |
| CIITAMad | 157 | CGGCAGACCUG | 200 | GUCAAAAGACCUUUUUAAUUU | 99.93 |
| 7sgRNA43 | | AAGCACUGGA | | CUACUCUUGUAGAUCGGCAGA | |
| | | | | CCUGAAGCACUGGA | |

Lengthy table referenced here
US20260275385A1-20260917-T00001
Please refer to the end of the specification for access instructions.

Lengthy table referenced here
US20260275385A1-20260917-T00002
Please refer to the end of the specification for access instructions.

Lengthy table referenced here
US20260275385A1-20260917-T00003
Please refer to the end of the specification for access instructions.

Lengthy table referenced here
US20260275385A1-20260917-T00004
Please refer to the end of the specification for access instructions.

Lengthy table referenced here
US20260275385A1-20260917-T00005
Please refer to the end of the specification for access instructions.

Lengthy table referenced here
US20260275385A1-20260917-T00006
Please refer to the end of the specification for access instructions.

Lengthy table referenced here
US20260275385A1-20260917-T00007
Please refer to the end of the specification for access instructions.

Lengthy table referenced here
US20260275385A1-20260917-T00008
Please refer to the end of the specification for access instructions.

Lengthy table referenced here
US20260275385A1-20260917-T00009
Please refer to the end of the specification for access instructions.

Lengthy table referenced here
US20260275385A1-20260917-T00010
Please refer to the end of the specification for access instructions.

Lengthy table referenced here
US20260275385A1-20260917-T00011
Please refer to the end of the specification for access instructions.

Lengthy table referenced here
US20260275385A1-20260917-T00012
Please refer to the end of the specification for access instructions.

Lengthy table referenced here
US20260275385A1-20260917-T00013
Please refer to the end of the specification for access instructions.

Lengthy table referenced here
US20260275385A1-20260917-T00014
Please refer to the end of the specification for access instructions.

Lengthy table referenced here
US20260275385A1-20260917-T00015
Please refer to the end of the specification for access instructions.

Lengthy table referenced here
US20260275385A1-20260917-T00016
Please refer to the end of the specification for access instructions.

Lengthy table referenced here
US20260275385A1-20260917-T00017
Please refer to the end of the specification for access instructions.

Lengthy table referenced here
US20260275385A1-20260917-T00018
Please refer to the end of the specification for access instructions.

### Certain Embodiments

Embodiment 1. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

Embodiment 2. A method of selecting engineered cells suitable for use in a therapeutic product, the method comprising:
selecting one or more genetically engineered cells from a population of cells based on a level of one or more markers on the cell surface of the one or more genetically engineered cells, and
preparing the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

Embodiment 3. A method of treating a disease in a subject with a composition comprising genetically engineered cells, the method comprising:
selecting one or more genetically engineered cells from a population of cells based on a level of one or more markers on the cell surface of the one or more genetically engineered cells,
formulating the composition comprising the selected one or more genetically engineered cells for treating a disease in a subject, and
administering the formulated composition to a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

Embodiment 4. A method of producing a composition comprising engineered cells with increased purity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a β2 microglobulin (B2M) gene locus,
wherein the level of the one or more markers on the cell surface comprise a level of an MHC I molecule, and
wherein at least 30% of the genetically engineered cells in the formulated composition comprise the transgene encoding the first tolerogenic factor at the insertion site at the B2M gene locus and/or the first tolerogenic factor.

Embodiment 5. A method of producing a composition comprising genetically engineered cells with enhanced efficacy, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus,
wherein the level of the one or more markers on the cell surface comprise a level of an MHC I molecule and/or the first tolerogenic factor, and
wherein the composition with enhanced efficacy is more effective than a composition comprising cells that do not comprise the one or more genetic modifications.

Embodiment 6. A method of producing a composition with reduced host immune response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a β2 microglobulin (B2M) gene locus,
wherein the level of the one or more markers on the cell surface comprises a level of an MHC I molecule and/or the first tolerogenic factor on the cell surface of the one or more genetically engineered cells, and
wherein the composition with reduced host immune response elicits a reduced host immune response compared to a composition comprising comparable cells that do not comprise the one or more genetic modifications.

Embodiment 7. A method of formulating a composition with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus,
wherein the level of one or more markers on the cell surface comprises a level of an MHC I molecule and/or the first tolerogenic factor, and
wherein the composition with reduced immunogenicity elicits a reduced host immune response compared to a composition comprising comparable cells that do not comprise the one or more genetic modifications.

Embodiment 8. A method of producing a composition comprising genetically engineered cells with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor, and
wherein the composition with reduced immunogenicity elicits a reduced host immune response compared to a composition comprising comparable cells that do not comprise the one or more genetic modifications.

Embodiment 9. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

Embodiment 10. A method of selecting engineered cells suitable for use in a therapeutic product, the method comprising:
selecting one or more genetically engineered cells from a population of cells based on a level of one or more markers on the cell surface of the one or more genetically engineered cells, and
preparing the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

Embodiment 11. A method of treating a disease in a subject with a composition comprising genetically engineered cells, the method comprising:
selecting one or more genetically engineered cells from a population of cells based on a level of one or more markers on the cell surface of the one or more genetically engineered cells,
formulating the composition comprising the selected one or more genetically engineered cells for treating a disease in a subject, and
administering the formulated composition to a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

Embodiment 12. A method of producing a composition comprising engineered cells with increased purity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus,
wherein the level of the one or more markers on the cell surface comprise a level of an MHC II molecule and/or the first tolerogenic factor, and
wherein at least 30% of the genetically engineered cells in the formulated composition comprise the transgene encoding the first tolerogenic factor at the insertion site at the CIITA gene locus.

Embodiment 13. A method of producing a composition comprising genetically engineered cells with enhanced efficacy, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus,
wherein the level of the one or more markers on the cell surface comprise a level of an MHC II molecule and/or the first tolerogenic factor, and
wherein the composition with enhanced efficacy is more effective than a composition comprising cells that do not comprise the one or more genetic modifications.

Embodiment 14. A method of producing a composition with reduced host immune response, the method comprising:
selecting one or more genetically engineered cells from a population of cells based on a
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus,
wherein the level of the one or more markers on the cell surface comprises a level of an MHC II molecule and/or the first tolerogenic factor on the cell surface of the one or more genetically engineered cells, and
wherein the composition with reduced host immune response elicits a reduced host immune response compared to a composition comprising comparable cells that do not comprise the one or more genetic modifications.

Embodiment 15. A method of formulating a composition with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus,
wherein the level of one or more markers on the cell surface comprises a level of an MHC II molecule and/or the first tolerogenic factor, and
wherein the composition with reduced immunogenicity elicits a reduced host immune response compared to a composition comprising comparable cells that do not comprise the one or more genetic modifications.

Embodiment 16. A method of producing a composition comprising genetically engineered cells with reduced immunogenicity, the method comprising:
selecting one or more genetically engineered cells from a population of cells based on a
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor, and
wherein the composition with reduced immunogenicity elicits a reduced host immune response compared to a composition comprising comparable cells that do not comprise the one or more genetic modifications.

Embodiment 17. The method of any one of embodiments 6-8 or 14-16, wherein the host immune response is an immune response of the subject against the one or more genetically engineered cells.

Embodiment 18. The method of embodiment 17, wherein the reduced host immune response comprises reduced donor-specific antibodies in the subject.

Embodiment 19. The method of embodiments 6-8, 14-16, or 17, wherein the reduced host immune response comprises reduced IgM or IgG antibodies in the subject.

Embodiment 20. The method of embodiments 6-8, 14-16, or 17, wherein the reduced host immune response comprises reduced complement-dependent cytotoxicity (CDC) in the subject.

Embodiment 21. The method of embodiments 6-8, 14-16, or 17, wherein the reduced host immune response comprises reduced TH1 activation in the subject.

Embodiment 22. The method of embodiments 6-8, 14-16, or 17, wherein the reduced host immune response comprises reduced NK cell killing in the subject.

Embodiment 23. The method of embodiments 6-8, 14-16, or 17, wherein the reduced host immune response comprises reduced killing by whole blood PBMCs in the subject.

Embodiment 24. A method of producing a composition comprising genetically engineered cells with a reduced graft versus host response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
optionally wherein the level of the one or more markers on the cell surface comprise a level of an MHC I molecule and/or the first tolerogenic factor, and
wherein the one or more genetically engineered cells of the composition with a reduced graft versus host response have a reduced immune response against cells of the subject as compared to a composition comprising comparable cells that do not comprise the one or more genetic modifications.

Embodiment 25. A method of producing a composition comprising genetically engineered cells with a reduced graft versus host response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
optionally wherein the level of the one or more markers on the cell surface comprise a level of an MHC II molecule and/or the first tolerogenic factor, and
wherein the one or more genetically engineered cells of the composition with a reduced graft versus host response have a reduced immune response against cells of the subject as compared to a composition comprising comparable cells that do not comprise the one or more genetic modifications.

Embodiment 26. The method of any of the preceding embodiments, wherein the one or more genetic modifications comprises an inserted transgene encoding a first tolerogenic factor.

Embodiment 27. The method of any of the preceding embodiments, wherein the method comprises inserting a transgene encoding a first tolerogenic factor into an insertion site in the genome of one or more cells in the population.

Embodiment 28. The method of any of the preceding embodiments, wherein the transgene encoding the first tolerogenic factor is inserted at an insertion site at a B2M gene locus.

Embodiment 29. The method of any one of embodiments 1-27, wherein the transgene encoding the first tolerogenic factor is inserted at an insertion site at a CIITA gene locus.

Embodiment 30. The method of any one of embodiments 27-29, wherein the step of inserting comprises homology-directed repair (HDR)-mediated insertion using a genome-modifying protein.

Embodiment 31. The method of embodiment 30, wherein the step of inserting using a genome modifying protein comprises insertion by a CRISPR-associated transposase, prime editing, a TnpB polypeptide, or Programmable Addition via Site-specific Targeting Elements (PASTE).

Embodiment 32. The method of embodiment 30, wherein the step of inserting using a genome modifying protein comprises insertion by a site-directed nuclease.

Embodiment 33. The method of embodiment 32, wherein the site-directed nuclease is selected from a zinc finger nuclease (ZFN), a TAL-effector nuclease (TALEN), or a CRISPR-Cas combination, optionally wherein the Cas is selected from a Cas9 or a Cas12.

Embodiment 34. The method of embodiment 32 or 33, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

Embodiment 35. The method of any one of embodiments 27-34, wherein the step of inserting comprises homology-directed repair (HDR)-mediated insertion using a guide RNA (gRNA) and a CRISPR-associated (Cas) nuclease.

Embodiment 36. The method of embodiment 35, wherein the gRNA comprises a complementary region, wherein the complementary region comprises a nucleic acid sequence that is complementary to a target nucleic acid sequence within the B2M gene locus, and wherein the target nucleic acid sequence comprises the insertion site.

Embodiment 37. The method of embodiment 35, wherein the gRNA comprises a complementary region,
wherein the complementary region comprises a nucleic acid sequence that is complementary to a target nucleic acid sequence within the CIITA gene locus, and wherein the target nucleic acid sequence comprises the insertion site.

Embodiment 38. The method of any of the preceding embodiments, wherein the insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, or gttn, and wherein:
(i) r=a org,
(ii) y=c or t,
(iii) w=a or t,
(iv) v=a or c or g, and
(v) n=a, c, t, or g.

Embodiment 39. The method of any one of embodiments 27-38, wherein the step of inserting comprises homology-directed repair (HDR)-mediated insertion using SpCas9 and the PAM is ngg or nag, wherein n=a, c, t, or g.

Embodiment 40. The method of any one of embodiments 27-38, wherein the step of inserting comprises homology-directed repair (HDR)-mediated insertion using SaCas9 and the PAM is ngrrt or ngrrn, wherein:
(i) r=a org, and
(ii) n=a, c, t, or g.

Embodiment 41. The method of any one of embodiments 27-38, wherein the step of inserting comprises homology-directed repair (HDR)-mediated insertion using NmeCas9 and the PAM is nnnngatt, wherein n=a, c, t, or g.

Embodiment 42. The method of any one of embodiments 27-38, wherein the step of inserting comprises homology-directed repair (HDR)-mediated insertion using CjCas9 and the PAM is nnnnryac, wherein:
(i) r=a org,
(ii) y=c or t, and
(iii) n=a, c, t, or g.

Embodiment 43. The method of any one of embodiments 27-38, wherein the step of inserting comprises homology-directed repair (HDR)-mediated insertion using StCas9 and the PAM is nnagaaw wherein:
(i) w=a or t, and
(ii) n=a, c, t, or g.

Embodiment 44. The method of any one of embodiments 27-38, wherein the step of inserting comprises homology-directed repair (HDR)-mediated insertion using TdCas9 and the PAM is naaaac, wherein n=a, c, t, or g.

Embodiment 45. The method of one of embodiments 27-38, wherein the step of inserting comprises homology-directed repair (HDR)-mediated insertion using LbCas 12a and the PAM is tttv, wherein v=a or c or g.

Embodiment 46. The method of one of embodiments 27-38, wherein the step of inserting comprises homology-directed repair (HDR)-mediated insertion using AsCas 12a and the PAM is tttv, wherein v=a or c or g.

Embodiment 47. The method of one of embodiments 27-38, wherein the step of inserting comprises homology-directed repair (HDR)-mediated insertion using AacCas12b and the PAM is ttn, wherein n=a, c, t, or g.

Embodiment 49. The method of one of embodiments 27-38, wherein the step of inserting comprises homology-directed repair (HDR)-mediated insertion using BhCas12b and the PAM is attn., tttn, or gttn, wherein n=a, c, t, or g.

Embodiment 50. The method of any one of embodiments 32, 35, or 39-49, wherein homology-directed repair (HDR)-mediated insertion using a site-directed nuclease is performed with an HDR efficiency equal to or greater than HDR insertion using lentivirus.

Embodiment 51. The method of any one of embodiments 27-30, 32, 33, or 34, wherein the step of inserting comprises homology-directed repair (HDR)-mediated insertion using ZFN.

Embodiment 52. The method of any one of embodiments 27-30, 32, 33, 34, or 51, wherein the first insertion site is 25 nucleotides or less from a zinc finger binding sequence.

Embodiment 53. The method of any one of embodiments 27-30, 32, 33, or 34, wherein the step of inserting comprises homology-directed repair (HDR)-mediated insertion using TALEN.

Embodiment 54. The method of any one of embodiments 27-30, 32, 33, or 34, wherein the first insertion site is 25 nucleotides or less from a transcription activator-like effectors (TALE) binding sequence.

Embodiment 55. The method of any one of embodiments 27-32 or 34, wherein the step of inserting comprises homology-directed repair (HDR)-mediated insertion using a guide RNA (gRNA) and a TnpB polypeptide.

Embodiment 56. The method of any embodiment 55, wherein the gRNA comprises a complementary region,
wherein the complementary region comprises a nucleic acid sequence that is complementary to a target nucleic acid sequence within the B2M gene locus, and wherein the target nucleic acid sequence comprises the insertion site.

Embodiment 57. The method of embodiment 55, wherein the gRNA comprises a complementary region,
wherein the complementary region comprises a nucleic acid sequence that is complementary to a target nucleic acid sequence within the CIITA gene locus, and wherein the target nucleic acid sequence comprises the insertion site.

Embodiment 58. The method of any one of embodiments 55-57, wherein the insertion site is 25 nucleotides or less from a target adjacent motif (TAM) sequence, wherein the TAM sequence is tca, ttcan, ttgatn ataaa, or ttgat, and wherein:
(i) n=a, c, t, or g.

Embodiment 59. The method of any one of embodiments 55-58, wherein the step of inserting comprises homology-directed repair (HDR)-mediated insertion using TnpB polypeptide and the TAM is tca.

Embodiment 60. The method of any one of embodiments 55-58, wherein the step of inserting comprises homology-directed repair (HDR)-mediated insertion using TnpB polypeptide and the TAM is ttcan, wherein n=a, c, t, or g.

Embodiment 61. The method of any one of embodiments 55-58, wherein the step of inserting comprises homology-directed repair (HDR)-mediated insertion using TnpB polypeptide and the TAM is ttgatn, wherein n=a, c, t, or g.

Embodiment 62. The method of any one of embodiments 55-58, wherein the step of inserting comprises homology-directed repair (HDR)-mediated insertion using TnpB polypeptide and the TAM is ataaa.

Embodiment 63. The method of any one of embodiments 55-58, wherein the step of inserting comprises homology-directed repair (HDR)-mediated insertion using TnpB polypeptide and the TAM is ttgat.

Embodiment 64. The method of any of the preceding embodiments, wherein the insertion site is in an exon.

Embodiment 65. The method of any of the preceding embodiments, wherein the insertion site is in an intron.

Embodiment 66. The method of any of the preceding embodiments, wherein the insertion site is between an intron and an exon.

Embodiment 67. The method of any of the preceding embodiments, wherein the insertion site is in a regulatory region.

Embodiment 68. The method of any one of embodiments 1-8, 24, 26-28, 30-36, 38-56, or 58-67, wherein the transgene encoding the first tolerogenic factor at an insertion site at a B2M gene locus reduces expression of a functional B2M.

Embodiment 69. The method of any one of embodiments 1-8, 24, 26-28, 30-36, 38-56, or 58-68, wherein the transgene encoding the first tolerogenic factor at an insertion site at a B2M gene locus reduces expression of a functional MHC I molecule.

Embodiment 70. The method of any one of embodiments 1-8, 24, 26-28, 30-36, 38-56, or 58-69, wherein the transgene encoding the first tolerogenic factor at an insertion site at a B2M gene locus disrupts expression of a functional B2M.

Embodiment 71. The method of one of embodiments 1-8, 24, 26-28, 30-36, 38-56, or 58-70, wherein the transgene encoding the first tolerogenic factor at an insertion site at a B2M gene locus disrupts expression of a functional MHC I molecule.

Embodiment 72. The method of any one of embodiments 1-8, 24, 26-28, 30-36, 38-56, or 58-71, wherein the transgene encoding the first tolerogenic factor has a forward orientation (5′ to 3′) relative to the B2M gene locus.

Embodiment 73. The method of any one of embodiments 1-8, 24, 26-28, 30-36, 38-56, or 58-72, wherein the transgene encoding the first tolerogenic factor is in the same orientation as the B2M gene locus.

Embodiment 74. The method of any one of embodiments 1-8, 24, 26-28, 30-36, 38-56, or 58-71, wherein the transgene encoding the first tolerogenic factor has a reverse orientation (5′ to 3′) relative to the B2M gene locus.

Embodiment 75. The method of any one of embodiments 1-8, 24, 26-28, 30-36, 38-56, 58-71, or 74, wherein the transgene encoding the first tolerogenic factor is in the reverse orientation as the B2M gene locus.

Embodiment 76. The method of any one of embodiments 1-8, 24, 26-28, 30-36, or 38-75, wherein the B2M gene locus is an endogenous B2M locus.

Embodiment 77. The method of any one of embodiments 1-8, 24, 26-28, 30-36, or 38-76, wherein the B2M gene locus is chr15: 4,711,358-44,718,851.

Embodiment 78. The method of any one of embodiments 1-8, 24, 26-28, 30-36, 38-64, or 68-77, wherein the insertion site is within exon 1, exon 2, exon 3, or exon 4 at the B2M gene locus.

Embodiment 79. The method of any one of embodiments 1-8, 24, 26-28, 30-36, 38-64, or 68-77, wherein the insertion site is within exon 1 at the B2M gene locus.

Embodiment 80. The method of any one of embodiments 1-8, 24, 26-28, 30-36, 38-64, or 68-77, wherein the insertion site is within exon 2 at the B2M gene locus.

Embodiment 81. The method of any one of embodiments 1-8, 24, 26-28, 30-36, 38-64, or 68-77, wherein the insertion site is within exon 3 at the B2M gene locus.

Embodiment 82. The method of any one of embodiments 1-8, 24, 26-28, 30-36, 38-64, or 68-77, wherein the insertion site is within exon 4 at the B2M gene locus.

Embodiment 83. The method of any one of embodiments 1-8, 24, 26-28, 30-36, 38-63, 65, or 68-77, wherein the insertion site is within intron 1, intron 2, or intron 3 at the B2M gene locus. Embodiment 84. The method of any one of embodiments 1-8, 24, 26-28, 30-36, 38-63, 65, or 68-77, wherein the insertion site is within intron 1 at the B2M gene locus.

Embodiment 85. The method of any one of embodiments 1-8, 24, 26-28, 30-36, 38-63, 65, or 68-77, wherein the insertion site is within intron 2 at the B2M gene locus.

Embodiment 86. The method of any one of embodiments 1-8, 24, 26-28, 30-36, 38-63, 65, or 68-77, wherein the insertion site is within intron 3 at the B2M gene locus.

Embodiment 87. The method of any one of embodiments 1-8, 24, 26-28, 30-36, or 38-77, wherein the insertion site is within the 5′ UTR at the B2M gene locus.

Embodiment 88. The method of any one of embodiments 1-8, 24, 26-28, 30-36, or 38-77, wherein the insertion site is within the 3′ UTR at the B2M locus.

Embodiment 89. The method of any one of embodiments 1-8, 17-24, 26-28, 30-36, 38-56, 58-88, wherein the step of inserting comprises using an hB2M gRNA comprising a nucleic acid sequence selected from Table 7, Table 10, Table 12, Table 14, Table 16, Table 18, Table 20, Table 22, Table 24, Table 26, or Table 28.

Embodiment 90. The method of any one of embodiments 9-16, 25, 29, 37, or 57-67, wherein the transgene encoding the first tolerogenic factor at an insertion site at a CIITA gene locus reduces expression of a functional CIITA.

Embodiment 91. The method of any one of embodiments 9-16, 25, 29, 37, 57-67, or 90, wherein the transgene encoding the first tolerogenic factor at an insertion site at a CIITA gene locus reduces expression of a functional MHC II molecule.

Embodiment 92. The method of any one of embodiments 9-16, 25, 29, 37, 57-67, or 90-91, wherein the transgene encoding the first tolerogenic factor at an insertion site at a CIITA gene locus disrupts expression of a functional CIITA.

Embodiment 93. The method of any one of embodiments 9-16, 25, 29, 37, 57-67, or 90-92, wherein the transgene encoding the first tolerogenic factor at an insertion site at a CIITA gene locus disrupts expression of a functional MHC II molecule.

Embodiment 94. The method of any one of embodiments 9-16, 25, 29, 37, 57-67, or 90-93, wherein the transgene encoding the first tolerogenic factor has a forward orientation (5′ to 3′) relative to the CIITA gene locus.

Embodiment 95. The method of any one of embodiments 9-16, 25, 29, 37, 57-67, or 90-94, wherein the transgene encoding the first tolerogenic factor is in the same orientation as the CIITA gene locus.

Embodiment 96. The method of one of embodiments 9-16, 25, 29, 37, 57-67, or 90-93, wherein the transgene encoding the first tolerogenic factor has a reverse orientation (5′ to 3′) relative to the CIITA gene locus.

Embodiment 97. The method of any one of embodiments 9-16, 25, 29, 37, 57-67, 90-93, or 96, wherein the transgene encoding the first tolerogenic factor is in the reverse orientation as the CIITA gene locus.

Embodiment 98. The method of any one of embodiments 9-16, 25, 29, 37, 57-67, or 90-97, wherein the CIITA gene locus is an endogenous CIITA locus.

Embodiment 99. The method of one of embodiments 9-16, 25, 29, 37, 57-67, or 90-98, wherein the CIITA gene locus is chr16: 10,866,222-10,943,021.

Embodiment 100. The method of any one of embodiments 9-16, 25, 29, 37, 57-64, or 90-99, wherein the insertion site is within exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, or exon 20 at the CIITA gene locus.

Embodiment 101. The method of any one of embodiments 9-16, 25, 29, 37, 57-64, or 90-100, wherein the insertion site is within exon 1 at the CIITA gene locus.

Embodiment 102. The method of any one of embodiments 9-16, 25, 29, 37, 57-64, or 90-100, wherein the insertion site is within exon 2 at the CIITA gene locus.

Embodiment 103. The method of any one of embodiments 9-16, 25, 29, 37, 57-64, or 90-100, wherein the insertion site is within exon 3 at the CIITA gene locus.

Embodiment 104. The method of any one of embodiments 9-16, 25, 29, 37, 57-64, or 90-100, wherein the insertion site is within exon 4 at the CIITA gene locus.

Embodiment 105. The method of any one of embodiments 9-16, 25, 29, 37, 57-64, or 90-100, wherein the insertion site is within exon 5 at the CIITA gene locus.

Embodiment 106. The method of any one of embodiments 9-16, 25, 29, 37, 57-64, or 90-100, wherein the insertion site is within exon 6 at the CIITA gene locus.

Embodiment 107. The method of any one of embodiments 9-16, 25, 29, 37, 57-64, or 90-100, wherein the insertion site is within exon 7 at the CIITA gene locus.

Embodiment 108. The method of any one of embodiments 9-16, 25, 29, 37, 57-64, or 90-100, wherein the insertion site is within exon 8 at the CIITA gene locus.

Embodiment 109. The method of any one of embodiments 9-16, 25, 29, 37, 57-64, or 90-100, wherein the insertion site is within exon 9 at the CIITA gene locus.

Embodiment 110. The method of any one of embodiments 9-16, 25, 29, 37, 57-64, or 90-100, wherein the insertion site is within exon 10 at the CIITA gene locus.

Embodiment 111. The method of any one of embodiments 9-16, 25, 29, 37, 57-64, or 90-100, wherein the insertion site is within exon 11 at the CIITA gene locus.

Embodiment 112. The method of any one of embodiments 9-16, 25, 29, 37, 57-64, or 90-100, wherein the insertion site is within exon 12 at the CIITA gene locus.

Embodiment 113. The method of any one of embodiments 9-16, 25, 29, 37, 57-64, or 90-100, wherein the insertion site is within exon 13 at the CIITA gene locus.

Embodiment 114. The method of any one of embodiments 9-16, 25, 29, 37, 57-64, or 90-100, wherein the insertion site is within exon 14 at the CIITA gene locus.

Embodiment 115. The method of any one of embodiments 9-16, 25, 29, 37, 57-64, or 90-100, wherein the insertion site is within exon 15 at the CIITA gene locus.

Embodiment 116. The method of any one of embodiments 9-16, 25, 29, 37, 57-64, or 90-100, wherein the insertion site is within exon 16 at the CIITA gene locus.

Embodiment 117. The method of any one of embodiments 9-16, 25, 29, 37, 57-64, or 90-100, wherein the insertion site is within exon 17 at the CIITA gene locus.

Embodiment 118. The method of any one of embodiments 9-16, 25, 29, 37, 57-64, or 90-100, wherein the insertion site is within exon 18 at the CIITA gene locus.

Embodiment 119. The method of any one of embodiments 9-16, 25, 29, 37, 57-64, or 90-100, wherein the insertion site is within exon 19 at the CIITA gene locus.

Embodiment 120. The method of any one of embodiments 9-16, 25, 29, 37, 57-64, or 90-100, wherein the insertion site is within exon 20 at the CIITA gene locus.

Embodiment 121. The method of any one of embodiments 9-16, 25, 29, 37, 57-63, 65, or 90-99, wherein the insertion site is within intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, or intron 19 at the CIITA gene locus.

Embodiment 122. The method of any one of embodiments 9-16, 25, 29, 37, 57-63, 65, 90-99, or 121, wherein the insertion site is within intron 1 at the CIITA gene locus.

Embodiment 123. The method of any one of embodiments 9-16, 25, 29, 37, 57-63, 65, 90-99, or 121, wherein the insertion site is within intron 2 at the CIITA gene locus.

Embodiment 124. The method of any one of embodiments 9-16, 25, 29, 37, 57-63, 65, 90-99, or 121, wherein the insertion site is within intron 3 at the CIITA gene locus.

Embodiment 125. The method of any one of embodiments 9-16, 25, 29, 37, 57-63, 65, 90-99, or 121, wherein the insertion site is within intron 4 at the CIITA gene locus.

Embodiment 126. The method of any one of embodiments 9-16, 25, 29, 37, 57-63, 65, 90-99, or 121, wherein the insertion site is within intron 5 at the CIITA gene locus.

Embodiment 127. The method of one of embodiments 9-16, 25, 29, 37, 57-63, 65, 90-99, or 121, wherein the insertion site is within intron 6 at the CIITA gene locus.

Embodiment 128. The method of any one of embodiments 9-16, 25, 29, 37, 57-63, 65, 90-99, or 121, wherein the insertion site is within intron 7 at the CIITA gene locus.

Embodiment 129. The method of one of embodiments 9-16, 25, 29, 37, 57-63, 65, 90-99, or 121, wherein the insertion site is within intron 8 at the CIITA gene locus.

Embodiment 130. The method of one of embodiments 9-16, 25, 29, 37, 57-63, 65, 90-99, or 121, wherein the insertion site is within intron 9 at the CIITA gene locus.

Embodiment 131. The method of one of embodiments 9-16, 25, 29, 37, 57-63, 65, 90-99, or 121, wherein the insertion site is within intron 10 at the CIITA gene locus.

Embodiment 132. The method of one of embodiments 9-16, 25, 29, 37, 57-63, 65, 90-99, or 121, wherein the insertion site is within intron 11 at the CIITA gene locus.

Embodiment 133. The method of any one of embodiments 9-16, 25, 29, 37, 57-63, 65, 90-99, or 121, wherein the insertion site is within intron 12 at the CIITA gene locus.

Embodiment 134. The method of any one of embodiments 9-16, 25, 29, 37, 57-63, 65, 90-99, or 121, wherein the insertion site is within intron 13 at the CIITA gene locus.

Embodiment 135. The method of any one of embodiments 9-16, 25, 29, 37, 57-63, 65, 90-99, or 121, wherein the insertion site is within intron 14 at the CIITA gene locus.

Embodiment 136. The method of any one of embodiments 9-16, 25, 29, 37, 57-63, 65, 90-99, or 121, wherein the insertion site is within intron 15 at the CIITA gene locus.

Embodiment 137. The method of any one of embodiments 9-16, 25, 29, 37, 57-63, 65, 90-99, or 121, wherein the insertion site is within intron 16 at the CIITA gene locus.

Embodiment 138. The method of any one of embodiments 9-16, 25, 29, 37, 57-63, 65, 90-99, or 121, wherein the insertion site is within intron 17 at the CIITA gene locus.

Embodiment 139. The method of any one of embodiments 9-16, 25, 29, 37, 57-63, 65, 90-99, or 121, wherein the insertion site is within intron 18 at the CIITA gene locus.

Embodiment 140. The method of any one of embodiments 9-16, 25, 29, 37, 57-63, 65, 90-99, or 121, wherein the insertion site is within intron 19 at the CIITA gene locus.

Embodiment 141. The method of any one of embodiments 9-16, 25, 29, 37, 57-63, 67, or 90-99, wherein the insertion site is within the 5′ UTR at the CIITA gene locus.

Embodiment 142. The method of any one of embodiments 9-16, 25, 29, 37, 57-63, 67, or 90-99, wherein the insertion site is within the 3′ UTR at the CIITA gene locus.

Embodiment 143. The method of any one of embodiments 9-16, 17-23, 25-27, 29-35, 37-55, 57-67, 90-143, wherein the step of inserting comprises using an hCIITA gRNA comprising a nucleic acid sequence selected from Table 7, Table 11, Table 13, Table 15, Table 17, Table 19, Table 21, Table 23, Table 25, Table 27, or Table 29.

Embodiment 144. The method of any of the preceding embodiments, wherein the level of one or more markers on the cell surface comprises a level of the first tolerogenic factor on the cell surface of the one or more genetically engineered cells.

Embodiment 145. The method of any of the preceding embodiments, wherein the method comprises detecting a level of the first tolerogenic factor on the cell surface of the one or more genetically engineered cells.

Embodiment 146. The method of any of the preceding embodiments, wherein the one or more genetically engineered cells are selected if the first tolerogenic factor is detected on the cell surface of the one or more genetically engineered cells.

Embodiment 147. The method of any of the preceding embodiments, wherein the first tolerogenic factor is or comprises A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

Embodiment 148. The method of any of the preceding embodiments, wherein the first tolerogenic factor is or comprises CD47.

Embodiment 149. The method of any of the preceding embodiments, wherein the first tolerogenic factor is or comprises human CD47.

Embodiment 150. The method of any one of embodiments 147-149, wherein the CD47 comprises an amino acid sequence at least 80% identical to an amino acid sequence set forth in SEQ ID NO: 1 or SEQ ID NO:2.

Embodiment 151. The method of any of the preceding embodiments, wherein the transgene encoding the first tolerogenic factor is a transgene that encodes CD47 and the transgene comprises a nucleotide sequence at least 80% identical to a nucleotide sequence set forth in SEQ ID NO:3 or SEQ ID NO:4.

Embodiment 152. The method of any of the preceding embodiments, wherein the transgene encoding a first tolerogenic factor is a transgene that encodes CD47 and the nucleotide sequence of the transgene is codon-optimized.

Embodiment 153. The method of any of the preceding embodiments, wherein the transgene is at least 80% identical to a nucleotide sequence set forth in SEQ ID NO:5.

Embodiment 154. The method of any one of embodiments 1-8, 17-23, 24, 26-28, 30-36, 38-56, 58-89, or 144-154, wherein the method comprises detecting a level of B2M on the cell surface of the one or more genetically engineered cells.

Embodiment 155. The method of any one of embodiments 1-8, 17-23, 24, 26-28, 30-36, 38-56, 58-89, or 144-154, wherein the one or more genetically engineered cells are selected if B2M is not present at a detectable level on the cell surface of the one or more genetically engineered cells.

Embodiment 156. The method of any one of embodiments 1-8, 17-23, 24, 26-28, 30-36, 38-56, 58-89, or 144-155, wherein the method comprises detecting a level of an MHC-I molecule on the cell surface of the one or more genetically engineered cells.

Embodiment 157. The method of any one of embodiments 1-8, 17-23, 24, 26-28, 30-36, 38-56, 58-89, or 144-156, wherein the one or more genetically engineered cells are selected if an MHC-I molecule is not present at a detectable level on the cell surface of the one or more genetically engineered cells.

Embodiment 158. The method of any one of embodiments 9-23, 25-27, 29-35, 37-55, 57-67, or 90-153, wherein the method comprises detecting a level of an MHC-II molecule on the cell surface of the one or more genetically engineered cells.

Embodiment 159. The method of any one of embodiments 9-23, 25-27, 29-35, 37-55, 57-67, 90-153, or 158, wherein the one or more genetically engineered cells are selected if an MHC-II molecule is not present at a detectable level on the cell surface of the one or more genetically engineered cells.

Embodiment 160. The method of any of the preceding embodiments, wherein the level of one or more markers on the cell surface comprises a level of an MHC I molecule, an MHC II molecule, or both on the cell surface of the one or more genetically engineered cells.

Embodiment 161. The method of any of the preceding embodiments, wherein the method comprises detecting a level of the MHC I molecule, the MHC II molecule, or both on the cell surface of the one or more genetically engineered cells.

Embodiment 162. The method of any of the preceding embodiments, wherein the one or more genetically engineered cells are selected if the MHC I molecule, the MHC II molecule, or both, are not present at a detectable level on the cell surface of the one or more genetically engineered cells.

Embodiment 163. The method of any of the preceding embodiments, wherein the one or more genetic modifications comprise a modification at a T-cell receptor (TCR) locus, B2M locus, a TAP I locus, a NLRC5 locus, a CIITA locus, an HLA-A locus, an HLA-B locus, an HLA-C locus, an HLA-DP locus, an HLA-DM locus, an HLA-DOA locus, an HLA-DOB locus, an HLA-DQ locus, an HLA-DR locus, a RFX5 locus, a RFXANK locus, a RFXAP locus, an NFY-A locus, an NFY-B locus, an NFY-C locus, or a combination thereof.

Embodiment 164. The method of any of the preceding embodiments, wherein the one or more genetic modifications comprise a modification at a TCR gene locus.

Embodiment 165. The method of any of the preceding embodiments, wherein the modification at the TCR gene locus is a heterozygous modification.

Embodiment 166. The method of any of the preceding embodiments, wherein the modification at the TCR gene locus is a homozygous modification.

Embodiment 167. The method of any of the preceding embodiments, wherein the method comprises modifying a TCR gene locus.

Embodiment 168. The method of any of the preceding embodiments, wherein the modification at the TCR gene locus comprises a knock-out of the TCR gene locus.

Embodiment 169. The method of any of the preceding embodiments, wherein the method comprises knocking out the TCR gene locus.

Embodiment 170. The method of any of the preceding embodiments, wherein the one or more genetic modifications comprise a modification at an HLA-A locus, an HLA-B locus, an HLA-C locus, or a combination thereof.

Embodiment 171. The method of any of the preceding embodiments, wherein the modification at the HLA-A locus, the HLA-B locus, the HLA-C locus, or a combination thereof is a heterozygous modification.

Embodiment 172. The method of any of the preceding embodiments, wherein the modification at the HLA-A locus, the HLA-B locus, the HLA-C locus, or a combination thereof is a homozygous modification.

Embodiment 173. The method of any of the preceding embodiments, wherein the method comprises modifying an HLA-A locus, an HLA-B locus, an HLA-C locus, or a combination thereof.

Embodiment 174. The method of any of the preceding embodiments, wherein the modification at the HLA-A locus, the HLA-B locus, the HLA-C locus, or a combination thereof comprises a knock-out of the HLA-A locus, the HLA-B locus, the HLA-C locus, or a combination thereof.

Embodiment 175. The method of any of the preceding embodiments, wherein the method comprises knocking out an HLA-A locus, an HLA-B locus, an HLA-C locus, or a combination thereof.

Embodiment 176. The method of any of the preceding embodiments, wherein the one or more genetic modifications comprise a modification at an HLA-DM locus, an HLA-DO locus, an HLA-DP locus, an HLA-DQ locus, an HLA-DR locus, or a combination thereof.

Embodiment 177. The method of any of the preceding embodiments, wherein the modification at the HLA-DM locus, the HLA-DO locus, the HLA-DP locus, the HLA-DQ locus, the HLA-DR locus, or a combination thereof is a heterozygous modification.

Embodiment 178. The method of any of the preceding embodiments, wherein the modification at the HLA-DM locus, the HLA-DO locus, the HLA-DP locus, the HLA-DQ locus, the HLA-DR locus, or a combination thereof is a homozygous modification.

Embodiment 179. The method of any of the preceding embodiments, wherein the method comprises modifying an HLA-DM locus, an HLA-DO locus, an HLA-DP locus, an HLA-DQ locus, an HLA-DR locus, or a combination thereof.

Embodiment 180. The method of any of the preceding embodiments, wherein the modification at the HLA-DM locus, the HLA-DO locus, the HLA-DP locus, the HLA-DQ locus, the HLA-DR locus, or a combination thereof comprises a knock-out of the HLA-DM locus, the HLA-DO locus, the HLA-DP locus, the HLA-DQ locus, the HLA-DR locus, or a combination thereof.

Embodiment 181. The method of any of the preceding embodiments, wherein the method comprises knocking out an HLA-DM locus, an HLA-DO locus, an HLA-DP locus, an HLA-DQ locus, an HLA-DR locus, or a combination thereof.

Embodiment 182. The method of any of the preceding embodiments, wherein the one or more genetic modifications comprise a modification at a B2M gene locus.

Embodiment 183. The method of any of the preceding embodiments, wherein the modification at the B2M gene locus is a heterozygous modification.

Embodiment 184. The method of any of the preceding embodiments, wherein the modification at the B2M gene locus is a homozygous modification.

Embodiment 185. The method of any of the preceding embodiments, wherein the method comprises modifying a B2M locus.

Embodiment 186. The method of any of the preceding embodiments, wherein the modification at the B2M locus comprises a knock-out of the B2M locus.

Embodiment 187. The method of any of the preceding embodiments, wherein the method comprises knocking out the B2M gene locus.

Embodiment 188. The method of any of the preceding embodiments, wherein the one or more genetic modifications comprise a modification at a CIITA gene locus.

Embodiment 189. The method of any of the preceding embodiments, wherein the modification at the CIITA gene locus is a heterozygous modification.

Embodiment 190. The method of any of the preceding embodiments, wherein the modification at the CIITA gene locus is a homozygous modification.

Embodiment 191. The method of any of the preceding embodiments, wherein the method comprises modifying a CIITA gene locus.

Embodiment 192. The method of any of the preceding embodiments, wherein the modification at the CIITA gene locus comprises a knock-out of the CIITA gene locus.

Embodiment 193. The method of any of the preceding embodiments, wherein the method comprises knocking out the CIITA gene locus.

Embodiment 194. The method of any of the preceding embodiments, wherein the level of one or more markers on the cell surface comprises a level of an MHC I molecule, an MHC II molecule, or both, on the cell surface of the one or more genetically engineered cells.

Embodiment 195. The method of any of the preceding embodiments, wherein the method comprises detecting a level of the MHC I molecule, the MHC II molecule, or both, on the cell surface of the one or more genetically engineered cells.

Embodiment 196. The method of any of the preceding embodiments, wherein the one or more genetically engineered cells are selected if the MHC I molecule, the MHC II molecule, or both, are not present at a detectable level on the cell surface of the one or more genetically engineered cells.

Embodiment 197. The method of any of the preceding embodiments, wherein the one or more genetic modifications comprise a knock-out of: ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof.

Embodiment 198. The method of embodiment 197, wherein the protein that is involved in oxidative or ER stress is selected from the group consisting of TXNIP, PERK, IRE1α, and DJ-1 (PARK7).

Embodiment 199. The method of any of the preceding embodiments, wherein the level of one or more markers on the cell surface comprises a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

Embodiment 200. The method of any of the preceding embodiments, wherein the method comprises detecting a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

Embodiment 201. The method of any of the preceding embodiments, wherein the one or more genetically engineered cells are selected if ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, are not present at a detectable level on the cell surface of the one or more genetically engineered cells.

Embodiment 202. The method of any of the preceding embodiments, wherein the one or more genetic modifications comprise a second inserted transgene.

Embodiment 203. The method of embodiment 202, wherein the second transgene encodes a chimeric antigen receptor (CAR).

Embodiment 204. The method of any of the preceding embodiments, wherein the method comprises inserting a transgene encoding a CAR in the genome of one or more cells in the population.

Embodiment 205. The method of embodiment 204, wherein the transgene encoding a CAR is inserted at a safe harbor locus.

Embodiment 206. The method of embodiment 204 or 205, wherein the transgene encoding a CAR is inserted at a TRAC locus, a TRBC1 locus, a TRBC2 locus, a B2M locus, a CIITA locus, a MICA locus, a MICB locus, or a safe harbor locus.

Embodiment 207. The method of embodiment 204-205, wherein the transgene encoding a CAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 208. The method of embodiment 202, wherein the second transgene encodes a chimeric auto antigen receptor (CAAR).

Embodiment 209. The method of any of the preceding embodiments, wherein the method comprises inserting a transgene encoding a CAAR in the genome of one or more cells in the population.

Embodiment 210. The method of embodiment 208, wherein the transgene encoding a CAAR is inserted at a safe harbor locus.

Embodiment 211. The method of any one of embodiments 208-210, wherein the transgene encoding a CAAR is inserted at a TRAC locus, a TRBC1 locus, a TRBC2 locus, a B2M locus, a CIITA locus, a MICA locus, a MICB locus, or a safe harbor locus.

Embodiment 212. The method of any one of embodiments 208-210, wherein the transgene encoding a CAAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 213. The method of any one of embodiments 202-212, wherein the second transgene is inserted into the same site as the transgene encoding the first tolerogenic factor.

Embodiment 214. The method of any one of embodiments 202-213, wherein the second transgene and the first tolerogenic factor are encoded by two separate constructs.

Embodiment 215. The method of any one of embodiments 202-213, wherein the second transgene and the first tolerogenic factor are encoded by a polycistronic construct.

Embodiment 216. The method of embodiment 215, wherein the polycistronic construct is a bicistronic construct.

Embodiment 217. The method of any one of embodiments 203-207, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRa-specific CAR, a IL-13Ra-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a GD2-specific CAR, a GD3-specific CAR, a HMW-MAA-specific CAR, a IL-11Ra-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

Embodiment 218. The method of any one of embodiments 203-208, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.

Embodiment 219. The method of any one of embodiments 203-207, or 217-218, wherein the level of one or more markers on the cell surface comprises a level of the CAR on the cell surface of the one or more genetically engineered cells.

Embodiment 220. The method of any one of embodiments 203-207, or 217-219, wherein the method comprises detecting a level of the CAR on the cell surface of the one or more genetically engineered cells.

Embodiment 221. The method of any one of embodiments 203-207, or 217-220, wherein the one or more genetically engineered cells are selected if the CAR is detected on the cell surface of the one or more genetically engineered cells.

Embodiment 222. The method of any one of embodiments 208-212, wherein the CAAR comprises an antigen selected from the group consisting of a pancreatic β-cell antigen, synovial joint antigen, myelin basic protein, proteolipid protein, myelin oligodendritic glycoprotein, MuSK, keratinocyte adhesion protein desmoglein 3 (Dsg3), Ro-RNP complex, La antigen, myeloperoxidase, proteinase 3, cardiolipin, citrullinated proteins, carbamylated proteins, and α3 chain of basement membrane collagen.

Embodiment 223. The method of any one of embodiments 208-212, or 222, wherein the level of one or more markers on the cell surface comprises a level of the CAAR on the cell surface of the one or more genetically engineered cells.

Embodiment 224. The method of any one of embodiments 208-212, 222, or 223, wherein the method comprises detecting a level of the CAAR on the cell surface of the one or more genetically engineered cells.

Embodiment 225. The method of any one of embodiments one of embodiments 208-212, or 222-224, wherein the one or more genetically engineered cells are selected if the CAAR is detected on the cell surface of the one or more genetically engineered cells.

Embodiment 226. The method of any one of embodiments 202 or 213-216, wherein the second transgene encodes a second tolerogenic factor.

Embodiment 227. The method of embodiment 226, wherein the second transgene encoding the second tolerogenic factor is inserted at a TRAC locus, a TRBC1 locus, a TRBC2 locus, a B2M locus, a CIITA locus, a MICA locus, a MICB locus, a safe harbor locus, an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 228. The method of embodiment 226 or 227, wherein the second tolerogenic factor is or comprises A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

Embodiment 229. The method of any one of embodiments 226-228, wherein the first tolerogenic factor and the second tolerogenic factor are the same tolerogenic factor.

Embodiment 230. The method of any one of embodiments 226-228, wherein the first tolerogenic factor and the second tolerogenic factor are different tolerogenic factors.

Embodiment 231. The method of any one of embodiments 226-230, wherein the method comprises detecting a level of the second tolerogenic factor on the cell surface of the one or more genetically engineered cells, wherein the second tolerogenic factor is expressed at a higher level than endogenous expression levels of the second tolerogenic factor in a comparable cell that does not comprise the second transgene.

Embodiment 232. The method of any one of embodiments 226-231, wherein the one or more genetically engineered cells are selected if the second tolerogenic factor is detected on the cell surface of the one or more genetically engineered cells at a higher level of expression than endogenous expression levels of the second tolerogenic factor in a comparable cell that does not comprise the second transgene.

Embodiment 233. The method of any one of embodiments 226-232, wherein the one or more genetically engineered cells are selected from a population of cells based on a level of two or more markers on the cell surface of the one or more genetically engineered cells.

Embodiment 234. The method of any one of embodiments 226-233, wherein the one or more genetically engineered cells are selected from a population of cells based on a level of three or more markers on the cell surface of the one or more genetically engineered cells.

Embodiment 235. The method of any one of embodiments 226-234, wherein the one or more genetically engineered cells are selected from a population of cells based on a level of four or more markers on the cell surface of the one or more genetically engineered cells.

Embodiment 236. The method of any of the preceding embodiments, wherein each of the one or more markers on the cell surface of the one or more genetically engineered cells is associated with at least one of the one or more genetic modifications.

Embodiment 237. The method of any of the preceding embodiments, wherein each of the one or more genetic modifications impacts the level of at least one of the one or more markers on the cell surface of the one or more genetically engineered cells.

Embodiment 238. The method of any of the preceding embodiments, wherein one or more of: (i) the transgene encoding the first tolerogenic factor, (ii) the transgene encoding the CAR, or (iii) the transgene encoding the second tolerogenic factor comprise a promoter, an insulator, an enhancer, a polyadenylation (poly(A)) tail, a ubiquitous chromatin opening element, or a combination thereof.

Embodiment 239. The method of any of the preceding embodiments, wherein one or more of: (i) the transgene encoding the first tolerogenic factor, (ii) the transgene encoding the CAR, or (iii) the transgene encoding the second tolerogenic factor comprise a promoter and the promoter is a constitutive promoter.

Embodiment 240. The method of embodiment 239, wherein the constitutive promoter is an EF1α, EF1α short, CMV, SV40, PGK, adenovirus late, vaccinia virus 7.5K, SV40, HSV tk, mouse mammary tumor virus (MMTV), HIV LTR, moloney virus, Esptein Barr virus (EBV), Rous sarcoma virus (RSV), UBC CAG, MND, SSFV, or ICOS promoter.

Embodiment 241. The method of any of the preceding embodiments, wherein the population of cells are human cells or non-human animal cells.

Embodiment 242. The method of embodiment 241, wherein non-human animal cells are porcine, bovine or ovine cells.

Embodiment 243. The method of any of the preceding embodiments, wherein the population of cells are human cells.

Embodiment 244. The method of any of the preceding embodiments, wherein the population of cells are differentiated cells derived from stem cells or progenitor cells.

Embodiment 245. The method of embodiment 244, wherein the stem cells are pluripotent stem cells.

Embodiment 246. The method of embodiment 245, wherein the pluripotent stem cells are induced pluripotent stem cells (iPSC).

Embodiment 247. The method of embodiment 245, wherein the pluripotent stem cells are embryonic stem cells (ESC).

Embodiment 248. The method of any of the preceding embodiments, wherein the population of cells are primary cells isolated from a donor.

Embodiment 249. The method of embodiment 248, wherein the donor is a single donor or multiple donors.

Embodiment 250. The method of embodiment 248 or 249, wherein the donor is healthy and/or is not suspected of having a disease or condition at the time the primary cells are obtained from the donor.

Embodiment 251. The method of any of the preceding embodiments, wherein the population of cells are islet cells, beta islet cells, pancreatic islet cells, immune cells, B cells, T cells, natural killer (NK) cells, natural killer T (NKT) cells, macrophage cells, endothelial cells, muscle cells, cardiac muscle cells, smooth muscle cells, skeletal muscle cells, dopaminergic neurons, retinal pigmented epithelium cells, optic cells, hepatocytes, thyroid cells, skin cells, glial progenitor cells, neural cells, cardiac cells, stem cells, hematopoietic stem cells, induced pluripotent stem cells (iPSCs), mesenchymal stem cells (MSCs), embryonic stem cells (ESCs), pluripotent stem cell (PSCs), blood cells, or a combination thereof.

Embodiment 252. The method of any of the preceding embodiments, wherein the population of cells are T-cells.

Embodiment 253. The method of embodiment 252, wherein the T-cells are CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells, or a combination thereof.

Embodiment 254. The method of embodiment 252 or 253, wherein the T cells are cytotoxic T-cells, helper T-cells, memory T-cells, regulatory T-cells, tumor infiltrating lymphocytes, or a combination thereof.

Embodiment 255. The method of any of the preceding embodiments, wherein the population of cells are human T-cells.

Embodiment 256. The method of any of the preceding embodiments, wherein the population of cells are autologous T-cells.

Embodiment 257. The method of any of the preceding embodiments, wherein the population of cells are allogeneic T-cells.

Embodiment 258. The method of embodiment 257, wherein the allogeneic T cells are primary T cells.

Embodiment 259. The method of embodiment 258 or 259, wherein the allogeneic T cells have been differentiated from embryonic stem cells (ESCs) or an induced pluripotent stem cells (iPSCs).

Embodiment 260. The method of any of the preceding embodiments, wherein the population of cells are T-cells, and wherein, after the steps of inserting the transgene encoding the first tolerogenic factor and modifying an HLA-A locus, an HLA-B locus, an HLA-C locus, or a combination thereof, at least 30% of the population of T-cells each have (a) reduced cell surface expression of MHC I and/or MHC II molecules as compared to comparable T-cells that have not been genetically engineered, and (b) increased expression of the first tolerogenic factor encoded by the first transgene as compared to comparable T-cells that have not been genetically engineered.

Embodiment 261. The method of any of the preceding embodiments, wherein the population of cells are T-cells and the tolerogenic factor is CD47, and wherein, after the steps of inserting the transgene encoding the first tolerogenic factor and knocking out the B2M locus and/or the CIITA locus, at least 30% of the population of T-cells each have (a) a B2M locus and/or a CIITA locus knocked-out, and (b) increased expression of CD47 as compared to comparable T-cells that have not been genetically engineered.

Embodiment 262. The method of any of the preceding embodiments, wherein the population of cells are T-cells and the first tolerogenic factor is CD47, and wherein, after the steps of inserting the transgene encoding the first tolerogenic factor and knocking out the B2M locus and/or the CIITA locus, at least 30% of the population of T-cells each have (a) reduced cell surface expression of MHC I and/or MHC II molecules as compared to T-cells that have not been genetically engineered, and (b) increased expression of CD47 as compared to comparable T-cells that have not been genetically engineered.

Embodiment 263. The method of any one of embodiments 260-262, wherein at least 35% of the population of T-cells each have (a) and (b).

Embodiment 264. The method of one of embodiments 1-259, wherein the population of cells are T-cells and the tolerogenic factor is CD47, and wherein, after the steps of inserting the transgene encoding the first tolerogenic factor and knocking out the B2M locus and/or the CIITA locus, at least 20% of the population of T-cells each have (a) reduced expression of B2M as compared to comparable T-cells that have not been genetically engineered, (b) reduced expression of CIITA as compared to comparable T-cells that have not been genetically engineered, and (c) increased expression of CD47 as compared to comparable T-cells that have not been genetically engineered.

Embodiment 265. The method of embodiment 264, wherein at least 35% of the T-cells each have (a) and (b).

Embodiment 266. The method of embodiment 264 or 265, wherein at least 35% of the population of T-cells each have (a), (b), and (c).

Embodiment 267. The method of any of the preceding embodiments, further comprising storing the cells.

Embodiment 268. The method of embodiment 267, wherein storing the cells comprises freezing the cells.

Embodiment 269. The method of any of the preceding embodiments, wherein the one or more genetically engineered cells are stored after being selected based on a level of one or more markers on the cell surface of the one or more genetically engineered cells.

Embodiment 270. The method of any one of embodiments 267-269, wherein the one or more genetically engineered cells are stored after one or more genetic modifications are introduced.

Embodiment 271. The method of any one of embodiments 267-270, wherein the one or more genetically engineered cells are stored before being selected based on a level of one or more markers on the cell surface of the one or more genetically engineered cells.

Embodiment 272. The method of any one of embodiments 267-269, wherein the one or more genetically engineered cells are stored before one or more genetic modifications are introduced.

Embodiment 273. The method of any one of embodiments 268-272, further comprising thawing the cells.

Embodiment 274. The method of embodiment 273, wherein the one or more genetically engineered cells are thawed prior to one or more genetic modifications being introduced.

Embodiment 275. The method of embodiment 273 or 274, wherein the one or more genetically engineered cells are formulated in the composition after thawing.

Embodiment 276. The method of embodiment 273 or 274, wherein the one or more genetically engineered cells are formulated in the composition before thawing.

Embodiment 277. The method of any of the preceding embodiments, wherein the composition is suitable for use in a subject.

Embodiment 278. The method of any of the preceding embodiments, wherein the composition is a therapeutic composition.

Embodiment 279. The method of any of the preceding embodiments, wherein the composition is a cell therapy composition.

Embodiment 280. The method of any of the preceding embodiments, wherein the composition comprises a pharmaceutically acceptable additive, carrier, diluent, or excipient.

Embodiment 281. The method of any of the preceding embodiments, wherein the composition comprises a buffered solution.

Embodiment 282. The method of any of the preceding embodiments, wherein the composition comprises a pharmaceutically acceptable buffer.

Embodiment 283. The method of embodiment 282, wherein the pharmaceutically acceptable buffer comprises neutral buffer saline or phosphate buffered saline.

Embodiment 284. The method of any of the preceding embodiments, wherein the composition comprises Plasma-Lyte A®, dextrose, dextran, sodium chloride, human serum albumin (HSA), dimethylsulfoxide (DMSO), or a combination thereof.

Embodiment 285. The method of any of the preceding embodiments, wherein the composition comprises a cryoprotectant.

Embodiment 286. A population of genetically engineered cells produced by the method of any one of embodiments 1-285.

Embodiment 287. A population of cells that have been genetically engineered to comprise a transgene encoding a first tolerogenic factor, wherein at least 30% of the cells have increased cell surface expression of a first tolerogenic factor as compared to a comparable cell that has not been genetically engineered.

Embodiment 288. The population of cells of embodiment 287, wherein the transgene encoding the first tolerogenic factor is inserted at an insertion site at a B2M gene locus.

Embodiment 289. The population of cells of embodiment 287, wherein the transgene encoding the first tolerogenic factor is inserted at an insertion site at a CIITA gene locus.

Embodiment 290. The population of cells of any one of embodiments 287-289, wherein the insertion site is in an exon.

Embodiment 291. The population of cells of any one of embodiments 287-289, wherein the insertion site is in an intron.

Embodiment 292. The population of cells of any one of embodiments 287-289, wherein the insertion site is between an intron and an exon.

Embodiment 293. The population of cells of any one of embodiments 287-289, wherein the insertion site is in a regulatory region.

Embodiment 294. The population of cells of any one of embodiments 287, 288, or 290, wherein the insertion site is within exon 1, exon 2, exon 3, or exon 4 at the B2M gene locus.

Embodiment 295. The population of cells of any one of embodiments 287, 288, 290, or 294, wherein the insertion site is within exon 1 at the B2M gene locus.

Embodiment 296. The population of cells of any one of embodiments 287, 288, 290, or 294, wherein the insertion site is within exon 2 at the B2M gene locus.

Embodiment 297. The population of cells of any one of embodiments 287, 288, 290, or 294, wherein the insertion site is within exon 3 at the B2M gene locus.

Embodiment 298. The population of cells of any one of embodiments 287, 288, 290, or 294, wherein the insertion site is within exon 4 at the B2M gene locus.

Embodiment 299. The population of cells of any one of embodiments 287, 288, or 291, wherein the insertion site is within intron 1, intron 2, or intron 3 at the B2M gene locus.

Embodiment 300. The population of cells of any one of embodiments 287, 288, 291, or 302, wherein the insertion site is within intron 1 at the B2M gene locus.

Embodiment 301. The population of cells of any one of embodiments 287, 288, 291, or 302, wherein the insertion site is within intron 2 at the B2M gene locus.

Embodiment 302. The population of cells of any one of embodiments 287, 288, 291, or 302, wherein the insertion site is within intron 3 at the B2M gene locus.

Embodiment 303. The population of cells of any one of embodiments 287, 288, or 293, wherein the insertion site is within the 5′ UTR at the B2M gene locus.

Embodiment 304. The population of cells of any one of embodiments 287, 288, or 293, wherein the insertion site is within the 3′ UTR at the B2M locus.

Embodiment 305. The population of cells of any one of embodiments 287, 289, or 290, wherein the insertion site is within exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, or exon 20 at the CIITA gene locus.

Embodiment 306. The population of cells of any one of embodiments 287, 289, 290, or 305, wherein the insertion site is within exon 1 at the CIITA gene locus.

Embodiment 307. The population of cells of any one of embodiments 287, 289, 290, or 305, wherein the insertion site is within exon 2 at the CIITA gene locus.

Embodiment 308. The population of cells of any one of embodiments 287, 289, 290, or 305, wherein the insertion site is within exon 3 at the CIITA gene locus.

Embodiment 309. The population of cells of any one of embodiments 287, 289, 290, or 305, wherein the insertion site is within exon 4 at the CIITA gene locus.

Embodiment 310. The population of cells of any one of embodiments 287, 289, 290, or 305, wherein the insertion site is within exon 5 at the CIITA gene locus.

Embodiment 311. The population of cells of any one of embodiments 287, 289, 290, or 305, wherein the insertion site is within exon 6 at the CIITA gene locus.

Embodiment 312. The population of cells of any one of embodiments 287, 289, 290, or 305, wherein the insertion site is within exon 7 at the CIITA gene locus.

Embodiment 313. The population of cells of any one of embodiments 287, 289, 290, or 305, wherein the insertion site is within exon 8 at the CIITA gene locus.

Embodiment 314. The population of cells of any one of embodiments 287, 289, 290, or 305, wherein the insertion site is within exon 9 at the CIITA gene locus.

Embodiment 315. The population of cells of any one of embodiments 287, 289, 290, or 305, wherein the insertion site is within exon 10 at the CIITA gene locus.

Embodiment 316. The population of cells of any one of embodiments 287, 289, 290, or 305, wherein the insertion site is within exon 11 at the CIITA gene locus.

Embodiment 317. The population of cells of any one of embodiments 287, 289, 290, or 305, wherein the insertion site is within exon 12 at the CIITA gene locus.

Embodiment 318. The population of cells of any one of embodiments 287, 289, 290, or 305, wherein the insertion site is within exon 13 at the CIITA gene locus.

Embodiment 319. The population of cells of any one of embodiments 287, 289, 290, or 305, wherein the insertion site is within exon 14 at the CIITA gene locus.

Embodiment 320. The population of cells of any one of embodiments 287, 289, 290, or 305, wherein the insertion site is within exon 15 at the CIITA gene locus.

Embodiment 321. The population of cells of any one of embodiments 287, 289, 290, or 305, wherein the insertion site is within exon 16 at the CIITA gene locus.

Embodiment 322. The population of cells of any one of embodiments 287, 289, 290, or 305, wherein the insertion site is within exon 17 at the CIITA gene locus.

Embodiment 323. The population of cells of any one of embodiments 287, 289, 290, or 305, wherein the insertion site is within exon 18 at the CIITA gene locus.

Embodiment 324. The population of cells of any one of embodiments 287, 289, 290, or 305, wherein the insertion site is within exon 19 at the CIITA gene locus.

Embodiment 325. The population of cells of any one of embodiments 287, 289, 290, or 305, wherein the insertion site is within exon 20 at the CIITA gene locus.

Embodiment 326. The population of cells of any one of embodiments 287, 289, or 291, wherein the insertion site is within intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, or intron 19 at the CIITA gene locus.

Embodiment 327. The population of cells of any one of embodiments 287, 289, 291, or 326, wherein the insertion site is within intron 1 at the CIITA gene locus.

Embodiment 328. The population of cells of any one of embodiments 287, 289, 291, or 326, wherein the insertion site is within intron 2 at the CIITA gene locus.

Embodiment 329. The population of cells of any one of embodiments 287, 289, 291, or 326, wherein the insertion site is within intron 3 at the CIITA gene locus.

Embodiment 330. The population of cells of any one of embodiments 287, 289, 291, or 326, wherein the insertion site is within intron 4 at the CIITA gene locus.

Embodiment 331. The population of cells of any one of embodiments 287, 289, 291, or 326, wherein the insertion site is within intron 5 at the CIITA gene locus.

Embodiment 332. The population of cells of any one of embodiments 287, 289, 291, or 326, wherein the insertion site is within intron 6 at the CIITA gene locus.

Embodiment 333. The population of cells of any one of embodiments 287, 289, 291, or 326, wherein the insertion site is within intron 7 at the CIITA gene locus.

Embodiment 334. The population of cells of any one of embodiments 287, 289, 291, or 326, wherein the insertion site is within intron 8 at the CIITA gene locus.

Embodiment 335. The population of cells of any one of embodiments 287, 289, 291, or 326, wherein the insertion site is within intron 9 at the CIITA gene locus.

Embodiment 336. The population of cells of any one of embodiments 287, 289, 291, or 326, wherein the insertion site is within intron 10 at the CIITA gene locus.

Embodiment 337. The population of cells of any one of embodiments 287, 289, 291, or 326, wherein the insertion site is within intron 11 at the CIITA gene locus.

Embodiment 338. The population of cells of any one of embodiments 287, 289, 291, or 326, wherein the insertion site is within intron 12 at the CIITA gene locus.

Embodiment 339. The population of cells of any one of embodiments 287, 289, 291, or 326, wherein the insertion site is within intron 13 at the CIITA gene locus.

Embodiment 340. The population of cells of any one of embodiments 287, 289, 291, or 326, wherein the insertion site is within intron 14 at the CIITA gene locus.

Embodiment 341. The population of cells of any one of embodiments 287, 289, 291, or 326, wherein the insertion site is within intron 15 at the CIITA gene locus.

Embodiment 342. The population of cells of any one of embodiments 287, 289, 291, or 326, wherein the insertion site is within intron 16 at the CIITA gene locus.

Embodiment 343. The population of cells of any one of embodiments 287, 289, 291, or 326, wherein the insertion site is within intron 17 at the CIITA gene locus.

Embodiment 344. The population of cells of any one of embodiments 287, 289, 291, or 326, wherein the insertion site is within intron 18 at the CIITA gene locus.

Embodiment 345. The population of cells of any one of embodiments 287, 289, 291, or 326, wherein the insertion site is within intron 19 at the CIITA gene locus.

Embodiment 346. The population of cells of any one of embodiments 287, 289, or 293, wherein the insertion site is within the 5′ UTR at the CIITA gene locus.

Embodiment 347. The population of cells of any one of embodiments 287, 289, or 293, wherein the insertion site is within the 3′ UTR at the CIITA gene locus.

Embodiment 348. The population of cells of any one of embodiments 287-347, wherein at least 35% of the cells have increased cell surface expression of a first tolerogenic factor as compared to a comparable cell that has not been genetically engineered.

Embodiment 349. The population of cells according to any one of embodiments 287-348, wherein the tolerogenic factor is CD47.

Embodiment 350. The population of cells of any one of embodiments 287, 288, 290-304, 348, or 349, wherein at least 30% of the cells have decreased cell surface expression of B2M as compared to a comparable cell that has not been genetically engineered.

Embodiment 351. The population of cells of any one of embodiments 287, 288, 290-304, 348, 349, or 350, wherein at least 35% of the cells have decreased cell surface expression of B2M as compared to a comparable cell that has not been genetically engineered.

Embodiment 352. The population of cells of any one of embodiments 287-351, wherein the cells have been genetically engineered to knock-out an HLA-A locus, an HLA-B locus, an HLA-C locus, or a combination thereof.

Embodiment 353. The population of cells of any one of embodiments 287-352, wherein the cells have been genetically engineered to knock-out an HLA-DM locus, an HLA-DO locus, an HLA-DP locus, an HLA-DQ locus, an HLA-DR locus, or a combination thereof.

Embodiment 354. The population of cells of any one of embodiments 287-353, wherein the cells have been genetically engineered to knock-out a B2M locus.

Embodiment 355. The population of cells of any one of embodiments 287-354, wherein the cells have been genetically engineered to knock-out a CIITA locus.

Embodiment 356. The population of cells of any one of embodiments 287-355, wherein the cells have been genetically engineered to knock-out a TCR locus.

Embodiment 357. The population of cells of any one of embodiments 287-356, wherein at least 30% of the cells have decreased cell surface expression of an MHC I molecule, an MHC II molecule, or both, as compared to a comparable cell that has not been genetically engineered.

Embodiment 358. The population of cells of any one of embodiments 287-357, wherein at least 35% of the cells have decreased cell surface expression of an MHC I molecule, an MHC II molecule, or both, as compared to a comparable cell that has not been genetically engineered.

Embodiment 359. The population of cells of any one of embodiments 287-358, wherein the cells have been genetically engineered to comprise a transgene encoding a CAR.

Embodiment 360. The population of cells of embodiment 359, wherein at least 35% of the cells have cell surface expression of the CAR.

Embodiment 361. The population of cells of any one of embodiments 287-360, wherein the cells have been genetically engineered to comprise a transgene encoding a CAAR.

Embodiment 362. The population of cells of embodiment 361, wherein at least 35% of the cells have cell surface expression of the CAAR.

Embodiment 363. A composition comprising a population of cells according to any one of embodiments 287-362.

Embodiment 364. A pharmaceutical composition comprising (i) a population of cells according to any one of embodiments 287-362, and (ii) a pharmaceutically acceptable excipient.

Embodiment 365. A method comprising administering to a subject a population of cells according to any one of embodiments 287-362, a composition of embodiment 363, or a pharmaceutical composition of embodiment 364.

Embodiment 366. The method of embodiment 365, wherein the method is a method of treating a disease in a subject.

Embodiment 367. A population of cells of any one of embodiments 287-362 for use in treating a disease in a subject.

Embodiment 368. A composition of embodiment 363 for use in treating a disease in a subject.

Embodiment 369. A pharmaceutical composition of embodiment 364 for use in treating a disease in a subject.

Embodiment 370. Use of a population of cells of any one of embodiments 287-362, a composition of embodiment 363 or 368, or a pharmaceutical composition of embodiment 364 or 368 for use in treating a disease in a subject.

Embodiment 371. Use of a population of cells of any one of embodiments 287-362, a composition of embodiment 363 or 368, or a pharmaceutical composition of embodiment 364 or 368 in the manufacture of a medicament for the treatment of a disease.

Embodiment 372. The method, the population of cells, the composition, the pharmaceutical composition or the use of any of the preceding embodiments, wherein the disease is cancer.

Embodiment 373. The method, the population of cells, the composition, the pharmaceutical composition or the use of any of the preceding embodiments, wherein the cancer is associated with CD5, CD19, CD20, CD22, CD23, CD30, CD33, CD70, Kappa, Lambda, B cell maturation agent (BCMA), G-protein coupled receptor family C group 5 member D (GPRC5D), CD123, LeY, NKG2D ligand, WT1, GD2, HER2, EGFR, EGFRVIII, B7H3, PSMA, PSCA, CAIX, CD171, CEA, CSPG4, EPHA2, FAP, FRα, IL-13Ra, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, GD2, GD3, HMW-MAA, IL-11Ra, KDR, Lewis Y, L1-cell adhesion molecule, MAGE-A1, Oncofetal antigen (h5T4), and/or TAG-72 expression.

Embodiment 374. The method, the population of cells, the composition, the pharmaceutical composition or the use of any of the preceding embodiments, wherein the cancer is a hematologic malignancy.

Embodiment 375. The method, the population of cells, the composition, the pharmaceutical composition or the use of any of the preceding embodiments, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 376. The method, the population of cells, the composition, the pharmaceutical composition or the use of any of the preceding embodiments, wherein the cancer is solid malignancy.

Embodiment 377. The method, the population of cells, the composition, the pharmaceutical composition or the use of any of the preceding embodiments, wherein the solid malignancy is selected breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

Embodiment 378. The method of any of the preceding embodiments, the population of cells of any of the preceding embodiments, the composition of any of the preceding embodiments, the pharmaceutical composition of any of the preceding embodiments, or the use of any of the preceding embodiments, wherein the disease is an autoimmune disease.

Embodiment 379. The method, the population of cells, the composition, the pharmaceutical composition or the use of any of the preceding embodiments, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 380. The method of any of the preceding embodiments, the population of cells of any of the preceding embodiments, the composition of any of the preceding embodiments, the pharmaceutical composition of any of the preceding embodiments, or the use of any of the preceding embodiments, wherein the disease is diabetes mellitus.

Embodiment 381. The method, the population of cells, the composition, the pharmaceutical composition or the use of any of the preceding embodiments, wherein the diabetes is selected from the group consisting Type I diabetes, Type II diabetes, prediabetes, and gestational diabetes.

Embodiment 382. The method of any of the preceding embodiments, the population of cells of any of the preceding embodiments, the composition of any of the preceding embodiments, the pharmaceutical composition of any of the preceding embodiments, or the use of any of the preceding embodiments, wherein the disease is a neurological disease.

Embodiment 383. The method, the population of cells, the composition, the pharmaceutical composition or the use of any of the preceding embodiments, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 384. A method of identifying a site for inserting a first transgene at a β2 microglobulin (B2M) gene locus, comprising the steps of:
(a) identifying a protospacer adjacent motif (PAM) sequence or target adjacent motif (TAM) sequence in (i) the B2M gene locus, (ii) the 100 bp upstream of the 5′ end of the B2M gene locus, or (iii) the 100 bp downstream of the 3′ end of the B2M gene locus, and
(b) generating a gRNA comprising a complementary region, wherein the complementary region comprises a nucleic acid sequence that is complementary to a target nucleic acid sequence within the B2M gene locus, wherein the target nucleic acid sequence comprises the first insertion site, and wherein the first insertion site is 25 nucleotides or less from a PAM sequence or a TAM sequence.

Embodiment 385. A method of identifying a site for inserting a first transgene at a class II transactivator (CIITA) gene locus, comprising the steps of:
(a) identifying a protospacer adjacent motif (PAM) sequence or target adjacent motif (TAM) sequence in (i) the CIITA gene locus, (ii) the 100 bp upstream of the 5′ end of the CIITA gene locus, or (iii) the 100 bp downstream of the 3′ end of the CIITA gene locus, and
(b) generating a gRNA comprising a complementary region, wherein the complementary region comprises a nucleic acid sequence that is complementary to a target nucleic acid sequence within the CIITA gene locus, wherein the target nucleic acid sequence comprises the first insertion site, and wherein the first insertion site is 25 nucleotides or less from a PAM sequence or a TAM sequence.

Embodiment 386. An engineered cell comprising one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a cell of the same cell type that does not comprise the modifications.

Embodiment 386a. An engineered cell comprising one or more modifications, wherein the modifications
(a) inactivate or disrupt one or more alleles of:
   (i) one or more MHC class I molecules and/or one or more molecules that regulate expression of the one or more MHC class I molecules, and/or
   (ii) one or more MHC class II molecules and/or one or more molecules that regulate expression of the one or more MHC class II molecules, and/or
(b) increase expression of one or more tolerogenic factors,
wherein the increased expression of (ii) is relative to an islet cell that does not comprise the modifications.

Embodiment 387. The engineered cell of embodiment 388, wherein the one or more modifications in (i) reduce expression of:
a. one or more MHC class I molecules
b. one or more MHC class II molecules; or
c. one or more MHC class I molecules and one or more MHC class II molecules.

Embodiment 388. The engineered cell of embodiment 388 or embodiment 389, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

Embodiment 389. The engineered cell of embodiment 390, wherein the engineered cell does not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.

Embodiment 390. The engineered cell of any of embodiments 388-391, wherein the one or more modifications that increase expression comprise increased cell surface expression, and/or the one or more modifications that reduce expression comprise reduced cell surface expression.

Embodiment 391. The engineered cell of any of embodiments 388-392, wherein the one or more modifications in (i) reduce expression of one or more MHC class I molecules.

Embodiment 392. The engineered cell of any of embodiments 388-393, wherein the one or more modifications in (i) reduce expression of B2M.

Embodiment 393. The engineered cell of any of embodiments 388-394, wherein the one or more modifications in (i) reduce expression of HLA-A, HLA-B, and/or HLA-C.

Embodiment 394. The engineered cell of any of embodiments 388-395, wherein the one or more modifications in (i) reduce expression of one or more MHC class II molecules.

Embodiment 395. The engineered cell of any of embodiments 388-396, wherein the one or more modifications in (i) reduce expression of CIITA.

Embodiment 396. The engineered cell of any of embodiments 388-397, wherein the one or more modifications in (i) reduce expression of HLA-DM, HLA-DO, HLA-DP, HLA-DQ, HLA-DR, RFX5, RFXANK, and/or RFXAP.

Embodiment 397. The engineered cell of any of embodiments 388-398, wherein the one or more tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, A20/TNFAIP3, CD39, CR1, HLA-F, IL15-RF, MANF, and any combination thereof.

Embodiment 398. The engineered cell of any of embodiments 388-399, wherein the one or more tolerogenic factors comprise CD47.

Embodiment 399. The engineered cell of any of embodiments 388-400, wherein the one or more tolerogenic factors comprise CCL22.

Embodiment 400. The engineered cell of any of embodiments 388-401, wherein the one or more tolerogenic factors comprise CD16 or CD16 Fc receptor.

Embodiment 401. The engineered cell of any of embodiments 388-402, wherein the one or more tolerogenic factors comprise CD24.

Embodiment 402. The engineered cell of any of embodiments 388-403, wherein the one or more tolerogenic factors comprise CD39.

Embodiment 403. The engineered cell of any of embodiments 388-404, wherein the one or more tolerogenic factors comprise CR1.

Embodiment 404. The engineered cell of any of embodiments 388, wherein the one or more tolerogenic factors comprise CD52.

Embodiment 405. The engineered cell of any of embodiments 388-406, wherein the one or more tolerogenic factors comprise CD55.

Embodiment 406. The engineered cell of any of embodiments 388-407, wherein the one or more tolerogenic factors comprise CD200.

Embodiment 407. The engineered cell of any of embodiments 388-408, wherein the one or more tolerogenic factors comprise DUX4.

Embodiment 408. The engineered cell of any of embodiments 388-409, wherein the one or more tolerogenic factors comprise HLA-E.

Embodiment 409. The engineered cell of any of embodiments 388-410, wherein the one or more tolerogenic factors comprise HLA-G.

Embodiment 410. The engineered cell of any of embodiments 388-411, wherein the one or more tolerogenic factors comprise IDO1.

Embodiment 411. The engineered cell of any of embodiments 388-412, wherein the one or more tolerogenic factors comprise IL15-RF.

Embodiment 412. The engineered cell of any of embodiments 388-413, wherein the one or more tolerogenic factors comprise IL35.

Embodiment 413. The engineered cell of any of embodiments 388-414, wherein the one or more tolerogenic factors comprise PD-L1.

Embodiment 414. The engineered cell of any of embodiments 388-415, wherein the one or more tolerogenic factors comprise MANF.

Embodiment 415. The engineered cell of any of embodiments 388-416, wherein the one or more tolerogenic factors comprise A20/TNFAIP3.

Embodiment 416. The engineered cell of any of embodiments 388-417, wherein the one or more tolerogenic factors comprise HLA-E and CD47.

Embodiment 417. The engineered cell of any of embodiments 388-31, wherein the one or more tolerogenic factors comprise two or more tolerogenic factors selected from the group consisting of CD47, CD46, and CD59, optionally wherein the one or more tolerogenic factors comprise CD47, CD46, and CD59.

Embodiment 418. The engineered cell of any of embodiments 388-419, wherein the one or more tolerogenic factors comprise two or more tolerogenic factors selected from the group consisting of CD47 and CD39, optionally wherein the one or more tolerogenic factors comprise CD47 and CD39.

Embodiment 419. The engineered cell of any of embodiments 388-420, wherein the one or more tolerogenic factors comprise two or more tolerogenic factors selected from the group consisting of CD47 and CCL22, optionally wherein the one or more tolerogenic factors comprise CD47 and CCL22.

Embodiment 420. The engineered cell of any of embodiments 388-421, wherein the one or more tolerogenic factors comprise two or more tolerogenic factors selected from the group consisting of CD47, HLA-G and PD-L1, optionally wherein the one or more tolerogenic factors comprise CD47 and PD-L1, and optionally wherein the one or more tolerogenic factors comprise CD47, HLA-G and PD-L1.

Embodiment 421. The engineered cell of any of embodiments 388-422, wherein the one or more tolerogenic factors comprise two or more tolerogenic factors selected from the group consisting of CD24, CD47, and PD-L1, optionally wherein the one or more tolerogenic factors comprise CD24, CD47, and PD-L1.

Embodiment 422. The engineered cell of any of embodiments 388-423, wherein the one or more tolerogenic factors comprise two or more tolerogenic factors selected from the group consisting of HLA-E, CD24, CD47, and PD-L1, optionally wherein the one or more tolerogenic factors comprise HLA-E, CD24, CD47, and PD-L1.

Embodiment 423. The engineered cell of any of embodiments 388-424, wherein the one or more tolerogenic factors comprise two or more tolerogenic factors selected from the group consisting of CD46, CD55, CD59, and CR1, optionally wherein the one or more tolerogenic factors comprise CD46, CD55, CD59, and CR1.

Embodiment 424. The engineered cell of any of embodiments 388-425, wherein the one or more tolerogenic factors comprise two or more tolerogenic factors selected from the group consisting of HLA-E, CD46, CD55, CD59, and CR1, optionally wherein the one or more tolerogenic factors comprise HLA-E, CD46, CD55, CD59, and CR1.

Embodiment 425. The engineered cell of any of embodiments 388-426, wherein the one or more tolerogenic factors comprise two or more tolerogenic factors selected from the group consisting of HLA-E, CD24, CD47, PD-L1, CD46, CD55, CD59, and CR1, optionally wherein the one or more tolerogenic factors comprise HLA-E, CD24, CD47, PD-L1, CD46, CD55, CD59, and CR1.

Embodiment 426. The engineered cell of any of embodiments 388-427, wherein the one or more tolerogenic factors comprise HLA-E and PD-L1.

Embodiment 427. The engineered cell of any of embodiments 388-428, wherein the one or more tolerogenic factors comprise two or more tolerogenic factors selected from the group consisting of HLA-E, PD-L1, and A20/TNFAIP, optionally wherein the one or more tolerogenic factors comprise HLA-E, PD-L1, and A20/TNFAIP.

Embodiment 428. The engineered cell of any of embodiments 388-429, wherein the one or more tolerogenic factors comprise two or more tolerogenic factors selected from the group consisting of HLA-E, PD-L1, and MANF, optionally wherein the one or more tolerogenic factors comprise HLA-E, PD-L1, and MANF.

Embodiment 429. The engineered cell of any of embodiments 388-430, wherein the one or more tolerogenic factors comprise two or more tolerogenic factors selected from the group consisting of HLA-E, PD-L1, A20/TNFAIP, and MANF, optionally wherein the one or more tolerogenic factors comprise HLA-E, PD-L1, A20/TNFAIP, and MANF.

Embodiment 430. An engineered cell comprising one or more modifications that (i) reduce expression of one or more MHC class I molecules and one or more MHC class II molecules, and (ii) increase expression of CD47, wherein the reduced expression of (i) and the increased expression of (ii) is relative to a cell of the same cell type that does not comprise the modifications.

Embodiment 431. The engineered cell of embodiment 432, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

Embodiment 432. The engineered cell of embodiment 432 or embodiment 46, wherein the one or more modifications in (i) reduce expression of B2M.

Embodiment 433. The engineered cell of any of embodiments 432-434, wherein the one or more modifications in (i) reduce expression of HLA-A, HLA-B, and/or HLA-C.

Embodiment 434. The engineered cell of any of embodiments 432-435, wherein the one or more modifications in (i) reduce expression of CIITA.

Embodiment 435. The engineered cell of any of embodiments 432-435, wherein the one or more modifications in (i) reduce expression of HLA-DP, HLA-DR, and/or HLA-DQ.

Embodiment 436. The engineered cell of any of embodiments 388-437, wherein the engineered cell further comprises one or more modifications that increase expression of one or more additional tolerogenic factors.

Embodiment 437. The engineered cell embodiment 438, wherein the one or more additional tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD64, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, A20/TNFAIP3, CD39, CR1, HLA-F, IL15-RF, MANF, and any combination thereof.

Embodiment 438. The engineered cell of embodiment 439, wherein the one or more additional tolerogenic factors comprise CD47.

Embodiment 439. The engineered cell of any one of embodiments 388-440, wherein the engineered cell further comprises one or more modifications that reduce expression of one or more additional molecules.

Embodiment 440. The engineered cell of embodiment 441, wherein the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

Embodiment 441. The engineered cell of embodiment 441 or 442, wherein the one or more additional molecules comprise one or more Y chromosome proteins, optionally Protocadherin-11 Y-linked (PCDH11Y) and/or Neuroligin-4 Y-linked (NLGN4Y).

Embodiment 442. The engineered cell of any of embodiments 441-443, wherein the one or more additional molecules comprise one or more NK cell ligands, optionally MIC-A and/or MIC-B.

Embodiment 443. The engineered cell of any of embodiments 441-444, wherein the one or more additional molecules comprise one or more proteins involved in oxidative or ER stress, optionally thioredoxin-interacting protein (TXNIP), PKR-like ER kinase (PERK), inositol-requiring enzyme 1α (IRE1α), and/or DJ-1 (PARK7).

Embodiment 444. The engineered cell of any of embodiments 441-446, wherein the one or more additional molecules comprise one or more blood antigen proteins, optionally ABO, FUT1 and/or RHD.

Embodiment 445. The engineered cell of any one of embodiments 388-446, wherein the engineered cell further comprises one or more modifications that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

Embodiment 446. The engineered cell of embodiment 447, wherein TRB is TRBC1, TRBC2, or TRBC1 and TRBC2.

Embodiment 447. The engineered cell of any of embodiments 388-448, wherein reduced expression comprises no cell surface expression or no detectable cell surface expression.

Embodiment 448. The engineered cell of any of embodiments 388-449, wherein reduced expression comprises reduced mRNA expression, optionally wherein reduced expression comprises no detectable mRNA expression.

Embodiment 449. The engineered cell of any of embodiments 388-460, wherein reduced expression comprises reduced protein expression or reduced protein activity, optionally wherein reduced expression comprises no detectable protein expression or protein activity.

Embodiment 450. The engineered cell of any of embodiments 388-451, wherein reduced expression comprises eliminating activity of a gene encoding or regulating the expression of i) the one or more MHC class I molecules and/or the one or more MHC class II molecules, or ii) the one or more additional molecules.

Embodiment 451. The engineered cell of any of embodiments 388-452, wherein reduced expression comprises inactivation or disruption of an allele of a gene encoding or regulating the expression of i) the one or more MHC class I molecules and/or the one or more MHC class II molecules, or ii) the one or more additional molecules.

Embodiment 452. The engineered cell of any of embodiments 388-453, wherein reduced expression comprises inactivation or disruption of both alleles of a gene encoding or regulating the expression of i) the one or more MHC class I molecules and/or the one or more MHC class II molecules, or ii) the one or more additional molecules.

Embodiment 453. The engineered cell of any of embodiments 388-454, wherein the one or more modifications to reduce expression comprises an indel in a gene encoding or regulating the expression of i) the one or more MHC class I molecules and/or the one or more MHC class II molecules, or ii) the one or more additional molecules.

Embodiment 454. The engineered cell of any of embodiments 388-455, wherein the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.

Embodiment 455. The engineered cell of any of embodiments 388-456, wherein the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.

Embodiment 456. The engineered cell of any of embodiments 388-456, wherein the one or more modifications to reduce expression comprises knocking out a gene encoding or regulating the expression of i) the one or more MHC class I molecules and/or the one or more MHC class II molecules, or ii) the one or more additional molecules.

Embodiment 457. The engineered cell of any of embodiments 388-458, wherein the engineered cell comprises one or more modifications that:
a. reduce expression of MHC class I and/or MHC class II molecules;
b. increase expression of CD47; and
c. increase expression of CCL22.

Embodiment 458. The engineered cell of any of embodiments 388-458, wherein the engineered cell comprises one or more modifications that:
a. reduce expression of MHC class I and/or MHC class II molecules;
b. increase expression of CD47; and
c. increase expression of CD39.

Embodiment 459. The engineered cell of any of embodiments 388-458, wherein the engineered cell comprises one or more modifications that:
a. reduce expression of MHC class I and/or MHC class II molecules;
b. increase expression of CD47; and
c. increase expression of CD46 and CD59.

Embodiment 460. The engineered cell of any of embodiments 388-458, wherein the engineered cell comprises one or more modifications that:
a. reduce expression of MHC class I and/or MHC class II molecules;
b. increase expression of CD47; and
c. increase expression of PD-L1.

Embodiment 461. The engineered cell of any of embodiments 388-458, wherein the engineered cell comprises one or more modifications that:
a. reduce expression of MHC class I and/or MHC class II molecules;
b. increase expression of CD47; and
c. increase expression of HLA-G and PD-L1.

Embodiment 462. The engineered cell of any of embodiments 388-458, wherein the engineered cell comprises one or more modifications that:
a. reduce expression of MHC class I and/or MHC class II molecules;
b. increase expression of CD47; and
c. reduced expression of CD142.

Embodiment 463. The engineered cell of any of embodiments 388-458, wherein the engineered cell comprises one or more modifications that:
a. reduce expression of MHC class I and/or MHC class II molecules;
b. increase expression of CD47; and
c. reduced expression of MIC-A and/or MIC-B.

Embodiment 464. The engineered cell of any of embodiments 388-458, wherein the engineered cell comprises one or more modifications that:
a. reduce expression of MHC class I and/or MHC class II molecules; and
b. increase expression of CD24.

Embodiment 465. The engineered cell of any of embodiments 388-458, wherein the engineered cell comprises one or more modifications that:
a. reduce expression of MHC class I and/or MHC class II molecules; and
b. increase expression of CD200.

Embodiment 466. The engineered cell of any of embodiments 388-458, wherein the engineered cell comprises one or more modifications that:
a. reduce expression of MHC class I and/or MHC class II molecules; and
b. increase expression of CD52.

Embodiment 467. The engineered cell of any of embodiments 388-458, wherein the engineered cell comprises one or more modifications that:
a. reduce expression of MHC class I and/or MHC class II molecules; and
b. increase expression of DUX4.

Embodiment 468. The engineered cell of any of embodiments 388-458, wherein the engineered cell comprises one or more modifications that:
a. reduce expression of MHC class I and/or MHC class II molecules; and
b. increase expression of IDO1.

Embodiment 469. The engineered cell of any of embodiments 388-458, wherein the engineered cell comprises one or more modifications that:
a. reduce expression of MHC class I and/or MHC class II molecules; and
b. increase expression of IL-35.

Embodiment 470. The engineered cell of any of embodiments 388-458, wherein the engineered cell comprises one or more modifications that:
a. reduce expression of MHC class I and/or MHC class II molecules; and
b. increase expression of PD-L1.

Embodiment 471. The engineered cell of any of embodiments 388-458, wherein the engineered cell comprises one or more modifications that:
a. reduce expression of MHC class I and/or MHC class II molecules; and
b. increase expression of HLA-E.

Embodiment 472. The engineered cell of any of embodiments 388-458, wherein the engineered cell comprises one or more modifications that:
a. reduce expression of MHC class I and/or MHC class II molecules; and
b. increase expression of HLA-G.

Embodiment 473. The engineered cell of any of embodiments 388-458, wherein the engineered cell comprises one or more modifications that:
a. reduce expression of MHC class I and/or MHC class II molecules;
b. reduce expression of CD155; and
c. increase expression of HLA-E.

Embodiment 474. The engineered cell of any of embodiments 388-458, wherein the engineered cell comprises one or more modifications that:
a. reduce expression of MHC class I molecules;
b. reduce expression of RFXANK;
c. increase expression of HLA-E.

Embodiment 475. The engineered cell of any of embodiments 388-458, wherein the engineered cell comprises one or more modifications that:
a. reduce expression of MHC class I and/or MHC class II molecules;
b. reduce expression of MIC-A and/or MIC-B;
c. increase expression of one or more of CD47, CD24 and PD-L1; and
d. increase expression of CD46, CD55, CD59 and CR1.

Embodiment 476. The engineered cell of any of embodiments 388-458, wherein the engineered cell comprises one or more modifications that:
a. reduce expression of MHC class I molecules;
b. reduce expression of MIC-A and/or MIC-B;
c. reduce expression of TXNIP; and
d. increase expression of PD-L1 and HLA-E.

Embodiment 477. The engineered cell of embodiment 477, wherein the modifications further increase expression of A20/TNFAIP3 and MANF.

Embodiment 478. The engineered of any one of embodiments 388-479, wherein the one or more modifications that reduce expression of MHC class I and/or MHC class II molecules consist of one or more modifications that reduce expression of MHC class I molecules.

Embodiment 479. The engineered of any one of embodiments 388-479, wherein the one or more modifications that reduce expression of MHC class I and/or MHC class II molecules consist of one or more modifications that reduce expression of MHC class II molecules.

Embodiment 480. The engineered of any one of embodiments 388-479, wherein the one or more modifications that reduce expression of MHC class I and/or MHC class II molecules consist of one or more modifications that reduce expression of MHC class I molecules and MHC class II molecules.

Embodiment 481. The engineered cell of embodiment 388-482, wherein increased expression comprises increased mRNA expression.

Embodiment 482. The engineered cell of embodiment 388-483, wherein increased expression comprises increased protein expression or protein activity.

Embodiment 483. The engineered cell of any one of embodiments 388-484, wherein increased expression comprises increasing activity of a gene encoding or regulating the expression of i) the one or more tolerogenic factors, or ii) the one or more additional tolerogenic factors.

Embodiment 484. The engineered cell of embodiment 485, wherein the gene is an endogenous gene and the one or more modifications comprise one or more modifications of an endogenous promoter.

Embodiment 485. The engineered cell of embodiment 485, wherein the gene is an endogenous gene and the one or more modifications comprise introduction of a heterologous promoter.

Embodiment 486. The engineered cell of embodiment 487, wherein the heterologous promoter is selected from the group consisting of a CAG promoter, cytomegalovirus (CMV) promoter, EFla promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 487. The engineered cell of any of embodiments 388-481, wherein the engineered cell comprises one or more transgenes.

Embodiment 488. The engineered cell of embodiment 489, wherein the one or more transgenes encode at least one of the one or more tolerogenic factors or the one or more additional tolerogenic factors.

Embodiment 489. The engineered cell of embodiment 489 or 490, wherein the one or more transgenes encode at least one of the one or more additional tolerogenic factors.

Embodiment 490. The engineered cell of any one of embodiments 489-491, wherein the one or more transgenes encode one or more additional molecules.

Embodiment 491. The engineered cell of any of embodiments 489-492, wherein the one or more transgenes comprise one or more regulatory elements.

Embodiment 492. The engineered cell of any of embodiments 489-493, wherein the one or more transgenes are operably linked to the one or more regulatory elements.

Embodiment 493. The engineered cell of embodiment 493 or embodiment 10⁷, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.

Embodiment 494. The engineered cell of embodiment 493, embodiment 495, or embodiment 107, wherein the one or more regulatory elements comprise a promoter.

Embodiment 497. The engineered cell of embodiment 495 or 496, wherein the promoter is selected from the group consisting of a CAG promoter, cytomegalovirus (CMV) promoter, EF1α promoter, EF1a short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 498. The engineered cell of any of embodiments 489-497, wherein the engineered cell comprises one or more vectors encoding the one or more transgenes.

Embodiment 499. The engineered cell of embodiment 498, wherein at least one of the one or more vectors is a multicistronic vector.

Embodiment 500. The engineered cell of embodiment 499, wherein the multicistronic vector encodes at least one of the one or more tolerogenic factors or the one or more additional tolerogenic factors.

Embodiment 501. The engineered cell of embodiment 499 or embodiment 113, wherein the multicistronic vector further encodes at least one of the one or more tolerogenic factors or the one or more additional tolerogenic factors.

Embodiment 502. The engineered cell of embodiment of embodiment 500 or embodiment 501, wherein the multicistronic vector further encodes at least one of the one or more additional molecules.

Embodiment 503. The engineered cell of any one of embodiments 489-502, wherein the one or more transgenes are separated by one or more linker sequences.

Embodiment 504. The engineered cell of embodiment 503, wherein the one or more linker sequences comprise an IRES sequence or a cleavable peptide sequence.

Embodiment 505. The engineered cell of embodiment 504, wherein the cleavable peptide sequence comprises a self-cleavable peptide, optionally a 2A peptide.

Embodiment 506. The engineered cell of embodiment 505, wherein the 2A peptide is selected from the group consisting of a F2A sequence, an E2A sequence, a P2A sequence, and a T2A sequence.

Embodiment 507. The engineered cell of any of embodiments 504-506, wherein the cleavable peptide sequence comprises a protease cleavable sequence or a chemically cleavable sequence.

Embodiment 508. The engineered cell of any of embodiments 500-507, wherein the one or more tolerogenic factors, the one or more additional tolerogenic factors, and/or the one or more additional molecules are operably linked to the same promoter.

Embodiment 509. The engineered cell of any of embodiment 508, wherein the promoter is a constitutive promoter.

Embodiment 510. The engineered cell of embodiment 508 or 509, wherein the promoter is selected from the group consisting of a CAG promoter, cytomegalovirus (CMV) promoter, EF1α promoter, EF1a short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 511. The engineered cell of any of embodiments 492-510, wherein the one or more additional molecules comprise a chimeric antigen receptor (CAR).

Embodiment 512. The engineered cell of embodiment 511, wherein the CAR comprises a signal peptide, an extracellular binding domain specific to CD19, a hinge domain, a transmembrane domain, an intracellular costimulatory domain, and/or an intracellular signaling domain.

Embodiment 513. The engineered cell of embodiment 511 or embodiment 125, wherein the CAR is specific for CD19, CD20, CD22, CD38, CD123, CD138, BCMA, or any combination thereof.

Embodiment 514. The engineered cell of embodiment 513, wherein the CAR is a CD19/CD22-bispecific CAR.

Embodiment 515. The engineered cell of any of embodiments 492-514, wherein the one or more additional molecules comprise one or more safety switches.

Embodiment 516. The engineered cell of embodiment 515, wherein the one or more safety switches are capable of controlled killing of the engineered cell.

Embodiment 517. The engineered cell of embodiment 515 or 516, wherein the one or more safety switches induce controlled cell death in the presence of a drug or prodrug, or upon activation by a selective exogenous compound.

Embodiment 518. The engineered cell of any of embodiments 515-517, wherein the one or more safety switches comprise is an inducible protein capable of inducing apoptosis of the engineered cell.

Embodiment 519. The engineered cell of embodiment 518, wherein the inducible protein capable of inducing apoptosis of the engineered cell is a caspase protein.

Embodiment 520. The engineered cell of embodiment 519, wherein the caspase protein is caspase 9.

Embodiment 521. The engineered cell of any of embodiments 515-520, wherein the one or more safety switches comprise one or more suicide genes.

Embodiment 522. The engineered cell of embodiment 521, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 523. The engineered cell of any of embodiments 489-522, wherein at least one of the one or more transgenes are integrated into the genome of the engineered cell.

Embodiment 524. The engineered cell of embodiment 523, wherein integration is by non-targeted insertion into the genome of the engineered cell.

Embodiment 525. The engineered cell of embodiment 524, wherein integration is by non-targeted insertion into the genome of the engineered cell using a lentiviral vector.

Embodiment 526. The engineered cell of embodiment 523, wherein integration is by targeted insertion into a target genomic locus of the engineered cell.

Embodiment 527. The engineered cell of embodiment 526, wherein targeted insertion is by nuclease-mediated gene editing with homology-directed repair.

Embodiment 528. The engineered cell of embodiment 526 or 527, wherein the target genomic locus is selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

Embodiment 529. The engineered cell of any of embodiments 388-528, wherein the genome of the engineered cell comprises on or more gene edits in one or more genes encoding the one or more molecules of any of embodiments 388-141 having reduced expression.

Embodiment 530. The engineered cell of any of embodiments 388-529, wherein the engineered cell comprises a genome editing complex.

Embodiment 531. The engineered cell of embodiment 530, wherein the genome editing complex comprises a genome targeting entity and a genome modifying entity.

Embodiment 532. The engineered cell of embodiment 531, wherein the genome targeting entity localizes the genome editing complex to the target locus, optionally wherein the genome targeting entity is a nucleic acid-guided targeting entity.

Embodiment 533. The engineered cell of embodiment 531 or embodiment 532, wherein the genome targeting entity comprises a transcription activator-like effector (TALE) binding protein, a zinc finger (ZF) binding protein, a Meganuclease, a Cas protein, a TnpB protein, a homing endonuclease, an endonuclease-deficient-Cas protein, an enzymatically inactive Cas protein, a nucleic acid programmable DNA binding protein, or a functional portion thereof.

Embodiment 534. The engineered cell of any of embodiments 531-533, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, or a functional portion thereof.

Embodiment 535. The engineered cell of embodiment 531, wherein the genome modifying entity cleaves, deaminates, nicks, polymerizes, interrogates, integrates, cuts, unwinds, breaks, alters, methylates, demethylates, or otherwise destabilizes the target locus.

Embodiment 536. The engineered cell of embodiment 531 or embodiment 535, wherein the genome modifying entity comprises a recombinase, integrase, transposase, endonuclease, exonuclease, nickase, helicase, polymerase, reverse transcriptase, deaminase, flippase, methylase, demethylase, acetylase, a nucleic acid modifying protein, an RNA modifying protein, a DNA modifying protein, Argonaute protein, an epigenetic modifying protein, a histone modifying protein, or a functional portion thereof.

Embodiment 537. The engineered cell of embodiment 536, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, APOBEC1, cytidine deaminase, adenosine deaminase, uracil glycosylase inhibitor (UGI), adenine base editors (ABE), cytosine base editors (CBE), reverse transcriptase, serine integrase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

Embodiment 538. The engineered cell of any of embodiments 531-537, wherein the genome targeting entity and the genome modifying entity are different domains of a single polypeptide.

Embodiment 539. The engineered cell of any of embodiments 531-538, wherein the genome editing entity and genome modifying entity are two different polypeptides that are operably linked together.

Embodiment 540. The engineered cell of any of embodiments 531-538, wherein the genome editing entity and genome modifying entity are two different polypeptides that are not linked together.

Embodiment 541. The engineered cell of any of embodiments 462-469, wherein the modification is by a genome-modifying protein.

Embodiment 542. The engineered cell of any of embodiments 470, wherein the modification by a genome-modifying protein is modification by a CRISPR-associated transposase, prime editing, or Programmable Addition via Site-specific Targeting Elements (PASTE).

Embodiment 543. The engineered cell of any of embodiments 470-471, wherein the modification by the genome-modifying protein is nuclease-mediated gene editing.

Embodiment 544. The engineered cell of embodiment 472, wherein the nuclease-mediated gene editing is by a zinc finger nuclease (ZFN), a TAL-effector nuclease (TALEN), or a CRISPR-Cas combination that targets the B2M gene, optionally wherein the Cas is selected from a Cas9 or a Cas12.

Embodiment 545. The engineered cell of any of embodiments 470-472, wherein the modification by the genome-modifying protein is performed by one or more proteins selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 546. The engineered cell of embodiment 473, wherein the nuclease-mediated gene editing is by a CRISPR-Cas combination and the CRISPR-Cas combination comprises a guide RNA (gRNA) having a targeting domain that is complementary to at least one target site within the B2M gene.

Embodiment 547. The engineered cell of embodiment 475, wherein the CRISPR-Cas combination is a ribonucleoprotein (RNP) complex comprising the gRNA and a Cas protein.

Embodiment 548. The engineered cell of any of embodiments 1-547, wherein the engineered cell is a human cell or an animal cell.

Embodiment 549. The engineered cell of embodiment 548, wherein the animal cell is a porcine cell, a bovine cell, or an ovine cell.

Embodiment 550. The engineered cell of embodiment 548, wherein the engineered cell is a human cell.

Embodiment 551. The engineered cell of any of embodiments 388-550, wherein the engineered cell is a stem cell or progenitor cell.

Embodiment 552. The engineered cell of embodiment 551, wherein the engineered cell is a differentiated cell derived from the stem cell or progenitor cell.

Embodiment 553. The engineered cell of embodiment 551 or 552, wherein the stem cell or progenitor cell is selected from the group consisting of an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell.

Embodiment 554. The engineered cell of any of embodiments 388-550, wherein the engineered cell is a differentiated cell derived from a pluripotent stem cell or a progeny thereof.

Embodiment 555. The engineered cell of embodiment 554, wherein the pluripotent stem cell is an induced pluripotent stem cell.

Embodiment 556. The engineered cell of any of embodiments 388-550, wherein the engineered cell is a primary cell isolated from a donor subject.

Embodiment 557. The engineered cell of embodiment 556, wherein the donor subject is healthy or is not suspected of having a disease or condition at the time the donor sample is obtained from the individual donor.

Embodiment 558. The engineered cell of any of embodiments 388-557, wherein the engineered cell is selected from the group consisting of an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a cardiac cell, a stem cell, a hematopoietic stem cell, an induced pluripotent stem cell (iPSC), a mesenchymal stem cell (MSC), an embryonic stem cell (ESC), a pluripotent stem cell (PSC), and a blood cell.

Embodiment 559. The engineered cell of any of embodiments 388-558, wherein the cell is ABO blood group type O.

Embodiment 560. The engineered cell of any of embodiments 388-559, wherein the cell comprises a functional ABO A allele and/or a functional ABO B allele.

Embodiment 561. The engineered cell of any of embodiments 388-560, wherein the cell is Rhesus factor negative (Rh−).

Embodiment 562. The engineered cell of any of embodiments 388-560, wherein the cell is Rhesus factor positive (Rh+).

Embodiment 563. A method of generating the engineered cell of any of embodiments 388-562 comprising
a. obtaining a cell; and
b. introducing the one or more modifications of any of embodiments 388-562 into the cell.

Embodiment 564. The method of embodiment 563, wherein the method further comprises selecting the engineered cell from a population of cells based on the presence and/or level of one or more of the modifications.

Embodiment 565. The method of embodiment 563 or 564, wherein the cell is a stem cell or a progenitor cell and the method further comprises differentiating the stem cell or the progenitor cell.

Embodiment 566. The method of embodiment 563 or 564, wherein the cell is a pluripotent stem cell or a progeny thereof and the method comprises differentiating the pluripotent stem cell or progeny thereof.

Embodiment 567. The method of embodiment 563 or 564, wherein the cell is a primary cell.

Embodiment 568. The method of any of embodiments 563-567, wherein the method comprises introducing one or more gene edits into the genome of the cell.

Embodiment 569. The method of embodiment 568, wherein the one or more gene edits are introduced into the genome of the cell by non-targeted insertion.

Embodiment 570. The method of embodiment 568, wherein the one or more gene edits are introduced into the genome of the cell by targeted insertion.

Embodiment 571. The method of embodiment 568 or 570, wherein the one or more gene edits are introduced into one or more genes encoding the one or more molecules of any of embodiments 388-561.

Embodiment 572. The method of embodiment 571, wherein the engineered cell has increased expression of the one or more molecules encoded by the one or more edited genes.

Embodiment 573. The method of embodiment 571 or 572, wherein the engineered cell has reduced expression of the one or more molecules encoded by the one or more edited genes.

Embodiment 574. The method of any of embodiments 568-185, wherein the one or more gene edits are introduced into the genome of cell using at least one of the genome editing complexes of any of embodiments 530-547.

Embodiment 575. The method of any of embodiments 568-574, wherein the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

Embodiment 576. An engineered cell produced according to the method of any of embodiments 563-575.

Embodiment 577. The engineered cell of any of embodiments 388-562 and 576, wherein the engineered cell, or progeny or differentiated cells have increased capability to evade NK cell mediated cytotoxicity upon administration to a subject as compared to a cell of the same type that does not comprise the one or more modifications.

Embodiment 578. The engineered cell of any of embodiments 388-562, 576 and 577, wherein the engineered cell, or progeny or differentiated cells derived from the engineered cell undergo reduced cell lysis by mature NK cells upon administration to a subject as compared to a cell of the same type that does not comprise the one or more modifications.

Embodiment 579. The engineered cell of any of embodiments 388-562 and 576-578, wherein the engineered cell, or progeny or differentiated cells derived from the engineered cell induce a reduced immune response upon administration to a subject as compared to a cell of the same type that does not comprise the one or more modifications.

Embodiment 580. The engineered cell of any of embodiments 388-562 and 576-579, wherein the engineered cell, or progeny or differentiated cells derived from the engineered cell induce a reduced systemic inflammatory response upon administration to a subject as compared to a cell of the same type that does not comprise the one or more modifications.

Embodiment 581. The engineered cell of any of embodiments 388-562 and 576-580, wherein the engineered cell, or progeny or differentiated cells derived from the engineered cell induce a reduced local inflammatory response upon administration to a subject as compared to a cell of the same type that does not comprise the one or more modifications.

Embodiment 582. The engineered cell of any of embodiments 388-562 and 576-581, wherein the engineered cell, or progeny or differentiated cells derived from the engineered cell induce reduced complement pathway activation upon administration to a subject as compared to a cell [of the same type] that does not comprise the one or more modifications.

Embodiment 583. The engineered cell of any of embodiments 388-562 and 576-582, wherein the engineered cell, or progeny or differentiated cells derived from the engineered cell retain the ability to engraft and function upon administration to a subject.

Embodiment 584. The engineered cell of any of embodiments 388-562 and 576-583, wherein the engineered cell, or progeny or differentiated cells derived from the engineered cell has increased ability to engraft and function upon administration to a subject as compared to a cell [of the same type] that does not comprise the one or more modifications.

Embodiment 585. A population of engineered cells comprising a plurality of the engineered cells of any of embodiments 388-562 and 576-584.

Embodiment 586. The population of engineered cells of embodiment 585, wherein at least about 30% of cells in the population comprise the plurality of the engineered cells.

Embodiment 587. The population of engineered cells of embodiment 585 or embodiment 586, wherein the plurality of the engineered cells are primary cells isolated from more than one donor subject.

Embodiment 588. The population of engineered cells of embodiment 587, wherein each donor subject is healthy or is not suspected of having a disease or condition at the time the donor sample is obtained from the individual donor.

Embodiment 589. A method of producing a composition comprising the engineered cell of any of embodiments 1-562 and 576-196 or the population of engineered cells of any of embodiments 585-588 comprising
a. obtaining the cell of any of embodiments 548-562;
b. introducing the one or more modifications of any of embodiments 388-562 into the cell;
c. selecting the engineered cell or selecting the population of engineered cells from a population of cells based on a level of the one or more of the modifications; and
d. formulating the composition comprising the selected engineered cell or the selected population of engineered cells.

Embodiment 590. The method of embodiment 589, wherein method comprises selecting the engineered cell or the population of engineered cells based on the level of cell surface expression of the one or more modified molecules in any of embodiments 388-561.

Embodiment 591. The method of embodiment 589 or embodiment 590, wherein the engineered cell or the population of engineered cells are selected based on a level of the one or more modified molecules having reduced expression in the engineered cell or the population of engineered cells.

Embodiment 592. The method of any of embodiments 589-591, wherein the engineered cell or the population of engineered cells are selected based on a level of the one or more modified molecules having increased expression in the engineered cell or the population of engineered cells.

Embodiment 593. The method of any of embodiments 589-592, wherein the method comprises formulating the composition in a pharmaceutically acceptable additive, carrier, diluent, or excipient.

Embodiment 594. The method of embodiment 593, wherein the pharmaceutically acceptable additive, carrier, diluent, or excipient comprises a pharmaceutically acceptable buffer.

Embodiment 595. The method of embodiment 594, wherein the pharmaceutically acceptable buffer comprises neutral buffer saline or phosphate buffered saline.

Embodiment 596. The method of any of embodiments 589-595, wherein the method comprises formulating the composition with Plasma-Lyte A®, dextrose, dextran, sodium chloride, human serum albumin (HSA), dimethylsulfoxide (DMSO), or a combination thereof.

Embodiment 597. The method of any of embodiments 589-596, wherein the method comprises formulating the composition with a cryoprotectant.

Embodiment 598. The method of any of embodiments 589-597, wherein the method comprises formulating the composition in a serum-free cryopreservation medium comprising a cryoprotectant.

Embodiment 599. The method of embodiment 597 or embodiment 598, wherein the cryoprotectant comprises DMSO.

Embodiment 600. The method of embodiment 598 or embodiment 599, wherein the serum-free cryopreservation medium comprises about 5% to about 10% DMSO (v/v).

Embodiment 601. The method of any of embodiments 598-600, wherein the serum-free cryopreservation medium comprises about 10% DMSO (v/v).

Embodiment 602. The method of any of embodiments 589-601, wherein the method further comprises storing the composition in a container.

Embodiment 603. The method of any of embodiments 589-602, wherein the method further comprises thawing the cell before step (b).

Embodiment 604. The method of any of embodiments 589-603, wherein the method further comprises freezing the engineered cell, the population of engineered cells, or the composition.

Embodiment 605. The method of embodiment 604, wherein the engineered cell or the population of engineered cells are frozen after step (b).

Embodiment 606. The method of embodiment 605, wherein the engineered cell or the population of engineered cells are thawed before step (c).

Embodiment 607. The method of embodiment 604, wherein the engineered cell or the population of engineered cells are frozen after step (c).

Embodiment 608. The method of embodiment 607, wherein the engineered cell or the population of engineered cells are thawed before step (d).

Embodiment 609. The method of embodiment 604, wherein the engineered cell or the population of engineered cells are frozen after step (c).

Embodiment 610. The method of any of embodiments 589-609, wherein the composition is frozen after step (d).

Embodiment 611. A composition comprising the engineered cell of any of embodiments 1-562 and 576-196 or the population of engineered cells of any of embodiments 585-588.

Embodiment 612. A composition produced by the method of any one of embodiments 589-610.

Embodiment 613. The composition of embodiment 611 or embodiment 612, wherein the composition comprises a pharmaceutically acceptable additive, carrier, diluent, or excipient.

Embodiment 614. The composition of any of embodiments 611-613, wherein the composition is sterile.

Embodiment 615. A container comprising the composition of any of embodiments 612-614.

Embodiment 616. The container of embodiment 615, wherein the container is a sterile bag.

Embodiment 617. The container of embodiment 616, wherein the sterile bag is a cryopreservation-compatible bag.

Embodiment 618. A kit comprising the composition of any of embodiments 612-614 or the container of any of embodiments 615-617.

Embodiment 619. The kit of embodiment 618, wherein the kit further comprises instructions for using the engineered cells or the population of engineered cells.

Embodiment 620. A method of treating a condition or disease in a subject in need thereof comprising administering to the subject an effective amount of the engineered cell of any of embodiments 1-562 and 576-196, the population of engineered cells of any of embodiments 585-588, or the composition of any of embodiments 611-613, optionally wherein the disease or condition is a cellular deficiency.

Embodiment 621. The method of embodiment 620, wherein the condition or disease is selected from the group consisting of diabetes, cancer, vascularization disorders, ocular disease, thyroid disease, skin diseases, and liver diseases.

Embodiment 622. The method of embodiment 620 or 621, wherein the condition or disease is associated with diabetes or is diabetes, optionally wherein the diabetes is Type I diabetes.

Embodiment 623. The method of embodiment 622, wherein the population of engineered cells is a population of islet cells, including beta islet cells.

Embodiment 624. The method of embodiment 623, wherein the islet cells are selected from the group consisting of an islet progenitor cell, an immature islet cell, and a mature islet cell.

Embodiment 625. The method of embodiment 620, wherein the condition or disease is associated with a vascular condition or disease or is a vascular condition or disease.

Embodiment 626. The method of embodiment 625, wherein the engineered cell or the population of engineered cells comprises an endothelial cell.

Embodiment 627. The method of embodiment 620, wherein the condition or disease is associated with autoimmune thyroiditis or is autoimmune thyroiditis.

Embodiment 628. The method of embodiment 627, wherein the engineered cell or the population of engineered cells comprise a thyroid progenitor cell.

Embodiment 629. The method of embodiment 620, wherein the condition or disease is associated with a liver disease or is liver disease.

Embodiment 630. The method of embodiment 629, wherein the liver disease comprises cirrhosis of the liver.

Embodiment 631. The method of embodiment 629 or 630, wherein the engineered cell or the population of engineered cells comprise a hepatocyte or a hepatic progenitor cell.

Embodiment 632. The method of embodiment 620, wherein the condition or disease is associated with a corneal disease or is corneal disease.

Embodiment 633. The method of embodiment 632, wherein the corneal disease is Fuchs dystrophy or congenital hereditary endothelial dystrophy.

Embodiment 634. The method of embodiment 632 or 633, wherein engineered cell or the population of engineered cells comprise a corneal endothelial progenitor cell or a corneal endothelial cells.

Embodiment 635. The method of embodiment 620, wherein the condition or disease is associated with a kidney disease or is kidney disease.

Embodiment 636. The method of embodiment 635, wherein the engineered cell or the population of engineered cells comprise a renal precursor cell or a renal cell.

Embodiment 637. The method of embodiment 620, wherein the condition or disease is associated with a cancer or is cancer.

Embodiment 638. The method of embodiment 637, wherein the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

Embodiment 639. The method of embodiment 637 or 638, wherein the engineered cell or the population of engineered cells comprise a T cell, an NK cell, or an NKT cell.

Embodiment 640. The method of embodiment 620, wherein the condition or disease is associated with a hematopoietic disease or disorder or is a hematopoietic disease or disorder.

Embodiment 641. The method of embodiment 640, wherein the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.

Embodiment 642. The method of embodiment 620, wherein the condition or disease is associated with leukemia or myeloma or is leukemia or myeloma.

Embodiment 643. The method of embodiment 620, wherein the condition or disease is associated with an autoimmune disease or condition or is an autoimmune disease or condition.

Embodiment 644. The method of embodiment 643, wherein the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopeni purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenia purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

Embodiment 645. The method of any of embodiments 640-644, wherein engineered cell or the population of engineered cells comprises a hematopoietic stem cell (HSC) or a derivative thereof.

Embodiment 646. The method of embodiment 620, wherein the condition or disease is associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS), or wherein the disease or condition is Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS).

Embodiment 647. The method of embodiment 646, wherein the engineered cell or the population of engineered cells comprise a neural cell or a glial cell.

Embodiment 648. The method of any of embodiments 620-647, wherein the engineered cell or the population of engineered cells are expanded and cryopreserved prior to administration.

Embodiment 649. The method of any of embodiments 620-648, wherein the method comprises intravenous injection, intramuscular injection, intravascular injection, or transplantation of the engineered cell, the population of engineered cells, or the composition.

Embodiment 650. The method of embodiment 649, wherein transplantation comprises intravascular injection or intramuscular injection.

Embodiment 651. The method of any of embodiments 620-650, wherein the method further comprises administering one or more immunosuppressive agents to the subject.

Embodiment 652. The method of any of embodiments 620-651, wherein the subject has been administered one or more immunosuppressive agents.

Embodiment 653. The method of embodiment 651 or embodiment 652, wherein the one or more immunosuppressive agents are a small molecule or an antibody.

Embodiment 654. The method of any of embodiments 651-653, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), an immunomodulatory agent, and an immunosuppressive antibody.

Embodiment 655. The method of any of embodiments 651-654, wherein the one or more immunosuppressive agents comprise cyclosporine.

Embodiment 656. The method of any of embodiments 651-654, wherein the one or more immunosuppressive agents comprise mycophenolate mofetil.

Embodiment 657. The method of any of embodiments 651-654, wherein the one or more immunosuppressive agents comprise a corticosteroid.

Embodiment 658. The method of any of embodiments 651-654, wherein the one or more immunosuppressive agents comprise cyclophosphamide.

Embodiment 659. The method of any of embodiments 651-654, wherein the one or more immunosuppressive agents comprise rapamycin.

Embodiment 660. The method of any of embodiments 651-654, wherein the one or more immunosuppressive agents comprise tacrolimus (FK-506).

Embodiment 661. The method of any of embodiments 651-654, wherein the one or more immunosuppressive agents comprise anti-thymocyte globulin.

Embodiment 662. The method of any of embodiments 651-654, wherein the one or more immunosuppressive agents are one or more immunomodulatory agents.

Embodiment 663. The method of embodiment 662, wherein the one or more immunomodulatory agents are a small molecule or an antibody.

Embodiment 664. The method of embodiment 662 or embodiment 663, wherein the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

Embodiment 665. The method of any of embodiments 651-664, wherein the one or more immunosuppressive agents are or have been administered to the subject prior to administration of the engineered cell, the population of engineered cells, or the composition.

Embodiment 666. The method of any of embodiments 651-665, wherein the one or more immunosuppressive agents are or have been administered to the subject at least 1, 2, 3, 4, 5, 6, 7, 8, 9, 10, 11, 12, 13, or 14 days prior to administration of the engineered cell, the population of engineered cells, or the composition.

Embodiment 667. The method of any of embodiments 651-666, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the engineered cell, the population of engineered cells, or the composition.

Embodiment 668. The method of any of embodiments 651-664, wherein the one or more immunosuppressive agents are or have been administered to the subject after administration of the engineered cell, the population of engineered cells, or the composition.

Embodiment 669. The method of any of embodiments 651-664 and 668, wherein the one or more immunosuppressive agents are or have been administered to the subject at least 1, 2, 3, 4, 5, 6, 7, 8, 9, 10, 11, 12, 13, or 14 days after administration of the engineered cell, the population of engineered cells, or the composition.

Embodiment 670. The method of any of embodiments 651-664, 668 and 669, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the engineered cell, the population of engineered cells, or the composition.

Embodiment 671. The method of any of embodiments 651-664, wherein the one or more immunosuppressive agents are or have been administered to the subject on the same day as the first administration of the engineered cell, the population of engineered cells, or the composition.

Embodiment 672. The method of any of embodiments 651-664, wherein the one or more immunosuppressive agents are or have been administered to the subject after administration of a first and/or second administration of the engineered cell, the population of engineered cells, or the composition.

Embodiment 673. The method of any of embodiments 651-664, wherein the one or more immunosuppressive agents are or have been administered to the subject prior to administration of a first and/or second administration of the engineered cell, the population of engineered cells, or the composition.

Embodiment 674. The method of any of embodiments 651-664, wherein the one or more immunosuppressive agents are or have been administered to the subject at least 1, 2, 3, 4, 5, 6, 7, 8, 9, 10, 11, 12, 13, or 14 days prior to administration of a first and/or second administration of the engineered cell, the population of engineered cells, or the composition.

Embodiment 675. The method of any of embodiments 651-664, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the engineered cell, the population of engineered cells, or the composition.

Embodiment 676. The method of any of embodiments 651-664, wherein the one or more immunosuppressive agents are or have been administered to the subject at least 1, 2, 3, 4, 5, 6, 7, 8, 9, 10, 11, 12, 13, or 14 days after administration of a first and/or second administration of the engineered cell, the population of engineered cells, or the composition.

Embodiment 677. The method of any of embodiments 651-664, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the engineered cell, the population of engineered cells, or the composition.

Embodiment 678. The method of any of embodiments 651-677, wherein the one or more immunosuppressive agents are administered at a lower dosage as compared to the dosage administered to reduce immune rejection of a cell that does not comprise the one or more modifications of the engineered cell or the population of engineered cells.

Embodiment 679. The method of any of embodiments 620-678, wherein the method further comprises activating the safety switch to induce controlled cell death after the administration of the the engineered cell, the population of engineered cells, or the composition to the subject.

Embodiment 680. The method of any of embodiments 620-679, wherein the suicide gene or the suicide switch is activated to induce controlled cell death after the administration of the one or more immunosuppressive agents to the subject.

Embodiment 681. The method of any of embodiments 620-679, wherein the suicide gene or the suicide switch is activated to induce controlled cell death prior to the administration of the one or more immunosuppressive agents to the subject.

Embodiment 682. The method of any of embodiments 620-681, wherein the safety switch is activated to induce controlled cell death in the event of cytotoxicity or other negative consequences to the subject.

Embodiment 683. The method of any of embodiments 620-682, wherein the method comprises administering an agent that allows for depletion of the engineered cell, the population of engineered cells, or the composition.

Embodiment 684. The method of embodiment 683, wherein the agent that allows for depletion of the engineered cell is an antibody that recognizes a protein expressed on the cell surface.

Embodiment 685. The method of embodiment 684, wherein the antibody is selected from the group consisting of an antibody that recognizes CCR4, CD16, CD19, CD20, CD30, EGFR, GD2, HER1, HER2, MUC1, PSMA, and RQR8.

Embodiment 686. The method of embodiment 684 or embodiment 685, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

Embodiment 687. The method of any of embodiments 620-684, wherein the method comprises administering an agent that recognizes the one or more tolerogenic factors or the one or more additional tolerogenic factors on the cell surface.

Embodiment 688. The method of any of embodiments 620-687, wherein the method further comprises administering one or more additional therapeutic agents to the subject.

Embodiment 689. The method of any of embodiments 620-687, wherein the subject has been administered one or more additional therapeutic agents.

Embodiment 690. The method of any of embodiments 620-689, wherein the method further comprises monitoring the therapeutic efficacy of the method.

Embodiment 691. The method of any of embodiments 620-690, further comprising monitoring the prophylactic efficacy of the method.

Embodiment 692. The method of embodiment 690 or embodiment 691, wherein the method is repeated until a desired suppression of one or more disease symptoms occurs.

Embodiment 693. An engineered cell comprising, in its genome, a first transgene encoding a tolerogenic factor, wherein the first transgene is located at a first insertion site at a β2 microglobulin (B2M) gene locus.

Embodiment 694. An engineered cell comprising, in its genome, a first transgene encoding a tolerogenic factor, wherein the first transgene is located at a first insertion site at a class II transactivator (CIITA) gene locus.

Embodiment 695. The engineered cell of embodiment 693 or 694, wherein the engineered cell is a human cell.

Embodiment 696. The engineered cell of any one of embodiments 693-695, wherein the engineered cell is a T-cell, an islet cell, a cardiomyocyte, a hepatocyte, or a stem cell.

Embodiment 697. The engineered cell of any one of embodiments 693-696, wherein the engineered cell is a T-cell.

Embodiment 698. The engineered cell of any one of embodiments 693-697, wherein the engineered cell is a human T-cell.

Embodiment 699. The engineered cell of any one of embodiments 693-698, wherein the engineered cell is an allogeneic T-cell.

Embodiment 700. The engineered cell of embodiment 699, wherein the allogeneic T-cell is a primary T-cell.

Embodiment 701. The engineered cell of embodiment 700, wherein the allogeneic T-cell has been differentiated from an embryonic stem cell (ESC) or an induced pluripotent stem cell (iPSC).

Embodiment 702. The engineered cell of any one of embodiments 693-701, wherein the tolerogenic factor is or comprises: CD16, CD16 Fc receptor, CD24, CD27, CD35, CD39, CD46, CD47, CD52, CD55, CD59, CD64, CD200, CCL21, CCL22, CTLA4-Ig, C1 inhibitor, FASL, IDO1, HLA-C, HLA-E, HLA-E heavy chain, HLA-F, HLA-G, H2-M3, IL-10, IL15-RF, IL-35, FasL, PD-L1, Serpinb9, DUX4, Mfge8, B2M-HLA-E, IL-39, A20/TNFAIP3, CR1, or MANF.

Embodiment 703. The engineered cell of any one of embodiments 693-702, wherein the tolerogenic factor is or comprises CD47.

Embodiment 704. The engineered cell of any one of embodiments 693-703, wherein the tolerogenic factor is or comprises human CD47.

Embodiment 705. The engineered cell of embodiment 703 or 704, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 706. The engineered cell of embodiment 703 or 704, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO: 4.

Embodiment 707. The engineered cell of embodiment 693-706, wherein the nucleotide sequence of the first transgene is codon-optimized.

Embodiment 708. The engineered cell of embodiment 707, wherein the first transgene is at least 80%, at least 85%, at least 90%, at least 95%, at least 99%, or 100% identical to a nucleotide sequence set forth in SEQ ID NO:5.

Embodiment 709. The engineered cell of any one of embodiments 693-708, wherein the B2M gene locus is or comprises an endogenous B2M gene locus.

Embodiment 710. The engineered cell of any one of embodiments 693-709, wherein the B2M gene locus is chr15: 4,711,358-44,718,851.

Embodiment 711. The engineered cell of any one of embodiments 693-710, wherein the engineered cell does not express a functional endogenous B2M.

Embodiment 712. The engineered cell of any one of embodiments 693-711, wherein the engineered cell does not express an endogenous MHC I molecule on its cell surface.

Embodiment 713. The engineered cell of any one of embodiments 693-712, wherein the engineered cell does not express a gene product from a B2M locus.

Embodiment 714. The engineered cell of any one of embodiments 693-708, wherein the CIITA gene locus is or comprises an endogenous CIITA gene locus.

Embodiment 715. The engineered cell of any one of embodiments 693-708 or 714, wherein the CIITA gene locus is chr16: 10,866,222-10,943,021.

Embodiment 716. The engineered cell of any one of embodiments 693-708, 714, or 715, wherein the engineered cell does not express a functional endogenous B2M.

Embodiment 717. The engineered cell of any one of embodiments 693-708, or 714-716, wherein the engineered cell does not express an endogenous MHC I molecule on its cell surface.

Embodiment 718. The engineered cell of any one of embodiments 693-708, or 714-717, wherein the engineered cell does not express a gene product from a B2M locus.

Embodiment 719. The engineered cell of any one of embodiments 693-718, wherein the first insertion site is in an exon.

Embodiment 720. The engineered cell of any one of embodiments 693-718, wherein the first insertion site is in an intron.

Embodiment 721. The engineered cell of any one of embodiments 693-718, wherein the first insertion site is between an intron and an exon.

Embodiment 722. The engineered cell of any one of embodiments 693-718, wherein the first insertion site is in a regulatory region.

Embodiment 723. The engineered cell of any one of embodiments 693-722, wherein the engineered cell comprises a site-directed nuclease.

Embodiment 724. The engineered cell of embodiment 723, wherein the site-directed nuclease is capable of cleaving the engineered cell's genome at or adjacent to the first insertion site.

Embodiment 725. The engineered cell of embodiment 723 or 724, wherein the site-directed nuclease is capable of single strand DNA cleavage.

Embodiment 726. The engineered cell of embodiment 723 or 724, wherein the site-directed nuclease is capable of double strand DNA cleavage.

Embodiment 727. The engineered cell of any one of embodiments 723-726, wherein the site-directed nuclease is a Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, or a TnpB polypeptide.

Embodiment 728. The engine cell of any one of embodiments 723-727, wherein the site-directed nuclease is a Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a TnpB polypeptide, or CRISPR-associated transposase.

Embodiment 729. The engineered cell of any one of embodiments 693-728, further comprising a guide RNA (gRNA), wherein the gRNA comprises a complementary region that is complementary to a target nucleic acid sequence at the B2M gene locus.

Embodiment 730. The engineered cell of any one of embodiments 693-728, further comprising a guide RNA (gRNA), wherein the gRNA comprises a complementary region that is complementary to a target nucleic acid sequence at the CIITA gene locus.

Embodiment 731. The engineered cell of embodiment 729 or 730, wherein the target nucleic acid sequence comprises the first insertion site.

Embodiment 732. The engineered cell of any one of embodiments 693-731, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn, and wherein:
(i) r=a org,
(ii) y=c or t,
(iii) w=a or t,
(iv) v=a or c or g,
(v) n=a, c, t, or g.

Embodiment 733. The engineered cell of embodiment 732, wherein the engineered cell comprises SpCas9 and the PAM is ngg or nag, wherein n=a, c, t, or g.

Embodiment 734. The engineered cell of embodiment 732, wherein the engineered cell comprises SaCas9 and the PAM is ngrrt or ngrrn, wherein
(i) r=a or g, and
(ii) n=a, c, t, or g.

Embodiment 735. The engineered cell of embodiment 732, wherein the engineered cell comprises NmeCas9 and the PAM is nnnngatt, wherein n=a, c, t, or g.

Embodiment 736. The engineered cell of embodiment 732, wherein the engineered cell comprises CjCas9 and the PAM is nnnnryac, wherein:
(i) r=a org,
(ii) y=c or t, and
(iii) n=a, c, t, or g.

Embodiment 737. The engineered cell of embodiment 732, wherein the engineered cell comprises StCas9 and the PAM is nnagaaw, wherein:
(i) w=a or t, and
(ii) n=a, c, t, or g.

Embodiment 738. The engineered cell of embodiment 732, wherein the engineered cell comprises TdCas9 and the PAM is naaaac, wherein n=a, c, t, or g.

Embodiment 739. The engineered cell of embodiment 732, wherein the engineered cell comprises LbCas12a and the PAM is tttv, wherein v=a or c or g.

Embodiment 740. The engineered cell of embodiment 732, wherein the engineered cell comprises AsCas12a and the PAM is tttv, wherein v=a or c or g.

Embodiment 741. The engineered cell of embodiment 732, wherein the engineered cell comprises AacCas12b and the PAM is ttn, wherein n=a, c, t, or g.

Embodiment 742. The engineered cell of embodiment 732, wherein the engineered cell comprises BhCas12b and the PAM is attn, tttn, or gttn, wherein n=a, c, t, or g.

Embodiment 742a. The engineered cell of embodiment 732, wherein the engineered cell comprises MAD7 (ErCas12a) and the PAM is yttn, and wherein (i) y=c or t, and (ii) n=a, c, t, or g.

Embodiment 742b. The engineered cell of any one of embodiments 693-731, wherein the first insertion site is 25 nucleotides or less from a target adjacent motif (TAM) sequence, wherein the TAM sequence is tca, tcac, tcag, tcat, tcaa, ttcan, ttcaa, ttcag, or ttgat, and wherein n=a, c, t, or g.

Embodiment 742c. The engineered cell of embodiment 742b, wherein the engineered cell comprises TnpB and the TAM is tcac.

Embodiment 742d. The engineered cell of embodiment 742b, wherein the engineered cell comprises TnpB and the TAM is tcag.

Embodiment 742e. The engineered cell of embodiment 742b, wherein the engineered cell comprises TnpB and the TAM is tcat.

Embodiment 742f. The engineered cell of embodiment 742b, wherein the engineered cell comprises TnpB and the TAM is tcaa.

Embodiment 742g. The engineered cell of embodiment 742b, wherein the engineered cell comprises TnpB and the TAM is ttcan, wherein n=a, c, t, or g.

Embodiment 742h. The engineered cell of embodiment 742b, wherein the engineered cell comprises TnpB and the TAM is ttcaa.

Embodiment 742i. The engineered cell of embodiment 742b, wherein the engineered cell comprises TnpB and the TAM is ttcag.

Embodiment 742j. The engineered cell of embodiment 742b, wherein the engineered cell comprises TnpB and the TAM is ttgat.

Embodiment 743. The engineered cell of any one of embodiments 693-742, wherein the first insertion site is 25 nucleotides or less from a zinc finger binding sequence.

Embodiment 744. The engineered cell of any one of embodiments 723-727, wherein the site-directed nuclease is a ZFN.

Embodiment 745. The engineered cell of any one of embodiments 693-744, wherein the first insertion site is 25 nucleotides or less from a transcription activator-like effectors (TALE) binding sequence.

Embodiment 746. The engineered cell of embodiment 745, wherein the site-directed nuclease is a TALEN.

Embodiment 747. The engineered cell of any one of embodiments 693-746, wherein the first transgene comprises a promoter, an insulator, an enhancer, a polyadenylation (poly(A)) tail, and/or a ubiquitous chromatin opening element.

Embodiment 748. The engineered cell of embodiment 747, wherein the first transgene comprises a promoter and the promoter is a constitutive promoter.

Embodiment 749. The engineered cell of embodiment 748, wherein the constitutive promoter is an EF1α, CMV, SV40, PGK, UBC CAG, MND, SSFV, or ICOS promoter.

Embodiment 750. The engineered cell of any one of embodiments 693-749, wherein the engineered cell further comprises a second transgene encoding a tolerogenic factor in its genome.

Embodiment 751. The engineered cell of embodiment 750, wherein the second transgene encodes the same tolerogenic factor as the first transgene.

Embodiment 752. The engineered cell of embodiment 751, wherein the second transgene encodes a different tolerogenic factor than the first transgene.

Embodiment 753. The engineered cell of any one of embodiments 693-752, wherein the engineered cell has reduced expression of one or more major histocompatibility complex (MHC) class I (MHC I) molecules, one or more MHC class II molecules, or both on its cell surface as compared to a comparable wild-type human cell.

Embodiment 754. The engineered cell of any one of embodiments 693-753, wherein the engineered cell does not express major histocompatibility complex (MHC) class I (MHC I) molecules, MHC class II molecules, or both on its cell surface.

Embodiment 755. The engineered cell of any one of embodiments 693-754, wherein the engineered cell further comprises a modification at a TCR locus.

Embodiment 756. The engineered cell of embodiment 36, wherein the modification at a TCR locus comprises a knock-out of the TCR locus.

Embodiment 757. The engineered cell of embodiment 36 or 37, wherein the engineered cell is homozygous for the modification at a TCR locus.

Embodiment 758. The engineered cell of any one of embodiments 693-757, wherein the engineered cell further comprises a transgene encoding a chimeric antigen receptor (CAR) in its genome.

Embodiment 759. The engineered cell of embodiment 758, wherein the CAR comprises a CD19 CAR.

Embodiment 760. The engineered cell of embodiment 759, wherein the CD19 CAR comprises a signal peptide, an extracellular binding domain specific to CD19, a hinge domain, a transmembrane domain, an intracellular costimulatory domain, and/or an intracellular signaling domain.

Embodiment 761. The engineered cell of embodiment 758, wherein the CAR comprises a CD22 CAR.

Embodiment 762. The engineered cell of embodiment 761, wherein the CD22 CAR comprises a signal peptide, an extracellular binding domain specific to CD22, a hinge domain, a transmembrane domain, an intracellular costimulatory domain, and/or an intracellular signaling domain.

Embodiment 763. The engineered cell of embodiment 758, wherein the CAR comprises a BCMA CAR.

Embodiment 764. The engineered cell of embodiment 763, wherein the BCMA CAR comprises a signal peptide, an extracellular binding domain specific to BCMA, a hinge domain, a transmembrane domain, an intracellular costimulatory domain, and/or an intracellular signaling domain.

Embodiment 765. The engineered cell of embodiment 758, wherein the CAR comprises a CD19 CAR and a CD22 CAR connected by one or more cleavage sites.

Embodiment 766. The engineered cell of embodiment 758, wherein the first transgene and the transgene encoding the CAR are in the form of a polycistronic construct connected by one or more cleavage sites.

Embodiment 767. The engineered cell of embodiment 765 or 766, wherein the one or more cleavage sites comprise a self-cleaving site.

Embodiment 768. The engineered cell of any one of embodiments 765 or 766, wherein the one or more cleavage sites comprise a protease site.

Embodiment 769. The engineered cell of embodiment 768, wherein the protease site precedes the 2A site in the 5′ to 3′ order.

Embodiment 770. The engineered cell of any one of embodiments 758-769, wherein the transgene encoding a CAR is located at a random genomic locus.

Embodiment 771. The engineered cell of any one of embodiments 758-769, wherein the transgene encoding a CAR is located at a TRAC locus, a TRBC1 locus, a TRBC2 locus, a B2M locus, a CIITA locus, a safe harbor locus, an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 772. The engineered cell of embodiment 693, wherein the engineered cell is a T-cell, the tolerogenic factor is CD47, the first insertion site is a B2M locus, and the first insertion site is in an exon.

Embodiment 773. The engineered cell of embodiment 693, wherein the engineered cell is a T-cell, the tolerogenic factor is CD47, the first insertion site is a CIITA locus, and the first insertion site is in an exon.

Embodiment 774. A population of cells comprising one or more engineered cells of any one of embodiments 693-772.

Embodiment 775. A population of engineered cells according to any one of embodiments 693-774.

Embodiment 776. The population of cells according to embodiment 774, wherein the cells are T-cells, and wherein at least 50% of the T-cells each have (a) reduced cell surface expression of MHC I and/or MHC II molecules, and (b) increased expression of a tolerogenic factor encoded by the first transgene.

Embodiment 777. The population of cells according to embodiment 776, wherein the tolerogenic factor is CD47.

Embodiment 778. The population of cells according to embodiment 774, wherein the cells are T-cells, and wherein at least 50% of the T-cells each have (a) reduced expression of B2M and/or CIITA, and (b) increased expression of CD47 encoded by a transgene.

Embodiment 779. The population of cells according to embodiment 774, wherein the cells are T-cells, and wherein at least 50% of the T-cells each have (a) B2M and/or CIITA knocked-out, and (b) increased expression of CD47 encoded by a transgene.

Embodiment 780. The population of cells of any one of embodiments 776-779, wherein at least 55%, at least 60%, at least 65%, at least 70%, at least 75%, at least 80%, at least 85%, at least 90%, at least 95%, or at least 100% of the T-cells each have (a) and (b).

Embodiment 781. The population of cells according to embodiment 774, wherein the cells are T-cells, and wherein at least 50% of the T-cells each have (a) reduced expression of B2M, (b) reduced expression of CIITA, and (c) increased expression of CD47 encoded by a transgene.

Embodiment 782. The population of cells of embodiment 781, wherein at least 55%, at least 60%, at least 65%, at least 70%, at least 75%, at least 80%, at least 85%, at least 90%, at least 95%, or at least 100% of the T-cells each have (a) and (b).

Embodiment 783. The population of cells of embodiment 781 or 782, wherein at least 55%, at least 60%, at least 65%, at least 70%, at least 75%, at least 80%, at least 85%, at least 90%, at least 95%, or at least 100% of the T-cells each have (a), (b), and (c).

Embodiment 784. A composition comprising an engineered cell according to any one of embodiments 693-773 or a population of cells according to any of embodiments 774-783.

Embodiment 785. A pharmaceutical composition comprising (i) an engineered cell according to any one of embodiments 693-773 or a population of cells according to any of embodiments 774-783, and (ii) a pharmaceutically acceptable excipient.

Embodiment 786. A method comprising administering to a subject an engineered cell according to any one of embodiments 693-773, a population of cells according to any of embodiments 774-783, a composition according to embodiment 784, or a pharmaceutical composition according to embodiment 785.

Embodiment 787. The method of embodiment 786, wherein the method is a method of treating disease in a subject in need thereof.

Embodiment 788. An engineered cell according to any one of embodiments 693-773 for use in treating a disease in a subject in need thereof.

Embodiment 789. A population of cells according to any of embodiments 774-783 for use in treating a disease in a subject in need thereof.

Embodiment 790. A composition according to embodiment 784 for use in treating a disease in a subject in need thereof.

Embodiment 791. A pharmaceutical composition of embodiment 785 for use in treating a disease in a subject in need thereof.

Embodiment 792. Use of an engineered cell according to any one of embodiments 693-773, a population of cells according to any of embodiments 774-783, a composition according to embodiment 784, or a pharmaceutical composition according to embodiment 785 for use in treating a disease in a subject in need thereof.

Embodiment 793. Use of an engineered cell according to any one of embodiments 693-773, a population of cells according to any of embodiments 774-783, a composition according to embodiment 784, or a pharmaceutical composition according to embodiment 785 in the manufacture of a medicament for the treatment of a disease.

Embodiment 794. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the disease is cancer.

Embodiment 795. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the cancer is associated with CD19, CD22, and/or BCMA expression.

Embodiment 796. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the cancer is a hematologic malignancy.

Embodiment 797. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 798. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the disease is an autoimmune disease.

Embodiment 799. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 800. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the disease is diabetes mellitus.

Embodiment 801. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the diabetes is selected from the group consisting Type I diabetes, Type II diabetes, prediabetes, and gestational diabetes.

Embodiment 802. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the disease is a neurological disease.

Embodiment 803. The method of embodiment 786 or 787, the engineered cell of embodiment 788, the population of cells of embodiment 789, the composition of embodiment 790, the pharmaceutical composition of embodiment 791, or the use of embodiment 792 or 793, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 804. A method of generating a genetically engineered cell, the method comprising: inserting a first transgene encoding a tolerogenic factor at a first insertion site at a B2M gene locus.

Embodiment 805. A method of generating a genetically engineered cell, the method comprising: inserting a first transgene encoding a tolerogenic factor at a first insertion site at a CIITA gene locus.

Embodiment 806. The method of embodiment 804 or 805, wherein the step of inserting comprises homology-directed repair (HDR)-mediated insertion using a site-directed nuclease.

Embodiment 807. The method of embodiment 806, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a TnpB polypeptide, and a CRISPR-associated transposase.

Embodiment 808. The method of any one of embodiments 804-807, wherein the step of inserting comprises homology-directed repair (HDR)-mediated insertion using a guide RNA (gRNA) and a CRISPR-associated (Cas) nuclease.

Embodiment 809. The method of embodiment 808, wherein the gRNA comprises a complementary region, wherein the complementary region comprises a nucleic acid sequence that is complementary to a target nucleic acid sequence within the B2M gene locus, and wherein the target nucleic acid sequence comprises the first insertion site.

Embodiment 810. The method of embodiment 808, wherein the gRNA comprises a complementary region, wherein the complementary region comprises a nucleic acid sequence that is complementary to a target nucleic acid sequence within the CIITA gene locus, and wherein the target nucleic acid sequence comprises the first insertion site.

Embodiment 811. The method of embodiment 809 or 810, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn, and wherein:
(i) r=a org,
(ii) y=c or t,
(iii) w=a ort,
(iv) v=a or c or g,
(v) n=a, c, t, or g.

Embodiment 812. The method of embodiment 811, wherein the step of inserting comprises homology-directed repair (HDR)-mediated insertion using SpCas9 and the PAM is ngg or nag, wherein n=a, c, t, or g.

Embodiment 813. The method of embodiment 811, wherein the step of inserting comprises homology-directed repair (HDR)-mediated insertion using SaCas9 and the PAM is ngrrt or ngrrn, wherein
(i) r=a or g, and
(ii) n=a, c, t, or g.

Embodiment 814. The method of embodiment 811, wherein the step of inserting comprises homology-directed repair (HDR)-mediated insertion using NmeCas9 and the PAM is nnnngatt, wherein n=a, c, t, or g.

Embodiment 815. The method of embodiment 811, wherein the step of inserting comprises homology-directed repair (HDR)-mediated insertion using CjCas9 and the PAM is nnnnryac, wherein:
(i) r=a or g,
(ii) y=cor t, and
(iii) n=a, c, t, or g.

Embodiment 816. The method of embodiment 811, wherein the step of inserting comprises homology-directed repair (HDR)-mediated insertion using StCas9 and the PAM is nnagaaw wherein:
(i) w=a or t, and
(ii) n=a, c, t, or g.

Embodiment 817. The method of embodiment 811, wherein the step of inserting comprises homology-directed repair (HDR)-mediated insertion using TdCas9 and the PAM is naaaac, wherein n=a, c, t, or g.

Embodiment 818. The method of embodiment 811, wherein the step of inserting comprises homology-directed repair (HDR)-mediated insertion using LbCas12a and the PAM is tttv, wherein v=a or c or g.

Embodiment 819. The method of embodiment 811, wherein the step of inserting comprises homology-directed repair (HDR)-mediated insertion using AsCas12a and the PAM is tttv, wherein v=a or c or g.

Embodiment 820. The method of embodiment 811, wherein the step of inserting comprises homology-directed repair (HDR)-mediated insertion using AacCas 12b and the PAM is ttn, wherein n=a, c, t, or g.

Embodiment 821. The method of embodiment 811, wherein the step of inserting comprises homology-directed repair (HDR)-mediated insertion using BhCas12b and the PAM is attn., tttn, or gttn, wherein n=a, c, t, or g.

Embodiment 821a. The method of embodiment 811, wherein the step of inserting comprises homology-directed repair (HDR)-mediated insertion using MAD7 (ErCas12a) and the PAM is yttn, and wherein (i) y=c or t, and (ii) n=a, c, t, or g.

Embodiment 821b. The method of embodiment 809 or 810, wherein the first insertion site is 25 nucleotides or less from a target adjacent motif (TAM) sequence, wherein the TAM sequence is tca, tcac, tcag, tcat, tcaa, ttcan, ttcaa, ttcag, or ttgat, and wherein n=a, c, t, or g.

Embodiment 821c. The method of embodiment 821b, wherein the step of inserting comprises homology-directed repair (HDR)-mediated insertion using TnpB and the TAM is tcac.

Embodiment 821d. The method of embodiment 821b, wherein the step of inserting comprises homology-directed repair (HDR)-mediated insertion using TnpB and the TAM is tcag.

Embodiment 821e. The method of embodiment 821b, wherein the step of inserting comprises homology-directed repair (HDR)-mediated insertion using TnpB and the TAM is tcat.

Embodiment 821f. The method of embodiment 821b, wherein the step of inserting comprises homology-directed repair (HDR)-mediated insertion using TnpB and the TAM is tcaa.

Embodiment 821g. The method of embodiment 821b, wherein the step of inserting comprises homology-directed repair (HDR)-mediated insertion using TnpB and the TAM is ttcan, wherein n=a, c, t, or g.

Embodiment 821h. The method of embodiment 821b, wherein the step of inserting comprises homology-directed repair (HDR)-mediated insertion using TnpB and the TAM is ttcaa.

Embodiment 821i. The method of embodiment 821b, wherein the step of inserting comprises homology-directed repair (HDR)-mediated insertion using TnpB and the TAM is ttcag.

Embodiment 821j. The method of embodiment 821b, wherein the step of inserting comprises homology-directed repair (HDR)-mediated insertion using TnpB and the TAM is ttgat.

Embodiment 822. The method of any one of embodiments 804-821j, wherein the first insertion site is in an exon.

Embodiment 823. The method of any one of embodiments 804-821j, wherein the first insertion site is in an intron.

Embodiment 824. The method of any one of embodiments 804-821j, wherein the first insertion site is between an intron and an exon.

Embodiment 825. The method of any one of embodiments 804-821j, wherein the first insertion site is in a regulatory region.

Embodiment 826. The method of any one of embodiments 804-825, wherein the engineered cell is a human cell.

Embodiment 827. The method of any one of embodiments 804-826, wherein the engineered cell is a T-cell, an islet cell, a cardiomyocyte, a hepatocyte, or a stem cell.

Embodiment 828. The method of any one of embodiments 804-827, wherein the engineered cell is a T-cell.

Embodiment 829. The method of any one of embodiments 804-828, wherein the engineered cell is a human T-cell.

Embodiment 830. The method of any one of embodiments 826-829, wherein the engineered cell is an allogeneic T-cell.

Embodiment 831. The method of embodiment 830, wherein the allogeneic T-cell is a primary T-cell.

Embodiment 832. The method of embodiment 830, wherein the allogeneic T-cell has been differentiated from an embryonic stem cell (ESC) or an induced pluripotent stem cell (iPSC).

Embodiment 833. The method of any one of embodiments 804-832, wherein the tolerogenic factor is or comprises: CD16, CD16 Fc receptor, CD24, CD27, CD35, CD39, CD46, CD47, CD52, CD55, CD59, CD64, CD200, CCL21, CCL22, CTLA4-Ig, C1 inhibitor, FASL, IDO1, HLA-C, HLA-E, HLA-E heavy chain, HLA-F, HLA-G, H2-M3, IL-10, IL15-RF, IL-35, FasL, PD-L1, Serpinb9, DUX4, Mfge8, B2M-HLA-E, IL-39, A20/TNFAIP3, CR1, or MANF.

Embodiment 834. The method of any one of embodiments 804-833, wherein the tolerogenic factor is or comprises CD47.

Embodiment 835. The method of embodiments 804-834, wherein the tolerogenic factor is or comprises human CD47.

Embodiment 836. The method of embodiment 834 or 835, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 837. The method of any one of embodiments 804-836, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO: 4.

Embodiment 838. The method of embodiment 804-837, wherein the nucleotide sequence of the first transgene is codon-optimized.

Embodiment 839. The method of embodiment 838, wherein the first transgene is at least 80%, at least 85%, at least 90%, at least 95%, at least 99%, or 100% identical to a nucleotide sequence set forth in SEQ ID NO:5.

Embodiment 840. The method of any one of embodiments 804-839, wherein the B2M gene locus is or comprises an endogenous B2M gene locus.

Embodiment 841. The method of any one of embodiments 804-840, wherein the engineered cell does not express a functional endogenous B2M.

Embodiment 842. The method of any one of embodiments 804-841, wherein the insertion of the first transgene encoding a tolerogenic factor at a first insertion site at the B2M gene locus prevents expression of a functional B2M.

Embodiment 843. The method of any one of embodiments 804-842, wherein the insertion of the first transgene encoding a tolerogenic factor at a first insertion site at the B2M gene locus prevents expression of a functional MHC I molecule.

Embodiment 844. A method of generating a population of genetically engineered cells, the method comprising: inserting a first transgene encoding a tolerogenic factor at a first insertion site at a B2M gene locus in the genome of the cells.

Embodiment 845. The method of any one of embodiments 804-839, wherein the CIITA gene locus is or comprises an endogenous CIITA gene locus.

Embodiment 846. The method of any one of embodiments 804-839, wherein the engineered cell does not express a functional endogenous CIITA.

Embodiment 847. The method of any one of embodiments 804-839, wherein the engineered cell does not express a functional endogenous MHC II molecule.

Embodiment 848. The method of any one of embodiments 804-839, wherein the insertion of the first transgene encoding a tolerogenic factor at a first insertion site at the CIITA gene locus prevents expression of a functional CIITA.

Embodiment 849. A method of generating a population of genetically engineered cells, the method comprising: inserting a first transgene encoding a tolerogenic factor at a first insertion site at a CIITA gene locus in the genome of the cells.

Embodiment 850. The method of embodiment 849, wherein the step of inserting comprises homology-directed repair (HDR)-mediated insertion using a site-directed nuclease.

Embodiment 851. The method of embodiment 850, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2cl), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a TnpB polypeptide, and a CRISPR-associated transposase.

Embodiment 852. The method of any one of embodiments 849-851, wherein the step of inserting comprises homology-directed repair (HDR)-mediated insertion using a guide RNA (gRNA) and a CRISPR-associated (Cas) nuclease.

Embodiment 853. The method of embodiment 852, wherein the gRNA comprises a complementary region, wherein the complementary region comprises a nucleic acid sequence that is complementary to a target nucleic acid sequence within the TCR gene locus, and wherein the target nucleic acid sequence comprises the first insertion site.

Embodiment 854. The method of any one of embodiments 849-853, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn, and wherein:
(i) r=a org,
(ii) y=c or t,
(iii) w=a or t,
(iv) v=a or c or g,
(v) n=a, c, t, or g.

Embodiment 855. The method of embodiment 854, wherein the step of inserting comprises homology-directed repair (HDR)-mediated insertion using SpCas9 and the PAM is ngg or nag, wherein n=a, c, t, or g.

Embodiment 856. The method of embodiment 854, wherein the step of inserting comprises homology-directed repair (HDR)-mediated insertion using SaCas9 and the PAM is ngrrt or ngrrn, wherein
(i) r=a or g, and
(ii) n=a, c, t, or g.

Embodiment 857. The method of embodiment 854, wherein the step of inserting comprises homology-directed repair (HDR)-mediated insertion using NmeCas9 and the PAM is nnnngatt, wherein n=a, c, t, or g.

Embodiment 858. The method of embodiment 854, wherein the step of inserting comprises homology-directed repair (HDR)-mediated insertion using CjCas9 and the PAM is nnnnryac, wherein:
(i) r=a or g,
(ii) y=cort, and
(iii) n=a, c, t, or g.

Embodiment 859. The method of embodiment 854, wherein the step of inserting comprises homology-directed repair (HDR)-mediated insertion using StCas9 and the PAM is nnagaaw wherein:
(i) w=a or t, and
(ii) n=a, c, t, or g.

Embodiment 860. The method of embodiment 854, wherein the step of inserting comprises homology-directed repair (HDR)-mediated insertion using TdCas9 and the PAM is naaaac, wherein n=a, c, t, or g.

Embodiment 861. The method of embodiment 854, wherein the step of inserting comprises homology-directed repair (HDR)-mediated insertion using LbCas12a and the PAM is tttv, wherein v=a or c or g.

Embodiment 862. The method of embodiment 854, wherein the step of inserting comprises homology-directed repair (HDR)-mediated insertion using AsCas12a and the PAM is tttv, wherein v=a or c or g.

Embodiment 863. The method of embodiment 854, wherein the step of inserting comprises homology-directed repair (HDR)-mediated insertion using AacCas12b and the PAM is ttn, wherein n=a, c, t, or g.

Embodiment 864. The method of embodiment 854, wherein the step of inserting comprises homology-directed repair (HDR)-mediated insertion using BhCas12b and the PAM is attn., tttn, or gttn, wherein n=a, c, t, or g.

Embodiment 864a. The method of embodiment 854, wherein the step of inserting comprises homology-directed repair (HDR)-mediated insertion using MAD7 (ErCas12a) and the PAM is yttn, and wherein (i) y=c or t, and (ii) n=a, c, t, or g.

Embodiment 864b. The method of any one of embodiments 849-853, wherein the first insertion site is 25 nucleotides or less from a target adjacent motif (TAM) sequence, wherein the TAM sequence is tca, tcac, tcag, tcat, tcaa, ttcan, ttcaa, ttcag, or ttgat, and wherein n=a, c, t, or g.

Embodiment 864c. The method of embodiment 864b, wherein the step of inserting comprises homology-directed repair (HDR)-mediated insertion using TnpB and the TAM is tcac.

Embodiment 864d. The method of embodiment 864b, wherein the step of inserting comprises homology-directed repair (HDR)-mediated insertion using TnpB and the TAM is tcag.

Embodiment 864e. The method of embodiment 864b, wherein the step of inserting comprises homology-directed repair (HDR)-mediated insertion using TnpB and the TAM is tcat.

Embodiment 864f. The method of embodiment 864b, wherein the step of inserting comprises homology-directed repair (HDR)-mediated insertion using TnpB and the TAM is tcaa.

Embodiment 864g. The method of embodiment 864b, wherein the step of inserting comprises homology-directed repair (HDR)-mediated insertion using TnpB and the TAM is ttcan, wherein n=a, c, t, or g.

Embodiment 864h. The method of embodiment 864b, wherein the step of inserting comprises homology-directed repair (HDR)-mediated insertion using TnpB and the TAM is ttcaa.

Embodiment 864i. The method of embodiment 864b, wherein the step of inserting comprises homology-directed repair (HDR)-mediated insertion using TnpB and the TAM is ttcag.

Embodiment 864j. The method of embodiment 864b, wherein the step of inserting comprises homology-directed repair (HDR)-mediated insertion using TnpB and the TAM is ttgat.

Embodiment 865. The method of any one of embodiments 849-864j, wherein the first insertion site is in an exon.

Embodiment 866. The method of any one of embodiments 849-864j, wherein the first insertion site is in an intron.

Embodiment 867. The method of any one of embodiments 849-864j, wherein the first insertion site is between an intron and an exon.

Embodiment 868. The method of any one of embodiments 849-864j, wherein the first insertion site is in a regulatory region.

Embodiment 869. The method of any one of embodiments 849-868, wherein the insertion of the first transgene encoding a tolerogenic factor at a first insertion site at a T-cell receptor (TCR) gene locus prevents expression of a functional TCR.

Embodiment 870. The method of any one of embodiments 849-869, wherein the population of genetically engineered cells are human cells.

Embodiment 871. The method of any one of embodiments 849-870, wherein the population of genetically engineered cells are T-cells, islet cells, cardiomyocytes, hepatocytes, or stem cells.

Embodiment 872. The method of any one of embodiments 849-871, wherein the population of genetically engineered cells are T-cells.

Embodiment 873. The method of any one of embodiments 849-872, wherein the population of genetically engineered cells are human T-cells.

Embodiment 874. The method of any one of embodiments 849-873, wherein the population of genetically engineered cells are allogeneic T-cells.

Embodiment 875. The method of embodiment 874, wherein the allogeneic T-cells are primary T-cells.

Embodiment 876. The method of embodiment 874, wherein the allogeneic T-cells have been differentiated from embryonic stem cells (ESCs) or induced pluripotent stem cells (iPSCs).

Embodiment 877. The method of any one of embodiments 849-876, wherein the tolerogenic factor is or comprises: CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CC121, or Mfge8.

Embodiment 878. The method of any one of embodiments 849-877, wherein the tolerogenic factor is or comprises CD47.

Embodiment 879. The method of embodiments 849-878, wherein the tolerogenic factor is or comprises human CD47.

Embodiment 880. The method of embodiment 878 or 879, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 881. The method of any one of embodiments 849-880, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO: 4.

Embodiment 882. The method of embodiment 849-881, wherein the nucleotide sequence of the first transgene is codon-optimized.

Embodiment 883. The method of embodiment 882, wherein the first transgene is at least 80%, at least 85%, at least 90%, at least 95%, at least 99%, or 100% identical to a nucleotide sequence set forth in SEQ ID NO:5.

Embodiment 884. The method of any one of embodiments 849-883, wherein the B2M gene locus is or comprises an endogenous B2M gene locus.

Embodiment 885. The method of any one of embodiments 849-164, wherein the population of engineered cells do not express a functional endogenous B2M.

Embodiment 886. The method of any one of embodiments 849-164, wherein the population of engineered cells do not express a functional endogenous MHC I molecule.

Embodiment 887. The method of any one of embodiments 849-165, wherein the insertion of the first transgene encoding a tolerogenic factor at a first insertion site at a B2M gene locus prevents expression of a functional B2M.

Embodiment 888. The method of any one of embodiments 849-883, wherein the CIITA gene locus is or comprises an endogenous CIITA gene locus.

Embodiment 889. The method of any one of embodiments 849-164, wherein the population of engineered cells do not express a functional endogenous CIITA.

Embodiment 890. The method of any one of embodiments 849-164, wherein the population of engineered cells do not express a functional endogenous MHC II molecule.

Embodiment 891. The method of any one of embodiments 849-165, wherein the insertion of the first transgene encoding a tolerogenic factor at a first insertion site at a CIITA gene locus prevents expression of a functional CIITA.

Embodiment 892. The method of any one of embodiments 849-891, wherein the method further comprises reducing expression of major histocompatibility complex (MHC) class I (MHC I) molecules and/or MHC class II (MHC II) molecules in one or more cells in the population.

Embodiment 893. The method of embodiment 892, wherein reducing expression of MHC I molecules comprises modulation of a B2M locus of the one or more cells in the population.

Embodiment 894. The method of embodiment 892, wherein reducing expression of MHC II molecules comprises modulation of a CIITA locus of the one or more cells in the population.

Embodiment 895. The method of embodiment 893 or 894, wherein modulation of the B2M locus comprises knock-out of the B2M locus and/or modulation of the CIITA locus comprises knock-out of the CIITA locus.

Embodiment 896. The method of embodiment 895, wherein knock-out of the B2M locus and/or knock-out of the CIITA locus occurs at: both B2M alleles, both CIITA alleles, or combinations thereof.

Embodiment 897. The method of any one of embodiments 892-896, wherein the step of inserting the first transgene occurs before, after, or together with, the step of reducing expression of MHC I molecules and/or MHC II molecules.

Embodiment 898. The method of any one of embodiments 892-896, wherein the genetically engineered cells are T-cells, and wherein, after the steps of inserting the first transgene and reducing expression of MHC I and/or MHC II molecules, at least 50% of the T-cells each have (a) reduced cell surface expression of MHC I and/or MHC II molecules, and (b) increased expression of a tolerogenic factor encoded by the first transgene.

Embodiment 899. The population of cells according to embodiment 898, wherein the tolerogenic factor is CD47.

Embodiment 900. The method of any one of embodiments embodiment 893-897, wherein the genetically engineered cells are T-cells, and wherein after the steps of inserting the first transgene and modulating the B2M locus and/or the CIITA locus, at least 50% of the T-cells each have (a) B2M and/or CIITA knocked-out, and (b) increased expression of CD47 encoded by a transgene.

Embodiment 901. The method of embodiments 898-900, wherein at least 55%, at least 60%, at least 65%, at least 70%, at least 75%, at least 80%, at least 85%, at least 90%, at least 95%, or at least 100% of the T-cells each have (a) and (b).

Embodiment 902. The method of any one of embodiments 893-897, wherein the genetically engineered cells are T-cells and the tolerogenic factor is CD47, and wherein after the steps of inserting the first transgene and modulating the B2M locus and/or the CIITA locus, at least 50% of the T-cells each have (a) reduced expression of B2M, (b) reduced expression of CIITA, and (c) increased expression of CD47 encoded by a transgene.

Embodiment 903. The population of cells of embodiment 902, wherein at least 55%, at least 60%, at least 65%, at least 70%, at least 75%, at least 80%, at least 85%, at least 90%, at least 95%, or at least 100% of the T-cells each have (a) and (b).

Embodiment 904. The method of embodiment 902 or 903, wherein at least 55%, at least 60%, at least 65%, at least 70%, at least 75%, at least 80%, at least 85%, at least 90%, at least 95%, or at least 100% of the T-cells each have (a), (b), and (c).

Embodiment 905. The method of any one of embodiments 849-179, wherein the method further comprises selecting the one or more genetically engineered cells from the population of genetically engineered cells.

Embodiment 906. The method of embodiment 905, wherein the step of selecting comprises selecting for one or more genetically engineered cells based on a level of tolerogenic factor expressed on the cell surface.

Embodiment 907. The method of embodiment 906, wherein selecting for one or more genetically engineered cells based on a level of tolerogenic factor expressed on the cell surface comprises: affinity binding, flow cytometry, and/or immunomagnetic selection using antibodies and/or proteins that bind the tolerogenic factor.

Embodiment 908. The method of any one of embodiments 905-907, wherein the step of selecting comprises selecting for one or more genetically engineered cells based on a level of CD3 expressed on the cell surface.

Embodiment 909. The method of embodiment 908, wherein selecting for one or more genetically engineered cells based on a level of CD3 expressed on the cell surface comprises affinity binding, flow cytometry, and/or immunomagnetic selection using CD3-binding antibodies and/or CD3-binding proteins.

Embodiment 910. The method of any one of embodiments 905-909, wherein the step of selecting comprises selecting for one or more genetically engineered cells based on a level of MHC I and/or MHC II molecules expressed on the cell surface.

Embodiment 911. The method of embodiment 908, wherein selecting for one or more genetically engineered cells based on a level of MHC I and/or MHC II molecules expressed on the cell surface comprises affinity binding, flow cytometry, and/or immunomagnetic selection using CD3-binding antibodies and/or CD3-binding proteins.

Embodiment 912. The method of any one of embodiments 849-909, further comprising enriching for one or more genetically engineered cells that express higher levels of CD47 on their cell surface as compared to comparable wild-type cells.

Embodiment 913. The method of any one of embodiments 849-912, further comprising enriching for one or more genetically engineered cells that express lower levels of MHC I and/or MHC II on their cell surface as compared to comparable wild-type cells.

Embodiment 914. The method of any one of embodiments 849-913, further comprising enriching for one or more genetically engineered cells that express lower levels of CD3 on their cell surface as compared to comparable wild-type cells.

Embodiment 915. The method of any one of embodiments 849-913, further comprising enriching for one or more genetically engineered cells that express lower levels of B2M and/or CIITA as compared to comparable wild-type cells.

Embodiment 916. A method of identifying a site for inserting a first transgene at a B2M gene locus, comprising the steps of:
(a) identifying a PAM or TAM sequence in (i) the B2M gene locus, (ii) the 100 bp upstream of the 5′ end of B2M gene locus, or (iii) the 100 bp downstream of the 3′ end of B2M gene locus, and
(b) generating a gRNA comprising a complementary region, wherein the complementary region comprises a nucleic acid sequence that is complementary to a target nucleic acid sequence within the B2M gene locus, wherein the target nucleic acid sequence comprises the first insertion site, and wherein the first insertion site is 25 nucleotides or less from a protospacer adjacent motif (PAM) or TAM sequence.

Embodiment 917. A method of identifying a site for inserting a first transgene at a CIITA gene locus, comprising the steps of:
(a) identifying a PAM or TAM sequence in (i) the CIITA gene locus, (ii) the 100 bp upstream of the 5′ end of CIITA gene locus, or (iii) the 100 bp downstream of the 3′ end of CIITA gene locus, and
(b) generating a gRNA comprising a complementary region, wherein the complementary region comprises a nucleic acid sequence that is complementary to a target nucleic acid sequence within the B2M gene locus, wherein the target nucleic acid sequence comprises the first insertion site, and wherein the first insertion site is 25 nucleotides or less from a protospacer adjacent motif (PAM) or TAM sequence.

Embodiment 918. A method of generating an engineered cell for cell therapy, comprising inserting one or more transgenes encoding one or more tolerogenic factors, and optionally, one or more nucleotide sequences encoding one or more safety switches, into an endogenous β2 microglobulin (B2M) gene locus, an endogenous class II transactivator (CIITA) gene locus, or both loci, of a cell, wherein the engineered cell is immune evasive.

Embodiment 919. The method of embodiment 918, further comprising modifying the engineered cell to reduce or eliminate expression of one or more MHC class I molecules, one or more MHC class II molecules, or both.

Embodiment 920. The method of embodiment 919, wherein the modification is carried out by (i) knocking out or knocking down B2M, TAP1, or both to reduce or eliminate expression of one or more MHC I molecules, (ii) knocking out or knocking down CIITA, CD74, or both to reduce or eliminate expression of one or more MHC II molecules, or both (i) and (ii).

Embodiment 921. The method of any one of embodiments 918-920, wherein the surface expression of one or more MHC I and/or one or more MHC II molecules is reduced.

Embodiment 922. The method of any one of embodiments 918-921, wherein the surface trafficking of one or more MHC I and/or one or more MHC II molecules is reduced.

Embodiment 923. The method of any one of embodiments 918-922, wherein the one or more transgenes encoding one or more tolerogenic factors, and optionally, the one or more nucleotide sequences encoding one or more safety switches, are inserted into the B2M gene locus, and CIITA is knocked out or knocked down.

Embodiment 924. The method of embodiment 923, wherein the transgene encoding CD47 is inserted into the B2M gene locus, and CIITA, CD74, or both are knocked out or knocked down.

Embodiment 925. The method of any one of embodiments 918-922, wherein the one or more transgenes encoding one or more tolerogenic factors, and optionally, the one or more nucleotide sequences encoding one or more safety switches, are inserted into the CIITA gene locus, and B2M, TAP1, or both are knocked out or knocked down.

Embodiment 926. The method of embodiment 925, wherein the transgene encoding CD47 is inserted into the CIITA gene locus, and B2M, TAP1, or both are knocked out or knocked down.

Embodiment 927. The method of any one of embodiments 918-922, wherein one or more transgenes encoding one or more tolerogenic factors, and optionally, the one or more nucleotide sequences encoding one or more safety switches, are inserted into both B2M and CIITA gene loci.

Embodiment 928. The method of embodiment 927, wherein the same transgene encoding a tolerogenic factor is inserted into both B2M and CIITA gene loci.

Embodiment 929. The method of embodiment 927, wherein different transgenes encoding different tolerogenic factors are inserted into B2M and CIITA gene loci.

Embodiment 930. The method of any one of embodiments 918-929, wherein the one or more transgenes encoding one or more tolerogenic factors, and optionally, the one or more nucleotide sequences encoding one or more safety switches, are inserted into the B2M or CIITA locus of one allele.

Embodiment 931. The method of any one of embodiments 918-929, wherein the one or more transgenes encoding one or more tolerogenic factors, and optionally, the one or more nucleotide sequences encoding one or more safety switches, are inserted into the B2M or CIITA locus of both alleles.

Embodiment 932. The method of any one of embodiments 918-931, wherein the one or more transgenes encoding one or more tolerogenic factors, and optionally, the one or more nucleotide sequences encoding one or more safety switches, are inserted into exon 2 or another CDS of the B2M gene locus.

Embodiment 933. The method of any one of embodiments 918-932, wherein the one or more transgenes encoding one or more tolerogenic factors, and optionally, the one or more nucleotide sequences encoding one or more safety switches, are inserted into exon 3 or another CDS of the CIITA gene locus.

Embodiment 934. The method of any one of embodiments 918-933, wherein the one or more transgenes encoding one or more tolerogenic factors, and optionally, the one or more nucleotide sequences encoding one or more safety switches, are inserted into the B2M or CIITA locus by homology-directed repair (HDR)-mediated insertion using a site-directed nuclease.

Embodiment 935. The method of embodiment 934, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 936. The method of embodiments 918-935, wherein the one or more transgenes encoding one or more tolerogenic factors, and optionally, the one or more nucleotide sequences encoding one or more safety switches, are introduced into the cell by calcium phosphate or lipid-mediated transfection, electroporation, fusogens, or viral transduction.

Embodiment 937. The method of embodiment 936, wherein the virus is a retrovirus or an adeno-associated virus (AAV).

Embodiment 938. The method of embodiment 937, wherein the retrovirus is selected from the group consisting of Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV)), lentivirus, a Gammretrovirus, an Epsilonretrovirus, an Alpharetrovirus, a Betaretrovirus, a Deltaretrovirus, and a Spumaretrovirus.

Embodiment 939. The method of embodiment 937, wherein the AAV is AAV-6 or AAV-9.

Embodiment 940. The method of any one of embodiments 918-939, further comprising selecting for immune evasive cell that has the one or more transgenes inserted by selecting for expression of the one or more tolerogenic factors.

Embodiment 941. The method of embodiment 940, wherein the selection for expression of the one or more tolerogenic factors is carried out by affinity binding, flow cytometry, and/or immunomagnetic selection using antibodies and/or proteins that bind the one or more tolerogenic factors.

Embodiment 942. The method of any one of embodiments 918-941, wherein the tolerogenic factor is selected from the group consisting of CD16, CD16 Fc receptor, CD24, CD27, CD35, CD39, CD46, CD47, CD52, CD55, CD59, CD200, CCL21, CCL22, CTLA4-Ig, C1 inhibitor, FASL, IDO1, HLA-C, HLA-E, HLA-E heavy chain, HLA-F, HLA-G, H2-M3, IL-10, IL15-RF, IL-35, FasL, PD-L1, Serpinb9, DUX4, and Mfge8.

Embodiment 943. The method of embodiment 942, wherein the tolerogenic factor is CD47.

Embodiment 944. The method of embodiment 943, wherein the CD47 is human CD47.

Embodiment 945. The method of embodiment 944, wherein the human CD47 comprises an amino acid sequence that is at least 80% identical to the amino acid sequence set forth in SEQ ID NO: 1 or SEQ ID NO:2.

Embodiment 946. The method of embodiment 944 or embodiment 945, wherein the human CD47 further comprises a leader peptide.

Embodiment 947. The method of embodiment 944, wherein the transgene encoding the human CD47 comprises a nucleotide sequence that is at least 80%, at least 85%, at least 90%, at least 95%, or at least 100% identical to the nucleotide sequence set forth in SEQ ID NO:3 or SEQ ID NO:4.

Embodiment 948. The method of embodiment 944, wherein the transgene encoding the human CD47 comprises a nucleotide sequence that is at least 80%, at least 85%, at least 90%, at least 95%, or at least 100% identical to the nucleotide sequence set forth in SEQ ID NO:5.

Embodiment 949. The method of embodiment 947 or embodiment 948, wherein the nucleotide sequence further comprises a sequence encoding a leader peptide.

Embodiment 950. The method of any one of embodiments 947-949, wherein the nucleotide sequence is codon optimized.

Embodiment 951. The method of any one of embodiments 947-950, wherein the nucleotide sequence further comprises a promoter, an insulator, an enhancer, a polyadenylation (poly(A)) tail, and/or a ubiquitous chromatin opening element.

Embodiment 952. The method of embodiment 951, wherein the promoter is a constitutive promoter.

Embodiment 953. The method of embodiment 951 or embodiment 952, wherein the promoter is selected from the group consisting of an EF1α, a short EF1α, CMV, SV40, PGK, UBC, CAG, MND, SSFV, and ICOS promoters.

Embodiment 954. The method of any one of embodiments 918-953, wherein the transgenes encoding two or more tolerogenic factors are in the form of a polycistronic construct connected by one or more cleavage sites.

Embodiment 955. The method of embodiment 954, wherein the polycistronic construct further comprises one or more nucleotide sequences encoding one or more safety switches.

Embodiment 956. The method of embodiment 954 or embodiment 955, wherein the cleavage site is a self-cleaving site, and/or a protease site.

Embodiment 957. The method of embodiment 956, wherein the self-cleaving site is a 2A site.

Embodiment 958. The method of embodiment 957, wherein the 2A site is selected from the group consisting of a T2A site, a P2A site, an E2A site, and an F2A site.

Embodiment 959. The method of embodiment 956, wherein the protease site is a furin site.

Embodiment 960. The method of embodiment 959, wherein the furin site is selected from the group consisting of an FC1 site, an FC2 site, and an FC3 site.

Embodiment 961. The method of any one of embodiments 918-960, wherein the cell being engineered is collected from a subject who is going to be a recipient of the cell therapy.

Embodiment 962. The method of any one of embodiments 918-960, wherein the cell being engineered is a donor cell collected from a subject who is not a recipient of the cell therapy.

Embodiment 963. The method of embodiment 962, wherein the donor cell is a primary cell.

Embodiment 964. The method of embodiment 962, wherein the donor cell is a pluripotent stem cell (PSC).

Embodiment 965. The method of embodiment 964, wherein the PSC is an induced pluripotent stem cell (iPSC), or an embryonic stem cell (ESC).

Embodiment 966. The method of embodiment 964 or embodiment 965, wherein the engineered PSC is differentiated into a pancreatic islet cell, a retinal pigment epithelial cell, a T cell, a B cell, an NK cell, a thyroid cell, a skin cell, a blood cell, a plasma cell, a platelet, a renal cell, a hepatocyte, a neural cell, a neuronal cell, a glial progenitor cell, an epithelial cell, an endothelial cell, a cardiac cell, a cardiac progenitor cell, or a cardiomyocyte.

Embodiment 967. The method of any one of embodiments 918-966, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, RQR8, and a CD47-SIRPα blockade agent.

Embodiment 968. The method of embodiment 967, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein.

Embodiment 969. The method of embodiment 967 or embodiment 968, wherein the CD47-SIRPα blockade agent is a CD47-binding blockade agent, an SIRPα-binding blockade agent, or a CD47- and/or SIRP-containing fusion protein.

Embodiment 970. The method of embodiment 969, wherein the CD47-binding blockade agent is selected from the group consisting of magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics).

Embodiment 971. The method of embodiment 969, wherein the SIRPα-binding blockade agent is selected from the group consisting of ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University).

Embodiment 972. A method of generating a population of therapeutic cells for cell therapy, comprising inserting one or more transgenes encoding one or more tolerogenic factors, and optionally, one or more nucleotide sequences encoding one or more safety switches, into an endogenous β2 microglobulin (B2M) gene locus, an endogenous class II transactivator (CIITA) gene locus, or both loci, of cells, wherein the population of therapeutic cells comprises engineered cells that are immune evasive.

Embodiment 973. The method of embodiment 972, further comprising modifying the engineered cells to reduce or eliminate expression of one or more MHC I molecules, one or more MHC II molecules, or both.

Embodiment 974. The method of embodiment 973, wherein the modification is carried out by (i) knocking out or knocking down B2M, TAP1, or both to reduce or eliminate expression of one or more MHC I molecules, (ii) knocking out or knocking down CIITA to reduce or eliminate expression of one or more MHC II molecules, or both (i) and (ii).

Embodiment 975. The method of any one of embodiments 972-974, wherein the surface expression of one or more MHC I and/or one or more MHC II molecules is reduced.

Embodiment 976. The method of any one of embodiments 972-974, wherein the surface trafficking of one or more MHC I and/or one or more MHC II molecules is reduced.

Embodiment 977. The method of any one of embodiments 972-976, wherein the one or more transgenes encoding one or more tolerogenic factors, and optionally, the one or more nucleotide sequences encoding one or more safety switches, are inserted into the B2M gene locus, and CIITA or CD74 is knocked out or knocked down.

Embodiment 978. The method of embodiment 977, wherein the transgene encoding CD47 is inserted into the B2M gene locus, and CIITA or CD74 is knocked out or knocked down.

Embodiment 979. The method of any one of embodiments 972-976, wherein the one or more transgenes encoding one or more tolerogenic factors, and optionally, the one or more nucleotide sequences encoding one or more safety switches, are inserted into the CIITA gene locus, and B2M, TAP1, or both are knocked out or knocked down.

Embodiment 980. The method of embodiment 979, wherein the transgene encoding CD47 is inserted into the CIITA gene locus, and B2M, TAP1, or both are knocked out or knocked down.

Embodiment 981. The method of any one of embodiments 972-976, wherein one or more transgenes encoding one or more tolerogenic factors are inserted into both B2M and CIITA gene loci.

Embodiment 982. The method of embodiment 981, wherein the same transgene encoding a tolerogenic factor is inserted into both B2M and CIITA gene loci.

Embodiment 983. The method of embodiment 981, wherein different transgenes encoding different tolerogenic factors are inserted into B2M and CIITA gene loci.

Embodiment 984. The method of any one of embodiments 972-983, wherein the one or more transgenes encoding one or more tolerogenic factors, and optionally, the one or more nucleotide sequences encoding one or more safety switches, are inserted into the B2M or CIITA locus of one allele.

Embodiment 985. The method of any one of embodiments 972-983, and optionally, the one or more nucleotide sequences encoding one or more safety switches, wherein the one or more transgenes encoding one or more tolerogenic factors are inserted into the B2M or CIITA locus of both alleles.

Embodiment 986. The method of any one of embodiments 972-985, wherein the one or more transgenes encoding one or more tolerogenic factors, and optionally, the one or more nucleotide sequences encoding one or more safety switches, are inserted into exon 2 or another CDS of the B2M gene locus.

Embodiment 987. The method of any one of embodiments 972-986, wherein the one or more transgenes encoding one or more tolerogenic factors, and optionally, the one or more nucleotide sequences encoding one or more safety switches, are inserted into exon 3 or another CDS of the CIITA gene locus.

Embodiment 988. The method of any one of embodiments 972-987, wherein the one or more transgenes encoding one or more tolerogenic factors, and optionally, the one or more nucleotide sequences encoding one or more safety switches, are inserted into the B2M or CIITA locus by homology-directed repair (HDR)-mediated insertion using a site-directed nuclease.

Embodiment 989. The method of embodiment 988, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 990. The method of embodiments 972-989, wherein the one or more transgenes encoding one or more tolerogenic factors, and optionally, the one or more nucleotide sequences encoding one or more safety switches, are introduced into the cell by calcium phosphate or lipid-mediated transfection, electroporation, fusogens, or viral transduction.

Embodiment 991. The method of embodiment 990, wherein the virus is a retrovirus or an adeno-associated virus (AAV).

Embodiment 992. The method of embodiment 991, wherein the retrovirus is selected from the group consisting of Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV)), lentivirus, a Gammretrovirus, an Epsilonretrovirus, an Alpharetrovirus, a Betaretrovirus, a Deltaretrovirus, and a Spumaretrovirus.

Embodiment 993. The method of embodiment 991, wherein the AAV is AAV-6 or AAV-9.

Embodiment 994. The method of any one of embodiments 972-993, further comprising selecting for immune evasive cells that have the one or more transgenes inserted by selecting for expression of the one or more tolerogenic factors.

Embodiment 995. The method of embodiment 994, wherein the selection for expression of the one or more tolerogenic factors is carried out by affinity binding, flow cytometry, and/or immunomagnetic selection using antibodies and/or proteins that bind the one or more tolerogenic factors.

Embodiment 996. The method of any one of embodiments 972-995, wherein the tolerogenic factor is selected from the group consisting of CD16, CD16 Fc receptor, CD24, CD27, CD35, CD39, CD46, CD47, CD52, CD55, CD59, CD200, CCL21, CCL22, CTLA4-Ig, C1 inhibitor, FASL, IDO1, HLA-C, HLA-E, HLA-E heavy chain, HLA-F, HLA-G, H2-M3, IL-10, IL15-RF, IL-35, FasL, PD-L1, Serpinb9, DUX4, and Mfge8.

Embodiment 997. The method of embodiment 996, wherein the tolerogenic factor is CD47.

Embodiment 998. The method of embodiment 997, wherein the CD47 is human CD47.

Embodiment 999. The method of embodiment 998, wherein the human CD47 comprises an amino acid sequence that is at least 80% identical to the amino acid sequence set forth in SEQ ID NO: 1 or SEQ ID NO:2.

Embodiment 1000. The method of embodiment 998 or embodiment 999, wherein the human CD47 further comprises a leader peptide.

Embodiment 1001. The method of embodiment 998, wherein the transgene encoding the human CD47 comprises a nucleotide sequence that is at least 80%, at least 85%, at least 90%, at least 95%, or at least 100% identical to the nucleotide sequence set forth in SEQ ID NO:3 or SEQ ID NO:4.

Embodiment 1002. The method of embodiment 998, wherein the transgene encoding the human CD47 comprises a nucleotide sequence that is at least 80%, at least 85%, at least 90%, at least 95%, or at least 100% identical to the nucleotide sequence set forth in SEQ ID NO:5.

Embodiment 1003. The method of embodiment 1001 or embodiment 1002, wherein the nucleotide sequence further comprises a sequence encoding a leader peptide.

Embodiment 1004. The method of any one of embodiments 1001-1003, wherein the nucleotide sequence is codon optimized.

Embodiment 1005. The method of any one of embodiments 1001-1004, wherein the nucleotide sequence further comprises a promoter, an insulator, an enhancer, a polyadenylation (poly(A)) tail, and/or a ubiquitous chromatin opening element.

Embodiment 1006. The method of embodiment 1005, wherein the promoter is a constitutive promoter.

Embodiment 1007. The method of embodiment 1005 or embodiment 1006, wherein the promoter is selected from the group consisting of an EF1α, a short EF1α, CMV, SV40, PGK, UBC, CAG, MND, SSFV, and ICOS promoters.

Embodiment 1008. The method of any one of embodiments 972-1007, wherein the transgenes encoding two or more tolerogenic factors are in the form of a polycistronic construct connected by one or more cleavage sites.

Embodiment 1009. The method of embodiment 1008, wherein the polycistronic construct further comprises one or more nucleotide sequences encoding one or more safety switches.

Embodiment 1010. The method of embodiment 1008 or embodiment 1009, wherein the cleavage site is a self-cleaving site, and/or a protease site.

Embodiment 1011. The method of embodiment 1010, wherein the self-cleaving site is a 2A site.

Embodiment 1012. The method of embodiment 1011, wherein the 2A site is selected from the group consisting of a T2A site, a P2A site, an E2A site, and an F2A site.

Embodiment 1013. The method of embodiment 1010, wherein the protease site is a furin site.

Embodiment 1014. The method of embodiment 1013, wherein the furin site is selected from the group consisting of an FC1 site, an FC2 site, and an FC3 site.

Embodiment 1015. The method of any one of embodiments 972-1014, wherein the cells are collected from a subject who is going to be a recipient of the cell therapy.

Embodiment 1016. The method of any one of embodiments 972-1014, wherein the cells are donor cells collected from one or more subjects who are not a recipient of the cell therapy.

Embodiment 1017. The method of embodiment 1016, wherein the donor cells are primary cells.

Embodiment 1018. The method of embodiment 1016, wherein the donor cells are pluripotent stem cells (PSCs).

Embodiment 1019. The method of embodiment 1018, wherein the PSCs are induced pluripotent stem cells (iPSCs), or embryonic stem cells (ESCs).

Embodiment 1020. The method of embodiment 1018 or embodiment 1019, wherein the engineered PSCs are differentiated into pancreatic islet cells, retinal pigment epithelial cells, T cells, B cells, NK cells, thyroid cells, skin cells, blood cells, plasma cells, platelets, renal cells, hepatocytes, neural cells, neuronal cells, glial progenitor cells, epithelial cells, endothelial cells, cardiac cells, cardiac progenitor cells, or cardiomyocytes.

Embodiment 1021. The method of any one of embodiments 972-1020, wherein at least 50%, at least 55%, at least 60%, at least 65%, at least 70%, at least 75%, at least 80%, at least 85%, at least 90%, at least 95%, or 100% of the cells in the population of therapeutic cells have one or more tolerogenic factors inserted into the B2M or CIITA gene locus.

Embodiment 1022. The method of any one of embodiments 972-1020, wherein at least 50%, at least 55%, at least 60%, at least 65%, at least 70%, at least 75%, at least 80%, at least 85%, at least 90%, at least 95%, or 100% of the cells in the population of therapeutic cells have (a) increased expression of one or more tolerogenic factors encoded by one or more transgenes, and/or (b) reduced expression of one or more MHC I molecules and/or one or more MHC II molecules.

Embodiment 1023. The method of any one of embodiments 972-1020, wherein at least 50%, at least 55%, at least 60%, at least 65%, at least 70%, at least 75%, at least 80%, at least 85%, at least 90%, at least 95%, or 100% of the cells in the population of therapeutic cells have (a) increased expression of CD47 encoded by a transgene, and/or (b) reduced expression of one or more MHC I molecules and/or one or more MHC II molecules.

Embodiment 1024. The method of any one of embodiments 972-1020, wherein at least 50%, at least 55%, at least 60%, at least 65%, at least 70%, at least 75%, at least 80%, at least 85%, at least 90%, at least 95%, or 100% of the cells in the population of therapeutic cells have (a) increased expression of CD47 encoded by a transgene, and/or (b) reduced expression of B2M, TAP1, CD74, and/or CIITA.

Embodiment 1025. The method of any one of embodiments 972-1020, wherein at least 50%, at least 55%, at least 60%, at least 65%, at least 70%, at least 75%, at least 80%, at least 85%, at least 90%, at least 95%, or 100% of the cells in the population of therapeutic cells have (a) increased expression of CD47 encoded by a transgene, (b) reduced expression of B2M, TAP1, or both, and one or more MHC I molecules, and/or (c) reduced expression of CIITA, CD74, or both, and one or more MHC II molecules.

Embodiment 1026. The method of any one of embodiments 972-1025, wherein the population of therapeutic cells comprises two or more types or subtypes of immune evasive cells.

Embodiment 1027. The method of any one of embodiments 972-1025, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, RQR8, and a CD47-SIRPα blockade agent.

Embodiment 1028. The method of embodiment 1027, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein.

Embodiment 1029. The method of embodiment 1027 or embodiment 1028, wherein the CD47-SIRPα blockade agent is a CD47-binding blockade agent, an SIRPα-binding blockade agent, or a CD47- and/or SIRP-containing fusion protein.

Embodiment 1030. The method of embodiment 1029, wherein the CD47-binding blockade agent is selected from the group consisting of magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics).

Embodiment 1031. The method of embodiment 1029, wherein the SIRPα-binding blockade agent is selected from the group consisting of ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University).

Embodiment 1032. An engineered cell generated by the method of any one of embodiments 918-971.

Embodiment 1033. The engineered cell of embodiment 1032, wherein the engineered cell is a B2Mindel/indel cell, TAPlindel/indel cell, CD74indel/indel cell, and/or CIITAindel/indel cell.

Embodiment 1034. The engineered cell of embodiment 1032 or embodiment 1033, wherein the engineered cell expresses one or more exogenous tolerogenic factors.

Embodiment 1035. The engineered cell of embodiment 1034, wherein the engineered cell has one or more exogenous tolerogenic factors inserted at a B2M gene locus, a CIITA gene locus, or both.

Embodiment 1036. The engineered cell of embodiment 1034 or embodiment 1035, wherein the tolerogenic factor is selected from the group consisting of CD16, CD16 Fc receptor, CD24, CD27, CD35, CD39, CD46, CD47, CD52, CD55, CD59, CD200, CCL21, CCL22, CTLA4-Ig, C1 inhibitor, FASL, IDO1, HLA-C, HLA-E, HLA-E heavy chain, HLA-F, HLA-G, H2-M3, IL-10, IL15-RF, IL-35, FasL, PD-L1, Serpinb9, DUX4, and Mfge8.

Embodiment 1037. The engineered cell of embodiment 1036, wherein the tolerogenic factor is CD47.

Embodiment 1038. The engineered cell of embodiment 1037, wherein the CD47 is human CD47.

Embodiment 1039. The engineered cell of embodiment 1038, wherein the human CD47 comprises an amino acid sequence that is at least 80% identical to the amino acid sequence set forth in SEQ ID NO:1 or SEQ ID NO:2.

Embodiment 1040. The engineered cell of embodiment 1037 or embodiment 1038, wherein the human CD47 further comprises a leader peptide.

Embodiment 1041. A population of therapeutic cells generated by the method of any one of embodiments 972-1021.

Embodiment 1042. The population of therapeutic cells of embodiment 1041, wherein the population comprises allogeneic immune evasive cells.

Embodiment 1043. The population of therapeutic cells of embodiment 1041 or embodiment 1042, wherein the population comprises two or more types or subtypes of immune evasive cells.

Embodiment 1044. The population of therapeutic cells of any one of embodiments 1041-1043, wherein the population comprises one or more immune evasive cell types selected from the group consisting of pancreatic islet cells, retinal pigment epithelial cells, T cells, B cells, NK cells, thyroid cells, cells producing factors, skin cells, blood cells, plasma cells, platelets, renal cells, hepatocytes, neural cells, neuronal cells, glial progenitor cells, epithelial cells, endothelial cells, cardiac cells, cardiac progenitor cells, and cardiomyocytes.

Embodiment 1045. The population of therapeutic cells of any one of embodiments 1041-1044, wherein the population comprises immune evasive primary cells, immune evasive cells derived from PSCs, or both.

Embodiment 1046. The population of therapeutic cells of any one of embodiments 1041-1045, wherein the population comprises immune evasive cardiomyocytes.

Embodiment 1047. The population of therapeutic cells of any one of embodiments 1041-1045, wherein the population comprises immune evasive cardiac progenitor cells (CPCs), immune evasive epicardial cells, or both.

Embodiment 1048. The population of therapeutic cells of any one of embodiments 1041-1045, wherein the population comprises immune evasive primary T cells, immune evasive T cells derived from iPSCs, or both.

Embodiment 1049. The population of therapeutic cells of any one of embodiments 1041-1045, wherein the population comprises two or more immune evasive T cell subtypes.

Embodiment 1050. The population of therapeutic cells of any one of embodiments 1041-1045, wherein the population comprises immune evasive primary pancreatic islet cells, immune evasive pancreatic islet cells derived from iPSCs, or both.

Embodiment 1051. The population of therapeutic cells of embodiment 1050, wherein the pancreatic islet cells are pancreatic β islet cells.

Embodiment 1052. The population of therapeutic cells of any one of embodiments 1041-1051, wherein at least 50%, at least 55%, at least 60%, at least 65%, at least 70%, at least 75%, at least 80%, at least 85%, at least 90%, at least 95%, or 100% of the cells in the population have one or more tolerogenic factors inserted into the B2M or CIITA gene locus.

Embodiment 1053. The population of therapeutic cells of any one of embodiments 1041-1052, wherein at least 50%, at least 55%, at least 60%, at least 65%, at least 70%, at least 75%, at least 80%, at least 85%, at least 90%, at least 95%, or 100% of the cells have (a) increased expression of one or more tolerogenic factors encoded by one or more transgenes, and/or (b) reduced expression of one or more MHC I molecules and/or one or more MHC II molecules.

Embodiment 1054. The population of therapeutic cells of any one of embodiments 1041-1053, wherein at least 50%, at least 55%, at least 60%, at least 65%, at least 70%, at least 75%, at least 80%, at least 85%, at least 90%, at least 95%, or 100% of the cells in the population have (a) increased expression of CD47 encoded by a transgene, and/or (b) reduced expression of one or more MHC I molecules and/or one or more MHC II molecules.

Embodiment 1055. The population of therapeutic cells of any one of embodiments 1041-1054, wherein at least 50%, at least 55%, at least 60%, at least 65%, at least 70%, at least 75%, at least 80%, at least 85%, at least 90%, at least 95%, or 100% of the cells have (a) increased expression of CD47 encoded by a transgene, and/or (b) reduced expression of B2M, TAP1, CD74, and/or CIITA.

Embodiment 1056. The population of therapeutic cells of any one of embodiments 1041-1055, wherein at least 50%, at least 55%, at least 60%, at least 65%, at least 70%, at least 75%, at least 80%, at least 85%, at least 90%, at least 95%, or 100% of the cells in the population have (a) increased expression of CD47 encoded by a transgene, (b) reduced expression of B2M, TAP1, or both, and one or more MHC I molecules, and/or (c) reduced expression of CIITA, CD74, or both, and one or more MHC II molecules.

Embodiment 1057. The population of therapeutic cells of any one of embodiments 1041-1056, wherein at least 50%, at least 55%, at least 60%, at least 65%, at least 70%, at least 75%, at least 80%, at least 85%, at least 90%, at least 95%, or 100% of the cells in the population have (a) increased expression of CD47 encoded by a transgene, and/or (b) B2M, TAP1, CD74, and/or CIITA knocked-out.

Embodiment 1058. The population of therapeutic cells of any one of embodiments 1041-1057, wherein each of the remainder cells in the population has one of:
(i) reduced expression of one or more MHC I molecules,
(ii) reduced expression of one or more MHC II molecules,
(iii) increased expression of CD47,
(iv) reduced expression of one or more MHC I molecules and one or more MHC II molecules,
(v) reduced expression of one or more MHC I molecules and increased expression of CD47,
(vi) reduced expression of one or more MHC II molecules and increased expression of CD47, or
(vii) endogenous expression of MHC I molecules, MHC II molecules, and CD47.

Embodiment 1059. A pharmaceutical composition comprising the population of therapeutic cells of any one of embodiments 1041-1058.

Embodiment 1060. The pharmaceutical composition of embodiment 1059, wherein the population comprises allogeneic immune evasive cells.

Embodiment 1061. The pharmaceutical composition of embodiment 1059 or embodiment 1060, wherein the population comprises two or more types or subtypes of immune evasive cells.

Embodiment 1062. The pharmaceutical composition of any one of embodiments 1059-1061, wherein the population comprises one or more immune evasive cell types selected from the group consisting of pancreatic islet cells, retinal pigment epithelial cells, T cells, B cells, NK cells, thyroid cells, cells producing factors, skin cells, blood cells, plasma cells, platelets, renal cells, hepatocytes, neural cells, neuronal cells, glial progenitor cells, epithelial cells, endothelial cells, cardiac cells, cardiac progenitor cells, and cardiomyocytes.

Embodiment 1063. The pharmaceutical composition of any one of embodiments 1059-1062, wherein the population comprises immune evasive primary cells, immune evasive cells derived from PSCs, or both.

Embodiment 1064. The population of therapeutic cells of any one of embodiments 1059-1063, wherein the population comprises immune evasive cardiomyocytes.

Embodiment 1065. The pharmaceutical composition of any one of embodiments 1059-1063, wherein the population comprises immune evasive cardiac progenitor cells (CPCs), immune evasive epicardial cells, or both.

Embodiment 1066. The pharmaceutical composition of any one of embodiments 1059-1063, wherein the population comprises immune evasive primary T cells, immune evasive T cells derived from iPSCs, or both.

Embodiment 1067. The pharmaceutical composition of any one of embodiments 1059-1063, wherein the population comprises two or more immune evasive T cell subtypes.

Embodiment 1068. The pharmaceutical composition of any one of embodiments 1059-1063, wherein the population comprises immune evasive primary pancreatic islet cells, immune evasive pancreatic islet cells derived from iPSCs, or both.

Embodiment 1069. The pharmaceutical composition of embodiment 1068, wherein the pancreatic islet cells are pancreatic β islet cells.

Embodiment 1070. A method of treating a cellular deficiency or treating a condition or disease in a tissue or organ in a subject, comprising administering to the subject the population of therapeutic cells of any one of embodiments 1041-1057, or the pharmaceutical composition of any one of embodiments 1058-1069, wherein the tissue or organ is selected from the group consisting of heart, lung, kidney, liver, pancreas, intestine, stomach, cornea, bone marrow, blood vessel, heart valve, brain, spinal cord, and bone.

Embodiment 1071. The method of embodiment 1070, further comprising activating the safety switch to kill or inhibit the therapeutic cells previously administered to the subject.

Embodiment 1072. The method of embodiment 1071, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, RQR8, and a CD47-SIRPα blockade agent.

Embodiment 1073. The method of any one of embodiments 1070-1072, further comprising administering one or more CD47-SIPRα blockade agents to the subject to kill or inhibit the therapeutic cells previously administered to the subject.

Embodiment 1074. The method of embodiment 1072 or embodiment 1073, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein.

Embodiment 1075. The method of any one of embodiments 1072-1074, wherein the CD47-SIRPα blockade agent is a CD47-binding blockade agent, an SIRPα-binding blockade agent, or a CD47- and/or SIRP-containing fusion protein.

Embodiment 1076. The method of embodiment 1075, wherein the CD47-binding blockade agent is selected from the group consisting of magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics).

Embodiment 1077. The method of embodiment 1075, wherein the SIRPα-binding blockade agent is selected from the group consisting of ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University).

Embodiment 1078. The method of any one of embodiments 1070-1077, wherein the cellular deficiency is selected from the group consisting of an autoimmune disease, a neurodegenerative disease, a cardiovascular condition or disease, a vascular condition or disease, a corneal condition or disease, a liver condition or disease, a thyroid condition or disease, and a kidney condition or disease.

Embodiment 1079. The method of embodiment 1078, wherein the autoimmune disease is selected from the group consisting of multiple sclerosis, myasthenia gravis, rheumatoid arthritis, diabetes, systemic lupus and erythematosus.

Embodiment 1080. The method of embodiment 1078, wherein the neurodegenerative disease is selected from the group consisting of leukodystrophy, Huntington's disease, Parkinson's disease, multiple sclerosis, transverse myelitis, and Pelizaeus-Merzbacher disease (PMD).

Embodiment 1081. The method of embodiment 1078, wherein the liver disease is cirrhosis of the liver.

Embodiment 1082. The method of embodiment 1078, wherein the corneal disease is Fuchs dystrophy or congenital hereditary endothelial dystrophy.

Embodiment 1083. The method of embodiment 1078, wherein the cardiovascular disease is myocardial infarction or congestive heart failure.

Embodiment 1084. A method of treating a disease or a condition in a subject in need thereof, comprising administering to the subject the population of therapeutic cells of any one of embodiments 1041-1057, or the pharmaceutical composition of any one of embodiments 1058-1069.

Embodiment 1085. The method of embodiment 1084, further comprising activating the safety switch to kill or inhibit the therapeutic cells previously administered to the subject.

Embodiment 1086. The method of embodiment 1085, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, RQR8, and a CD47-SIRPα blockade agent.

Embodiment 1087. The method of any one of embodiments 1084-1086, further comprising administering one or more CD47-SIPRα blockade agents to the subject to kill or inhibit the therapeutic cells previously administered to the subject.

Embodiment 1088. The method of embodiment 1086 or embodiment 1087, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein.

Embodiment 1089. The method of any one of embodiments 1086-1088, wherein the CD47-SIRPα blockade agent is a CD47-binding blockade agent, an SIRPα-binding blockade agent, or a CD47- and/or SIRP-containing fusion protein.

Embodiment 1090. The method of embodiment 1089, wherein the CD47-binding blockade agent is selected from the group consisting of magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics).

Embodiment 1091. The method of embodiment 1089, wherein the SIRPα-binding blockade agent is selected from the group consisting of ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University).

Embodiment 1092. The method of any one of embodiments 1084-1091, wherein the disease is cancer.

Embodiment 1093. The method of embodiment 1092, wherein the cancer is a hematologic malignancy, liver cancer, pancreatic cancer, breast cancer, ovarian cancer, colorectal cancer, lung cancer, non-small cell lung cancer, multiple myeloma, gastric cancer, gastric adenocarcinoma, pancreatic adenocarcinoma, glioblastoma, neuroblastoma, lung squamous cell carcinoma, hepatocellular carcinoma, or bladder cancer.

Embodiment 1094. The method of embodiment 1093, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 1095. The method of any one of embodiments 1084-1091, wherein the disease is an autoimmune disease.

Embodiment 1096. The method of embodiment 1095, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 1097. The method of any one of embodiments 1084-1091, wherein the disease is diabetes mellitus.

Embodiment 1098. The method of embodiment 1097, wherein the diabetes is selected from the group consisting Type I diabetes, Type II diabetes, prediabetes, and gestational diabetes.

Embodiment 1099. The method of any one of embodiments 1084-1091, wherein the disease is a neurological disease.

Embodiment 1100. The method of embodiment 1099, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 1101. A method of treating a patient who has received or is receiving a tissue or organ transplant, comprising administering to the subject the population of therapeutic cells of any one of embodiments 1041-1057, or the pharmaceutical composition of any one of embodiments 1058-1069.

Embodiment 1102. The method of embodiment 1101, further comprising activating the safety switch to kill or inhibit the therapeutic cells previously administered to the subject.

Embodiment 1103. The method of embodiment 1102, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase such as rapaCasp9, CCR4, CD16, CD19, CD20, CD30, EGFR, GD2, HER1, HER2, MUC1, PSMA, RQR8, and a CD47-SIRPα blockade agent.

Embodiment 1104. The method of any one of embodiments 1101-1103, further comprising administering one or more CD47-SIPRα blockade agents to the subject to kill or inhibit the therapeutic cells previously administered to the subject.

Embodiment 1105. The method of embodiment 1103 or embodiment 1104, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein.

Embodiment 1106. The method of any one of embodiments 1103-1105, wherein the CD47-SIRPα blockade agent is a CD47-binding blockade agent, an SIRPα-binding blockade agent, or a CD47- and/or SIRP-containing fusion protein.

Embodiment 1107. The method of embodiment 1106, wherein the CD47-binding blockade agent is selected from the group consisting of magrolimab ((Hu5F9-G4)) (Forty Seven, Inc.; Gilead Sciences, Inc.), urabrelimab, CC-90002 (Celgene; Bristol-Myers Squibb), IBI-188 (letaplimab, Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), STI-6643 (Sorrento), and IMC-002 (Samsung Biologics; ImmuneOncia Therapeutics).

Embodiment 1108. The method of embodiment 1106, wherein the SIRPα-binding blockade agent is selected from the group consisting of ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University).

Embodiment 1109. The method of any one of embodiments 1101-1108, wherein the organ or tissue transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and a partial cornea transplant.

Embodiment 1110. The method of any one of embodiments 1101-1109, wherein the organ or tissue transplant is an allograft transplant.

Embodiment 1111. The method of any one of embodiments 1101-1109, wherein the organ or tissue transplant is an autograft transplant.

LENGTHY TABLES
The patent application contains a lengthy table section. A copy of the table is available in electronic form from the USPTO web site (https://seqdata.uspto.gov/docdetail?docId=US20260275385A1). An electronic copy of the table will also be available from the USPTO upon request and payment of the fee set forth in 37 CFR 1.19(b)(3).

1. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

2. A method of selecting engineered cells suitable for use in a therapeutic product, the method comprising:
selecting one or more genetically engineered cells from a population of cells based on a level of one or more markers on the cell surface of the one or more genetically engineered cells, and
preparing the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

3. A method of treating a disease in a subject with a composition comprising genetically engineered cells, the method comprising:
selecting one or more genetically engineered cells from a population of cells based on a level of one or more markers on the cell surface of the one or more genetically engineered cells,
formulating the composition comprising the selected one or more genetically engineered cells for treating a disease in a subject, and
administering the formulated composition to a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor.

4. A method of producing a composition comprising engineered cells with increased purity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a β2 microglobulin (B2M) gene locus,
wherein the level of the one or more markers on the cell surface comprise a level of an MHC I molecule, and
wherein at least 30% of the genetically engineered cells in the formulated composition comprise the transgene encoding the first tolerogenic factor at the insertion site at the B2M gene locus and/or the first tolerogenic factor.

5. A method of producing a composition comprising genetically engineered cells with enhanced efficacy, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus,
wherein the level of the one or more markers on the cell surface comprise a level of an MHC I molecule and/or the first tolerogenic factor, and
wherein the composition with enhanced efficacy is more effective than a composition comprising cells that do not comprise the one or more genetic modifications.

6. A method of producing a composition with reduced host immune response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a B2 microglobulin (B2M) gene locus,
wherein the level of the one or more markers on the cell surface comprises a level of an MHC I molecule and/or the first tolerogenic factor on the cell surface of the one or more genetically engineered cells, and
wherein the composition with reduced host immune response elicits a reduced host immune response compared to a composition comprising comparable cells that do not comprise the one or more genetic modifications.

7. A method of formulating a composition with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus,
wherein the level of one or more markers on the cell surface comprises a level of an MHC I molecule and/or the first tolerogenic factor, and
wherein the composition with reduced immunogenicity elicits a reduced host immune response compared to a composition comprising comparable cells that do not comprise the one or more genetic modifications.

8. A method of producing a composition comprising genetically engineered cells with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
wherein the level of the one or more markers on the cell surface comprise a level of an MHC I molecule and/or the first tolerogenic factor, and
wherein the composition with reduced immunogenicity elicits a reduced host immune response compared to a composition comprising comparable cells that do not comprise the one or more genetic modifications.

9. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

10. A method of selecting engineered cells suitable for use in a therapeutic product, the method comprising:
selecting one or more genetically engineered cells from a population of cells based on a level of one or more markers on the cell surface of the one or more genetically engineered cells, and
preparing the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

11. A method of treating a disease in a subject with a composition comprising genetically engineered cells, the method comprising:
selecting one or more genetically engineered cells from a population of cells based on a level of one or more markers on the cell surface of the one or more genetically engineered cells,
formulating the composition comprising the selected one or more genetically engineered cells for treating a disease in a subject, and
administering the formulated composition to a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor.

12. A method of producing a composition comprising engineered cells with increased purity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus,
wherein the level of the one or more markers on the cell surface comprise a level of an MHC II molecule and/or the first tolerogenic factor, and
wherein at least 30% of the genetically engineered cells in the formulated composition comprise the transgene encoding the first tolerogenic factor at the insertion site at the CIITA gene locus.

13. A method of producing a composition comprising genetically engineered cells with enhanced efficacy, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus,
wherein the level of the one or more markers on the cell surface comprise a level of an MHC II molecule and/or the first tolerogenic factor, and
wherein the composition with enhanced efficacy is more effective than a composition comprising cells that do not comprise the one or more genetic modifications.

14. A method of producing a composition with reduced host immune response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a class II transactivator (CIITA) gene locus,
wherein the level of the one or more markers on the cell surface comprises a level of an MHC II molecule and/or the first tolerogenic factor on the cell surface of the one or more genetically engineered cells, and
wherein the composition with reduced host immune response elicits a reduced host immune response compared to a composition comprising comparable cells that do not comprise the one or more genetic modifications.

15. A method of formulating a composition with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus,
wherein the level of one or more markers on the cell surface comprises a level of an MHC II molecule and/or the first tolerogenic factor, and
wherein the composition with reduced immunogenicity elicits a reduced host immune response compared to a composition comprising comparable cells that do not comprise the one or more genetic modifications.

16. A method of producing a composition comprising genetically engineered cells with reduced immunogenicity, the method comprising:
selecting one or more genetically engineered cells from a population of cells based on a formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
wherein the level of the one or more markers on the cell surface comprise a level of an MHC II molecule and/or the first tolerogenic factor, and
wherein the composition with reduced immunogenicity elicits a reduced host immune response compared to a composition comprising comparable cells that do not comprise the one or more genetic modifications.

17. The method of any one of claim 6-8 or 14-16, wherein the host immune response is an immune response of the subject against the one or more genetically engineered cells.

18. The method of claim 17, wherein the reduced host immune response comprises reduced donor-specific antibodies in the subject.

19. The method of claim 6-8, 14-16, or 17, wherein the reduced host immune response comprises reduced IgM or IgG antibodies in the subject.

20. The method of claim 6-8, 14-16, or 17, wherein the reduced host immune response comprises reduced complement-dependent cytotoxicity (CDC) in the subject.

21. The method of claim 6-8, 14-16, or 17, wherein the reduced host immune response comprises reduced TH1 activation in the subject.

22. The method of claim 6-8, 14-16, or 17, wherein the reduced host immune response comprises reduced NK cell killing in the subject.

23. The method of claim 6-8, 14-16, or 17, wherein the reduced host immune response comprises reduced killing by whole blood PBMCs in the subject.

24. A method of producing a composition comprising genetically engineered cells with a reduced graft versus host response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a β2 microglobulin (B2M) gene locus, and
optionally wherein the level of the one or more markers on the cell surface comprise a level of an MHC I molecule and/or the first tolerogenic factor, and
wherein the one or more genetically engineered cells of the composition with a reduced graft versus host response have a reduced immune response against cells of the subject as compared to a composition comprising comparable cells that do not comprise the one or more genetic modifications.

25. A method of producing a composition comprising genetically engineered cells with a reduced graft versus host response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a class II transactivator (CIITA) gene locus, and
optionally wherein the level of the one or more markers on the cell surface comprise a level of an MHC II molecule and/or the first tolerogenic factor, and
wherein the one or more genetically engineered cells of the composition with a reduced graft versus host response have a reduced immune response against cells of the subject as compared to a composition comprising comparable cells that do not comprise the one or more genetic modifications.

26. The method of any of the preceding claims, wherein the one or more genetic modifications comprises an inserted transgene encoding a first tolerogenic factor.

27. The method of any of the preceding claims, wherein the method comprises inserting a transgene encoding a first tolerogenic factor into an insertion site in the genome of one or more cells in the population.

28. The method of any of the preceding claims, wherein the transgene encoding the first tolerogenic factor is inserted at an insertion site at a B2M gene locus.

29. The method of any one of claims 1-27, wherein the transgene encoding the first tolerogenic factor is inserted at an insertion site at a CIITA gene locus.

30. The method of any one of claims 27-29, wherein the step of inserting comprises homology-directed repair (HDR)-mediated insertion using a genome-modifying protein.

31. The method of claim 30, wherein the step of inserting using a genome modifying protein comprises insertion by a CRISPR-associated transposase, prime editing, a TnpB polypeptide, or Programmable Addition via Site-specific Targeting Elements (PASTE).

32. The method of claim 30, wherein the step of inserting using a genome modifying protein comprises insertion by a site-directed nuclease.

33. The method of claim 32, wherein the site-directed nuclease is selected from a zinc finger nuclease (ZFN), a TAL-effector nuclease (TALEN), or a CRISPR-Cas combination, optionally wherein the Cas is selected from a Cas9 or a Cas12.

34. The method of claim 32 or 33, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

35. The method of any one of claims 27-34, wherein the step of inserting comprises homology-directed repair (HDR)-mediated insertion using a guide RNA (gRNA) and a CRISPR-associated (Cas) nuclease.

36. The method of claim 35, wherein the gRNA comprises a complementary region,
wherein the complementary region comprises a nucleic acid sequence that is complementary to a target nucleic acid sequence within the B2M gene locus, and wherein the target nucleic acid sequence comprises the insertion site.

37. The method of claim 35, wherein the gRNA comprises a complementary region,
wherein the complementary region comprises a nucleic acid sequence that is complementary to a target nucleic acid sequence within the CIITA gene locus, and wherein the target nucleic acid sequence comprises the insertion site.

38. The method of any of the preceding claims, wherein the insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, gttn, or yttn and wherein:
(i) r=a org,
(ii) y=c or t,
(iii) w=a or t,
(iv) v=a or c or g, and
(v) n=a, c, t, or g.

39. The method of any one of claims 27-38, wherein the step of inserting comprises homology-directed repair (HDR)-mediated insertion using SpCas9 and the PAM is ngg or nag, wherein n=a, c, t, or g.

40. The method of any one of claims 27-38, wherein the step of inserting comprises homology-directed repair (HDR)-mediated insertion using SaCas9 and the PAM is ngrrt or ngrrn, wherein:
(i) r=a org, and
(ii) n=a, c, t, or g.

41. The method of any one of claims 27-38, wherein the step of inserting comprises homology-directed repair (HDR)-mediated insertion using NmeCas9 and the PAM is nnnngatt, wherein n=a, c, t, or g.

42. The method of any one of claims 27-38, wherein the step of inserting comprises homology-directed repair (HDR)-mediated insertion using CjCas9 and the PAM is nnnnryac, wherein:
(i) r=a org,
(ii) y=c or t, and
(iii) n=a, c, t, or g.

43. The method of any one of claims 27-38, wherein the step of inserting comprises homology-directed repair (HDR)-mediated insertion using StCas9 and the PAM is nnagaaw wherein:
(i) w=a or t, and
(ii) n=a, c, t, or g.

44. The method of any one of claims 27-38, wherein the step of inserting comprises homology-directed repair (HDR)-mediated insertion using TdCas9 and the PAM is naaaac, wherein n=a, c, t, or g.

45. The method of one of claims 27-38, wherein the step of inserting comprises homology-directed repair (HDR)-mediated insertion using LbCas12a and the PAM is tttv, wherein v=a or c or g.

46. The method of one of claims 27-38, wherein the step of inserting comprises homology-directed repair (HDR)-mediated insertion using AsCas12a and the PAM is tttv, wherein v=a or c or g.

47. The method of one of claims 27-38, wherein the step of inserting comprises homology-directed repair (HDR)-mediated insertion using AacCas 12b and the PAM is ttn, wherein n=a, c, t, or g.

49. The method of one of claims 27-38, wherein the step of inserting comprises homology-directed repair (HDR)-mediated insertion using BhCas12b and the PAM is attn, tttn, or gttn, wherein n=a, c, t, or g.

50. The method of one of claims 27-38, wherein the step of inserting comprises homology-directed repair (HDR)-mediated insertion using MAD7 (ErCas12a) and the PAM is yttn, wherein:
(i) y=c or t, and
(ii) n=a, c, t, or g.

51. The method of any one of claim 32, 35, or 39-50, wherein homology-directed repair (HDR)-mediated insertion using a site-directed nuclease is performed with an HDR efficiency equal to or greater than HDR insertion using lentivirus.

52. The method of any one of claim 27-30, 32, 33, or 34, wherein the step of inserting comprises homology-directed repair (HDR)-mediated insertion using ZFN.

53. The method of any one of claim 27-30, 32, 33, 34, or 52, wherein the first insertion site is 25 nucleotides or less from a zinc finger binding sequence.

54. The method of any one of claim 27-30, 32, 33, or 34, wherein the step of inserting comprises homology-directed repair (HDR)-mediated insertion using TALEN.

55. The method of any one of claim 27-30, 32, 33, or 34, wherein the first insertion site is 25 nucleotides or less from a transcription activator-like effectors (TALE) binding sequence.

56. The method of any one of claim 27-32 or 34, wherein the step of inserting comprises homology-directed repair (HDR)-mediated insertion using a guide RNA (gRNA) and a TnpB polypeptide.

57. The method of any claim 56, wherein the gRNA comprises a complementary region, wherein the complementary region comprises a nucleic acid sequence that is complementary to a target nucleic acid sequence within the B2M gene locus, and wherein the target nucleic acid sequence comprises the insertion site.

58. The method of claim 56, wherein the gRNA comprises a complementary region, wherein the complementary region comprises a nucleic acid sequence that is complementary to a target nucleic acid sequence within the CIITA gene locus, and wherein the target nucleic acid sequence comprises the insertion site.

59. The method of any one of claims 56-58, wherein the insertion site is 25 nucleotides or less from a target adjacent motif (TAM) sequence, wherein the TAM sequence is tca, tcac, tcag, tcat, tcaa, ttcan, ttcaa, ttcag, or ttgat, and wherein:
(i) n=a, c, t, or g.

60. The method of any one of claims 56-59, wherein the step of inserting comprises homology-directed repair (HDR)-mediated insertion using TnpB polypeptide and the TAM is tca.

61. The method of any one of claims 56-59, wherein the step of inserting comprises homology-directed repair (HDR)-mediated insertion using TnpB polypeptide and the TAM is tcac.

62. The method of any one of claims 56-59, wherein the step of inserting comprises homology-directed repair (HDR)-mediated insertion using TnpB polypeptide and the TAM is tcag.

63. The method of any one of claims 56-59, wherein the step of inserting comprises homology-directed repair (HDR)-mediated insertion using TnpB polypeptide and the TAM is tcat.

64. The method of any one of claims 56-59, wherein the step of inserting comprises homology-directed repair (HDR)-mediated insertion using TnpB polypeptide and the TAM is tcaa.

65. The method of any one of claims 56-59, wherein the step of inserting comprises homology-directed repair (HDR)-mediated insertion using TnpB polypeptide and the TAM is ttcan, wherein n=a, c, t, or g.

66. The method of any one of claims 56-59, wherein the step of inserting comprises homology-directed repair (HDR)-mediated insertion using TnpB polypeptide and the TAM is ttcaa.

67. The method of any one of claims 56-59, wherein the step of inserting comprises homology-directed repair (HDR)-mediated insertion using TnpB polypeptide and the TAM is ttcag.

68. The method of any one of claims 56-59, wherein the step of inserting comprises homology-directed repair (HDR)-mediated insertion using TnpB polypeptide and the TAM is ttgat.

69. The method of any of the preceding claims, wherein the insertion site is in an exon.

70. The method of any of the preceding claims, wherein the insertion site is in an intron.

71. The method of any of the preceding claims, wherein the insertion site is between an intron and an exon.

72. The method of any of the preceding claims, wherein the insertion site is in a regulatory region.

73. The method of any one of claim 1-8, 24, 26-28, 30-36, 38-57, or 59-72, wherein the transgene encoding the first tolerogenic factor at an insertion site at a B2M gene locus reduces expression of a functional B2M.

74. The method of any one of claim 1-8, 24, 26-28, 30-36, 38-57, or 59-72, wherein the transgene encoding the first tolerogenic factor at an insertion site at a B2M gene locus reduces expression of a functional MHC I molecule.

75. The method of any one of claim 1-8, 24, 26-28, 30-36, 38-57, or 59-72, wherein the transgene encoding the first tolerogenic factor at an insertion site at a B2M gene locus disrupts expression of a functional B2M.

76. The method of one of claim 1-8, 24, 26-28, 30-36, 38-57, or 59-75, wherein the transgene encoding the first tolerogenic factor at an insertion site at a B2M gene locus disrupts expression of a functional MHC I molecule.

77. The method of any one of claim 1-8, 24, 26-28, 30-36, 38-57, or 59-76, wherein the transgene encoding the first tolerogenic factor has a forward orientation (5′ to 3′) relative to the B2M gene locus.

78. The method of any one of claim 1-8, 24, 26-28, 30-36, 38-57, or 59-77, wherein the transgene encoding the first tolerogenic factor is in the same orientation as the B2M gene locus.

79. The method of any one of claim 1-8, 24, 26-28, 30-36, 38-57, or 59-76, wherein the transgene encoding the first tolerogenic factor has a reverse orientation (5′ to 3′) relative to the B2M gene locus.

80. The method of any one of claim 1-8, 24, 26-28, 30-36, 38-57, 59-76, or 79, wherein the transgene encoding the first tolerogenic factor is in the reverse orientation as the B2M gene locus.

81. The method of any one of claim 1-8, 24, 26-28, 30-36, or 38-80, wherein the B2M gene locus is an endogenous B2M locus.

82. The method of any one of claim 1-8, 24, 26-28, 30-36, or 38-81, wherein the B2M gene locus is chr15: 4,711,358-44,718,851.

83. The method of any one of claim 1-8, 24, 26-28, 30-36, 38-69, or 73-82, wherein the insertion site is within exon 1, exon 2, exon 3, or exon 4 at the B2M gene locus.

84. The method of any one of claim 1-8, 24, 26-28, 30-36, 38-69, or 73-82, wherein the insertion site is within exon 1 at the B2M gene locus.

85. The method of any one of claim 1-8, 24, 26-28, 30-36, 38-69, or 73-82, wherein the insertion site is within exon 2 at the B2M gene locus.

86. The method of any one of claim 1-8, 24, 26-28, 30-36, 38-69, or 73-82, wherein the insertion site is within exon 3 at the B2M gene locus.

87. The method of any one of claim 1-8, 24, 26-28, 30-36, 38-69, or 73-82, wherein the insertion site is within exon 4 at the B2M gene locus.

88. The method of any one of claim 1-8, 24, 26-28, 30-36, 38-68, 70, or 73-82, wherein the insertion site is within intron 1, intron 2, or intron 3 at the B2M gene locus.

89. The method of any one of claim 1-8, 24, 26-28, 30-36, 38-68, 70, or 73-82, wherein the insertion site is within intron 1 at the B2M gene locus.

90. The method of any one of claim 1-8, 24, 26-28, 30-36, 38-68, 70, or 73-82, wherein the insertion site is within intron 2 at the B2M gene locus.

91. The method of any one of claim 1-8, 24, 26-28, 30-36, 38-68, 70, or 73-82, wherein the insertion site is within intron 3 at the B2M gene locus.

92. The method of any one of claim 1-8, 24, 26-28, 30-36, or 38-82, wherein the insertion site is within the 5′ UTR at the B2M gene locus.

93. The method of any one of claim 1-8, 24, 26-28, 30-36, or 38-82, wherein the insertion site is within the 3′ UTR at the B2M locus.

94. The method of any one of claims 1-8, 17-24, 26-28, 30-36, 38-57, 59-93, wherein the step of inserting comprises using an hB2M gRNA comprising a nucleic acid sequence selected from Table 7, Table 10, Table 12, Table 14, Table 16, Table 18, Table 20, Table 22, Table 24, Table 26, or Table 28.

95. The method of any one of claim 9-16, 25, 29, 37, or 58-72, wherein the transgene encoding the first tolerogenic factor at an insertion site at a CIITA gene locus reduces expression of a functional CIITA.

96. The method of any one of claim 9-16, 25, 29, 37, 58-72, or 95, wherein the transgene encoding the first tolerogenic factor at an insertion site at a CIITA gene locus reduces expression of a functional MHC II molecule.

97. The method of any one of claim 9-16, 25, 29, 37, 58-72, or 95-96, wherein the transgene encoding the first tolerogenic factor at an insertion site at a CIITA gene locus disrupts expression of a functional CIITA.

98. The method of any one of claim 9-16, 25, 29, 37, 58-72, or 95-97, wherein the transgene encoding the first tolerogenic factor at an insertion site at a CIITA gene locus disrupts expression of a functional MHC II molecule.

99. The method of any one of claim 9-16, 25, 29, 37, 58-72, or 95-98, wherein the transgene encoding the first tolerogenic factor has a forward orientation (5′ to 3′) relative to the CIITA gene locus.

100. The method of any one of claim 9-16, 25, 29, 37, 58-72, or 95-99, wherein the transgene encoding the first tolerogenic factor is in the same orientation as the CIITA gene locus.

101. The method of one of claim 9-16, 25, 29, 37, 58-72, or 95-98, wherein the transgene encoding the first tolerogenic factor has a reverse orientation (5′ to 3′) relative to the CIITA gene locus.

102. The method of any one of claim 9-16, 25, 29, 37, 58-72, 95-98, or 101, wherein the transgene encoding the first tolerogenic factor is in the reverse orientation as the CIITA gene locus.

103. The method of any one of claim 9-16, 25, 29, 37, 58-72, or 95-102, wherein the CIITA gene locus is an endogenous CIITA locus.

104. The method of one of claim 9-16, 25, 29, 37, 58-72, or 95-103, wherein the CIITA gene locus is chr16: 10,866,222-10,943,021.

105. The method of any one of claim 9-16, 25, 29, 37, 58-64, or 95-104, wherein the insertion site is within exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, or exon 20 at the CIITA gene locus.

106. The method of any one of claim 9-16, 25, 29, 37, 58-69, or 95-10⁵, wherein the insertion site is within exon 1 at the CIITA gene locus.

107. The method of any one of claim 9-16, 25, 29, 37, 58-69, or 95-10⁵, wherein the insertion site is within exon 2 at the CIITA gene locus.

108. The method of any one of claim 9-16, 25, 29, 37, 58-69, or 95-10⁵, wherein the insertion site is within exon 3 at the CIITA gene locus.

109. The method of any one of claim 9-16, 25, 29, 37, 58-69, or 95-10⁵, wherein the insertion site is within exon 4 at the CIITA gene locus.

110. The method of any one of claim 9-16, 25, 29, 37, 58-69, or 95-10⁵, wherein the insertion site is within exon 5 at the CIITA gene locus.

111. The method of any one of claim 9-16, 25, 29, 37, 58-69, or 95-10⁵, wherein the insertion site is within exon 6 at the CIITA gene locus.

112. The method of any one of claim 9-16, 25, 29, 37, 58-69, or 95-10⁵, wherein the insertion site is within exon 7 at the CIITA gene locus.

113. The method of any one of claim 9-16, 25, 29, 37, 58-69, or 95-10⁵, wherein the insertion site is within exon 8 at the CIITA gene locus.

114. The method of any one of claim 9-16, 25, 29, 37, 58-69, or 95-10⁵, wherein the insertion site is within exon 9 at the CIITA gene locus.

115. The method of any one of claim 9-16, 25, 29, 37, 58-69, or 95-10⁵, wherein the insertion site is within exon 10 at the CIITA gene locus.

116. The method of any one of claim 9-16, 25, 29, 37, 58-69, or 95-10⁵, wherein the insertion site is within exon 11 at the CIITA gene locus.

117. The method of any one of claim 9-16, 25, 29, 37, 58-69, or 95-10⁵, wherein the insertion site is within exon 12 at the CIITA gene locus.

118. The method of any one of claim 9-16, 25, 29, 37, 58-69, or 95-10⁵, wherein the insertion site is within exon 13 at the CIITA gene locus.

119. The method of any one of claim 9-16, 25, 29, 37, 58-69, or 95-10⁵, wherein the insertion site is within exon 14 at the CIITA gene locus.

120. The method of any one of claim 9-16, 25, 29, 37, 58-69, or 95-10⁵, wherein the insertion site is within exon 15 at the CIITA gene locus.

121. The method of any one of claim 9-16, 25, 29, 37, 58-69, or 95-10⁵, wherein the insertion site is within exon 16 at the CIITA gene locus.

122. The method of any one of claim 9-16, 25, 29, 37, 58-69, or 95-10⁵, wherein the insertion site is within exon 17 at the CIITA gene locus.

123. The method of any one of claim 9-16, 25, 29, 37, 58-69, or 95-10⁵, wherein the insertion site is within exon 18 at the CIITA gene locus.

124. The method of any one of claim 9-16, 25, 29, 37, 58-69, or 95-10⁵, wherein the insertion site is within exon 19 at the CIITA gene locus.

125. The method of any one of claim 9-16, 25, 29, 37, 58-69, or 95-10⁵, wherein the insertion site is within exon 20 at the CIITA gene locus.

126. The method of any one of claim 9-16, 25, 29, 37, 58-68, 70, or 95-104, wherein the insertion site is within intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, or intron 19 at the CIITA gene locus.

127. The method of any one of claim 9-16, 25, 29, 37, 58-68, 70, 95-104, or 126, wherein the insertion site is within intron 1 at the CIITA gene locus.

128. The method of any one of claim 9-16, 25, 29, 37, 58-68, 70, 95-104, or 126, wherein the insertion site is within intron 2 at the CIITA gene locus.

129. The method of any one of claim 9-16, 25, 29, 37, 58-68, 70, 95-104, or 126, wherein the insertion site is within intron 3 at the CIITA gene locus.

130. The method of any one of claim 9-16, 25, 29, 37, 58-68, 70, 95-104, or 126, wherein the insertion site is within intron 4 at the CIITA gene locus.

131. The method of any one of claim 9-16, 25, 29, 37, 58-68, 70, 95-104, or 126, wherein the insertion site is within intron 5 at the CIITA gene locus.

132. The method of one of claim 9-16, 25, 29, 37, 58-68, 70, 95-104, or 126, wherein the insertion site is within intron 6 at the CIITA gene locus.

133. The method of any one of claim 9-16, 25, 29, 37, 58-68, 70, 95-104, or 126, wherein the insertion site is within intron 7 at the CIITA gene locus.

134. The method of one of claim 9-16, 25, 29, 37, 58-68, 70, 95-104, or 126, wherein the insertion site is within intron 8 at the CIITA gene locus.

135. The method of one of claim 9-16, 25, 29, 37, 58-68, 70, 95-104, or 126, wherein the insertion site is within intron 9 at the CIITA gene locus.

136. The method of one of claim 9-16, 25, 29, 37, 58-68, 70, 95-104, or 126, wherein the insertion site is within intron 10 at the CIITA gene locus.

137. The method of one of claim 9-16, 25, 29, 37, 58-68, 70, 95-104, or 126, wherein the insertion site is within intron 11 at the CIITA gene locus.

138. The method of any one of claim 9-16, 25, 29, 37, 58-68, 70, 95-104, or 126, wherein the insertion site is within intron 12 at the CIITA gene locus.

139. The method of any one of claim 9-16, 25, 29, 37, 58-68, 70, 95-104, or 126, wherein the insertion site is within intron 13 at the CIITA gene locus.

140. The method of any one of claim 9-16, 25, 29, 37, 58-68, 70, 95-104, or 126, wherein the insertion site is within intron 14 at the CIITA gene locus.

141. The method of any one of claim 9-16, 25, 29, 37, 58-68, 70, 95-104, or 126, wherein the insertion site is within intron 15 at the CIITA gene locus.

142. The method of any one of claim 9-16, 25, 29, 37, 58-68, 70, 95-104, or 126, wherein the insertion site is within intron 16 at the CIITA gene locus.

143. The method of any one of claim 9-16, 25, 29, 37, 58-68, 70, 95-104, or 126, wherein the insertion site is within intron 17 at the CIITA gene locus.

144. The method of any one of claim 9-16, 25, 29, 37, 58-68, 70, 95-104, or 126, wherein the insertion site is within intron 18 at the CIITA gene locus.

145. The method of any one of claim 9-16, 25, 29, 37, 58-68, 70, 95-104, or 126, wherein the insertion site is within intron 19 at the CIITA gene locus.

146. The method of any one of claim 9-16, 25, 29, 37, 58-68, 67, or 95-104, wherein the insertion site is within the 5′ UTR at the CIITA gene locus.

147. The method of any one of claim 9-16, 25, 29, 37, 58-68, 67, or 95-104, wherein the insertion site is within the 3′ UTR at the CIITA gene locus.

148. The method of any one of claims 9-16, 17-23, 25-27, 29-35, 37-56, 58-72, 95-147, wherein the step of inserting comprises using an hCIITA gRNA comprising a nucleic acid sequence selected from Table 7, Table 11, Table 13, Table 15, Table 17, Table 19, Table 21, Table 23, Table 25, Table 27, or Table 29.

149. The method of any of the preceding claims, wherein the level of one or more markers on the cell surface comprises a level of the first tolerogenic factor on the cell surface of the one or more genetically engineered cells.

150. The method of any of the preceding claims, wherein the method comprises detecting a level of the first tolerogenic factor on the cell surface of the one or more genetically engineered cells.

151. The method of any of the preceding claims, wherein the one or more genetically engineered cells are selected if the first tolerogenic factor is detected on the cell surface of the one or more genetically engineered cells.

152. The method of any of the preceding claims, wherein the first tolerogenic factor is or comprises A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

153. The method of any of the preceding claims, wherein the first tolerogenic factor is or comprises CD47.

154. The method of any of the preceding claims, wherein the first tolerogenic factor is or comprises human CD47.

155. The method of any one of claims 152-154, wherein the CD47 comprises an amino acid sequence at least 80% identical to an amino acid sequence set forth in SEQ ID NO:1 or SEQ ID NO: 2.

156. The method of any of the preceding claims, wherein the transgene encoding the first tolerogenic factor is a transgene that encodes CD47 and the transgene comprises a nucleotide sequence at least 80% identical to a nucleotide sequence set forth in SEQ ID NO:3 or SEQ ID NO: 4.

157. The method of any of the preceding claims, wherein the transgene encoding a first tolerogenic factor is a transgene that encodes CD47 and the nucleotide sequence of the transgene is codon-optimized.

158. The method of any of the preceding claims, wherein the transgene is at least 80% identical to a nucleotide sequence set forth in SEQ ID NO:5.

159. The method of any one of claim 1-8, 17-23, 24, 26-28, 30-36, 38-57, 59-94, or 149-159, wherein the method comprises detecting a level of B2M on the cell surface of the one or more genetically engineered cells.

160. The method of any one of claim 1-8, 17-23, 24, 26-28, 30-36, 38-57, 59-94, or 149-159, wherein the one or more genetically engineered cells are selected if B2M is not present at a detectable level on the cell surface of the one or more genetically engineered cells.

161. The method of any one of claim 1-8, 17-23, 24, 26-28, 30-36, 38-57, 59-94, or 149-159, wherein the method comprises detecting a level of an MHC-I molecule on the cell surface of the one or more genetically engineered cells.

162. The method of any one of claim 1-8, 17-23, 24, 26-28, 30-36, 38-57, 59-94, or 149-159, wherein the one or more genetically engineered cells are selected if an MHC-I molecule is not present at a detectable level on the cell surface of the one or more genetically engineered cells.

163. The method of any one of claim 9-23, 25-27, 29-35, 37-56, 58-72, or 95-158, wherein the method comprises detecting a level of an MHC-II molecule on the cell surface of the one or more genetically engineered cells.

164. The method of any one of claim 9-23, 25-27, 29-35, 37-56, 58-72, 95-158, or 163, wherein the one or more genetically engineered cells are selected if an MHC-II molecule is not present at a detectable level on the cell surface of the one or more genetically engineered cells.

165. The method of any of the preceding claims, wherein the level of one or more markers on the cell surface comprises a level of an MHC I molecule, an MHC II molecule, or both on the cell surface of the one or more genetically engineered cells.

166. The method of any of the preceding claims, wherein the method comprises detecting a level of the MHC I molecule, the MHC II molecule, or both on the cell surface of the one or more genetically engineered cells.

167. The method of any of the preceding claims, wherein the one or more genetically engineered cells are selected if the MHC I molecule, the MHC II molecule, or both, are not present at a detectable level on the cell surface of the one or more genetically engineered cells.

168. The method of any of the preceding claims, wherein the method comprises detecting a level of the first tolerogenic factor on the cell surface of the one or more genetically engineered cells.

169. The method of any of the preceding claims, wherein the one or more genetically engineered cells are selected if the first tolerogenic factor is not present at a detectable level on the cell surface of the one or more genetically engineered cells.

170. The method of any of the preceding claims, wherein the one or more genetically engineered cells are selected if the first tolerogenic factor is present at a detectable level on the cell surface of the one or more genetically engineered cells.

171. The method of any of the preceding claims, wherein the level of one or more markers on the cell surface comprises a level of the first tolerogenic factor on the cell surface of the one or more genetically engineered cells.

172. The method of any of the preceding claims, wherein the one or more genetic modifications comprise a modification at a T-cell receptor (TCR) locus, B2M locus, a TAP I locus, a NLRC5 locus, a CIITA locus, an HLA-A locus, an HLA-B locus, an HLA-C locus, an HLA-DP locus, an HLA-DM locus, an HLA-DOA locus, an HLA-DOB locus, an HLA-DQ locus, an HLA-DR locus, a RFX5 locus, a RFXANK locus, a RFXAP locus, an NFY-A locus, an NFY-B locus, an NFY-C locus, or a combination thereof.

173. The method of any of the preceding claims, wherein the one or more genetic modifications comprise a modification at a TCR gene locus.

174. The method of any of the preceding claims, wherein the modification at the TCR gene locus is a heterozygous modification.

175. The method of any of the preceding claims, wherein the modification at the TCR gene locus is a homozygous modification.

176. The method of any of the preceding claims, wherein the method comprises modifying a TCR gene locus.

177. The method of any of the preceding claims, wherein the modification at the TCR gene locus comprises a knock-out of the TCR gene locus.

178. The method of any of the preceding claims, wherein the method comprises knocking out the TCR gene locus.

179. The method of any of the preceding claims, wherein the one or more genetic modifications comprise a modification at an HLA-A locus, an HLA-B locus, an HLA-C locus, or a combination thereof.

180. The method of any of the preceding claims, wherein the modification at the HLA-A locus, the HLA-B locus, the HLA-C locus, or a combination thereof is a heterozygous modification.

181. The method of any of the preceding claims, wherein the modification at the HLA-A locus, the HLA-B locus, the HLA-C locus, or a combination thereof is a homozygous modification.

182. The method of any of the preceding claims, wherein the method comprises modifying an HLA-A locus, an HLA-B locus, an HLA-C locus, or a combination thereof.

183. The method of any of the preceding claims, wherein the modification at the HLA-A locus, the HLA-B locus, the HLA-C locus, or a combination thereof comprises a knock-out of the HLA-A locus, the HLA-B locus, the HLA-C locus, or a combination thereof.

184. The method of any of the preceding claims, wherein the method comprises knocking out an HLA-A locus, an HLA-B locus, an HLA-C locus, or a combination thereof.

185. The method of any of the preceding claims, wherein the one or more genetic modifications comprise a modification at an HLA-DM locus, an HLA-DO locus, an HLA-DP locus, an HLA-DQ locus, an HLA-DR locus, or a combination thereof.

186. The method of any of the preceding claims, wherein the modification at the HLA-DM locus, the HLA-DO locus, the HLA-DP locus, the HLA-DQ locus, the HLA-DR locus, or a combination thereof is a heterozygous modification.

187. The method of any of the preceding claims, wherein the modification at the HLA-DM locus, the HLA-DO locus, the HLA-DP locus, the HLA-DQ locus, the HLA-DR locus, or a combination thereof is a homozygous modification.

188. The method of any of the preceding claims, wherein the method comprises modifying an HLA-DM locus, an HLA-DO locus, an HLA-DP locus, an HLA-DQ locus, an HLA-DR locus, or a combination thereof.

189. The method of any of the preceding claims, wherein the modification at the HLA-DM locus, the HLA-DO locus, the HLA-DP locus, the HLA-DQ locus, the HLA-DR locus, or a combination thereof comprises a knock-out of the HLA-DM locus, the HLA-DO locus, the HLA-DP locus, the HLA-DQ locus, the HLA-DR locus, or a combination thereof.

190. The method of any of the preceding claims, wherein the method comprises knocking out an HLA-DM locus, an HLA-DO locus, an HLA-DP locus, an HLA-DQ locus, an HLA-DR locus, or a combination thereof.

191. The method of any of the preceding claims, wherein the one or more genetic modifications comprise a modification at a B2M gene locus.

192. The method of any of the preceding claims, wherein the modification at the B2M gene locus is a heterozygous modification.

193. The method of any of the preceding claims, wherein the modification at the B2M gene locus is a homozygous modification.

194. The method of any of the preceding claims, wherein the method comprises modifying a B2M locus.

195. The method of any of the preceding claims, wherein the modification at the B2M locus comprises a knock-out of the B2M locus.

196. The method of any of the preceding claims, wherein the method comprises knocking out the B2M gene locus.

197. The method of any of the preceding claims, wherein the one or more genetic modifications comprise a modification at a CIITA gene locus.

198. The method of any of the preceding claims, wherein the modification at the CIITA gene locus is a heterozygous modification.

199. The method of any of the preceding claims, wherein the modification at the CIITA gene locus is a homozygous modification.

200. The method of any of the preceding claims, wherein the method comprises modifying a CIITA gene locus.

201. The method of any of the preceding claims, wherein the modification at the CIITA gene locus comprises a knock-out of the CIITA gene locus.

202. The method of any of the preceding claims, wherein the method comprises knocking out the CIITA gene locus.

203. The method of any of the preceding claims, wherein the level of one or more markers on the cell surface comprises a level of an MHC I molecule, an MHC II molecule, or both, on the cell surface of the one or more genetically engineered cells.

204. The method of any of the preceding claims, wherein the method comprises detecting a level of the MHC I molecule, the MHC II molecule, or both, on the cell surface of the one or more genetically engineered cells.

205. The method of any of the preceding claims, wherein the one or more genetically engineered cells are selected if the MHC I molecule, the MHC II molecule, or both, are not present at a detectable level on the cell surface of the one or more genetically engineered cells.

206. The method of any of the preceding claims, wherein the one or more genetic modifications comprise a knock-out of: ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof.

207. The method of claim 206, wherein the protein that is involved in oxidative or ER stress is selected from the group consisting of TXNIP, PERK, IRE1α, and DJ-1 (PARK7).

208. The method of any of the preceding claims, wherein the level of one or more markers on the cell surface comprises a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

209. The method of any of the preceding claims, wherein the method comprises detecting a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

210. The method of any of the preceding claims, wherein the one or more genetically engineered cells are selected if ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, are not present at a detectable level on the cell surface of the one or more genetically engineered cells.

211. The method of any of the preceding claims, wherein the one or more genetic modifications comprise a second inserted transgene.

212. The method of claim 211, wherein the second transgene encodes a chimeric antigen receptor (CAR).

213. The method of any of the preceding claims, wherein the method comprises inserting a transgene encoding a CAR in the genome of one or more cells in the population.

214. The method of claim 213, wherein the transgene encoding a CAR is inserted at a safe harbor locus.

215. The method of claim 213 or 214, wherein the transgene encoding a CAR is inserted at a TRAC locus, a TRBC1 locus, a TRBC2 locus, a B2M locus, a CIITA locus, a MICA locus, a MICB locus, or a safe harbor locus.

216. The method of claim 213-214, wherein the transgene encoding a CAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

217. The method of claim 211, wherein the second transgene encodes a chimeric auto antigen receptor (CAAR).

218. The method of any of the preceding claims, wherein the method comprises inserting a transgene encoding a CAAR in the genome of one or more cells in the population.

219. The method of claim 217, wherein the transgene encoding a CAAR is inserted at a safe harbor locus.

220. The method of any one of claims 217-219, wherein the transgene encoding a CAAR is inserted at a TRAC locus, a TRBC1 locus, a TRBC2 locus, a B2M locus, a CIITA locus, a MICA locus, a MICB locus, or a safe harbor locus.

221. The method of any one of claims 217-219, wherein the transgene encoding a CAAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

222. The method of any one of claims 211-221, wherein the second transgene is inserted into the same site as the transgene encoding the first tolerogenic factor.

223. The method of any one of claims 211-222, wherein the second transgene and the first tolerogenic factor are encoded by two separate constructs.

224. The method of any one of claims 211-222, wherein the second transgene and the first tolerogenic factor are encoded by a polycistronic construct.

225. The method of claim 224, wherein the polycistronic construct is a bicistronic construct.

226. The method of any one of claims 212-216, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL 13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a G_{D2}-specific CAR, a G_{D3}-specific CAR, a HMW-MAA-specific CAR, a IL-11Ra-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

227. The method of any one of claims 212-217, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.

228. The method of any one of claim 212-216, or 226-227, wherein the level of one or more markers on the cell surface comprises a level of the CAR on the cell surface of the one or more genetically engineered cells.

229. The method of any one of claim 212-216, or 226-228, wherein the method comprises detecting a level of the CAR on the cell surface of the one or more genetically engineered cells.

230. The method of any one of claim 212-216, or 226-229, wherein the one or more genetically engineered cells are selected if the CAR is detected on the cell surface of the one or more genetically engineered cells.

231. The method of any one of claims 217-221, wherein the CAAR comprises an antigen selected from the group consisting of a pancreatic β-cell antigen, synovial joint antigen, myelin basic protein, proteolipid protein, myelin oligodendritic glycoprotein, MuSK, keratinocyte adhesion protein desmoglein 3 (Dsg3), Ro-RNP complex, La antigen, myeloperoxidase, proteinase 3, cardiolipin, citrullinated proteins, carbamylated proteins, and α3 chain of basement membrane collagen.

232. The method of any one of claim 217-221, or 231, wherein the level of one or more markers on the cell surface comprises a level of the CAAR on the cell surface of the one or more genetically engineered cells.

233. The method of any one of claim 217-221, 231, or 232, wherein the method comprises detecting a level of the CAAR on the cell surface of the one or more genetically engineered cells.

234. The method of any one of claims one of claim 217-221, or 231-233, wherein the one or more genetically engineered cells are selected if the CAAR is detected on the cell surface of the one or more genetically engineered cells.

235. The method of any one of claim 211 or 222-225, wherein the second transgene encodes a second tolerogenic factor.

236. The method of claim 235, wherein the second transgene encoding the second tolerogenic factor is inserted at a TRAC locus, a TRBC1 locus, a TRBC2 locus, a B2M locus, a CIITA locus, a MICA locus, a MICB locus, a safe harbor locus, an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

237. The method of claim 235 or 236, wherein the second tolerogenic factor is or comprises A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

238. The method of any one of claims 235-237, wherein the first tolerogenic factor and the second tolerogenic factor are the same tolerogenic factor.

239. The method of any one of claims 235-237, wherein the first tolerogenic factor and the second tolerogenic factor are different tolerogenic factors.

240. The method of any one of claims 235-239, wherein the method comprises detecting a level of the second tolerogenic factor on the cell surface of the one or more genetically engineered cells, wherein the second tolerogenic factor is expressed at a higher level than endogenous expression levels of the second tolerogenic factor in a comparable cell that does not comprise the second transgene.

241. The method of any one of claims 235-240, wherein the one or more genetically engineered cells are selected if the second tolerogenic factor is detected on the cell surface of the one or more genetically engineered cells at a higher level of expression than endogenous expression levels of the second tolerogenic factor in a comparable cell that does not comprise the second transgene.

242. The method of any one of claims 235-241, wherein the one or more genetically engineered cells are selected from a population of cells based on a level of two or more markers on the cell surface of the one or more genetically engineered cells.

243. The method of any one of claims 235-242, wherein the one or more genetically engineered cells are selected from a population of cells based on a level of three or more markers on the cell surface of the one or more genetically engineered cells.

244. The method of any one of claims 235-243, wherein the one or more genetically engineered cells are selected from a population of cells based on a level of four or more markers on the cell surface of the one or more genetically engineered cells.

245. The method of any of the preceding claims, wherein each of the one or more markers on the cell surface of the one or more genetically engineered cells is associated with at least one of the one or more genetic modifications.

246. The method of any of the preceding claims, wherein each of the one or more genetic modifications impacts the level of at least one of the one or more markers on the cell surface of the one or more genetically engineered cells.

247. The method of any of the preceding claims, wherein one or more of: (i) the transgene encoding the first tolerogenic factor, (ii) the transgene encoding the CAR, or (iii) the transgene encoding the second tolerogenic factor comprise a promoter, an insulator, an enhancer, a polyadenylation (poly(A)) tail, a ubiquitous chromatin opening element, or a combination thereof.

248. The method of any of the preceding claims, wherein one or more of: (i) the transgene encoding the first tolerogenic factor, (ii) the transgene encoding the CAR, or (iii) the transgene encoding the second tolerogenic factor comprise a promoter and the promoter is a constitutive promoter.

249. The method of claim 248, wherein the constitutive promoter is an EF1α, EF1α short, CMV, SV40, PGK, adenovirus late, vaccinia virus 7.5K, SV40, HSV tk, mouse mammary tumor virus (MMTV), HIV LTR, moloney virus, Esptein Barr virus (EBV), Rous sarcoma virus (RSV), UBC CAG, MND, SSFV, or ICOS promoter.

250. The method of any of the preceding claims, wherein the population of cells are human cells or non-human animal cells.

251. The method of claim 250, wherein non-human animal cells are porcine, bovine or ovine cells.

252. The method of any of the preceding claims, wherein the population of cells are human cells.

253. The method of any of the preceding claims, wherein the population of cells are differentiated cells derived from stem cells or progenitor cells.

254. The method of claim 253, wherein the stem cells are pluripotent stem cells.

255. The method of claim 254, wherein the pluripotent stem cells are induced pluripotent stem cells (iPSC).

256. The method of claim 254, wherein the pluripotent stem cells are embryonic stem cells (ESC).

257. The method of any of the preceding claims, wherein the population of cells are primary cells isolated from a donor.

258. The method of claim 257, wherein the donor is a single donor or multiple donors.

259. The method of claim 257 or 258, wherein the donor is healthy and/or is not suspected of having a disease or condition at the time the primary cells are obtained from the donor.

260. The method of any of the preceding claims, wherein the population of cells are islet cells, beta islet cells, pancreatic islet cells, immune cells, B cells, T cells, natural killer (NK) cells, natural killer T (NKT) cells, macrophage cells, endothelial cells, muscle cells, cardiac muscle cells, smooth muscle cells, skeletal muscle cells, dopaminergic neurons, retinal pigmented epithelium cells, optic cells, hepatocytes, thyroid cells, skin cells, glial progenitor cells, neural cells, cardiac cells, stem cells, hematopoietic stem cells, induced pluripotent stem cells (iPSCs), mesenchymal stem cells (MSCs), embryonic stem cells (ESCs), pluripotent stem cell (PSCs), blood cells, or a combination thereof.

261. The method of any of the preceding claims, wherein the population of cells are T-cells.

262. The method of claim 261, wherein the T-cells are CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells, or a combination thereof.

263. The method of claim 261 or 262, wherein the T cells are cytotoxic T-cells, helper T-cells, memory T-cells, regulatory T-cells, tumor infiltrating lymphocytes, or a combination thereof.

264. The method of any of the preceding claims, wherein the population of cells are human T-cells.

265. The method of any of the preceding claims, wherein the population of cells are autologous T-cells.

266. The method of any of the preceding claims, wherein the population of cells are allogeneic T-cells.

267. The method of claim 266, wherein the allogeneic T cells are primary T cells.

268. The method of claim 266 or 267, wherein the allogeneic T cells have been differentiated from embryonic stem cells (ESCs) or an induced pluripotent stem cells (iPSCs).

269. The method of any of the preceding claims, wherein the population of cells are T-cells, and wherein, after the steps of inserting the transgene encoding the first tolerogenic factor and modifying an HLA-A locus, an HLA-B locus, an HLA-C locus, or a combination thereof, at least 30% of the population of T-cells each have (a) reduced cell surface expression of MHC I and/or MHC II molecules as compared to comparable T-cells that have not been genetically engineered, and (b) increased expression of the first tolerogenic factor encoded by the first transgene as compared to comparable T-cells that have not been genetically engineered.

270. The method of any of the preceding claims, wherein the population of cells are T-cells and the tolerogenic factor is CD47, and wherein, after the steps of inserting the transgene encoding the first tolerogenic factor and knocking out the B2M locus and/or the CIITA locus, at least 30% of the population of T-cells each have (a) a B2M locus and/or a CIITA locus knocked-out, and (b) increased expression of CD47 as compared to comparable T-cells that have not been genetically engineered.

271. The method of any of the preceding claims, wherein the population of cells are T-cells and the first tolerogenic factor is CD47, and wherein, after the steps of inserting the transgene encoding the first tolerogenic factor and knocking out the B2M locus and/or the CIITA locus, at least 30% of the population of T-cells each have (a) reduced cell surface expression of MHC I and/or MHC II molecules as compared to T-cells that have not been genetically engineered, and (b) increased expression of CD47 as compared to comparable T-cells that have not been genetically engineered.

272. The method of any one of claims 269-271, wherein at least 35% of the population of T-cells each have (a) and (b).

273. The method of one of claims 1-268, wherein the population of cells are T-cells and the tolerogenic factor is CD47, and wherein, after the steps of inserting the transgene encoding the first tolerogenic factor and knocking out the B2M locus and/or the CIITA locus, at least 20% of the population of T-cells each have (a) reduced expression of B2M as compared to comparable T-cells that have not been genetically engineered, (b) reduced expression of CIITA as compared to comparable T-cells that have not been genetically engineered, and (c) increased expression of CD47 as compared to comparable T-cells that have not been genetically engineered.

274. The method of claim 273, wherein at least 35% of the T-cells each have (a) and (b).

275. The method of claim 273 or 274, wherein at least 35% of the population of T-cells each have (a), (b), and (c).

276. The method of any of the preceding claims, further comprising storing the cells.

277. The method of claim 276, wherein storing the cells comprises freezing the cells.

278. The method of any of the preceding claims, wherein the one or more genetically engineered cells are stored after being selected based on a level of one or more markers on the cell surface of the one or more genetically engineered cells.

279. The method of any one of claims 276-278, wherein the one or more genetically engineered cells are stored after one or more genetic modifications are introduced.

280. The method of any one of claims 276-279, wherein the one or more genetically engineered cells are stored before being selected based on a level of one or more markers on the cell surface of the one or more genetically engineered cells.

281. The method of any one of claims 276-278, wherein the one or more genetically engineered cells are stored before one or more genetic modifications are introduced.

282. The method of any one of claims 277-281, further comprising thawing the cells.

283. The method of claim 282, wherein the one or more genetically engineered cells are thawed prior to one or more genetic modifications being introduced.

284. The method of claim 282 or 283, wherein the one or more genetically engineered cells are formulated in the composition after thawing.

285. The method of claim 282 or 283, wherein the one or more genetically engineered cells are formulated in the composition before thawing.

286. The method of any of the preceding claims, wherein the composition is suitable for use in a subject.

287. The method of any of the preceding claims, wherein the composition is a therapeutic composition.

288. The method of any of the preceding claims, wherein the composition is a cell therapy composition.

289. The method of any of the preceding claims, wherein the composition comprises a pharmaceutically acceptable additive, carrier, diluent, or excipient.

290. The method of any of the preceding claims, wherein the composition comprises a buffered solution.

291. The method of any of the preceding claims, wherein the composition comprises a pharmaceutically acceptable buffer.

292. The method of claim 291, wherein the pharmaceutically acceptable buffer comprises neutral buffer saline or phosphate buffered saline.

293. The method of any of the preceding claims, wherein the composition comprises Plasma-Lyte A®, dextrose, dextran, sodium chloride, human serum albumin (HSA), dimethylsulfoxide (DMSO), or a combination thereof.

294. The method of any of the preceding claims, wherein the composition comprises a cryoprotectant.

295. A population of genetically engineered cells produced by the method of any one of claims 1-294.

296. A population of cells that have been genetically engineered to comprise a transgene encoding a first tolerogenic factor, wherein at least 30% of the cells have increased cell surface expression of a first tolerogenic factor as compared to a comparable cell that has not been genetically engineered.

297. The population of cells of claim 296, wherein the transgene encoding the first tolerogenic factor is inserted at an insertion site at a B2M gene locus.

298. The population of cells of claim 296, wherein the transgene encoding the first tolerogenic factor is inserted at an insertion site at a CIITA gene locus.

299. The population of cells of any one of claims 296-298, wherein the insertion site is in an exon.

300. The population of cells of any one of claims 296-298, wherein the insertion site is in an intron.

301. The population of cells of any one of claims 296-298, wherein the insertion site is between an intron and an exon.

302. The population of cells of any one of claims 296-298, wherein the insertion site is in a regulatory region.

303. The population of cells of any one of claim 296, 297, or 299, wherein the insertion site is within exon 1, exon 2, exon 3, or exon 4 at the B2M gene locus.

304. The population of cells of any one of claim 296, 297, 299, or 303, wherein the insertion site is within exon 1 at the B2M gene locus.

305. The population of cells of any one of claim 296, 297, 299, or 303, wherein the insertion site is within exon 2 at the B2M gene locus.

306. The population of cells of any one of claim 296, 297, 299, or 303, wherein the insertion site is within exon 3 at the B2M gene locus.

307. The population of cells of any one of claim 296, 297, 299, or 303, wherein the insertion site is within exon 4 at the B2M gene locus.

308. The population of cells of any one of claim 296, 297, or 300, wherein the insertion site is within intron 1, intron 2, or intron 3 at the B2M gene locus.

309. The population of cells of any one of claim 296, 297, 300, or 308, wherein the insertion site is within intron 1 at the B2M gene locus.

310. The population of cells of any one of claim 296, 297, 300, or 308, wherein the insertion site is within intron 2 at the B2M gene locus.

311. The population of cells of any one of claim 296, 297, 300, or 308, wherein the insertion site is within intron 3 at the B2M gene locus.

312. The population of cells of any one of claim 296, 297, or 302, wherein the insertion site is within the 5′ UTR at the B2M gene locus.

313. The population of cells of any one of claim 296, 297, or 302, wherein the insertion site is within the 3′ UTR at the B2M locus.

314. The population of cells of any one of claim 296, 298, or 299, wherein the insertion site is within exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, or exon 20 at the CIITA gene locus.

315. The population of cells of any one of claim 296, 298, 299, or 314, wherein the insertion site is within exon 1 at the CIITA gene locus.

316. The population of cells of any one of claim 296, 298, 299, or 314, wherein the insertion site is within exon 2 at the CIITA gene locus.

317. The population of cells of any one of claim 296, 298, 299, or 314, wherein the insertion site is within exon 3 at the CIITA gene locus.

318. The population of cells of any one of claim 296, 298, 299, or 314, wherein the insertion site is within exon 4 at the CIITA gene locus.

319. The population of cells of any one of claim 296, 298, 299, or 314, wherein the insertion site is within exon 5 at the CIITA gene locus.

320. The population of cells of any one of claim 296, 298, 299, or 314, wherein the insertion site is within exon 6 at the CIITA gene locus.

321. The population of cells of any one of claim 296, 298, 299, or 314, wherein the insertion site is within exon 7 at the CIITA gene locus.

322. The population of cells of any one of claim 296, 298, 299, or 314, wherein the insertion site is within exon 8 at the CIITA gene locus.

323. The population of cells of any one of claim 296, 298, 299, or 314, wherein the insertion site is within exon 9 at the CIITA gene locus.

324. The population of cells of any one of claim 296, 298, 299, or 314, wherein the insertion site is within exon 10 at the CIITA gene locus.

325. The population of cells of any one of claim 296, 298, 299, or 314, wherein the insertion site is within exon 11 at the CIITA gene locus.

326. The population of cells of any one of claim 296, 298, 299, or 314, wherein the insertion site is within exon 12 at the CIITA gene locus.

327. The population of cells of any one of claim 296, 298, 299, or 314, wherein the insertion site is within exon 13 at the CIITA gene locus.

328. The population of cells of any one of claim 296, 298, 299, or 314, wherein the insertion site is within exon 14 at the CIITA gene locus.

329. The population of cells of any one of claim 296, 298, 299, or 314, wherein the insertion site is within exon 15 at the CIITA gene locus.

330. The population of cells of any one of claim 296, 298, 299, or 314, wherein the insertion site is within exon 16 at the CIITA gene locus.

331. The population of cells of any one of claim 296, 298, 299, or 314, wherein the insertion site is within exon 17 at the CIITA gene locus.

332. The population of cells of any one of claim 296, 298, 299, or 314, wherein the insertion site is within exon 18 at the CIITA gene locus.

333. The population of cells of any one of claim 296, 298, 299, or 314, wherein the insertion site is within exon 19 at the CIITA gene locus.

334. The population of cells of any one of claim 296, 298, 299, or 314, wherein the insertion site is within exon 20 at the CIITA gene locus.

335. The population of cells of any one of claim 296, 298, or 300, wherein the insertion site is within intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, or intron 19 at the CIITA gene locus.

336. The population of cells of any one of claim 296, 298, 300, or 335, wherein the insertion site is within intron 1 at the CIITA gene locus.

337. The population of cells of any one of claim 296, 298, 300, or 335, wherein the insertion site is within intron 2 at the CIITA gene locus.

338. The population of cells of any one of claim 296, 298, 300, or 335, wherein the insertion site is within intron 3 at the CIITA gene locus.

339. The population of cells of any one of claim 296, 298, 300, or 335, wherein the insertion site is within intron 4 at the CIITA gene locus.

340. The population of cells of any one of claim 296, 298, 300, or 335, wherein the insertion site is within intron 5 at the CIITA gene locus.

341. The population of cells of any one of claim 296, 298, 300, or 335, wherein the insertion site is within intron 6 at the CIITA gene locus.

342. The population of cells of any one of claim 296, 298, 300, or 335, wherein the insertion site is within intron 7 at the CIITA gene locus.

343. The population of cells of any one of claim 296, 298, 300, or 335, wherein the insertion site is within intron 8 at the CIITA gene locus.

344. The population of cells of any one of claim 296, 298, 300, or 335, wherein the insertion site is within intron 9 at the CIITA gene locus.

345. The population of cells of any one of claim 296, 298, 300, or 335, wherein the insertion site is within intron 10 at the CIITA gene locus.

346. The population of cells of any one of claim 296, 298, 300, or 335, wherein the insertion site is within intron 11 at the CIITA gene locus.

347. The population of cells of any one of claim 296, 298, 300, or 335, wherein the insertion site is within intron 12 at the CIITA gene locus.

348. The population of cells of any one of claim 296, 298, 300, or 335, wherein the insertion site is within intron 13 at the CIITA gene locus.

349. The population of cells of any one of claim 296, 298, 300, or 335, wherein the insertion site is within intron 14 at the CIITA gene locus.

350. The population of cells of any one of claim 296, 298, 300, or 335, wherein the insertion site is within intron 15 at the CIITA gene locus.

351. The population of cells of any one of claim 296, 298, 300, or 335, wherein the insertion site is within intron 16 at the CIITA gene locus.

352. The population of cells of any one of claim 296, 298, 300, or 335, wherein the insertion site is within intron 17 at the CIITA gene locus.

353. The population of cells of any one of claim 296, 298, 300, or 335, wherein the insertion site is within intron 18 at the CIITA gene locus.

354. The population of cells of any one of claim 296, 298, 300, or 335, wherein the insertion site is within intron 19 at the CIITA gene locus.

355. The population of cells of any one of claim 296, 298, or 302, wherein the insertion site is within the 5′ UTR at the CIITA gene locus.

356. The population of cells of any one of claim 296, 298, or 302, wherein the insertion site is within the 3′ UTR at the CIITA gene locus.

357. The population of cells of any one of claims 296-356, wherein at least 35% of the cells have increased cell surface expression of a first tolerogenic factor as compared to a comparable cell that has not been genetically engineered.

358. The population of cells according to any one of claims 296-357, wherein the tolerogenic factor is CD47.

359. The population of cells of any one of claim 296, 297, 299-313, 357, or 349, wherein at least 30% of the cells have decreased cell surface expression of B2M as compared to a comparable cell that has not been genetically engineered.

360. The population of cells of any one of claim 296, 297, 299-313, 357, 358, or 359, wherein at least 35% of the cells have decreased cell surface expression of B2M as compared to a comparable cell that has not been genetically engineered.

361. The population of cells of any one of claims 296-360, wherein the cells have been genetically engineered to knock-out an HLA-A locus, an HLA-B locus, an HLA-C locus, or a combination thereof.

362. The population of cells of any one of claims 296-361, wherein the cells have been genetically engineered to knock-out an HLA-DM locus, an HLA-DO locus, an HLA-DP locus, an HLA-DQ locus, an HLA-DR locus, or a combination thereof.

363. The population of cells of any one of claims 296-362, wherein the cells have been genetically engineered to knock-out a B2M locus.

364. The population of cells of any one of claims 296-363, wherein the cells have been genetically engineered to knock-out a CIITA locus.

365. The population of cells of any one of claims 296-364, wherein the cells have been genetically engineered to knock-out a TCR locus.

366. The population of cells of any one of claims 296-365, wherein at least 30% of the cells have decreased cell surface expression of an MHC I molecule, an MHC II molecule, or both, as compared to a comparable cell that has not been genetically engineered.

367. The population of cells of any one of claims 296-366, wherein at least 35% of the cells have decreased cell surface expression of an MHC I molecule, an MHC II molecule, or both, as compared to a comparable cell that has not been genetically engineered.

368. The population of cells of any one of claims 296-367, wherein the cells have been genetically engineered to comprise a transgene encoding a CAR.

369. The population of cells of claim 368, wherein at least 35% of the cells have cell surface expression of the CAR.

370. The population of cells of any one of claims 296-369, wherein the cells have been genetically engineered to comprise a transgene encoding a CAAR.

371. The population of cells of claim 370, wherein at least 35% of the cells have cell surface expression of the CAAR.

372. A composition comprising a population of cells according to any one of claims 296-371.

373. A pharmaceutical composition comprising (i) a population of cells according to any one of claims 296-371, and (ii) a pharmaceutically acceptable excipient.

374. A method comprising administering to a subject a population of cells according to any one of claims 296-371, a composition of claim 372, or a pharmaceutical composition of claim 373.

375. The method of claim 374, wherein the method is a method of treating a disease in a subject.

376. A population of cells of any one of claims 296-371 for use in treating a disease in a subject.

377. A composition of claim 372 for use in treating a disease in a subject.

378. A pharmaceutical composition of claim 373 for use in treating a disease in a subject.

379. Use of a population of cells of any one of claims 296-371, a composition of claim 363 or 368, or a pharmaceutical composition of claim 364 or 368 for use in treating a disease in a subject.

380. Use of a population of cells of any one of claims 296-371, a composition of claim 363 or 368, or a pharmaceutical composition of claim 364 or 368 in the manufacture of a medicament for the treatment of a disease.

381. The method, the population of cells, the composition, the pharmaceutical composition or the use of any of the preceding claims, wherein the disease is cancer.

382. The method, the population of cells, the composition, the pharmaceutical composition or the use of any of the preceding claims, wherein the cancer is associated with CD5, CD19, CD20, CD22, CD23, CD30, CD33, CD70, Kappa, Lambda, B cell maturation agent (BCMA), G-protein coupled receptor family C group 5 member D (GPRC5D), CD123, LeY, NKG2D ligand, WT1, GD2, HER2, EGFR, EGFRVIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acetylcholine e receptor, GD2, GD3, HMW-MAA, IL-11Rα, KDR, Lewis Y, L1-cell adhesion molecule, MAGE-A1, Oncofetal antigen (h5T4), and/or TAG-72 expression.

383. The method, the population of cells, the composition, the pharmaceutical composition or the use of any of the preceding claims, wherein the cancer is a hematologic malignancy.

384. The method, the population of cells, the composition, the pharmaceutical composition or the use of any of the preceding claims, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

385. The method, the population of cells, the composition, the pharmaceutical composition or the use of any of the preceding claims, wherein the cancer is solid malignancy.

386. The method, the population of cells, the composition, the pharmaceutical composition or the use of any of the preceding claims, wherein the solid malignancy is selected breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

387. The method of any of the preceding claims, the population of cells of any of the preceding claims, the composition of any of the preceding claims, the pharmaceutical composition of any of the preceding claims, or the use of any of the preceding claims, wherein the disease is an autoimmune disease.

388. The method, the population of cells, the composition, the pharmaceutical composition or the use of any of the preceding claims, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

389. The method of any of the preceding claims, the population of cells of any of the preceding claims, the composition of any of the preceding claims, the pharmaceutical composition of any of the preceding claims, or the use of any of the preceding claims, wherein the disease is diabetes mellitus.

390. The method, the population of cells, the composition, the pharmaceutical composition or the use of any of the preceding claims, wherein the diabetes is selected from the group consisting Type I diabetes, Type II diabetes, prediabetes, and gestational diabetes.

391. The method of any of the preceding claims, the population of cells of any of the preceding claims, the composition of any of the preceding claims, the pharmaceutical composition of any of the preceding claims, or the use of any of the preceding claims, wherein the disease is a neurological disease.

392. The method, the population of cells, the composition, the pharmaceutical composition or the use of any of the preceding claims, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

393. A method of identifying a site for inserting a first transgene at a β2 microglobulin (B2M) gene locus, comprising the steps of:
(a) identifying a protospacer adjacent motif (PAM) sequence or target adjacent motif (TAM) sequence in (i) the B2M gene locus, (ii) the 100 bp upstream of the 5′ end of the B2M gene locus, or (iii) the 100 bp downstream of the 3′ end of the B2M gene locus, and
(b) generating a gRNA comprising a complementary region, wherein the complementary region comprises a nucleic acid sequence that is complementary to a target nucleic acid sequence within the B2M gene locus, wherein the target nucleic acid sequence comprises the first insertion site, and wherein the first insertion site is 25 nucleotides or less from a PAM sequence or a TAM sequence.

394. A method of identifying a site for inserting a first transgene at a class II transactivator (CIITA) gene locus, comprising the steps of:
(a) identifying a protospacer adjacent motif (PAM) sequence or target adjacent motif (TAM) sequence in (i) the CIITA gene locus, (ii) the 100 bp upstream of the 5′ end of the CIITA gene locus, or (iii) the 100 bp downstream of the 3′ end of the CIITA gene locus, and
(b) generating a gRNA comprising a complementary region, wherein the complementary region comprises a nucleic acid sequence that is complementary to a target nucleic acid sequence within the CIITA gene locus, wherein the target nucleic acid sequence comprises the first insertion site, and wherein the first insertion site is 25 nucleotides or less from a PAM sequence or a TAM sequence.
